SEQUENCE LISTING (93 sequences)

SEQ ID NO: 1 - DNA, 2958 bp, synthetic construct
  source 1..2958: mol_type = other DNA
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagaactagtggatcccccgggctgcaggaattcgatatcaagcttatcgataccgtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 2 - DNA, 2961 bp, synthetic construct
  source 1..2961: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 3 - DNA, 18967 bp, synthetic construct
  source 1..18967: mol_type = other DNA
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 4 - protein, 21 aa, synthetic construct
GSGEGRGSLLTCGDVEENPGP

SEQ ID NO: 5 - protein, 22 aa, synthetic construct
GSGATNFSLLKQAGDVEENPGP

SEQ ID NO: 6 - protein, 23 aa, synthetic construct
GSGQCTNYALLKLAGDVESNPGP

SEQ ID NO: 7 - protein, 25 aa, synthetic construct
GSGVKQTLNFDLLKLAGDVESNPGP

SEQ ID NO: 8 - protein, 18 aa, Thosea asigna virus
EGRGSLLTCGDVEENPGP

SEQ ID NO: 9 - protein, 19 aa, Teschovirus A
ATNFSLLKQAGDVEENPGP

SEQ ID NO: 10 - protein, 20 aa, Equine rhinitis A virus
QCTNYALLKLAGDVESNPGP

SEQ ID NO: 11 - protein, 22 aa, Foot-and-mouth disease virus
VKQTLNFDLLKLAGDVESNPGP

SEQ ID NO: 12-13 - skipped (2 entries)

SEQ ID NO: 14 - protein, 4 aa, synthetic construct
RGRR

SEQ ID NO: 15 - protein, 4 aa, synthetic construct
RARR

SEQ ID NO: 16 - protein, 4 aa, synthetic construct
RLRR

SEQ ID NO: 17 - protein, 4 aa, synthetic construct
RMRR

SEQ ID NO: 18 - protein, 4 aa, synthetic construct
RFRR

SEQ ID NO: 19 - protein, 4 aa, synthetic construct
RWRR

SEQ ID NO: 20 - protein, 4 aa, synthetic construct
RKRR

SEQ ID NO: 21 - protein, 4 aa, synthetic construct
RQRR

SEQ ID NO: 22 - protein, 4 aa, synthetic construct
RERR

SEQ ID NO: 23 - protein, 4 aa, synthetic construct
RSRR

SEQ ID NO: 24 - protein, 4 aa, synthetic construct
RPRR

SEQ ID NO: 25 - protein, 4 aa, synthetic construct
RVRR

SEQ ID NO: 26 - protein, 4 aa, synthetic construct
RIRR

SEQ ID NO: 27 - protein, 4 aa, synthetic construct
RCRR

SEQ ID NO: 28 - protein, 4 aa, synthetic construct
RYRR

SEQ ID NO: 29 - protein, 4 aa, synthetic construct
RHRR

SEQ ID NO: 30 - protein, 4 aa, synthetic construct
RRRR

SEQ ID NO: 31 - protein, 4 aa, synthetic construct
RNRR

SEQ ID NO: 32 - protein, 4 aa, synthetic construct
RDRR

SEQ ID NO: 33 - protein, 4 aa, synthetic construct
RTRR

SEQ ID NO: 34 - protein, 4 aa, synthetic construct
RGKR

SEQ ID NO: 35 - protein, 4 aa, synthetic construct
RAKR

SEQ ID NO: 36 - protein, 4 aa, synthetic construct
RLKR

SEQ ID NO: 37 - protein, 4 aa, synthetic construct
RMKR

SEQ ID NO: 38 - protein, 4 aa, synthetic construct
RFKR

SEQ ID NO: 39 - protein, 4 aa, synthetic construct
RWKR

SEQ ID NO: 40 - protein, 4 aa, synthetic construct
RKKR

SEQ ID NO: 41 - protein, 4 aa, synthetic construct
RQKR

SEQ ID NO: 42 - protein, 4 aa, synthetic construct
REKR

SEQ ID NO: 43 - protein, 4 aa, synthetic construct
RSKR

SEQ ID NO: 44 - protein, 4 aa, synthetic construct
RPKR

SEQ ID NO: 45 - protein, 4 aa, synthetic construct
RVKR

SEQ ID NO: 46 - protein, 4 aa, synthetic construct
RIKR

SEQ ID NO: 47 - protein, 4 aa, synthetic construct
RCKR

SEQ ID NO: 48 - protein, 4 aa, synthetic construct
RYKR

SEQ ID NO: 49 - protein, 4 aa, synthetic construct
RHKR

SEQ ID NO: 50 - protein, 4 aa, synthetic construct
RRKR

SEQ ID NO: 51 - protein, 4 aa, synthetic construct
RNKR

SEQ ID NO: 52 - protein, 4 aa, synthetic construct
RDKR

SEQ ID NO: 53 - protein, 4 aa, synthetic construct
RTKR

SEQ ID NO: 54 - protein, 907 aa, Human gammaherpesvirus 4
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 55 - protein, 777 aa, Human gammaherpesvirus 4
MGRSRERARGRGRGRGEKRPRSPSSQSSSSGSPPRRPPPGRRPFFHPVGDADYFEYLQEGGPDGEPDVPPGAIEQGPTDDPGEGPSTGPRGQGDGGRRKKGGWFGKHRGQGGSNPKFENIAEGLRVLLARSHVERTTEEGNWVAGVFVYGGSKTSLYNLRRGIALAVPQCRITPLSRLPFGMAPGPGPQPGPLRESIVCYFMVFLQTHIFAEVLKDAIKDLVMTKPAPTCNIKVTVCSFDDGVDLPPWFPPMVEGAAAEGDDGDDGDEGGDGDEGEEGQEMGSLEMVPMGAGPPSPGGDPDGDDGGNNSQYPSASGSSGNTPTPPNDEERESNEEPPPPYEDPYWGNGDRHSDFQPLGTQDQSLYLGLQHDGNDGLPPPPYSPRDDSSQHIFEEAGRGSMNPVCLPVIVAPYLFWLAAIAASCFTASVSTVVTATGLALSLLLLAAVASSYAAAQRKLLTPVTVLTAVVTFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFVLWLSSPGGLGTLGAALLTLAAALALLASLILGTLNLTTMFLLMLLWTLVVLLICSSCSSCPLSKILLARLFLYALALLLLASALIAGGSILQTNFKSLSSTEFIPNLFCMLLLIVAGILFILAILTEWGSGNRTYGPVFMCLGGLLTMVAGAVWLTVMTNTLLSAWILTAGFLIFLIGFALFGVIRCCRYCCYYCLTLESEERPPTPYRNTV

SEQ ID NO: 56 - protein, 706 aa, Human gammaherpesvirus 4
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHVVLAIILYFIAFALGIFLVHKIVMFFL

SEQ ID NO: 57 - protein, 137 aa, Human gammaherpesvirus 4
MRAVGVFLAICLVTIFVLPTWGNWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGG

SEQ ID NO: 58 - protein, 223 aa, Human gammaherpesvirus 4
MVSFKQVRVPLFTAIALVIVLLLAYFLPPRVRGGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 59 - DNA, 18732 bp, synthetic construct
  source 1..18732: mol_type = other DNA
accaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacggccaccatggaggccgctttgctggtgtgtcagtacacaattcagagcctgatccacttgacgggagaggaccctggctttttcaacgttgagatccccgaattcccattttatccaacctgcaacgtgtgtactgcagacgtaaacgttactattaatttcgacgtgggaggcaagaaacaccaactggatctcgactttggacagctgactccccacaccaaagctgtttaccaacccagaggggcttttggcggctctgaaaacgctaccaacctcttcctgctcgagcttctgggagctggcgagcttgctctgactatgcgctctaaaaagctcccgattaacgtcacaacaggggaggaacaacaggtgagcttggagtccgtagacgtgtacttccaggacgtgttcggaacaatgtggtgtcatcacgctgagatgcagaatcccgtgtacctcattccagagacggtgccttatataaagtgggataactgtaactcaacgaatatcacagcagtggttagggcgcaaggtctggacgtcactcttcctctctcactcccaacttccgctcaagacagcaatttctcagtgaaaacagaaatgctgggcaacgagatagatattgagtgtataatggaagacggggagattagtcaagtgctgcctggcgacaacaaatttaatatcacctgtagcggttacgagagtcacgtcccgtccggggggattctgaccagcactagccctgtcgccactcccattccgggaactggatacgcctatagccttaggcttacacccagacctgtgtcacggtttctgggcaacaacagcattctgtacgtgttctacagtggcaacgggcctaaagcctctggtggcgactattgtattcaaagcaacatcgtcttctccgacgagatccctgcaagccaggacatgcccactaataccaccgacattacctacgtaggggacaacgctacttactctgtcccgatggtgactagcgaggatgctaattctccaaacgttaccgtaaccgccttttgggcttggcccaataatacagagacggatttcaaatgcaagtggactttgacttccggcaccccctccggctgtgaaaatatttctggcgctttcgccagcaacaggacgttcgacatcacagtctctggtcttggcactgccccaaagaccctgatcatcacccggactgccactaacgcgacaactacaactcataaggttatttttagcaaggcaccagagagcaccactacatctcccaccctgaacactacgggctttgctgacccgaataccaccacgggccttccctcatcaacccatgtcccaaccaacttgacagcaccagcatcaacgggtcccaccgtctccacagcagacgtaacctctcctacacctgccggcaccacaagtggcgctagtcctgtgaccccttccccatctccttgggataatggcacagagtcaaaagcccctgatatgactagctccaccagtcctgtgacgacaccaaccccaaacgctacctcccccactccagccgttacaactccaacgccaaacgctacctcccctactcctgccgtaaccacgcctacccctaatgccaccagtcctactttgggcaagacctccccgacaagcgctgtgacaacccctacgcccaacgcaacttctcctacactcggaaagacctcacctaccagtgctgtcaccacacctacaccaaacgccactagccccacattgggtaagacatcccccacttccgcagttacgactcccacaccgaacgcaaccggacctactgtcggtgagacatcccctcaggccaacgccacaaatcacacactgggcggcacctcaccaactccagtagtgacctcccagccaaaaaacgccacctctgctgtgaccaccggtcagcataacatcacaagttcctccacttctagcatgagtttgcgcccctcatccaacccagaaacgctgagcccttcaacatctgacaactcaacaagccatatgcctctgctcactagtgctcacccaacagggggagaaaacattacacaggtgacacccgccagcatctctacacaccacgtgagcacaagctctcccgctcctcggccaggtacaacctcccaagcctctgggcctggaaattcctcaacctccaccaaacccggtgaggtcaacgtcaccaagggaacacccccccagaacgcaacgtcacctcaagcacccagcggacagaagaccgctgtgcctactgtaacctctaccggagggaaagctaatagtacgactggaggtaagcacactacgggccacggtgccagaaccagtacagagcctaccactgattacggcggagatagtacaaccccccgccctagatacaacgccacaacttatttgcctccctcaacgtcctccaagctgagacccaggtggacgtttacgtctcccccagtcacaactgcgcaggcaaccgtgccagttccacctacaagtcagccgcgcttcagcaatctctcaatgctcgtgctgcagtgggcctctctggcagtactgaccctcttgcttctcttggtgatggctgactgtgcctttcggagaaacctgagcacctcacacacttataccacacctccctatgacgatgccgagacatatgtataagcgcgcagcgcttagacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 60 - DNA, 21210 bp, synthetic construct
  source 1..21210: mol_type = other DNA
accaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacggccaccatggaggccgctttgctggtgtgtcagtacacaattcagagcctgatccacttgacgggagaggaccctggctttttcaacgttgagatccccgaattcccattttatccaacctgcaacgtgtgtactgcagacgtaaacgttactattaatttcgacgtgggaggcaagaaacaccaactggatctcgactttggacagctgactccccacaccaaagctgtttaccaacccagaggggcttttggcggctctgaaaacgctaccaacctcttcctgctcgagcttctgggagctggcgagcttgctctgactatgcgctctaaaaagctcccgattaacgtcacaacaggggaggaacaacaggtgagcttggagtccgtagacgtgtacttccaggacgtgttcggaacaatgtggtgtcatcacgctgagatgcagaatcccgtgtacctcattccagagacggtgccttatataaagtgggataactgtaactcaacgaatatcacagcagtggttagggcgcaaggtctggacgtcactcttcctctctcactcccaacttccgctcaagacagcaatttctcagtgaaaacagaaatgctgggcaacgagatagatattgagtgtataatggaagacggggagattagtcaagtgctgcctggcgacaacaaatttaatatcacctgtagcggttacgagagtcacgtcccgtccggggggattctgaccagcactagccctgtcgccactcccattccgggaactggatacgcctatagccttaggcttacacccagacctgtgtcacggtttctgggcaacaacagcattctgtacgtgttctacagtggcaacgggcctaaagcctctggtggcgactattgtattcaaagcaacatcgtcttctccgacgagatccctgcaagccaggacatgcccactaataccaccgacattacctacgtaggggacaacgctacttactctgtcccgatggtgactagcgaggatgctaattctccaaacgttaccgtaaccgccttttgggcttggcccaataatacagagacggatttcaaatgcaagtggactttgacttccggcaccccctccggctgtgaaaatatttctggcgctttcgccagcaacaggacgttcgacatcacagtctctggtcttggcactgccccaaagaccctgatcatcacccggactgccactaacgcgacaactacaactcataaggttatttttagcaaggcaccagagagcaccactacatctcccaccctgaacactacgggctttgctgacccgaataccaccacgggccttccctcatcaacccatgtcccaaccaacttgacagcaccagcatcaacgggtcccaccgtctccacagcagacgtaacctctcctacacctgccggcaccacaagtggcgctagtcctgtgaccccttccccatctccttgggataatggcacagagtcaaaagcccctgatatgactagctccaccagtcctgtgacgacaccaaccccaaacgctacctcccccactccagccgttacaactccaacgccaaacgctacctcccctactcctgccgtaaccacgcctacccctaatgccaccagtcctactttgggcaagacctccccgacaagcgctgtgacaacccctacgcccaacgcaacttctcctacactcggaaagacctcacctaccagtgctgtcaccacacctacaccaaacgccactagccccacattgggtaagacatcccccacttccgcagttacgactcccacaccgaacgcaaccggacctactgtcggtgagacatcccctcaggccaacgccacaaatcacacactgggcggcacctcaccaactccagtagtgacctcccagccaaaaaacgccacctctgctgtgaccaccggtcagcataacatcacaagttcctccacttctagcatgagtttgcgcccctcatccaacccagaaacgctgagcccttcaacatctgacaactcaacaagccatatgcctctgctcactagtgctcacccaacagggggagaaaacattacacaggtgacacccgccagcatctctacacaccacgtgagcacaagctctcccgctcctcggccaggtacaacctcccaagcctctgggcctggaaattcctcaacctccaccaaacccggtgaggtcaacgtcaccaagggaacacccccccagaacgcaacgtcacctcaagcacccagcggacagaagaccgctgtgcctactgtaacctctaccggagggaaagctaatagtacgactggaggtaagcacactacgggccacggtgccagaaccagtacagagcctaccactgattacggcggagatagtacaaccccccgccctagatacaacgccacaacttatttgcctccctcaacgtcctccaagctgagacccaggtggacgtttacgtctcccccagtcacaactgcgcaggcaaccgtgccagttccacctacaagtcagccgcgcttcagcaatctctcaatgctcgtgctgcagtgggcctctctggcagtactgaccctcttgcttctcttggtgatggctgactgtgcctttcggagaaacctgagcacctcacacacttataccacacctccctacgacgacgccgagacatacgtataattcgaactacagctcaacttacctgccaaccccatgccagtcgacccaactagtacaacctaaatccattataaaaaacttaggaaccaggtccacacagccgccagcccatcaaccatccactcccacgattggaggccggccatgggtagatcaagagaaagagccagaggacggggtagaggcagaggagagaagagacctcgcagccctagcagccagagttctagctcaggttctcctccaagaagacctcctccagggaggagacccttctttcatccggtcggagacgccgattactttgagtacttgcaggaaggcggtccagacggagaaccagacgtcccaccgggagctatcgagcaaggacctaccgacgacccaggagaaggaccaagcacgggtccaagaggtcagggagacggaggacggagaaagaagggcggctggtttggaaagcatcgcgggcaaggcggatcaaaccctaaattcgagaacattgccgagggactgagagtgctcctggccagatcacacgtggagaggactactgaggaaggtaactgggtagccggggtattcgtttacggcgggagcaaaacatcactctacaatctcagacgaggaattgctctcgcggtaccacagtgcaggattacgcctctgtctcggttgccatttggcatggcacctggtcctggacctcaacctggtcctctcagggagtccatagtgtgttatttcatggtgtttctccagacgcatatcttcgcggaagtattgaaggacgctataaaggacctggtgatgactaagccagcccccacatgtaacataaaggtgacagtgtgcagttttgacgacggcgttgatctgcctccttggttccctccaatggtagaaggagcggcggcagaaggagacgacggcgacgacggggacgagggtggagacggtgacgaaggtgaagaagggcaggaaatgggctccctggaaatggtgcctatgggtgctgggccaccaagtccgggaggtgatccagacggagacgacgggggtaacaatagtcagtacccctccgcctcaggcagtagtggtaacaccccgacccccccaaacgacgaagaaagggagtccaacgaagagccgcctcctccatacgaggacccgtattggggcaacggagatcgccattctgacttccagcctctgggaacgcaggaccaatctctgtaccttggactgcagcacgatggtaacgacggccttccacctccaccatattcacctagggacgatagtagccagcatattttcgaagaagcgggcagagggtccatgaatccagtttgtctgccggtgattgtggctccgtatctcttctggctcgcggcaatagctgcctcctgtttcaccgcttctgtgtcaaccgttgtgacagcaactggactcgcgctgagccttctgctcctcgctgcagtcgcctcaagttacgcagcggcccaacgcaaacttctcacgccagtcaccgtgctgactgcagtggtcacattctttgccatatgtttgacttggaggattgaggacccaccctttaattcactgctgtttgccctccttgcagcggccggaggacttcaaggaatttacgtcctggtgatgctggttctcctgatcctcgcgtacagacgaaggtggcggaggcttacagtatgtggagggattatgtttctggcctgtgtgctggtcttgatcgttgacgccgttctccaattgagtcccctgttgggtgctgtcaccgtcgtatctatgacactgctgcttttggctttcgtcctctggttgtccagccctggaggattgggtaccctcggagcagctttgctgaccttggcagccgcactggctctcctggctagtctcatcctgggcacgctcaacctcactacaatgttcctgctgatgctgctgtggacccttgtggtgcttctcatttgcagctcttgtagctcttgccccctgtcaaagatccttctcgccagattgttcctgtacgcactcgctctcttgttgctggccagcgctctcatcgcaggtggaagtatactccagacgaatttcaaaagtctgagtagcacagagttcatcccaaacctgttctgtatgttgttgctgatcgtagctggaatcctcttcatcttggcaatcttgacagagtggggatccgggaaccggacatacggacctgtattcatgtgtctgggaggccttcttacaatggttgcgggagctgtttggcttaccgtgatgactaacaccctgctgagtgcctggattctgactgctggatttttgatttttttgatcggctttgctctcttcggcgtcatcaggtgctgtaggtattgctgttattattgcctgacattggaaagcgaggaaagaccacccacaccttatagaaatacggtgtgagcgcgcagcgcttagacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 61 - DNA, 19464 bp, synthetic construct
  source 1..19464: mol_type = other DNA
accaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacggccaccatgcaactcctctgcgtcttttgcttggtgcttttgtgggaggtaggagcggcctcattgtcagaagttaagctgcatctcgatattgaaggccacgcttcacattacactatcccttggaccgaactcatggctaaggtgccgggtttgtctccggaagcactttggagagaggcaaacgttaccgaagacctggcctcaatgctgaatcgctacaagctgatatacaagacttccggaaccctgggtatagcactcgcagagcccgtcgatatccccgctgtcagcgaaggtagcatgcaggtggacgctagtaaagttcacccaggagttatctcaggtctgaatagcccagcttgtatgctgagcgctccactggagaaacagctcttctactatataggcacaatgctccctaacactcgacctcacagctacgtcttctatcagctgcgctgtcacctgtcatacgtagccttgtccattaatggtgataagttccagtacacaggtgccatgacctccaagttccttatggggacctataaacgggtcactgagaaaggagacgagcatgtccttagtttggtttttgggaagacaaaagacctgccggatctgaggggcccgttctcttacccctcactcacatctgcacagagtggggactattcattggtgattgtgacaaccttcgttcactacgccaattttcacaactattttgtcccgaaccttaaagacatgttttcacgcgccgtgaccatgacagctgcttcctacgctaggtatgtgctccaaaagctggtgctgctggagatgaagggcggatgcagggaacctgaactggacacagagactctcaccactatgttcgaggtttccgtggcgttcttcaaggtgggtcatgctgtgggggagactgggaacggctgcgtagatcttaggtggctggctaaatctttcttcgagctgaccgtgttgaaagatataattggaatttgctacggggctacggttaaaggaatgcagtcttacgggctggaaaggctggccgctatgctcatggctactgtgaagatggaggaactcggccacctcaccacagaaaaacaggagtacgctttgcgcctggccactgttggctaccctaaggcaggcgtttactctggattgattgggggggccacatctgtgctcttgtcagcctacaatcgccatcccctgttccagcctctgcacacggtgatgcgagagactttgtttattggcagccacgttgtgctgagagaactgcgccttaacgttaccactcagggacctaaccttgcgctgtatcagctgcttagcaccgctttgtgctccgctcttgaaattggagaagtactcagagggctcgcactgggtactgagtcagggctgttctccccctgctatctgtctctccggttcgacctcactcgggataagctgttgtcaatggctccgcaggaagccacattggaccaggccgcagtgagtaacgcagtggacgggttcctgggtagactgtctcttgagagggaagacagagacgcttggcatctgcctgcgtataagtgtgttgatagactggacaaagtactgatgatcattcccctgattaatgtgacatttattatatctagtgaccgagaggtgagaggctccgctctctatgaagcatccacaacgtacttgagcagttccctgtttctgtcaccggttattatgaacaaatgttctcagggagctgttgctggtgaaccgcgacagattccaaagattcagaacttcactaggacacaaaagagctgcattttctgcgggttcgcactgctgagttacgacgaaaaggagggactggagactactacatatattacttcacaggaggtgcagaatagcatcctgagctccaattattttgactttgacaacctccatgtccattatttgctgttgaccacaaacggcactgtgatggagatcgctggcctgtacgaggagcgagcccacgtagtactggccattattctgtacttcatcgctttcgcacttgggattttcctcgtccacaaaattgttatgtttttcctctaatgattcgaactacagctcaacttacctgccaaccccatgccagtcgacccaactagtacaacctaaatccattataaaaaacttaggaaccaggtccacacagccgccagcccatcaaccatccactcccacgattggaggccggccatggtgagttttaagcaggtccgggtacctctgttcaccgccattgcgctggtgattgttctgctgttggcctactttcttccacctagagtcagaggaggtggcagggttgctgcagctgctattacctgggtaccgaaacccaacgtggaggtttggccagtggatccgcccccaccagtgaactttaataaaacagccgaacaagaatacggggataaagaagtaaagctgccacactggacccctaccctccacactttccaggtgcctcaaaactatacaaaggccaattgcacatactgtaacacaagagaatacactttttcttataaagggtgctgcttctacttcactaagaagaagcatacttggaacggatgtttccaggcatgcgctgagttgtatccctgtacctatttctacggtcctacaccagacattctgccggtcgtgactcgcaatctgaacgccatagaatctctgtgggtcggggtgtatcgggttggggaaggaaattggactagcctggacggcggtactttcaaggtgtaccaaatattcggcagtcactgtacttacgtgtcaaaattctcaacagttcccgtatcccaccacgagtgttcttttttgaagccttgcctgtgcgtgtcacagaggtcaaattcctaactattcgaactactcagctagtctacgcgccgtagcctaccctccatcattgttataaaaaacttaggagcaaagtccacacagccgccgataaggtccacaatgagagccgtaggtgtctttctggctatttgtctggtaacaatcttcgtgctgcccacttggggcaactgggcctatccttgctgccatgttacacagttgagagcgcagcacctcctggcgttggagaacatttcagacatctacctggtgagtaaccaaacctgcgacgggttctctttggccagtctcaatagtccaaaaaacggaagcaatcagctggtaatttcacggtgtgcaaacggacttaacgtggtctctttttttatatcaatcctgaaacgcagttcatctgcattgaccggccacttgagggagctcttgaccactctcgagacactttacgggtcattctccgtggaggatctgttcggggcaaatctgaaccggtacgcctggcatagaggcgggtaagcgcgcagcgcttagacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 62 - DNA, 25 bp, synthetic construct
  source 1..25: mol_type = other DNA
cgaagactgccatgccccaacatac

SEQ ID NO: 63 - DNA, 25 bp, synthetic construct
  source 1..25: mol_type = other DNA
tagcgagtccataacggggaaccac

SEQ ID NO: 64 - DNA, 22 bp, synthetic construct
  source 1..22: mol_type = other DNA
ttctttctccgtcctccgtctc

SEQ ID NO: 65 - DNA, 23 bp, synthetic construct
  source 1..23: mol_type = other DNA
cttctagtgcacttaggattcaa

SEQ ID NO: 66 - DNA, 11 bp, synthetic construct
  source 1..11: mol_type = other DNA
cttctagtgca

SEQ ID NO: 67-68 - skipped (2 entries)

SEQ ID NO: 69 - DNA, 31 bp, synthetic construct
  source 1..31: mol_type = other DNA
gttataaaaaacttaggaaccaggtccacac

SEQ ID NO: 70 - DNA, 11 bp, synthetic construct
  source 1..11: mol_type = other DNA
gttataaaaaa

SEQ ID NO: 71 - DNA, 17 bp, synthetic construct
  source 1..17: mol_type = other DNA
aggaaccaggtccacac

SEQ ID NO: 72 - DNA, 31 bp, synthetic construct
  source 1..31: mol_type = other DNA
attataaaaaacttaggagcaaagtgattgc

SEQ ID NO: 73 - DNA, 11 bp, synthetic construct
  source 1..11: mol_type = other DNA
attataaaaaa

SEQ ID NO: 74 - DNA, 17 bp, synthetic construct
  source 1..17: mol_type = other DNA
aggagcaaagtgattgc

SEQ ID NO: 75 - DNA, 31 bp, synthetic construct
  source 1..31: mol_type = other DNA
attataaaaaacttaggaaccaggtccacac

SEQ ID NO: 76 - DNA, 17 bp, synthetic construct
  source 1..17: mol_type = other DNA
aggagcaaagtccacac

SEQ ID NO: 77 - DNA, 17 bp, synthetic construct
  source 1..17: mol_type = other DNA
aggatccaagagcatac

SEQ ID NO: 78 - DNA, 31 bp, synthetic construct
  source 1..31: mol_type = other DNA
gttataaaaaacttaggagcaaagtccacac

SEQ ID NO: 79 - DNA, 31 bp, synthetic construct
  source 1..31: mol_type = other DNA
gttataaaaaacttaggatccaagagcatac

SEQ ID NO: 80-81 - skipped (2 entries)

SEQ ID NO: 82 - DNA, 18732 bp, synthetic construct
  source 1..18732: mol_type = other DNA
accaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacggccaccatggaggccgctttgctggtgtgtcagtacacaattcagagcctgatccacttgacgggagaggaccctggctttttcaacgttgagatccccgaattcccattttatccaacctgcaacgtgtgtactgcagacgtaaacgttactattaatttcgacgtgggaggcaagaaacaccaactggatctcgactttggacagctgactccccacaccaaagctgtttaccaacccagaggggcttttggcggctctgaaaacgctaccaacctcttcctgctcgagcttctgggagctggcgagcttgctctgactatgcgctctaaaaagctcccgattaacgtcacaacaggggaggaacaacaggtgagcttggagtccgtagacgtgtacttccaggacgtgttcggaacaatgtggtgtcatcacgctgagatgcagaatcccgtgtacctcattccagagacggtgccttatataaagtgggataactgtaactcaacgaatatcacagcagtggttagggcgcaaggtctggacgtcactcttcctctctcactcccaacttccgctcaagacagcaatttctcagtgaaaacagaaatgctgggcaacgagatagatattgagtgtataatggaagacggggagattagtcaagtgctgcctggcgacaacaaatttaatatcacctgtagcggttacgagagtcacgtcccgtccggggggattctgaccagcactagccctgtcgccactcccattccgggaactggatacgcctatagccttaggcttacacccagacctgtgtcacggtttctgggcaacaacagcattctgtacgtgttctacagtggcaacgggcctaaagcctctggtggcgactattgtattcaaagcaacatcgtcttctccgacgagatccctgcaagccaggacatgcccactaataccaccgacattacctacgtaggggacaacgctacttactctgtcccgatggtgactagcgaggatgctaattctccaaacgttaccgtaaccgccttttgggcttggcccaataatacagagacggatttcaaatgcaagtggactttgacttccggcaccccctccggctgtgaaaatatttctggcgctttcgccagcaacaggacgttcgacatcacagtctctggtcttggcactgccccaaagaccctgatcatcacccggactgccactaacgcgacaactacaactcataaggttatttttagcaaggcaccagagagcaccactacatctcccaccctgaacactacgggctttgctgacccgaataccaccacgggccttccctcatcaacccatgtcccaaccaacttgacagcaccagcatcaacgggtcccaccgtctccacagcagacgtaacctctcctacacctgccggcaccacaagtggcgctagtcctgtgaccccttccccatctccttgggataatggcacagagtcaaaagcccctgatatgactagctccaccagtcctgtgacgacaccaaccccaaacgctacctcccccactccagccgttacaactccaacgccaaacgctacctcccctactcctgccgtaaccacgcctacccctaatgccaccagtcctactttgggcaagacctccccgacaagcgctgtgacaacccctacgcccaacgcaacttctcctacactcggaaagacctcacctaccagtgctgtcaccacacctacaccaaacgccactagccccacattgggtaagacatcccccacttccgcagttacgactcccacaccgaacgcaaccggacctactgtcggtgagacatcccctcaggccaacgccacaaatcacacactgggcggcacctcaccaactccagtagtgacctcccagccaaaaaacgccacctctgctgtgaccaccggtcagcataacatcacaagttcctccacttctagcatgagtttgcgcccctcatccaacccagaaacgctgagcccttcaacatctgacaactcaacaagccatatgcctctgctcactagtgctcacccaacagggggagaaaacattacacaggtgacacccgccagcatctctacacaccacgtgagcacaagctctcccgctcctcggccaggtacaacctcccaagcctctgggcctggaaattcctcaacctccaccaaacccggtgaggtcaacgtcaccaagggaacacccccccagaacgcaacgtcacctcaagcacccagcggacagaagaccgctgtgcctactgtaacctctaccggagggaaagctaatagtacgactggaggtaagcacactacgggccacggtgccagaaccagtacagagcctaccactgattacggcggagatagtacaaccccccgccctagatacaacgccacaacttatttgcctccctcaacgtcctccaagctgagacccaggtggacgtttacgtctcccccagtcacaactgcgcaggcaaccgtgccagttccacctacaagtcagccgcgcttcagcaatctctcaatgctcgtgctgcagtgggcctctctggcagtactgaccctcttgcttctcttggtgatggctgactgtgcctttcggagaaacctgagcacctcacacacttataccacacctccctatgacgatgccgagacatatgtataagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 83 - DNA, 19464 bp, synthetic construct
  source 1..19464: mol_type = other DNA
accaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacggccaccatgcaactcctctgcgtcttttgcttggtgcttttgtgggaggtaggagcggcctcattgtcagaagttaagctgcatctcgatattgaaggccacgcttcacattacactatcccttggaccgaactcatggctaaggtgccgggtttgtctccggaagcactttggagagaggcaaacgttaccgaagacctggcctcaatgctgaatcgctacaagctgatatacaagacttccggaaccctgggtatagcactcgcagagcccgtcgatatccccgctgtcagcgaaggtagcatgcaggtggacgctagtaaagttcacccaggagttatctcaggtctgaatagcccagcttgtatgctgagcgctccactggagaaacagctcttctactatataggcacaatgctccctaacactcgacctcacagctacgtcttctatcagctgcgctgtcacctgtcatacgtagccttgtccattaatggtgataagttccagtacacaggtgccatgacctccaagttccttatggggacctataaacgggtcactgagaaaggagacgagcatgtccttagtttggtttttgggaagacaaaagacctgccggatctgaggggcccgttctcttacccctcactcacatctgcacagagtggggactattcattggtgattgtgacaaccttcgttcactacgccaattttcacaactattttgtcccgaaccttaaagacatgttttcacgcgccgtgaccatgacagctgcttcctacgctaggtatgtgctccaaaagctggtgctgctggagatgaagggcggatgcagggaacctgaactggacacagagactctcaccactatgttcgaggtttccgtggcgttcttcaaggtgggtcatgctgtgggggagactgggaacggctgcgtagatcttaggtggctggctaaatctttcttcgagctgaccgtgttgaaagatataattggaatttgctacggggctacggttaaaggaatgcagtcttacgggctggaaaggctggccgctatgctcatggctactgtgaagatggaggaactcggccacctcaccacagaaaaacaggagtacgctttgcgcctggccactgttggctaccctaaggcaggcgtttactctggattgattgggggggccacatctgtgctcttgtcagcctacaatcgccatcccctgttccagcctctgcacacggtgatgcgagagactttgtttattggcagccacgttgtgctgagagaactgcgccttaacgttaccactcagggacctaaccttgcgctgtatcagctgcttagcaccgctttgtgctccgctcttgaaattggagaagtactcagagggctcgcactgggtactgagtcagggctgttctccccctgctatctgtctctccggttcgacctcactcgggataagctgttgtcaatggctccgcaggaagccacattggaccaggccgcagtgagtaacgcagtggacgggttcctgggtagactgtctcttgagagggaagacagagacgcttggcatctgcctgcgtataagtgtgttgatagactggacaaagtactgatgatcattcccctgattaatgtgacatttattatatctagtgaccgagaggtgagaggctccgctctctatgaagcatccacaacgtacttgagcagttccctgtttctgtcaccggttattatgaacaaatgttctcagggagctgttgctggtgaaccgcgacagattccaaagattcagaacttcactaggacacaaaagagctgcattttctgcgggttcgcactgctgagttacgacgaaaaggagggactggagactactacatatattacttcacaggaggtgcagaatagcatcctgagctccaattattttgactttgacaacctccatgtccattatttgctgttgaccacaaacggcactgtgatggagatcgctggcctgtacgaggagcgagcccacgtagtactggccattattctgtacttcatcgctttcgcacttgggattttcctcgtccacaaaattgttatgtttttcctctaatgattcgaactacagctcaacttacctgccaaccccatgccagtcgacccaactagtacaacctaaatccattataaaaaacttaggaaccaggtccacacagccgccagcccatcaaccatccactcccacgattggaggccggccatggtgagttttaagcaggtccgggtacctctgttcaccgccattgcgctggtgattgttctgctgttggcctactttcttccacctagagtcagaggaggtggcagggttgctgcagctgctattacctgggtaccgaaacccaacgtggaggtttggccagtggatccgcccccaccagtgaactttaataaaacagccgaacaagaatacggggataaagaagtaaagctgccacactggacccctaccctccacactttccaggtgcctcaaaactatacaaaggccaattgcacatactgtaacacaagagaatacactttttcttataaagggtgctgcttctacttcactaagaagaagcatacttggaacggatgtttccaggcatgcgctgagttgtatccctgtacctatttctacggtcctacaccagacattctgccggtcgtgactcgcaatctgaacgccatagaatctctgtgggtcggggtgtatcgggttggggaaggaaattggactagcctggacggcggtactttcaaggtgtaccaaatattcggcagtcactgtacttacgtgtcaaaattctcaacagttcccgtatcccaccacgagtgttcttttttgaagccttgcctgtgcgtgtcacagaggtcaaattcctaactattcgaactactcagctagtctacgcgccgtagcctaccctccatcattgttataaaaaacttaggagcaaagtccacacagccgccgataaggtccacaatgagagccgtaggtgtctttctggctatttgtctggtaacaatcttcgtgctgcccacttggggcaactgggcctatccttgctgccatgttacacagttgagagcgcagcacctcctggcgttggagaacatttcagacatctacctggtgagtaaccaaacctgcgacgggttctctttggccagtctcaatagtccaaaaaacggaagcaatcagctggtaatttcacggtgtgcaaacggacttaacgtggtctctttttttatatcaatcctgaaacgcagttcatctgcattgaccggccacttgagggagctcttgaccactctcgagacactttacgggtcattctccgtggaggatctgttcggggcaaatctgaaccggtacgcctggcatagaggcgggtaagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 84 - DNA, 19464 bp, synthetic construct
  source 1..19464: mol_type = other DNA
accaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggcatcaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagatcaagttccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacggccaccatgcaactcctctgcgtcttttgcttggtgcttttgtgggaggtaggagcggcctcattgtcagaagttaagctgcatctcgatattgaaggccacgcttcacattacactatcccttggaccgaactcatggctaaggtgccgggtttgtctccggaagcactttggagagaggcaaacgttaccgaagacctggcctcaatgctgaatcgctacaagctgatatacaagacttccggaaccctgggtatagcactcgcagagcccgtcgatatccccgctgtcagcgaaggtagcatgcaggtggacgctagtaaagttcacccaggagttatctcaggtctgaatagcccagcttgtatgctgagcgctccactggagaaacagctcttctactatataggcacaatgctccctaacactcgacctcacagctacgtcttctatcagctgcgctgtcacctgtcatacgtagccttgtccattaatggtgataagttccagtacacaggtgccatgacctccaagttccttatggggacctataaacgggtcactgagaaaggagacgagcatgtccttagtttggtttttgggaagacaaaagacctgccggatctgaggggcccgttctcttacccctcactcacatctgcacagagtggggactattcattggtgattgtgacaaccttcgttcactacgccaattttcacaactattttgtcccgaaccttaaagacatgttttcacgcgccgtgaccatgacagctgcttcctacgctaggtatgtgctccaaaagctggtgctgctggagatgaagggcggatgcagggaacctgaactggacacagagactctcaccactatgttcgaggtttccgtggcgttcttcaaggtgggtcatgctgtgggggagactgggaacggctgcgtagatcttaggtggctggctaaatctttcttcgagctgaccgtgttgaaagatataattggaatttgctacggggctacggttaaaggaatgcagtcttacgggctggaaaggctggccgctatgctcatggctactgtgaagatggaggaactcggccacctcaccacagaaaaacaggagtacgctttgcgcctggccactgttggctaccctaaggcaggcgtttactctggattgattgggggggccacatctgtgctcttgtcagcctacaatcgccatcccctgttccagcctctgcacacggtgatgcgagagactttgtttattggcagccacgttgtgctgagagaactgcgccttaacgttaccactcagggacctaaccttgcgctgtatcagctgcttagcaccgctttgtgctccgctcttgaaattggagaagtactcagagggctcgcactgggtactgagtcagggctgttctccccctgctatctgtctctccggttcgacctcactcgggataagctgttgtcaatggctccgcaggaagccacattggaccaggccgcagtgagtaacgcagtggacgggttcctgggtagactgtctcttgagagggaagacagagacgcttggcatctgcctgcgtataagtgtgttgatagactggacaaagtactgatgatcattcccctgattaatgtgacatttattatatctagtgaccgagaggtgagaggctccgctctctatgaagcatccacaacgtacttgagcagttccctgtttctgtcaccggttattatgaacaaatgttctcagggagctgttgctggtgaaccgcgacagattccaaagattcagaacttcactaggacacaaaagagctgcattttctgcgggttcgcactgctgagttacgacgaaaaggagggactggagactactacatatattacttcacaggaggtgcagaatagcatcctgagctccaattattttgactttgacaacctccatgtccattatttgctgttgaccacaaacggcactgtgatggagatcgctggcctgtacgaggagcgagcccacgtagtactggccattattctgtacttcatcgctttcgcacttgggattttcctcgtccacaaaattgttatgtttttcctctaatgattcgaactacagctcaacttacctgccaaccccatgccagtcgacccaactagtacaacctaaatccattataaaaaacttaggaaccaggtccacacagccgccagcccatcaaccatccactcccacgattggaggccggccatgagagccgtaggtgtctttctggctatttgtctggtaacaatcttcgtgctgcccacttggggcaactgggcctatccttgctgccatgttacacagttgagagcgcagcacctcctggcgttggagaacatttcagacatctacctggtgagtaaccaaacctgcgacgggttctctttggccagtctcaatagtccaaaaaacggaagcaatcagctggtaatttcacggtgtgcaaacggacttaacgtggtctctttttttatatcaatcctgaaacgcagttcatctgcattgaccggccacttgagggagctcttgaccactctcgagacactttacgggtcattctccgtggaggatctgttcggggcaaatctgaaccggtacgcctggcatagaggcgggtaactattcgaactactcagctagtctacgcgccgtagcctaccctccatcattgttataaaaaacttaggatccaagagcatacagccgccgataaggtccaccatggtgagttttaagcaggtccgggtacctctgttcaccgccattgcgctggtgattgttctgctgttggcctactttcttccacctagagtcagaggaggtggcagggttgctgcagctgctattacctgggtaccgaaacccaacgtggaggtttggccagtggatccgcccccaccagtgaactttaataaaacagccgaacaagaatacggggataaagaagtaaagctgccacactggacccctaccctccacactttccaggtgcctcaaaactatacaaaggccaattgcacatactgtaacacaagagaatacactttttcttataaagggtgctgcttctacttcactaagaagaagcatacttggaacggatgtttccaggcatgcgctgagttgtatccctgtacctatttctacggtcctacaccagacattctgccggtcgtgactcgcaatctgaacgccatagaatctctgtgggtcggggtgtatcgggttggggaaggaaattggactagcctggacggcggtactttcaaggtgtaccaaatattcggcagtcactgtacttacgtgtcaaaattctcaacagttcccgtatcccaccacgagtgttcttttttgaagccttgcctgtgcgtgtcacagaggtcaaattcctaagcgcgcagcgcttagacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 85 - DNA, 19464 bp, synthetic construct
  source 1..19464: mol_type = other DNA
accaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggcatcaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagatcaagttccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacggccaccatggtgagttttaagcaggtccgggtacctctgttcaccgccattgcgctggtgattgttctgctgttggcctactttcttccacctagagtcagaggaggtggcagggttgctgcagctgctattacctgggtaccgaaacccaacgtggaggtttggccagtggatccgcccccaccagtgaactttaataaaacagccgaacaagaatacggggataaagaagtaaagctgccacactggacccctaccctccacactttccaggtgcctcaaaactatacaaaggccaattgcacatactgtaacacaagagaatacactttttcttataaagggtgctgcttctacttcactaagaagaagcatacttggaacggatgtttccaggcatgcgctgagttgtatccctgtacctatttctacggtcctacaccagacattctgccggtcgtgactcgcaatctgaacgccatagaatctctgtgggtcggggtgtatcgggttggggaaggaaattggactagcctggacggcggtactttcaaggtgtaccaaatattcggcagtcactgtacttacgtgtcaaaattctcaacagttcccgtatcccaccacgagtgttcttttttgaagccttgcctgtgcgtgtcacagaggtcaaattcctaattcgaactacagctcaacttacctgccaaccccatgccagtcgacccaactagtacaacctaaatccattataaaaaacttaggaaccaggtccacacagccgccagcccatcaaccatccactcccacgattggaggccggccatgcaactcctctgcgtcttttgcttggtgcttttgtgggaggtaggagcggcctcattgtcagaagttaagctgcatctcgatattgaaggccacgcttcacattacactatcccttggaccgaactcatggctaaggtgccgggtttgtctccggaagcactttggagagaggcaaacgttaccgaagacctggcctcaatgctgaatcgctacaagctgatatacaagacttccggaaccctgggtatagcactcgcagagcccgtcgatatccccgctgtcagcgaaggtagcatgcaggtggacgctagtaaagttcacccaggagttatctcaggtctgaatagcccagcttgtatgctgagcgctccactggagaaacagctcttctactatataggcacaatgctccctaacactcgacctcacagctacgtcttctatcagctgcgctgtcacctgtcatacgtagccttgtccattaatggtgataagttccagtacacaggtgccatgacctccaagttccttatggggacctataaacgggtcactgagaaaggagacgagcatgtccttagtttggtttttgggaagacaaaagacctgccggatctgaggggcccgttctcttacccctcactcacatctgcacagagtggggactattcattggtgattgtgacaaccttcgttcactacgccaattttcacaactattttgtcccgaaccttaaagacatgttttcacgcgccgtgaccatgacagctgcttcctacgctaggtatgtgctccaaaagctggtgctgctggagatgaagggcggatgcagggaacctgaactggacacagagactctcaccactatgttcgaggtttccgtggcgttcttcaaggtgggtcatgctgtgggggagactgggaacggctgcgtagatcttaggtggctggctaaatctttcttcgagctgaccgtgttgaaagatataattggaatttgctacggggctacggttaaaggaatgcagtcttacgggctggaaaggctggccgctatgctcatggctactgtgaagatggaggaactcggccacctcaccacagaaaaacaggagtacgctttgcgcctggccactgttggctaccctaaggcaggcgtttactctggattgattgggggggccacatctgtgctcttgtcagcctacaatcgccatcccctgttccagcctctgcacacggtgatgcgagagactttgtttattggcagccacgttgtgctgagagaactgcgccttaacgttaccactcagggacctaaccttgcgctgtatcagctgcttagcaccgctttgtgctccgctcttgaaattggagaagtactcagagggctcgcactgggtactgagtcagggctgttctccccctgctatctgtctctccggttcgacctcactcgggataagctgttgtcaatggctccgcaggaagccacattggaccaggccgcagtgagtaacgcagtggacgggttcctgggtagactgtctcttgagagggaagacagagacgcttggcatctgcctgcgtataagtgtgttgatagactggacaaagtactgatgatcattcccctgattaatgtgacatttattatatctagtgaccgagaggtgagaggctccgctctctatgaagcatccacaacgtacttgagcagttccctgtttctgtcaccggttattatgaacaaatgttctcagggagctgttgctggtgaaccgcgacagattccaaagattcagaacttcactaggacacaaaagagctgcattttctgcgggttcgcactgctgagttacgacgaaaaggagggactggagactactacatatattacttcacaggaggtgcagaatagcatcctgagctccaattattttgactttgacaacctccatgtccattatttgctgttgaccacaaacggcactgtgatggagatcgctggcctgtacgaggagcgagcccacgtagtactggccattattctgtacttcatcgctttcgcacttgggattttcctcgtccacaaaattgttatgtttttcctctaatgactattcgaactactcagctagtctacgcgccgtagcctaccctccatcattgttataaaaaacttaggatccaagagcatacagccgccgataaggtccacaatgagagccgtaggtgtctttctggctatttgtctggtaacaatcttcgtgctgcccacttggggcaactgggcctatccttgctgccatgttacacagttgagagcgcagcacctcctggcgttggagaacatttcagacatctacctggtgagtaaccaaacctgcgacgggttctctttggccagtctcaatagtccaaaaaacggaagcaatcagctggtaatttcacggtgtgcaaacggacttaacgtggtctctttttttatatcaatcctgaaacgcagttcatctgcattgaccggccacttgagggagctcttgaccactctcgagacactttacgggtcattctccgtggaggatctgttcggggcaaatctgaaccggtacgcctggcatagaggcgggtaagcgcgcagcgcttagacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 86 - DNA, 19464 bp, synthetic construct
  source 1..19464: mol_type = other DNA
accaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggcatcaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagatcaagttccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacggccaccatgcaactcctctgcgtcttttgcttggtgcttttgtgggaggtaggagcggcctcattgtcagaagttaagctgcatctcgatattgaaggccacgcttcacattacactatcccttggaccgaactcatggctaaggtgccgggtttgtctccggaagcactttggagagaggcaaacgttaccgaagacctggcctcaatgctgaatcgctacaagctgatatacaagacttccggaaccctgggtatagcactcgcagagcccgtcgatatccccgctgtcagcgaaggtagcatgcaggtggacgctagtaaagttcacccaggagttatctcaggtctgaatagcccagcttgtatgctgagcgctccactggagaaacagctcttctactatataggcacaatgctccctaacactcgacctcacagctacgtcttctatcagctgcgctgtcacctgtcatacgtagccttgtccattaatggtgataagttccagtacacaggtgccatgacctccaagttccttatggggacctataaacgggtcactgagaaaggagacgagcatgtccttagtttggtttttgggaagacaaaagacctgccggatctgaggggcccgttctcttacccctcactcacatctgcacagagtggggactattcattggtgattgtgacaaccttcgttcactacgccaattttcacaactattttgtcccgaaccttaaagacatgttttcacgcgccgtgaccatgacagctgcttcctacgctaggtatgtgctccaaaagctggtgctgctggagatgaagggcggatgcagggaacctgaactggacacagagactctcaccactatgttcgaggtttccgtggcgttcttcaaggtgggtcatgctgtgggggagactgggaacggctgcgtagatcttaggtggctggctaaatctttcttcgagctgaccgtgttgaaagatataattggaatttgctacggggctacggttaaaggaatgcagtcttacgggctggaaaggctggccgctatgctcatggctactgtgaagatggaggaactcggccacctcaccacagaaaaacaggagtacgctttgcgcctggccactgttggctaccctaaggcaggcgtttactctggattgattgggggggccacatctgtgctcttgtcagcctacaatcgccatcccctgttccagcctctgcacacggtgatgcgagagactttgtttattggcagccacgttgtgctgagagaactgcgccttaacgttaccactcagggacctaaccttgcgctgtatcagctgcttagcaccgctttgtgctccgctcttgaaattggagaagtactcagagggctcgcactgggtactgagtcagggctgttctccccctgctatctgtctctccggttcgacctcactcgggataagctgttgtcaatggctccgcaggaagccacattggaccaggccgcagtgagtaacgcagtggacgggttcctgggtagactgtctcttgagagggaagacagagacgcttggcatctgcctgcgtataagtgtgttgatagactggacaaagtactgatgatcattcccctgattaatgtgacatttattatatctagtgaccgagaggtgagaggctccgctctctatgaagcatccacaacgtacttgagcagttccctgtttctgtcaccggttattatgaacaaatgttctcagggagctgttgctggtgaaccgcgacagattccaaagattcagaacttcactaggacacaaaagagctgcattttctgcgggttcgcactgctgagttacgacgaaaaggagggactggagactactacatatattacttcacaggaggtgcagaatagcatcctgagctccaattattttgactttgacaacctccatgtccattatttgctgttgaccacaaacggcactgtgatggagatcgctggcctgtacgaggagcgagcccacgtagtactggccattattctgtacttcatcgctttcgcacttgggattttcctcgtccacaaaattgttatgtttttcctctaatgattcgaactacagctcaacttacctgccaaccccatgccagtcgacccaactagtacaacctaaatccattataaaaaacttaggaaccaggtccacacagccgccagcccatcaaccatccactcccacgattggaggccggccatggtgagttttaagcaggtccgggtacctctgttcaccgccattgcgctggtgattgttctgctgttggcctactttcttccacctagagtcagaggaggtggcagggttgctgcagctgctattacctgggtaccgaaacccaacgtggaggtttggccagtggatccgcccccaccagtgaactttaataaaacagccgaacaagaatacggggataaagaagtaaagctgccacactggacccctaccctccacactttccaggtgcctcaaaactatacaaaggccaattgcacatactgtaacacaagagaatacactttttcttataaagggtgctgcttctacttcactaagaagaagcatacttggaacggatgtttccaggcatgcgctgagttgtatccctgtacctatttctacggtcctacaccagacattctgccggtcgtgactcgcaatctgaacgccatagaatctctgtgggtcggggtgtatcgggttggggaaggaaattggactagcctggacggcggtactttcaaggtgtaccaaatattcggcagtcactgtacttacgtgtcaaaattctcaacagttcccgtatcccaccacgagtgttcttttttgaagccttgcctgtgcgtgtcacagaggtcaaattcctaactattcgaactactcagctagtctacgcgccgtagcctaccctccatcattgttataaaaaacttaggatccaagagcatacagccgccgataaggtccacaatgagagccgtaggtgtctttctggctatttgtctggtaacaatcttcgtgctgcccacttggggcaactgggcctatccttgctgccatgttacacagttgagagcgcagcacctcctggcgttggagaacatttcagacatctacctggtgagtaaccaaacctgcgacgggttctctttggccagtctcaatagtccaaaaaacggaagcaatcagctggtaatttcacggtgtgcaaacggacttaacgtggtctctttttttatatcaatcctgaaacgcagttcatctgcattgaccggccacttgagggagctcttgaccactctcgagacactttacgggtcattctccgtggaggatctgttcggggcaaatctgaaccggtacgcctggcatagaggcgggtaagcgcgcagcgcttagacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 87 - DNA, 19337 bp, synthetic construct
  source 1..19337: mol_type = other DNA
accaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggcatcaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagatcaagttccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaaccgtacgatgcaactcctctgcgtcttttgcttggtgcttttgtgggaggtaggagcggcctcattgtcagaagttaagctgcatctcgatattgaaggccacgcttcacattacactatcccttggaccgaactcatggctaaggtgccgggtttgtctccggaagcactttggagagaggcaaacgttaccgaagacctggcctcaatgctgaatcgctacaagctgatatacaagacttccggaaccctgggtatagcactcgcagagcccgtcgatatccccgctgtcagcgaaggtagcatgcaggtggacgctagtaaagttcacccaggagttatctcaggtctgaatagcccagcttgtatgctgagcgctccactggagaaacagctcttctactatataggcacaatgctccctaacactcgacctcacagctacgtcttctatcagctgcgctgtcacctgtcatacgtagccttgtccattaatggtgataagttccagtacacaggtgccatgacctccaagttccttatggggacctataaacgggtcactgagaaaggagacgagcatgtccttagtttggtttttgggaagacaaaagacctgccggatctgaggggcccgttctcttacccctcactcacatctgcacagagtggggactattcattggtgattgtgacaaccttcgttcactacgccaattttcacaactattttgtcccgaaccttaaagacatgttttcacgcgccgtgaccatgacagctgcttcctacgctaggtatgtgctccaaaagctggtgctgctggagatgaagggcggatgcagggaacctgaactggacacagagactctcaccactatgttcgaggtttccgtggcgttcttcaaggtgggtcatgctgtgggggagactgggaacggctgcgtagatcttaggtggctggctaaatctttcttcgagctgaccgtgttgaaagatataattggaatttgctacggggctacggttaaaggaatgcagtcttacgggctggaaaggctggccgctatgctcatggctactgtgaagatggaggaactcggccacctcaccacagaaaaacaggagtacgctttgcgcctggccactgttggctaccctaaggcaggcgtttactctggattgattgggggggccacatctgtgctcttgtcagcctacaatcgccatcccctgttccagcctctgcacacggtgatgcgagagactttgtttattggcagccacgttgtgctgagagaactgcgccttaacgttaccactcagggacctaaccttgcgctgtatcagctgcttagcaccgctttgtgctccgctcttgaaattggagaagtactcagagggctcgcactgggtactgagtcagggctgttctccccctgctatctgtctctccggttcgacctcactcgggataagctgttgtcaatggctccgcaggaagccacattggaccaggccgcagtgagtaacgcagtggacgggttcctgggtagactgtctcttgagagggaagacagagacgcttggcatctgcctgcgtataagtgtgttgatagactggacaaagtactgatgatcattcccctgattaatgtgacatttattatatctagtgaccgagaggtgagaggctccgctctctatgaagcatccacaacgtacttgagcagttccctgtttctgtcaccggttattatgaacaaatgttctcagggagctgttgctggtgaaccgcgacagattccaaagattcagaacttcactaggacacaaaagagctgcattttctgcgggttcgcactgctgagttacgacgaaaaggagggactggagactactacatatattacttcacaggaggtgcagaatagcatcctgagctccaattattttgactttgacaacctccatgtccattatttgctgttgaccacaaacggcactgtgatggagatcgctggcctgtacgaggagcgagcccacgtagtactggccattattctgtacttcatcgctttcgcacttgggattttcctcgtccacaaaattgttatgtttttcctcagagcaaagagagcgacgaacttctcactgcttaagcaagcaggagacgtggaggagaatccgggcccaatgagagccgtaggtgtctttctggctatttgtctggtaacaatcttcgtgctgcccacttggggcaactgggcctatccttgctgccatgttacacagttgagagcgcagcacctcctggcgttggagaacatttcagacatctacctggtgagtaaccaaacctgcgacgggttctctttggccagtctcaatagtccaaaaaacggaagcaatcagctggtaatttcacggtgtgcaaacggacttaacgtggtctctttttttatatcaatcctgaaacgcagttcatctgcattgaccggccacttgagggagctcttgaccactctcgagacactttacgggtcattctccgtggaggatctgttcggggcaaatctgaaccggtacgcctggcatagaggcgggagagcaaagagagagggaagaggctccttgctgacctgcggtgacgtggaagagaatcccggccctatggtgagttttaagcaggtccgggtacctctgttcaccgccattgcgctggtgattgttctgctgttggcctactttcttccacctagagtcagaggaggtggcagggttgctgcagctgctattacctgggtaccgaaacccaacgtggaggtttggccagtggatccgcccccaccagtgaactttaataaaacagccgaacaagaatacggggataaagaagtaaagctgccacactggacccctaccctccacactttccaggtgcctcaaaactatacaaaggccaattgcacatactgtaacacaagagaatacactttttcttataaagggtgctgcttctacttcactaagaagaagcatacttggaacggatgtttccaggcatgcgctgagttgtatccctgtacctatttctacggtcctacaccagacattctgccggtcgtgactcgcaatctgaacgccatagaatctctgtgggtcggggtgtatcgggttggggaaggaaattggactagcctggacggcggtactttcaaggtgtaccaaatattcggcagtcactgtacttacgtgtcaaaattctcaacagttcccgtatcccaccacgagtgttcttttttgaagccttgcctgtgcgtgtcacagaggtcaaattcctaagcgcgcagcgcttagacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 88 - DNA, 19338 bp, synthetic construct
  source 1..19338: mol_type = other DNA
accaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgatgcaactcctctgcgtcttttgcttggtgcttttgtgggaggtaggagcggcctcattgtcagaagttaagctgcatctcgatattgaaggccacgcttcacattacactatcccttggaccgaactcatggctaaggtgccgggtttgtctccggaagcactttggagagaggcaaacgttaccgaagacctggcctcaatgctgaatcgctacaagctgatatacaagacttccggaaccctgggtatagcactcgcagagcccgtcgatatccccgctgtcagcgaaggtagcatgcaggtggacgctagtaaagttcacccaggagttatctcaggtctgaatagcccagcttgtatgctgagcgctccactggagaaacagctcttctactatataggcacaatgctccctaacactcgacctcacagctacgtcttctatcagctgcgctgtcacctgtcatacgtagccttgtccattaatggtgataagttccagtacacaggtgccatgacctccaagttccttatggggacctataaacgggtcactgagaaaggagacgagcatgtccttagtttggtttttgggaagacaaaagacctgccggatctgaggggcccgttctcttacccctcactcacatctgcacagagtggggactattcattggtgattgtgacaaccttcgttcactacgccaattttcacaactattttgtcccgaaccttaaagacatgttttcacgcgccgtgaccatgacagctgcttcctacgctaggtatgtgctccaaaagctggtgctgctggagatgaagggcggatgcagggaacctgaactggacacagagactctcaccactatgttcgaggtttccgtggcgttcttcaaggtgggtcatgctgtgggggagactgggaacggctgcgtagatcttaggtggctggctaaatctttcttcgagctgaccgtgttgaaagatataattggaatttgctacggggctacggttaaaggaatgcagtcttacgggctggaaaggctggccgctatgctcatggctactgtgaagatggaggaactcggccacctcaccacagaaaaacaggagtacgctttgcgcctggccactgttggctaccctaaggcaggcgtttactctggattgattgggggggccacatctgtgctcttgtcagcctacaatcgccatcccctgttccagcctctgcacacggtgatgcgagagactttgtttattggcagccacgttgtgctgagagaactgcgccttaacgttaccactcagggacctaaccttgcgctgtatcagctgcttagcaccgctttgtgctccgctcttgaaattggagaagtactcagagggctcgcactgggtactgagtcagggctgttctccccctgctatctgtctctccggttcgacctcactcgggataagctgttgtcaatggctccgcaggaagccacattggaccaggccgcagtgagtaacgcagtggacgggttcctgggtagactgtctcttgagagggaagacagagacgcttggcatctgcctgcgtataagtgtgttgatagactggacaaagtactgatgatcattcccctgattaatgtgacatttattatatctagtgaccgagaggtgagaggctccgctctctatgaagcatccacaacgtacttgagcagttccctgtttctgtcaccggttattatgaacaaatgttctcagggagctgttgctggtgaaccgcgacagattccaaagattcagaacttcactaggacacaaaagagctgcattttctgcgggttcgcactgctgagttacgacgaaaaggagggactggagactactacatatattacttcacaggaggtgcagaatagcatcctgagctccaattattttgactttgacaacctccatgtccattatttgctgttgaccacaaacggcactgtgatggagatcgctggcctgtacgaggagcgagcccacgtagtactggccattattctgtacttcatcgctttcgcacttgggattttcctcgtccacaaaattgttatgtttttcctcagagcaaagagagcgacgaacttctcactgcttaagcaagcaggagacgtggaggagaatccgggcccaatgagagccgtaggtgtctttctggctatttgtctggtaacaatcttcgtgctgcccacttggggcaactgggcctatccttgctgccatgttacacagttgagagcgcagcacctcctggcgttggagaacatttcagacatctacctggtgagtaaccaaacctgcgacgggttctctttggccagtctcaatagtccaaaaaacggaagcaatcagctggtaatttcacggtgtgcaaacggacttaacgtggtctctttttttatatcaatcctgaaacgcagttcatctgcattgaccggccacttgagggagctcttgaccactctcgagacactttacgggtcattctccgtggaggatctgttcggggcaaatctgaaccggtacgcctggcatagaggcgggagagcaaagagagagggaagaggctccttgctgacctgcggtgacgtggaagagaatcccggccctatggtgagttttaagcaggtccgggtacctctgttcaccgccattgcgctggtgattgttctgctgttggcctactttcttccacctagagtcagaggaggtggcagggttgctgcagctgctattacctgggtaccgaaacccaacgtggaggtttggccagtggatccgcccccaccagtgaactttaataaaacagccgaacaagaatacggggataaagaagtaaagctgccacactggacccctaccctccacactttccaggtgcctcaaaactatacaaaggccaattgcacatactgtaacacaagagaatacactttttcttataaagggtgctgcttctacttcactaagaagaagcatacttggaacggatgtttccaggcatgcgctgagttgtatccctgtacctatttctacggtcctacaccagacattctgccggtcgtgactcgcaatctgaacgccatagaatctctgtgggtcggggtgtatcgggttggggaaggaaattggactagcctggacggcggtactttcaaggtgtaccaaatattcggcagtcactgtacttacgtgtcaaaattctcaacagttcccgtatcccaccacgagtgttcttttttgaagccttgcctgtgcgtgtcacagaggtcaaattcctaagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 89 - DNA, 23067 bp, synthetic construct
  source 1..23067: mol_type = other DNA
accaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggcatcaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagatcaagttccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacgatgcaactcctctgcgtcttttgcttggtgcttttgtgggaggtaggagcggcctcattgtcagaagttaagctgcatctcgatattgaaggccacgcttcacattacactatcccttggaccgaactcatggctaaggtgccgggtttgtctccggaagcactttggagagaggcaaacgttaccgaagacctggcctcaatgctgaatcgctacaagctgatatacaagacttccggaaccctgggtatagcactcgcagagcccgtcgatatccccgctgtcagcgaaggtagcatgcaggtggacgctagtaaagttcacccaggagttatctcaggtctgaatagcccagcttgtatgctgagcgctccactggagaaacagctcttctactatataggcacaatgctccctaacactcgacctcacagctacgtcttctatcagctgcgctgtcacctgtcatacgtagccttgtccattaatggtgataagttccagtacacaggtgccatgacctccaagttccttatggggacctataaacgggtcactgagaaaggagacgagcatgtccttagtttggtttttgggaagacaaaagacctgccggatctgaggggcccgttctcttacccctcactcacatctgcacagagtggggactattcattggtgattgtgacaaccttcgttcactacgccaattttcacaactattttgtcccgaaccttaaagacatgttttcacgcgccgtgaccatgacagctgcttcctacgctaggtatgtgctccaaaagctggtgctgctggagatgaagggcggatgcagggaacctgaactggacacagagactctcaccactatgttcgaggtttccgtggcgttcttcaaggtgggtcatgctgtgggggagactgggaacggctgcgtagatcttaggtggctggctaaatctttcttcgagctgaccgtgttgaaagatataattggaatttgctacggggctacggttaaaggaatgcagtcttacgggctggaaaggctggccgctatgctcatggctactgtgaagatggaggaactcggccacctcaccacagaaaaacaggagtacgctttgcgcctggccactgttggctaccctaaggcaggcgtttactctggattgattgggggggccacatctgtgctcttgtcagcctacaatcgccatcccctgttccagcctctgcacacggtgatgcgagagactttgtttattggcagccacgttgtgctgagagaactgcgccttaacgttaccactcagggacctaaccttgcgctgtatcagctgcttagcaccgctttgtgctccgctcttgaaattggagaagtactcagagggctcgcactgggtactgagtcagggctgttctccccctgctatctgtctctccggttcgacctcactcgggataagctgttgtcaatggctccgcaggaagccacattggaccaggccgcagtgagtaacgcagtggacgggttcctgggtagactgtctcttgagagggaagacagagacgcttggcatctgcctgcgtataagtgtgttgatagactggacaaagtactgatgatcattcccctgattaatgtgacatttattatatctagtgaccgagaggtgagaggctccgctctctatgaagcatccacaacgtacttgagcagttccctgtttctgtcaccggttattatgaacaaatgttctcagggagctgttgctggtgaaccgcgacagattccaaagattcagaacttcactaggacacaaaagagctgcattttctgcgggttcgcactgctgagttacgacgaaaaggagggactggagactactacatatattacttcacaggaggtgcagaatagcatcctgagctccaattattttgactttgacaacctccatgtccattatttgctgttgaccacaaacggcactgtgatggagatcgctggcctgtacgaggagcgagcccacgtagtactggccattattctgtacttcatcgctttcgcacttgggattttcctcgtccacaaaattgttatgtttttcctcagagcaaagagagcgacgaacttctcactgcttaagcaagcaggagacgtggaggagaatccgggcccaatgagagccgtaggtgtctttctggctatttgtctggtaacaatcttcgtgctgcccacttggggcaactgggcctatccttgctgccatgttacacagttgagagcgcagcacctcctggcgttggagaacatttcagacatctacctggtgagtaaccaaacctgcgacgggttctctttggccagtctcaatagtccaaaaaacggaagcaatcagctggtaatttcacggtgtgcaaacggacttaacgtggtctctttttttatatcaatcctgaaacgcagttcatctgcattgaccggccacttgagggagctcttgaccactctcgagacactttacgggtcattctccgtggaggatctgttcggggcaaatctgaaccggtacgcctggcatagaggcgggagagcaaagagagagggaagaggctccttgctgacctgcggtgacgtggaagagaatcccggccctatggtgagttttaagcaggtccgggtacctctgttcaccgccattgcgctggtgattgttctgctgttggcctactttcttccacctagagtcagaggaggtggcagggttgctgcagctgctattacctgggtaccgaaacccaacgtggaggtttggccagtggatccgcccccaccagtgaactttaataaaacagccgaacaagaatacggggataaagaagtaaagctgccacactggacccctaccctccacactttccaggtgcctcaaaactatacaaaggccaattgcacatactgtaacacaagagaatacactttttcttataaagggtgctgcttctacttcactaagaagaagcatacttggaacggatgtttccaggcatgcgctgagttgtatccctgtacctatttctacggtcctacaccagacattctgccggtcgtgactcgcaatctgaacgccatagaatctctgtgggtcggggtgtatcgggttggggaaggaaattggactagcctggacggcggtactttcaaggtgtaccaaatattcggcagtcactgtacttacgtgtcaaaattctcaacagttcccgtatcccaccacgagtgttcttttttgaagccttgcctgtgcgtgtcacagaggtcaaattcctaagcgcgcagcgcttagacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccggtcgatcgacgatccggctgctaacaaagcccgaaaggaagctgagttggctgctgccaccgctgagcaataactagcataaccccttggggcctctaaacgggtcttgaggggttttttgctgaaaggaggaactatatccggatcgagatcaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcccgaaaggaagctgagttggctgctgccaccgctgagcaataactagcataaccccttggggcctctaaacgggtcttgaggggttttttgctgaaaggaggaacgcgcctgatgcggtattttctccttacgcatctgtgcggtatttcacaccgcatatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttacgcgtcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtgg

SEQ ID NO: 90 - DNA, 21 bp, synthetic construct
  source 1..21: mol_type = other DNA
tcgagtccctcacgcttacag

SEQ ID NO: 91 - DNA, 20 bp, synthetic construct
  source 1..20: mol_type = other DNA
ggcggtgcttgatgttctga

SEQ ID NO: 92 - DNA, 18 bp, synthetic construct
  source 1..18: mol_type = other DNA
ctggaggaccctacactg

SEQ ID NO: 93 - DNA, 18 bp, synthetic construct
  source 1..18: mol_type = other DNA
  modified_base 1: mod_base = OTHER; note = 6-carboxyfluorescein-cytosine
  modified_base 18: mod_base = OTHER; note = dihydrocyclopyrroloindole tripeptide-guanine
ctggaggaccctacactg

(19) United States
(12) Patent Application Publication
(10) Pub. No.: US 2026/0115277 A1
(43) Pub. Date: Apr. 30, 2026

(54) IMMUNOGENIC COMPOSITIONS FOR EPSTEIN-BARR VIRUS PROTEINS

(71) Applicant: MERCK SHARP & DOHME LLC, Rahway, NJ (US)

(72) Inventors: Constantine James KONSTANTOULAS, Vienna (AT); Sabrina SCHRAUF, Mörbisch am See (AT); Dai WANG, Blue Bell, PA (US)

(73) Assignee: MERCK SHARP & DOHME LLC, Rahway, NJ (US)

(21) Appl. No.: 18/874,278

(22) PCT Filed: Jun. 22, 2023
(86) PCT No.: PCT/US2023/025918

§ 371 Date: Dec. 12, 2024

Related U.S. Application Data
(60) Provisional application No. 63/354,861, filed on Jun. 23, 2022.
(60) Provisional application No. 63/376,330, filed on Sep. 20, 2022.

(51) Int. Cl. A61K 39/245; A61K 39/00; A61P 37/04; C07K 14/005

(52) U.S. Cl. CPC A61K 39/245; A61P 37/04; C07K 14/005; A61K 2039/5256

(57) ABSTRACT

The disclosure provides live attenuated measles vectors encoding in their genome one or more heterologous genes encoding Epstein-Barr Virus (EBV) proteins, e.g., gp350, gH, gL, and gp42, and variants thereof, as well as nucleic acid constructs encoding such measles vectors. The disclosure also relates to immunogenic compositions comprising live attenuated measles vectors encoding EBV proteins, immunogenic compositions comprising such measles vectors, and use of such measles vectors and immunogenic compositions to induce an immune response to EBV in subjects.

### CROSS-REFERENCE TO RELATED APPLICATIONS

This application claims the benefit of U.S. provisional patent application No. 63/354,861, filed Jun. 23, 2022; and U.S. provisional patent application No. 63/376,330, filed Sep. 20, 2022, each of which is incorporated by reference in its entirety herein.

### REFERENCE TO SEQUENCE LISTING SUBMITTED ELECTRONICALLY

The contents of the electronic sequence listing (25405-WO-SEQLIST-XX2023.xml; Size: 323,900 bytes; and Date of Creation: Nov. 22, 2022) are herein incorporated by reference in their entirety.

### FIELD

This disclosure relates generally to viral vector-based immunogenic compositions against Epstein-Barr Virus.

### BACKGROUND OF THE INVENTION

Epstein-Barr virus (EBV; also called human herpesvirus 4) is a double-stranded linear DNA virus in the Herpesviridae family. EBV is primarily transmitted orally but can also be transmitted via blood transfusions and organ transplants. EBV infects B cells of the immune system and epithelial cells and can remain latent in memory B cells after active infection.

EBV infection is the cause of infectious mononucleosis and is associated with lymphoproliferative diseases including post-transplant lympho-proliferative disease (PTLD), Burkitt lymphoma, hemophagocytic lymphohistiocytosis, Hodgkin's lymphoma, gastric cancer, and nasopharyngeal carcinoma. EBV is also associated with autoimmune disease including dermatomyositis, systemic lupus erythematosus, rheumatoid arthritis, and Sjögren's syndrome, and multiple sclerosis.

EBV is estimated to have infected 95% of adults worldwide, and about 50% of adolescents with primary EBV infection develop infectious mononucleosis. EBV-associated malignancies account for 1.8% of all cancer deaths worldwide, about 200,000 cases and 140,000 deaths annually. One out of every 1000 individuals who contract infectious mononucleosis develop Hodgkin's lymphoma. However, no EBV vaccine currently exists. Accordingly, there is a need for a vaccine that can reduce the prevalence of EBV infection.

### SUMMARY OF THE INVENTION

In a first aspect, the disclosure provides an isolated nucleic acid molecule comprising: a) a cDNA encoding a full length, antigenomic (+) RNA strand of an attenuated strain of measles virus (MV-cDNA); b) one or more cDNAs encoding an Epstein-Barr virus (EBV) protein (EBV cDNA) independently selected from the group consisting of gp350, LMP2, gH, gL, gp42, and variants thereof; c) an upstream additional transcriptional unit (ATU) cDNA operably linked to the EBV cDNA that is 5′ of the EBV cDNA (upstream ATU cDNA); and d) a downstream ATU cDNA operably linked to the EBV cDNA that is 3′ of the EBV cDNA (downstream ATU cDNA); wherein the upstream ATU cDNA, the EBV cDNA, and the downstream ATU cDNA are between the P and M genes of the MV-cDNA (ATU2) or between the H and L genes of the MV-cDNA (ATU3).

In some embodiments of the first aspect, each of the one or more EBV cDNAs in the first embodiment encodes an EBV protein sequence independently selected from the group consisting of SEQ ID NO: 54 (EBV gp350); SEQ ID NO: 55 (EBV LMP2); SEQ ID NO: 56 (EBV gH); SEQ ID NO: 57 (EBV gL); and SEQ ID NO: 58 (EBV gp42). In some embodiments, the upstream ATU cDNA, the EBV cDNA, and the downstream ATU cDNA are at ATU2 in the MV-cDNA. In some embodiments of the first aspect, the upstream ATU cDNA, the EBV cDNA, and the downstream ATU cDNA are at ATU3 in the MV-cDNA. In some embodiments of the first aspect, the upstream ATU cDNA sequence is set forth in SEQ ID NO: 69. In some embodiments of the first aspect, the downstream ATU cDNA sequence is set forth in SEQ ID NO: 72. In some embodiments of the first aspect, the isolated nucleic acid molecule comprises a sequence selected from the group consisting of SEQ ID NOs: 59, 60, 61, 83, 84, 85, 86, 87, 88.

In a second aspect, the disclosure provides an isolated nucleic acid molecule comprising: a) a cDNA encoding a full length, antigenomic (+) RNA strand of an attenuated strain of measles virus (MV-cDNA); b) a first cDNA encoding a Epstein-Barr virus (EBV) protein (EBV cDNA) selected from the group consisting of gp350, LMP2, gH, gL, gp42, and variants thereof; c) a second EBV cDNA selected from the group consisting of gp350, LMP2, gH, gL, gp42, and variants thereof, wherein the first and second EBV cDNAs do not have the same sequence; d) an upstream additional transcriptional unit (ATU) cDNA operably linked to the EBV cDNA that is 5′ of the first EBV cDNA (upstream ATU cDNA); e) a downstream ATU cDNA that is 3′ of the second EBV cDNA encoding the EBV protein; and f) an interstitial ATU cDNA between the first and second EBV cDNAs (interstitial ATU cDNA); wherein the upstream ATU cDNA, the first and second EBV cDNAs, the interstitial ATU cDNA and the downstream ATU cDNA are operably linked; and wherein the upstream ATU cDNA, the first and second EBV cDNAs, the interstitial ATU, and the downstream ATU cDNA are between the P and M genes of the MV-cDNA (ATU2) or between the H and L genes of the MV-cDNA (ATU3).

In some embodiments of the second aspect, the first and second EBV cDNA each encode an EBV protein sequence independently selected from the group consisting of: SEQ ID NO: 54 (EBV gp350); SEQ ID NO: 55 (EBV LMP2); SEQ ID NO: 56 (EBV gH); SEQ ID NO: 57 (EBV gL); and SEQ ID NO: 58 (EBV gp42). In some embodiments of the second aspect, the upstream ATU cDNA, the first and second EBV cDNA, the interstitial ATU cDNA and the downstream ATU cDNA are at ATU2 in the MV-cDNA. In some embodiments of the second aspect, the upstream ATU cDNA, the first and second EBV cDNA, the interstitial ATU cDNA and the downstream ATU cDNA are at ATU3 in the MV-cDNA. In some embodiments of the second aspect, the upstream ATU cDNA sequence is set forth in SEQ ID NO: 69. In some embodiments of the second aspect, the downstream ATU cDNA sequence is set forth in SEQ ID NO: 72. In some embodiments of the second aspect, the interstitial ATU cDNA sequence is selected from the group consisting of SEQ ID NOs: 65, 69, 72, 75, 78, and 79. In some embodiments of the second aspect, the isolated nucleic acid molecule comprises the sequence set forth in SEQ ID NO: 60.

In a third aspect, the disclosure provides an isolated nucleic acid molecule comprising: a) a cDNA encoding a full length, antigenomic (+) RNA strand of an attenuated strain of measles virus (MV-cDNA); b) a first cDNA encoding an Epstein-Barr virus (EBV) protein (EBV cDNA) selected from the group consisting of gp350, LMP2, gH, gL, gp42, and variants thereof; c) a second EBV cDNA selected from the group consisting of gp350, LMP2, gH, gL, gp42, and variants thereof, wherein the first and second EBV cDNAs do not have the same sequence; d) an upstream additional transcriptional unit (ATU) cDNA that is 5′ of the first EBV cDNA (upstream ATU cDNA); e) a downstream ATU cDNA that is 3′ of the second EBV cDNA (downstream ATU cDNA); and f) a furin cleavage site cDNA and 2A peptide cDNA (Fur-2A cDNA) between the first and second EBV cDNAs; wherein the upstream ATU cDNA, the first and second EBV cDNAs, the Fur-2A cDNA, and the downstream ATU cDNA are operably linked; and wherein the upstream ATU cDNA, the first and second EBV cDNAs, the Fur-2A cDNA, and the downstream ATU cDNA are between the P and M genes of the MV-cDNA (ATU2) or between the H and L genes of the MV-cDNA (ATU3).

In some embodiments of the third aspect, the first and second EBV cDNA each encode an EBV protein sequence independently selected from the group consisting of SEQ ID NO: 54 (EBV gp350); SEQ ID NO: 55 (EBV LMP2); SEQ ID NO: 56 (EBV gH); SEQ ID NO: 57 (EBV gL); and SEQ ID NO: 58 (EBV gp42). In some embodiments of the third aspect, the upstream ATU cDNA, the first and second EBV cDNAs, the Fur-2A cDNA, and the downstream ATU cDNA are at ATU2 in the MV-cDNA. In some embodiments of the third aspect, the upstream ATU cDNA, the first and second EBV cDNAs, the Fur-2A cDNA, and the downstream ATU cDNA are at ATU3 in the MV-cDNA. In some embodiments of the third aspect, the upstream ATU cDNA sequence is set forth in SEQ ID NO: 69. In some embodiments of the third aspect, the downstream ATU cDNA sequence is set forth in SEQ ID NO: 72. the furin cDNA of the Fur-2A cDNA encodes a protein sequence selected from the group consisting of SEQ ID NOs: 14-53, and wherein the 2A peptide cDNA of the Fur-2A cDNA encodes a protein sequence independently selected from the group consisting of SEQ ID NOs: 4-11.

In a fourth aspect, the disclosure provides an isolated nucleic acid molecule comprising: a) a cDNA encoding a full length, antigenomic (+) RNA strand of an attenuated strain of measles virus (MV-cDNA); b) a first cDNA encoding an Epstein-Barr virus (EBV) protein (EBV cDNA) selected from the group consisting of gp350, LMP2, gH, gL, gp42, and variants thereof, c) a second EBV cDNA selected from the group consisting of gp350, LMP2, gH, gL, gp42, and variants thereof; d) a third EBV cDNA selected from the group consisting of gp350, LMP2, gH, gL, gp42, and variants thereof, wherein the first, second, and third EBV cDNAs do not have the same sequence; e) an upstream additional transcriptional unit (ATU) cDNA that is 5′ of the first EBV cDNA (upstream ATU cDNA); f) a downstream ATU cDNA that is 3′ of the third EBV cDNA (downstream ATU cDNA); and g) a first interstitial ATU cDNA between the first and second EBV cDNAs (first interstitial ATU cDNA); h) a second interstitial ATU cDNA between the second and third EBV cDNAs (second interstitial ATU cDNA); wherein the upstream ATU cDNA, the first, second, and third EBV cDNAs, the interstitial ATU cDNAs, and the downstream ATU cDNA are operably linked; and wherein the upstream ATU cDNA, the first, second, and third EBV cDNAs, the first and second interstitial ATU cDNAs, and the downstream ATU cDNA are between the P and M genes of the MV-cDNA (ATU2) or between the H and L genes of the MV-cDNA (ATU3).

In some embodiments of the fourth aspect, the first, second, and third EBV cDNAs each encode an EBV protein sequence independently selected from the group consisting of: SEQ ID NO: 54 (EBV gp350); SEQ ID NO: 55 (EBV LMP2); SEQ ID NO: 56 (EBV gH); SEQ ID NO: 57 (EBV gL); and SEQ ID NO: 58 (EBV gp42). In some embodiments of the fourth aspect, the upstream ATU cDNA, the first, second, and third EBV cDNAs, the first and second interstitial ATU cDNAs, and the downstream ATU cDNA are at ATU2 in the MV-cDNA. In some embodiments of the fourth aspect, the upstream ATU cDNA, the first, second, and third EBV cDNAs, the first and second interstitial ATU cDNAs, and the downstream ATU cDNA are at ATU3 in the MV-cDNA. In some embodiments of the fourth aspect, the upstream ATU cDNA sequence is set forth in SEQ ID NO: 69. In some embodiments of the fourth aspect, the downstream ATU cDNA sequence is set forth in SEQ ID NO: 72. In some embodiments of the fourth aspect, the first and second interstitial ATU cDNA sequences are independently selected from the group consisting of SEQ ID NOs: 65, 69, 72, 75, 78, and 79. In some embodiments of the fourth aspect, the isolated nucleic acid molecule comprises a sequence selected from the group consisting of SEQ ID NOs: 61, 83, 84, 85, and 86.

In a fifth aspect, the disclosure provides an isolated nucleic acid molecule comprising: a) a cDNA encoding a full length, antigenomic (+) RNA strand of an attenuated strain of measles virus (MV-cDNA); b) a first cDNA encoding an Epstein-Barr virus (EBV) protein (EBV cDNA) selected from the group consisting of gp350, LMP2, gH, gL, gp42, and variants thereof, c) a second EBV cDNA selected from the group consisting of gp350, LMP2, gH, gL, gp42, and variants thereof, d) a third EBV cDNA selected from the group consisting of gp350, LMP2, gH, gL, gp42, and variants thereof, wherein the first, second, and third EBV cDNAs do not have the same sequence; e) an upstream additional transcriptional unit (ATU) cDNA that is 5′ of the first EBV cDNA (upstream ATU cDNA); f) a downstream ATU cDNA that is 3′ of the third EBV cDNA (downstream ATU cDNA); g) a first furin cleavage site cDNA and 2A peptide cDNA (first Fur-2A cDNA) between the first and second EBV cDNAs; and h) a second furin cleavage site cDNA and 2A peptide cDNA (second Fur-2A cDNA) between the second and third EBV cDNAs; wherein the upstream ATU cDNA, the first, second, and third EBV cDNAs, the first and second Fur-2A cDNAs, and the downstream ATU cDNA are operably linked; and wherein the upstream ATU cDNA, the first, second, and third EBV cDNAs, the first and second Fur-2A cDNAs, and the downstream ATU cDNA are between the P and M genes of the MV-cDNA (ATU2) or between the H and L genes of the MV-cDNA (ATU3).

In some embodiments of the fifth aspect, the first, second, and third EBV cDNAs each encode an EBV protein sequence selected from the group consisting of: SEQ ID NO: 54 (EBV gp350); SEQ ID NO: 55 (EBV LMP2); SEQ ID NO: 56 (EBV gH); SEQ ID NO: 57 (EBV gL); and SEQ ID NO: 58 (EBV gp42). In some embodiments of the fifth aspect, the upstream ATU cDNA, the first, second, and third EBV cDNAs, the Fur-2A cDNAs, and the downstream ATU cDNA are at ATU2 in the MV-cDNA. In some embodiments of the fifth aspect, the upstream ATU cDNA, the first, second, and third EBV cDNAs, the Fur-2A cDNAs, and the downstream ATU cDNA are at ATU3 in the MV-cDNA. In some embodiments of the fifth aspect, the upstream ATU cDNA sequence is set forth in SEQ ID NO: 69. In some embodiments of the fifth aspect, the downstream ATU cDNA sequence is set forth in SEQ ID NO: 72. In some embodiments of the fifth aspect, the furin cDNA of the Fur-2A cDNA encodes a protein sequence selected from the group consisting of SEQ ID NOs: 14-53, and wherein the 2A peptide cDNA of the Fur-2A cDNA encodes a protein sequence independently selected from the group consisting of SEQ ID NOs: 4-11. In some embodiments of the fifth aspect, the isolated nucleic acid molecule comprises the sequence set forth in SEQ ID NO: 87 or 88.

In a sixth aspect, the disclosure provides an isolated nucleic acid molecule comprising: a) a cDNA encoding a full length, antigenomic (+) RNA strand of an attenuated strain of measles virus (MV-cDNA); b) a first cDNA encoding a Epstein-Barr virus (EBV) protein (EBV cDNA) selected from the group consisting of gp350, LMP2, gH, gL, gp42, and variants thereof, c) a second EBV cDNA selected from the group consisting of gp350, LMP2, gH, gL, gp42, and variants thereof, d) a third EBV cDNA selected from the group consisting of gp350, LMP2, gH, gL, gp42, and variants thereof, wherein the first, second, and third EBV cDNAs do not have the same sequence; e) an upstream additional transcriptional unit (ATU) cDNA that is 5′ of the first EBV cDNA (upstream ATU cDNA); f) a downstream ATU cDNA that is 3′ of the third EBV cDNA (downstream ATU cDNA); and g) a furin cleavage site cDNA and 2A peptide cDNA (Fur-2A cDNA); and h) an interstitial ATU cDNA; wherein the upstream ATU cDNA, the first, second, and third EBV cDNAs, the Fur-2A cDNA, the interstitial ATU cDNA, and the downstream ATU cDNA are operably linked; wherein i) the Fur-2A cDNA is between the first and second EBV cDNA and the interstitial ATU cDNA is between the second and third EBV cDNA, or ii) the interstitial ATU cDNA is between the first and second EBV cDNA and the Fur-2A cDNA is between the second and third EBV cDNA; and wherein the upstream ATU cDNA, the first, second, and third EBV cDNAs, the Fur-2A cDNA, the interstitial ATU cDNA, and the downstream ATU cDNA are between the P and M genes of the MV-cDNA (ATU2) or between the H and L genes of the MV-cDNA (ATU3).

In some embodiments of the sixth aspect, the first, second, and third EBV cDNAs each encode an EBV protein sequence independently selected from the group consisting of: SEQ ID NO: 54 (EBV gp350); SEQ ID NO: 55 (EBV LMP2); SEQ ID NO: 56 (EBV gH); SEQ ID NO: 57 (EBV gL); and SEQ ID NO: 58 (EBV gp42). In some embodiments of the sixth aspect, the upstream ATU cDNA, the first, second, and third EBV cDNAs, the Fur-2A cDNA, the interstitial ATU cDNA, and the downstream ATU cDNA are at ATU2 in the MV-cDNA. In some embodiments of the sixth aspect, the upstream ATU cDNA, the first, second, and third EBV cDNAs, the Fur-2A cDNA, the interstitial ATU cDNA, and the downstream ATU cDNA are at ATU3 in the MV-cDNA. In some embodiments of the sixth aspect, the upstream ATU cDNA sequence is set forth in SEQ ID NO: 69, the downstream ATU cDNA sequence is set forth in SEQ ID NO: 72. In some embodiments of the sixth aspect, the furin cDNA of the Fur-2A cDNA encodes a protein sequence selected from the group consisting of SEQ ID NOs: 14-53, and wherein the 2A peptide cDNA of the Fur-2A cDNA encodes a protein sequence independently selected from the group consisting of SEQ ID NOs: 4-11. In some embodiments of the sixth aspect, the interstitial ATU cDNA sequence is selected from the group consisting of SEQ ID NOs: 65, 69, 72, 75, 78, and 79. In some embodiments of the sixth aspect, the Fur-2A cDNA is between the first and second EBV cDNA and the interstitial ATU cDNA is between the second and third EBV cDNA. In some embodiments of the sixth aspect, the interstitial ATU cDNA is between the first and second EBV cDNA and the Fur-2A cDNA is between the second and third EBV cDNA.

In a seventh aspect, the disclosure also provides a vector for the rescue of a recombinant measles virus, comprising the isolated nucleic acid molecule of any one of the first through sixth aspects or any one of the embodiments thereof. In some embodiments, the vector comprises a CMV promoter. In some embodiments, the vector comprises the sequence set forth in SEQ ID NO: 89. In some embodiments, the vector comprises a T7 promoter. In some embodiments, the vector comprises the sequence set forth in SEQ ID NO: 3.

In an eighth aspect, the disclosure also provides a recombinant measles virus comprising in its genome a cDNA sequence comprising the nucleic acid molecule of any one of the first through sixth aspects or any one of the embodiments thereof.

In a ninth aspect, the disclosure provides an immunogenic composition comprising (i) an effective amount of the recombinant measles virus of the eighth aspect, and (ii) a pharmaceutically acceptable carrier.

In a tenth aspect, the disclosure provides a method for treating or preventing an Epstein-Barr virus (EBV) infection in a subject, comprising administering an effective amount of the immunogenic composition according to the ninth aspect to the subject.

In an eleventh aspect, the disclosure provides a method for inducing a protective immune response against Epstein-Barr (EBV) in a subject, comprising administering an effective amount of the immunogenic composition of the ninth aspect to the subject.

In some embodiments of the ninth through eleventh aspects, the disclosure provides a method comprising a first administration of the immunogenic composition and a second administration of the immunogenic composition. In some embodiments, the protective immune response is a humoral immune response and/or a cellular immune response. In some embodiments, the second administration is performed from one month to two months after the first administration. In some embodiments, the subject is a human.

In a twelfth aspect, the disclosure provides use of the recombinant measles virus of the eighth aspect or the immunogenic composition of the ninth aspect for preventing or treating an EBV infection.

In a thirteenth aspect, the disclosure provides the recombinant measles virus of the eighth aspect or the immunogenic composition of the ninth aspect, for use in preventing or treating an EBV infection in a subject.

In a fourteenth aspect, the disclosure provides in vitro use of the recombinant measles virus of the eighth aspect or the immunogenic composition of the ninth aspect for expressing an EBV protein in eukaryotic cells.

The summary of the technology described above is non-limiting and other features and advantages of the technology will be apparent from the following detailed description, and from the claims.

### BRIEF DESCRIPTION OF THE DRAWINGS

FIG. 1 shows a schematic diagram of a recombinant measles vector genome from 5′ to 3′ direction. Additional transcriptional units (ATUs) are marked. ATU1 is positioned before the measles N gene; ATU2 is positioned between the measles P and M genes, and ATU3 is positioned between the measles H and L genes.

FIG. 2 shows line graphs comparing growth kinetics of MV expressing monovalent (M-01; gp350), bivalent (B-02; gp350/LMP2), and trivalent (T-03; gH/gL/gp42) EBV antigens from the ATU3 position in MV. Virus release from cells (TCID50/mL) (FIG. 2A) and extracellular RNA genomes (mean RNA copies/mL) (FIG. 2B) were both measured.

FIG. 3 shows a photograph of an agarose gel of PCR-amplified EBV inserts from MV-EBV constructs M-01, B-02, and T-03 at passage 4 to assess genetic stability of the MV constructs.

FIG. 4A-4C show dot plots of ELISA-measured serum neutralizing antibody titer against various EBV antigens in cotton rats that received MV-EBV constructs M-01, B-02, and T-03, as well as a positive control of gp350 protein and MPL-A and a negative control of M-Schwarz. The limit of detection (LOD) is marked by a dotted line. FIG. 4A shows a dot plot of the serum neutralizing antibody titer against gp350, FIG. 4B shows a dot plot of the serum neutralizing antibody titer against gp42, and FIG. 4C shows a dot plot of the serum neutralizing antibody titer against gH and gL.

FIGS. 5A and 5B show dot plots of EBV serum neutralization assays using B cells (FIG. 5A) or epithelial cells (FIG. 5B), using serum from cotton rats treated with MV-EBV constructs M-01, B-02, and T-03, as well as a positive control of gp350 protein and MPL-A and a negative control of M-Schwarz. The dotted line shows the threshold value of 25. Solid lines indicate the mean.

### DETAILED DESCRIPTION OF THE INVENTION

### Definitions and Abbreviations

As used throughout the specification and appended claims, the following abbreviations apply:

| | |
|---|---|
| ATU | additional transcriptional unit |
| bp | base pairs |
| BSA | bovine serum albumin |
| CDS | coding sequence |
| CMV | human cytomegalovirus immediate |
| | early enhancer and promoter |
| EBV | Epstein-Barr Virus |
| ELISA | enzyme-linked immunosorbent assay |
| ER | endoplasmic reticulum |
| ffu | fluorescent focus units |
| GE | gene end |
| GS | gene start |
| IM | intra-muscular |
| LOD | limit of detection |
| MOI | multiplicity of infection |
| MV | measles virus |
| MV-EBV | measles virus carrying one or more |
| | EBV proteins in its genome |
| nt | nucleotide(s) |
| NT | neutralization titer/percentage |
| ORF | open reading frame |
| PBS | phosphate buffered saline |
| PFU | plaque forming units |
| SNA | serum neutralizing antibody(ies) |
| WT | wild-type |

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, use of the term “including” as well as other forms, such as “include,” “includes,” and “included,” is not limiting.

As used herein, the term “about” in quantitative terms refers to plus or minus 10% of the value it modifies (rounded up to the nearest whole number if the value is not sub-dividable, such as a number of molecules or nucleotides).

All ranges disclosed herein are inclusive of the recited endpoints and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

### Attenuated Measles Virus

Measles virus (MV) is a non-segmented single-stranded, negative-sense enveloped RNA virus of the genus Morbilivirus within the family of Paramyxoviridae. Measles virus was isolated in 1954 (Enders, J. F., and T. C. Peebles. 1954. Propagation in tissue cultures of cytopathogenic agents from patients with measles. Proc. Soc. Exp. Biol. Med. 86:277-286), and live attenuated measles strains were derived from this virus to provide vaccines. Measles vaccines from live attenuated measles virus have been administered to hundreds of millions of children since 1963 and are well-known to be safe and efficacious in preventing measles infection. It is produced on a large scale in many countries and is distributed at low cost.

The disclosure describes attenuated recombinant measles virus particles that stably express one or more protein antigens of EBV (e.g., gp350, LMP2, gH, gL and gp42). The disclosure also describes nucleic acid constructs which comprise an isolated cDNA encoding a full-length, infectious, attenuated antigenomic (+) RNA strand of a measles virus (MV) and at least one EBV protein (e.g., gp350, LMP2, gH, gL or gp42), such that a rescued MV comprises the at least one EBV protein in its genome.

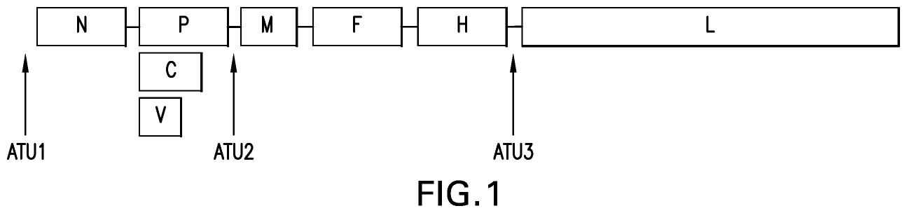

The non-segmented genome of measles virus (MV) has an anti-message polarity which results in a genomic RNA which is not translated in vivo or in vitro and is not infectious when purified. Transcription and replication of measles virus do not involve the nucleus of the infected cells but rather take place in the cytoplasm of infected cells. The genome of the measles virus comprises genes encoding six major structural proteins from the six genes (designated N, P, M, F, H and L) and an additional two non-structural proteins from the P gene (C and V). The gene order is the following: 3′, N, P (including C and V), M, F, H, and L (the L gene coding for the large polymerase protein at the 5′ end) (see schematic diagram of FIG. 1). The MV genome further comprises non-coding regions in the intergenic region M/F; this non-coding region contains approximately 1000 nucleotides of untranslated RNA. The MV genes respectively encode the proteins of the nucleocapsid of the virus, i.e., the nucleoprotein (N), the phosphoprotein (P), and the large protein (L) which assemble around the genome RNA to provide the nucleocapsid. The other genes encode the proteins of viral envelope including the hemagglutinin (H), the fusion (F) and the matrix (M) proteins.

In some embodiments, the MV used is an attenuated strain. As used herein, an “attenuated strain” of measles virus is a strain that is avirulent or less virulent than the parent strain in the same host, while maintaining immunogenicity and optionally adjuvanticity when administered to a host, i.e., preserving immunodominant T and B cell epitopes and possibly the adjuvanticity such as the induction of T cell costimulatory proteins or the cytokine IL-12.

An attenuated strain of a measles virus accordingly refers to a strain which has been serially passaged on selected cells and, possibly, adapted to other cells to produce seed strains suitable for the preparation of vaccine strains, harboring a stable genome which would not allow reversion to pathogenicity nor integration in host chromosomes. Particular strains of attenuated MV that can be used are the Schwarz strain, the Zagreb strain, the AIK-C strain and the Moraten strain. In specific embodiments, the attenuated strain of measles virus in any one of the embodiments or aspects herein is the Schwarz strain, the Zagreb strain, the AIK-C strain or the Moraten strain

In some embodiments of the invention, the vector for the rescue of a recombinant measles virus comprising the isolated nucleic acid molecule disclosed herein comprises a heterologous promoter sequence. Exemplary heterologous promoters include the CMV promoter sequence. In some embodiments, the vector is pBluescript KS (+) (GenBank X52331.1; SEQ ID NO: 1). In some embodiments, the vector is pBluescript II KS (+) (Agilent, Santa Clara, CA, United States, Cat. #212207, GenBank X52327.1; SEQ ID NO: 2). In some embodiments, the vector includes a T7 promoter sequence, a T7 terminator sequence, and a hammerhead ribozyme sequence. An exemplary sequence is that of plasmid pTM-MVSchw (SEQ ID NO: 3; see WO2004000876A1). The plasmid pTM-MVSchw is a Bluescript plasmid that comprises the polynucleotide coding for the full-length measles virus (+) RNA strand of the Schwarz strain placed under the control of the promoter of the T7 RNA polymerase.

In embodiments described herein, the MV-EBV cDNA includes EBV proteins inserted into an additional transcriptional unit (ATU). The term “additional transcriptional unit” or “ATU” in relation to the MV genome refers to an intergenic region of the MV genome having cis-acting 3′ and 5′ untranslated regions (UTRs) of the genes, which are composed of the MV non-coding sequences (NCS) and of conserved gene end (GE) and gene start (GS) signals necessary for the transcription of the immediately adjacent open reading frames. This “GE/GS stop-start signal” is comprised of a conserved GE sequence, a non-transcribed conserved trinucleotide sequence, and a conserved GS sequence (see Parks et al., J Virol. 2001 January; 75(2):921-33). During transcription, each gene in a transcription unit of the MV genome is sequentially transcribed into mRNA by the viral RNA-dependent RNA polymerase that starts the transcription process at the GS sequence. At each gene junction, transcription is interrupted as a result of the disengagement of the RNA polymerase at the GE sequence. Re-initiation of transcription occurs at the subsequent GS sequence.

To enable the MV cDNA to act as a vector for the expression of one or more EBV heterologous genes, a multiple-cloning-site cassette having one or more EBV genes can be cloned into the intergenic region of the MV so as to maintain the MV non-coding sequences and the conserved gene end (GE) and gene start signals (GS) of the immediately adjacent open reading frames of the intergenic transcription unit in which it is inserted; see, e.g., the ATU region described for the EdB-tag vector in Radecke et al., 1997 Rev. Med. Virol. 7:49-63, and Wei et al., 2019 Biochem. Biophys. Res. Comm. 508:1221-1226. Following cloning, the resulting ATU contains an additional GE/GS stop-start signal suitable for the transcription of the one or more inserted heterologous EBV genes. When multiple heterologous EBV genes are inserted, each heterologous HSV gene may be separated by an “interstitial ATU,” which is an additional GE/GS stop-start signal that separates the heterologous HSV genes. In specific embodiments, the additional GE/GS stop-start signal is the same as that found in an MV intergenic region. In specific embodiments, the GE/GS stop-start signal is a variant of that found in an MV intergenic region.

It is important in all cases that the ATU shall comply with the broader “rule of six” to allow for the expression of the one or more heterologous genes.

In this disclosure, locations of ATUs along the MV genome are numbered. ATU position 1 (ATU1) is in the MV leader sequence before the N gene. ATU position 2 (ATU2) is in the intergenic region between the P and M genes. ATU position 3 (ATU3) is in the intergenic region between the H and L genes. Insertion of heterologous transcription units ATU2 and ATU3 can be accomplished as disclosed herein or as elsewhere described in the literature, for example, in Combredet et al., 2003 J. Virol. 11546-11554.

In some embodiments, an ATU comprises a GS sequence for an N gene and a GE sequence, such as at ATU1 in MV, or suitable GS and GE variants that are capable of starting and ending transcription, respectively, in the MV. For example, in specific embodiments, the ATU cDNA comprises a GE/GS sequence (GE/GS stop-start signal) comprising CTTCTAGTGCACTTAGGATTCAA (SEQ ID NO: 65), wherein the GE sequence is CTTCTAGTGCA (SEQ ID NO: 66), the conserved trinucleotide sequence is GTT (SEQ ID NO: 67), and the GS sequence is AGGATTCAA (SEQ ID NO: 68). In some embodiments, the ATU cDNA comprises the GE of an N gene and the GS of a P gene. In some embodiments, the ATU cDNA comprises GTTATAAAAAACTTAGGAACCAGGTCCACAC (ATU upstream motif; SEQ ID NO: 69), wherein the GE sequence is GTTATAAAAAA (GE of N gene; SEQ ID NO: 70), the conserved trinucleotide sequence is GTT (SEQ ID NO: 67), and the GS sequence is AGGAACCAGGTCCACAC (GS of P gene; SEQ ID NO: 71). In some embodiments, the ATU cDNA comprises the GE of a P gene and the GS of a M gene. In some embodiments, the ATU cDNA comprises ATTATAAAAAACTTAGGAGCAAAGTGATTGC (ATU downstream motif; SEQ ID NO: 72), wherein the GE sequence is ATTATAAAAAA (GE of P gene; SEQ ID NO: 73), the conserved trinucleotide sequence is GTT (SEQ ID NO: 67), and the GS sequence is AGGAGCAAAGTGATTGC (GS of M gene; SEQ ID NO: 74). In some embodiments, a GE and GS sequence of the same gene combined with the conserved trinucleotide sequence. For example, in specific embodiments, the ATU cDNA comprises the GE and the GS of the P gene combined with a conserved trinucleotide sequence, i.e., ATTATAAAAAACTTAGGAACCAGGTCCACAC (ATUa; SEQ ID NO: 75). In some embodiments, the ATU cDNA comprises a hybrid GS sequence that combines portions of sequences from different MV intergenic regions. In some embodiments, the ATU cDNA comprises a hybrid GS sequence that is a combination of a GS sequence of an MV P gene and a GS sequence of an MV M gene, e.g., AGGAGCAAAGTCCACAC (SEQ ID NO: 76). In some embodiments, the ATU cDNA comprises a hybrid GS sequence combined with a GE from an N gene, e.g., GTTATAAAAAACTTAGGAGCAAAGTCCACAC (ATUb; SEQ ID NO: 77). In some embodiments, the ATU cDNA may be a consensus GS sequence, e.g., AGGATCCAAGAGCATAC (SEQ ID NO: 77). In some embodiments, the ATU cDNA comprises a hybrid GS sequence and a GE from an N gene, e.g., GTTATAAAAAACTTAGGATCCAAGAGCATAC (SEQ ID NO: 79).

ATU sequence that flanks the GE/GS sequence may be part or all of an intergenic region of an MV strain (e.g., the N-P, P-M or H-L intergenic region of the Schwarz, Zagreb, AIK-C. Moraten, or Rubeovax MV strain) that is duplicated in a different intergenic region of the MV (see FIG. 1).

In some embodiments of the MV described herein, the heterologous EBV gene may be preceded by a Kozak sequence. The term “Kozak sequence” refers to a nucleic acid motif that acts as a protein translation initiation site for the heterologous gene or genes and includes the ATG initiation codon. In some embodiments, the Kozak sequence in a cDNA may be the sequence GCCGCCATG (SEQ ID NO: 80) or the sequence GCCACCATG (SEQ ID NO: 81).

Complementary DNA (cDNA) encoding MV-EBV as described herein complies with the “rule of six” which is required in order to express infectious viral particles. The term “rule of six” as used herein refers the fact that the total number of nucleotides present in the MV cDNA is a multiple of six. This characteristic of the MV cDNA allows sufficient replication of genome RNA of the measles virus (see Fields B N et al. (ed.). Fields Virology. 3rd ed. Vol. 1. Raven Press; 1996 at p. 1197).

In some embodiments of the isolated nucleic acid molecules described herein, the EBV protein ORFs (the one or more EBV cDNAs encoding an EBV protein) are separated by a self-cleaving 2A peptide so that multiple separate peptides can be generated from a single ORF. The term “2A peptide”, “self-cleaving 2A peptide” or “2A self-cleaving peptide” refers to viral oligopeptides that are 18-22 amino acids in length and mediate cleavage of different polypeptides encoded by polycistronic mRNA during translation in eukaryotic cells. Coding sequences (CDS) for 2A peptides can be inserted between coding sequences for two polypeptides, and ribozyme skipping of the formation of glycyl-prolyl peptide bond at the C-terminus results in separation of the two polypeptides flanking the 2A peptide coding sequence (see Liu et al., Sci Rep. 2017 May 19; 7(1):2193). A 2A peptide may be derived from various viruses, including but not limited to: T2A (thosea asigna virus 2A; SEQ ID NO: 4, GSGEGRGSLLTCGDVEENPGP); P2A (porcine teschovirus-1 2A; SEQ ID NO: 5, GSGATNFSLLKQAGDVEENPGP); E2A (equine rhinitis A virus; SEQ ID NO: 6, GSGQCTNYALLKLAGDVESNPGP); and foot-and-mouth disease virus (F2A; SEQ ID NO: 7, GSGVKQTLNFDLLKLAGDVESNPGP). In some embodiments, the GSG sequence at the N-terminal residues 1-3 can be removed, although this can decrease cleavage efficiency: T2A—SEQ ID NO: 8, EGRGSLLTCGDVEENPGP; P2A—SEQ ID NO: 9, ATNFSLLKQAGDVEENPGP; E2A—SEQ ID NO: QCTNYALLKLAGDVESNPGP; F2A—SEQ ID NO: 10, VKQTLNFDLLKLAGDVESNPGP.

In some embodiments of the isolated nucleic acid molecules described herein, a furin cleavage sequence may be positioned between EBV antigen ORFs instead of a 2A peptide. In such embodiments, a peptide sequence is recognized by a furin enzyme in a cell and cleaved, allowing separation of polypeptides in the cell. Furin cleavage sequences are traditionally described by the consensus sequence RXRR (SEQ ID NO: 12) or RXKR (SEQ ID NO: 13), wherein X is any amino acid. In some embodiments, the furin-cleavage sequence may be SEQ ID NO: 14 (RGRR), SEQ ID NO: 15 (RARR), SEQ ID NO: 16 (RLRR), SEQ ID NO: 17 (RMRR), SEQ ID NO: 18 (RFRR), SEQ ID NO: 19 (RWRR), SEQ ID NO: 20 (RKRR), SEQ ID NO: 21 (RQRR), SEQ ID NO: 22 (RERR), SEQ ID NO: 23 (RSRR), SEQ ID NO: 24 (RPRR), SEQ ID NO: 25 (RVRR), SEQ ID NO: 26 (RIRR), SEQ ID NO: 27 (RCRR), SEQ ID NO: 28 (RYRR), SEQ ID NO: 29 (RHRR), SEQ ID NO: 30 (RRRR), SEQ ID NO: 31 (RNRR), SEQ ID NO: 32 (RDRR), SEQ ID NO: 33 (RTRR), SEQ ID NO: 34 (RGKR), SEQ ID NO: 35 (RAKR), SEQ ID NO: 36 (RLKR), SEQ ID NO: 37 (RMKR), SEQ ID NO: 38 (RFKR), SEQ ID NO: 39 (RWKR), SEQ ID NO: 40 (RKKR), SEQ ID NO: 41 (RQKR), SEQ ID NO: 42 (REKR), SEQ ID NO: 43 (RSKR), SEQ ID NO: 44 (RPKR), SEQ ID NO: 45 (RVKR), SEQ ID NO: 46 (RIKR), SEQ ID NO: 47 (RCKR), SEQ ID NO: 48 (RYKR), SEQ ID NO: 49 (RHKR), SEQ ID NO: 50 (RRKR), SEQ ID NO: 51 (RNKR), SEQ ID NO: 52 (RDKR), or SEQ ID NO: 53 (RTKR).

In some embodiments of the isolated nucleic acid molecules described herein, a furin cleavage sequence is used in combination with a 2A peptide to ensure that no additional 2A peptide sequence remains after self-cleavage by the 2A peptide. The furin cleavage sequence is adjacent to the 2A peptide, between an antigen and a 2A peptide sequence (see Fang et al., Nat Biotechnol. 2005 May; 23(5):584-90; and WO2015054639A1, each of which is incorporated herein by reference). In some embodiments, the GSG linker may be removed (see Chng et al., MAbs. 2015; 7(2):403-12, incorporated by reference herein).

Various combinations of ATUs, GE/GS sequence, 2A peptides, and 2A peptides with furin cleavage sequences are contemplated for the isolated nucleic acid molecules described herein. In some embodiments of the isolated nucleic acid molecules described herein, a single EBV protein ORF encoding a EBV protein (e.g., gp350, gH/gL, or gp42; see SEQ ID NOs: 54, 55, 56, 57, or 58) is flanked by an ATU upstream motif (SEQ ID NO: 69) and an ATU downstream motif (SEQ ID NO: 72). For example, cDNA encoding EBV gp350, gH/gL, or gp42 (see SEQ ID NOs: 54, 55, 56, 57, or 58) may be positioned at ATU2 (see e.g., SEQ ID NO: 82) or at ATU3 (see e.g., SEQ ID NO: 59).

In some embodiments, two EBV protein coding sequences (e.g., gp350, gH/gL, or gp42; see SEQ ID NOs: 54, 55, 56, 57, or 58) flanked by an ATU upstream motif (SEQ ID NO: 69) and an ATU downstream motif (SEQ ID NO: 72) may be separated by an ATUa motif (SEQ ID NO: 75) or ATUb motif (SEQ ID NO: 78) or an ATUc motif (SEQ ID NO: 79). In some embodiments, the two EBV protein coding sequences may be located at ATU2 or at ATU3 (e.g., SEQ ID NO: 60). In some embodiments, the two EBV protein coding sequences may be separated by a 2A peptide coding sequence (SEQ ID NOs: 4-11).

In some embodiments, three EBV protein coding sequences (e.g., gp350, gH/gL, or gp42; see SEQ ID NOs: 54, 55, 56, 57, or 58) flanked by an ATU upstream motif (SEQ ID NO: 69) and an ATU downstream motif (SEQ ID NO: 72) may be separated by an ATUa motif (SEQ ID NO: 75), an ATUb motif (SEQ ID NO: 78), an ATUc motif (SEQ ID NO: 79), a 2A peptide motif (SEQ ID NOs: 4-11), a furin cleavage site (SEQ ID NOs: 12-53) and a 2A peptide motif (SEQ ID NOs: 4-11), and combinations thereof. Such coding sequences may be located at ATU2 or at ATU3. For example, see SEQ ID NOs: 83 (EBV_gH_ATUa_gL_ATUb_gp42 at ATU2), 84 (EBV_gH_ATUa_gL_ATUc_gp42 at ATU3), 85 (EBV_gp42-ATUa_gH-ATUc_gL at ATU3), 86 (EBV_gH_ATUa_gp42_ATUc_gL at ATU3), 87 (EBV_gH_Fur-P2A_gL_Fur-T2A_gp42 at ATU3), and 88 (EBV_gH-Fur-P2A gL Fur-T2A_gp42 at ATU2).

### Nucleic Acids and Proteins

In some embodiments, the inventions disclosed herein refer to isolated cDNA encoding MV-EBV.

As used herein, the term “operably linked” refers to a functional relationship between two or more nucleic acid sequences. For example, DNA encoding a secretory leader (i.e., a signal peptide), is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the example of a secretory leader, in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “ORF” or “open reading frame” refers to a coding sequence of a gene that begins with the start codon, continues with the amino acid codons, and ends at a termination codon. A “gene” includes an ORF and includes sequences upstream of the start codon and downstream of the stop codon that may be useful for transcribing the ORF.

As used herein, the term “complementary DNA” or “cDNA” refers to a deoxyribonucleic acid (DNA) molecule obtained by reverse transcription of a ribonucleic acid (RNA) molecule, such as an mRNA molecule. The term “cDNA” refers to the fact that originally said molecule is obtained by reverse transcription of the full length genomic (−) RNA strand of the genome of viral particles of the measles virus. This should not be viewed as a limitation for the methods used for its preparation. Purified nucleic acids, including DNA are thus encompassed within the term cDNA.

As used herein, the term “isolated” used in the context of polypeptides or polynucleotides refers to polypeptides or polynucleotides that are at least partially free of other biological molecules from the cells or cell cultures in which they are produced. Such biological molecules include other nucleic acids, proteins, lipids, carbohydrates, or other material such as cellular debris and growth medium. It may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the polypeptides or polynucleotides.

In some embodiments of the MV-EBV constructs described herein, conservative amino acid substitutions may be used for the sequence of the encoded antigens. As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

In some embodiments of the EBV antigens encoded and expressed by the measles virus vector of the invention, the EBV antigens may have up to 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more conservative amino acid substitutions. In some embodiments, the measles vector polypeptides may have up to 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more conservative amino acid substitutions.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity,” as known in the art, refers to the degree of sequence relatedness between two sequences of polynucleotide or polypeptide molecules as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods.

The term “percent identity” or “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. Identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, or 99% but less than 100% sequence identity to that particular reference polynucleotide or polypeptide as determined by sequence alignment programs and parameters described herein and known to those skilled in the art. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, accounting for the number of gaps and the length of each gap, which needs to be introduced for optimal alignment of the two sequences. The comparison of sequences and determination of percent identity between two sequences can be accomplished using a mathematical algorithm in an alignment tool (e.g. the Needleman-Wunsch algorithm in an online tool).

As used herein, the term “global alignment” refers to an alignment of residues between two amino acid or nucleic acid sequences along their entire length, introducing gaps as necessary if the two sequences do not have the same length, to achieve a maximum percent identity. A global alignment can be created using the global alignment tool “Needle” from the online European Molecular Biology Open Software Suite (EMBOSS) (see www.ebi.ac.uk/Tools/psa/emboss_needle/) or the global alignment tool “BLASTR Global Alignment” from the National Center for Biotechnology Information (NCBI) (see blast.ncbi.nlm.nih.gov/Blast.cgi?PROGRAM=blastn&PAGE_TYPE=BlastSearch&PROG_DEF AULTS=on&BLAST_INIT=GlobalAln&BLAST_SPEC=GlobalAln&BLAST_PROGRAMS=bl astn). Both of these global alignment tools incorporate the Needleman-Wunsch algorithm (Needleman, S. B. & Wunsch, C. D. (1970) “A general method applicable to the search for similarities in the amino acid sequences of two proteins.” J. Mol. Biol. 48:443-453). In a preferred embodiment, a global alignment of nucleotide sequences using BLAST Global Alignment uses the following default parameters: match score=2; mismatch score=−3; Gap Cost Existence score=5; Gap Cost Extension Score=2. In a preferred embodiment, a global alignment of protein sequences using BLAST Global Alignment uses the following default parameters: Gap Cost Existence=11; Gap Cost Extension=1.

In some embodiments, codons encoding amino acid sequences may be substituted using wobble degenerate codons. As used herein, the term “wobble degenerate codon,” refers to a codon encoding a naturally occurring amino acid in either DNA or RNA. Wobble degenerate codons, when present in mRNA, are recognized by a natural tRNA anticodon through at least one non-Watson-Crick, or wobble base-pairing (e.g., A-C or G-U base-pairing). Watson-Crick base-pairing refers to either the G-C or A-U (RNA or DNA/RNA hybrid) or A-T (DNA) base-pairing. When used in the context of mRNA codon-tRNA anticodon base-pairing, Watson-Crick base-pairing means all codon-anticodon base-pairings are mediated through either G-C or A-U.

In some embodiments, the nucleic acids encoding the EBV proteins are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park CA), NovoPro Bioscience Inc. (Shanghai, China), and/or proprietary methods. In some embodiments, the sequence is optimized using optimization algorithms.

In some embodiments, a codon-optimized sequence shares less than 95% sequence identity, less than 90% sequence identity, less than 85% sequence identity, less than 80% sequence identity, or less than 75% sequence identity to a naturally occurring or wild-type sequence.

In some embodiments, a codon-optimized sequence shares between 65% and 85% (e.g., between about 67% and about 85%, or between about 67% and about 80%) sequence identity to a naturally occurring sequence or a wild-type sequence. In some embodiments, a codon-optimized sequence shares between 65% and 75%, or about 80% sequence identity to a naturally occurring sequence or wild-type sequence.

In some embodiments, nucleic acid sequence may be codon optimized for expression in cells from a particular animal species, such as a human (e.g., Homo sapiens) or other primate (e.g., Macaca mulatta or Macaca fascicularis). This optimization allows increasing the efficiency of chimeric infectious particles production in cells without impacting the expressed protein(s).

### EBV Proteins

EBV has a linear, double-stranded DNA genome that is approximately 170 kilobase pairs in length encoding more than 80 proteins. EBV envelope glycoproteins gH/gL, gB and gp350 are critical components for EBV infection of target cells. The glycoproteins gH/gL and gB mediate fusion with the cell membrane, with EBV gB forming a trimer and gH/gL forming a heterodimer. EBV protein gp350 allows efficient infection of B cells, while EBV envelope proteins gH/gL and gB are required for EBV infection of both B cells and epithelial cells (Cui X and Snapper C M, Front Immunol. 2021 Oct. 8; 12:734471). EBV uses gp42 complexed with gH/gL heterodimers to bind HLA class II and activate entry into B cells via glycoprotein B (gB) (Sathiyamoorthy et al., Proc Natl Acad Sci USA. 2017 Oct. 10; 114(41):E8703-E8710).

EBV infects B cells via binding of gp350 to the complement receptor 2 (CR2)/CD21. EBV gp42 then interacts with MHC-II at the host cell surface followed by association with gH/gL. EBV gH/gL then activates the EBV fusion protein gB resulting in cell endosomal membrane fusion. Epithelial cell infection by EBV uses EBV BMRF2 binding to integrins, followed by gH/gL binding to integrins and ephrin receptor A2. EBV gB is activated, resulting in fusion of the viral envelope to the plasma membrane of the epithelial cell.

The EBV glycoprotein gp350 is expressed on the EBV capsid and binds to cellular complement receptor type 2 (CR2 or CD21) in B cells. While a gp350-based vaccine previously reduced the incidence of infectious mononucleosis by 78%, it did not prevent asymptomatic EBV infection (Sokal et al., J Infect Dis. 2007 Dec. 15; 196(12):1749-53. doi: 10.1086/523813). Natural gp350 antibodies showed strong potency in neutralizing infection of B cells but not epithelial cells, which is the cell type EBV encounters first in a host.

In some embodiments disclosed herein, an antigenic polypeptide includes gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. Polypeptides may also comprise single chain polypeptides or multichain polypeptides, such as antibodies or insulin, and may be associated or linked to each other. Most commonly, disulfide linkages are found in multichain polypeptides. The term “polypeptide” may also apply to amino acid polymers in which at least one amino acid residue is an artificial chemical analogue of a corresponding naturally occurring amino acid.

### Methods of Treatment

Provided herein are compositions (e.g., pharmaceutical compositions), methods, kits and reagents for prevention and/or treatment of EBV viral infection in humans and other mammals. MV-EBV immunogenic compositions can be used as therapeutic or prophylactic agents. They may be used in medicine to prevent and/or treat infectious disease. In exemplary aspects, the MV-EBV immunogenic compositions of the present disclosure are used to provide prophylactic protection from EBV virus infection. Prophylactic protection from EBV virus can be achieved following administration of an MV-EBV immunogenic compositions of the present disclosure. Immunogenic compositions can be administered once, twice, three times, four times or more. It is possible, although less desirable, to administer the immunogenic compositions to an EBV-infected individual to achieve a therapeutic response. Dosing may need to be adjusted accordingly.

In some embodiments, the EBV immunogenic compositions of the present disclosure can be used as a method of preventing an EBV virus infection in a subject, the method comprising administering to the subject at least one MV-EBV immunogenic compositions as provided herein. In some embodiments, the MV-EBV immunogenic compositions of the present disclosure can be used as a method of treating an EBV virus infection in a subject, the method comprising administering to said subject at least one MV-EBV immunogenic compositions as provided herein. In some embodiments, the MV-EBV immunogenic compositions of the present disclosure can be used as a method of reducing an incidence of EBV virus infection in a subject, the method comprising administering to said subject at least one MV-EBV immunogenic compositions as provided herein. In some embodiments, the MV-EBV immunogenic compositions of the present disclosure can be used as a method of inhibiting spread of EBV virus from a first subject infected with EBV virus to a second subject not infected with EBV virus, the method comprising administering to at least one of said first subject and said second subject at least one MV-EBV immunogenic composition as provided herein.

A method of eliciting an immune response in a subject against an EBV virus is provided in aspects of the invention. The method involves administering to the subject an EBV immunogenic compositions described herein, thereby inducing in the subject an immune response specific to EBV virus antigenic polypeptide or an immunogenic fragment thereof.

A prophylactically effective dose is a therapeutically effective dose that prevents infection with the virus at a clinically acceptable level. In some embodiments the therapeutically effective dose is a dose listed in a package insert for the vaccine.

### Therapeutic and Prophylactic Compositions

Provided herein are compositions (e.g., pharmaceutical compositions), methods, kits and reagents for prevention, treatment or diagnosis of EBV infection in humans and other mammals, for example. MV-EBV immunogenic compositions can be used as therapeutic or prophylactic agents. They may be used in medicine to prevent and/or treat infectious disease. In some embodiments, immunogenic compositions in accordance with the present disclosure may be used for prevention and/or treatment of EBV infection.

MV-EBV immunogenic compositions may be administered prophylactically or therapeutically as part of an active immunization scheme to healthy individuals or early in infection during the incubation phase or during active infection after onset of symptoms. In some embodiments, the amount of immunogenic compositions of the present disclosure provided to a cell, a tissue or a subject may be an amount effective for immune prophylaxis.

MV-EBV immunogenic compositions may be administrated with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In some embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, MV-EBV immunogenic compositions may be administered intramuscularly or intradermally. In some embodiments, MV-EBV immunogenic compositions are administered intramuscularly.

MV-EBV immunogenic compositions may be utilized in various settings depending on the prevalence of the infection or the degree or level of unmet medical need. Immunogenic compositions have superior properties in that they produce much larger antibody titers and produce responses early than commercially available anti-viral agents/compositions.

Provided herein are pharmaceutical compositions including MV-EBV immunogenic compositions optionally in combination with one or more pharmaceutically acceptable excipients.

MV-EBV immunogenic compositions may be formulated or administered in combination with one or more pharmaceutically acceptable excipients. In some embodiments, immunogenic compositions comprise at least one additional active substances, such as, for example, a therapeutically active substance, a prophylactically active substance, or a combination of both. Immunogenic compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety). In some embodiments, MV-EBV immunogenic compositions are administered to humans, human patients or subjects.

Formulations of the MV-EBV immunogenic compositions described herein may be prepared by any method known or hereafter developed in the art of pharmacology. In general, such preparatory methods include the step of bringing the active ingredient (e.g., polypeptide or polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

### Modes of MV-EBV Immunogenic Composition Administration

MV-EBV immunogenic compositions may be administered by any route which results in a therapeutically effective outcome. These include, but are not limited, to intradermal, intramuscular, intranasal and/or subcutaneous administration. The present disclosure provides methods comprising administering immunogenic compositions to a subject in need thereof. The exact amount required will vary from subject to subject, depending on the species, age, and general condition of the subject, the severity of the disease, the particular composition, its mode of administration, its mode of activity, and the like. MV-EBV immunogenic compositions are typically formulated in dosage unit form for ease of administration and uniformity of dosage. It will be understood, however, that the total daily usage of immunogenic compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

An MV-EBV immunogenic pharmaceutical composition described herein can be formulated into a dosage form described herein, such as an intranasal, intratracheal, or injectable (e.g., intravenous, intraocular, intravitreal, intramuscular, intradermal, intracardiac, intraperitoneal, intranasal and subcutaneous).

### MV-EBV Virus Immunogenic Formulations and Methods of Use

Some aspects of the present disclosure provide formulations of the MV-EBV immunogenic composition, wherein the immunogenic composition is formulated in an effective amount to produce an antigen specific immune response in a subject (e.g., production of antibodies specific to an EBV antigenic polypeptide). “An effective amount” is a dose of a vaccine effective to produce an antigen-specific immune response. Also provided herein are methods of inducing an antigen-specific immune response in a subject.

In some embodiments, the antigen-specific immune response is characterized by measuring an anti-EBV antigenic polypeptide antibody titer produced in a subject administered an EBV immunogenic composition as provided herein. An antibody titer is a measurement of the level or concentration of antibodies within a sample from a subject, for example, antibodies that are specific to a particular antigen (e.g., an EBV antigenic polypeptide) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous immunogenic compositions was effective, and to identify any recent or prior infections. In accordance with the present disclosure, an antibody titer may be used to determine the strength of an immune response induced in a subject by an MV-EBV immunogenic composition.

The inventions of the disclosure are not limited in their application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

The following examples are meant to be illustrative and should not be construed as further limiting. The contents of the figures and all references, patents, and published patent applications cited throughout this application are expressly incorporated herein by reference.

### Example 1: Monovalent Recombinant Measles Viruses Carrying EBV Antigens

Monovalent, bivalent, and trivalent measles-based vectors expressing EBV antigens gp350, LMP2, gH, gL and gp42 were created, as shown in Table 2 below. The gp350 and LMP2 antigens of B-02 were separated by ATUa (SEQ ID NO: 75). For T-03, the gH and gL antigens were separated by ATUa (SEQ ID NO: 75), and the gL and gp42 antigens were separated by ATUb (SEQ ID NO: 78). For each protein, a codon-optimization tool (www.novoprolabs.com/tools/codon-optimization) was used to generate a DNA stretch that encodes the desired protein with optimized codon usage for Homo sapiens. Long protein frames encoded in other frames as well as MV-editing sequences were removed by changing the 3rd nucleotide of a codon. The codon-optimized sequence was then reviewed to confirm that the codon usage of the alternative codon was of similar frequency to the originally suggested codon using a Homo sapiens codon frequency table (www.researchgate.net/figure/Homo-sapiens-codon-usage_tbl1_322560620).

TABLE 2
MV-EBV constructs

| Vector name; | | |
|---|---|---|
| SEQ ID NO | Insert | ATU location |
| M-01 (SEQ ID | gp350 | ATU3 |
| NO: 59) | | |
| B-02 (SEQ ID | gp350_ATUa_{—} | ATU3 |
| NO: 60) | LMP2 | |
| T-03 (SEQ ID | gH_ATUa_gL_{—} | ATU3 |
| NO: 61) | ATUb_gp42 | |

### Antigen Expression

To assess expression of EBV antigens from the MV-EBV constructs, Vero cells were infected with constructs at passage 2. Cells were then stained for relevant EBV proteins: gp350 alone (SEQ ID NO: 54); gp350 and LMP2 (SEQ ID NO: 55); and gH (SEQ ID NO: 56), gL (SEQ ID NO: 57), and gp42 (SEQ ID NO: 58). Cells were also stained for MV NP protein as follows. Infected Vero cells were fixed, permeabilized, and blocked with PBS/0.05% Tween 20/1% BSA. After blocking, measles and EBV protein specific staining was performed using Abs to EBV gp350 (mouse anti-EBV gp350/250 Ab; Sigma; Cat. No.: MAB8183), EBV LMP-2A (rat anti-EBV LMP-2A Ab; Santa Cruz; Cat. No.: SC-101314), EBV gH/gL/gp42 complex (mouse anti-EBV gH/gL/gp42 conformational domain Ab; Antibodies-Online; Cat. No.: ABIN1605947) and MV NP (Rabbit anti-measles NP Ab, OniGene, AP55070SU-N) diluted in blocking solution. Measles NP expression was detected using either AlexaFluor 594 goat anti-rabbit secondary antibody (Life Technologies A11012) or AlexaFluor 488 goat anti-rabbit secondary antibody (Life Technologies A11008). EBV gH/gL/gp42 expression was detected using Alexa Fluor 488 AffiniPure Goat Anti-Mouse IgG (H+L) secondary antibody (Jackson Laboratories; 115-545-003). EBV LMP-2A expression was detected using AlexaFluor594 goat anti-rat secondary antibody (Life Technologies A11007). EMV gp350 expression was detected using either AlexaFluor 647 goat anti-mouse IgG(H+L) secondary antibody (Life Technologies A21235) or Alexa Fluor 488 AffiniPure Goat Anti-Mouse IgG (H+L) (Jackson Laboratories; 115-545-003). Images were captured using the ImageXpress Pico Automated Cell Imagine (Molecular devices).

Vero cells infected by all three MV-EBV constructs were positive for both MV NP protein and heterologous EBV proteins (photomicrographs not shown).

### Example 2: Growth Kinetics of MV-EBV Constructs

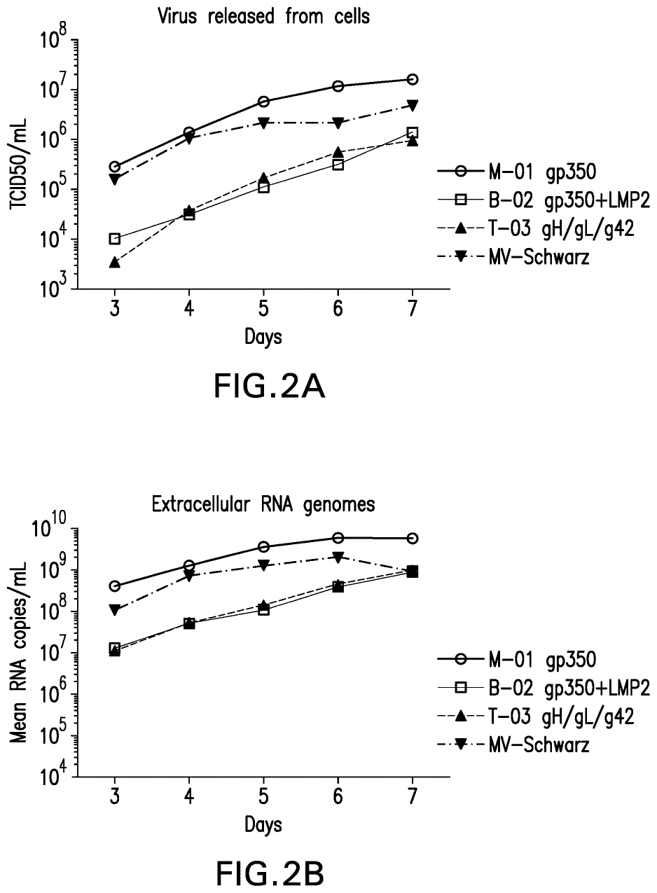

To assess the replication capacity of the various viruses, growth curve analysis was performed on MV-EBV constructs M-01 (gp350), B-02 (gp350_ATUa_LMP2), and T-03 (gH_ATUa_gL_ATUb_gp42). Virus material of passage 1 was used for analysis. Briefly, Vero cells seeded in T-25 flasks were infected with a defined MOI of 0.01. As a control, additional cell culture flasks were infected with the parental MV-Schwarz at the same MOI. Supernatants were then collected at different time points and the titer of virus released from cells was determined by TCID50 assay, as well as by detection of extracellular RNA genomes. RNA genomes were detected as follows. Viral RNA was isolated from supernatants using a QIAamp vRNA kit (Qiagen). vRNA (2 μl) was used in a one-step RT-PCR using Luna Universal Probe One-Step RT-qPCR kit (NEB; Cat. No.: E3006L) together with the following primers and probe: MV_F2 (5′-TCGAGTCCCTCACGCTTACAG-3′; SEQ ID NO: 89), MV_R2 (5′-GGCGGTGCTTGATGTTCTGA-3′; SEQ ID NO: 90) and MV_P2_Probe (5′-FAM-CTGGAGGACCCTACACTG-MGB-3′; SEQ ID NO: 91). Reactions were performed in a final volume of 20 μl in the presence of 200 nM probe and 400 nM (each) forward and reverse primers. Cycling conditions consisted of 55° C. for 10 min, 95° C. for 1 min, followed by 40 cycles of 95° C. for 10 sec and 56° C. for 30 sec. RNA standards were prepared by in vitro transcription of MV Genome Fragment DNA plasmid (pTOPO-T7MV9119-20as), linearized by BamHI digestion, using T7 RNA polymerase (MEGAscript T7 Transcription kit; Invitrogen; AM1334). The results are shown in FIGS. 2A and 2B.

All three MV-EBV constructs released further MV-EBV construct after cell infection, with both the TCID50/mL of virus released from cells (FIG. 2A) and mean RNA copies/mL of extracellular RNA genomes (FIG. 2B) for all three constructs increasing between days 3 and 7 (FIG. 2A). The size of each EBV insert appeared to influence the replication of the virus, with less virus released from cells infected with B-02 (gp350+LMP2; 2724 bp+2334 bp, respectively) and T-03 (gH/gL/gp42; 2124+672+414 bp, respectively), compared to M-01 (gp350; 2724 bp cDNA).

### Example 3: Genetic Stability of MV-EBV Constructs During Passaging

The genetic stability of the MV-EBV constructs M-01, B-02, and T-03 was tested in T-flasks. The virus was passaged five times. Infection of each passage was performed with a MOI of 0.01, and virus was propagated for 6 days at 32° C., before the supernatant was harvested. Harvested supernatants of each passage were subjected to RNA extraction, subsequent cDNA synthesis amplification of the genomic insert using primers ATU3-PCR-1F1 (SEQ ID NO: 62) and ATU3-PCR-1R1 (SEQ ID NO: 63) or Seq_Pr_LMP2_278 (SEQ ID NO: 64), and agarose gel electrophoresis to check for large-scale deletions in the MV-EBV genomic inserts. Passage 4 (p4) harvested supernatants from M-01 and T-03 MV vectors were also subjected to Sanger sequencing of the insert.

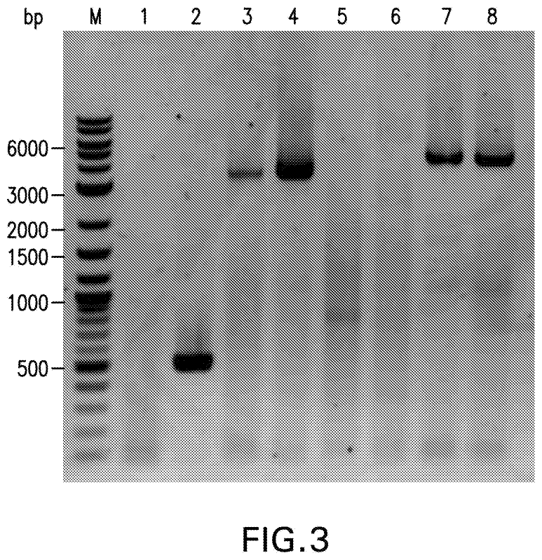

Amplicons of the genomic insert are shown in FIG. 3. Gel electrophoresis revealed that a PCR product of the anticipated size was readily detectable over the course of the virus passages for M-01 and T-03, but not for B-02. In addition, Sanger sequencing of the insert at Passage 4 confirmed the integrity of the insert. Together these data show that MV constructs M-01 and T-03 are genetically stable. Table 3 below lists the gel lanes, samples, and respective expected band size.

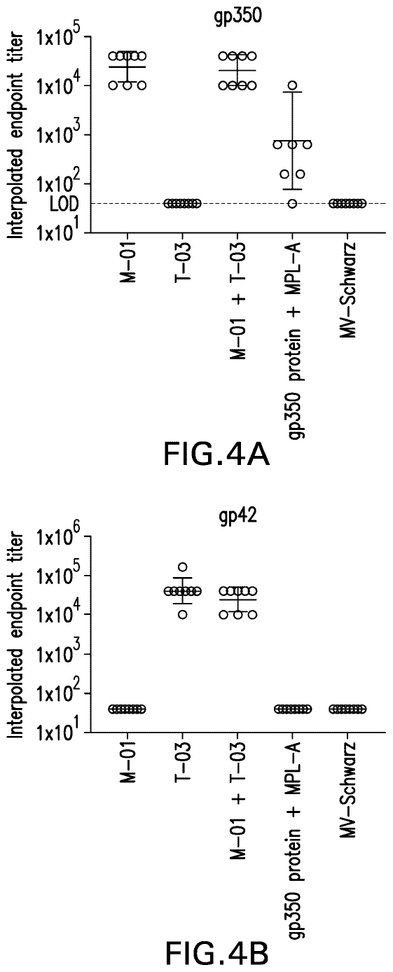

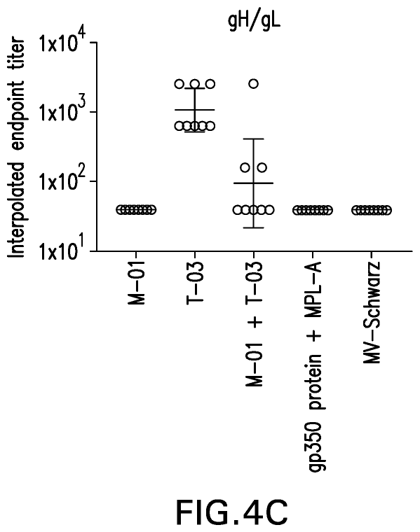

TABLE 3
Sample and expected band sizes for FIG. 3.

| Lane | Sample | Expected band size (bp) |
|---|---|---|
| M | DNA marker | As indicated in FIG. 4 |
| 1 | Neg. Control | — |
| 2 | MV-Schwarz p2 | 503 |
| 3 | M-01 p4 | 3341 |
| 4 | M-01 p4 | 3341 |
| 5 | B-02 p4 | 5819 |
| 6 | B-02 p4 | 5819 |
| 7 | T-03 p4 | 4073 |
| 8 | T-03 p4 | 4073 |

Vectors express the payload in its correct confirmation. The gH/gL/gp42 complex is formed upon replication in vitro as recognized by an EBV-specific antibody.

MV-gp350 and MV-gH/gL/gp42 alone or in combination induced robust antigen-specific antibody responses in cotton rats.

MV-gp350 and MV-gH/gL/gp42 alone or in combination could induce EBV neutralizing antibodies.

### Example 4: MV-EBV Immunogenicity in Cotton Rats

Mice and guinea pigs are well established models to test and screen EBV vaccines preclinically. However, these animal models are not permissive for measles virus infection. Cotton rats were selected as the preclinical small animal model because they are semi-permissive for measles virus infection and can be infected with EBV following vaginal challenge. Like guinea pigs, cotton rats can exhibit lesion formation after EBV infection and can experience spontaneous recurrent vaginal disease after recovering from initial infection. Therefore, cotton rats can serve as a model for testing vaccine efficacy to prevent reactivation and recurrent disease.

### Measuring Antibody Liters

Antibody titers were measured using ELISA methodologies. Maxisorp plates were coated with recombinantly expressed gp350, gp42, or gH/gL, and blocked with 3% milk in PBS-T. 4-fold serial serum dilutions were prepared starting from a 1:40 dilution in blocking buffer and transferred to assay plates. Binding was detected using species-specific HRP-conjugated secondary antibodies. Endpoint dilution titers of each serum sample to recombinant gp350, gp42, gH/gL were determined by direct ELISA. The endpoint titer is defined as the reciprocal of the highest analyte dilution that gives a reading above the background. Serum samples that did not generate a signal that crossed the threshold value were reported as a titer of 40.

Serum neutralization titers were determined using the method of Li et al., Vaccine. 2022 Jun. 9; 40(26):3638-3646. Serum neutralization titers were determined on Akata 4E3 B cells and HEK293T epithelial cells. Akata EBV-GFP was used as infection virus, 4E3 human B lymphoma cell and human embryo kidney HEK293T cells were used as cell substrates for B cell and epithelial cell tropic assays, respectively. 4E3 cells were seeded at 2.5×10⁴ cells/well and 293T cells were seeded at 1.5×10⁴ cells/well, in 50 μl/well Roswell Park Memorial Institute (RPMI) 1640 cell culture medium with no phenol red complete medium (Gibco/ThermoFisher Scientific, Waltham, MA USA, cat. #11835-030) in 96-well black wall transparent plates (Corning, Corning, NY USA, cat. #3904). The cell plates were then cultured for 4 hours for epithelial cell attachment at 37° C. and 5% CO₂. During the interval, serum or antibody samples were 2-fold serially diluted (60 μl to 60 μl format) in 96-well plates (Costar u-bottom well, Corning, Corning, NY USA, cat. #3799) with RPMI 1640 no phenol red complete medium as diluent by a Biomek 2000 liquid handler. A 60 μl volume of RPMI no phenol red complete medium containing Akata EBV-GFP virus at about 1.5×10⁵ fluorescent focus units (ffu) per ml was added to each well, for a total volume of 120 μl/well. The wells of column 12 were used as no antibody virus-only controls.

The antibody and virus mixture plates were briefly mixed for 5 min in a plate shaker and kept at room temperature for 1 hour. Those antibody and virus mixtures were then added to 4E3 and 293T cell plates at 50 μl/well by Biomek 2000 (Beckman Coulter, Indianapolis, IN USA), in a well-to-well plate replica format. The final culture medium in cell plates was 100 μl/well, and the resulting antibody dilution at this stage was the final dilution recorded. The output of the green EBV-GFP virus infected B cells in control wells after two days is usually around 1000 ffu. The plates were cultured in a 37° C., 5% CO₂ incubator for 2 days, then scanned by an Acumen™ Cellista Laser Scanning Imaging Cytometer (SPT Labs, Shanghai CN) to count the number of GFP expressing cells as fluorescent focus units (ffu), which reflects the EBV-GFP virus infections. NT (neutralization percentage) of each well was converted and recorded from raw readings using the formula: NT=((Control well ffu counts—Antibody well ffu counts)/Control well ffu counts)×100%. NT₅₀ titer values representing samples' neutralization potentials were extracted by a Merck & Co., Inc., Kenilworth, NJ, USA developed Excel/Solver based program with 4-parameter (y=d+(a−d)/(1+(x/c)\hat{ }b)) logistic curve fitting algorithm from the 11 neutralization points of serial dilutions. NT 0% or NT negative value means no neutralization, NT₅₀ being a calculated value means the titer in that half of the virus input were neutralized theoretically (NT 50%). NT 100% means complete virus neutralization.

### MV Vector Administration

Mono-, di-, or trivalent antigen measles virus vectors (M-01, B-02, T-03) were administered IM to cotton rats in two doses 28 days apart at 1×10⁵ TCID50. Sera were collected at four weeks post dose one (Day 28), and at two (Day 42) and three (Day 49) weeks post dose two. The gp350, gp42, or gH/gL specific IgG antibody titers were determined by ELISA using purified recombinant proteins as capture-antigen substrates. Serum antibodies capable of neutralizing EBV in the presence of exogenous complement also were quantified at the same time points.

### Results

As shown in FIG. 4A, gp350 antibody responses were only observed in MVs expressing the gp350 antigen, and were superior to the antibody response to gp350 protein administered with MPL-A. Similarly, the gp42 antibody-specific responses were only observed in MVs expressing the gp42 antigen (FIG. 4B). The virus-induced gH/gL antibody responses were also greater than the responses generated by the antibody response to gp350 protein administered with MPL-A (FIG. 4C).

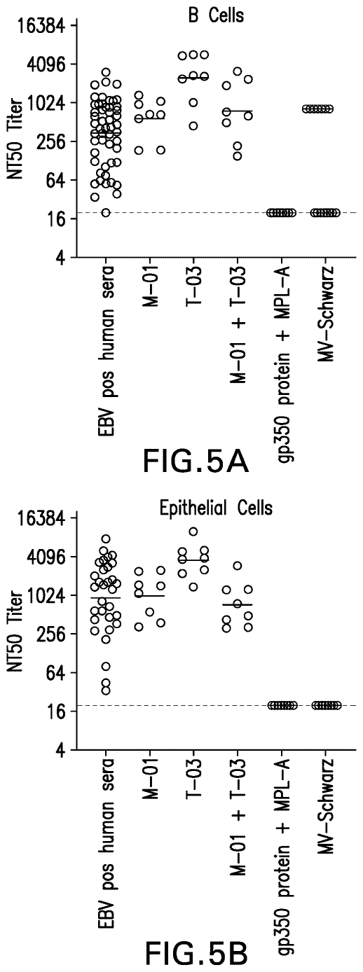

The EBV neutralization assay against B cells (FIG. 5A) and epithelial cells (FIG. 5B) showed that M-01, B-02, and T-03 administered to cotton rats all had superior neutralization ability from their serum antibodies. Collectively, these results demonstrate that the M-01, B-02, and T-03 induce robust antibody responses.

TABLE 7
Sequences

| SEQ ID | | |
|---|---|---|
| NO: | Description | Sequence |
| 1 | pBluescript KS | CACCTAAATTGTAAGCGTTAATATTTTGTTAAAATTCG |
| | (+) (GenBank | CGTTAAATTTTTGTTAAATCAGCTCATTTTTTAACCAAT |
| | X52331.1) | AGGCCGAAATCGGCAAAATCCCTTATAAATCAAAAGA |
| | | ATAGACCGAGATAGGGTTGAGTGTTGTTCCAGTTTGGA |
| | | ACAAGAGTCCACTATTAAAGAACGTGGACTCCAACGTC |
| | | AAAGGGCGAAAAACCGTCTATCAGGGCGATGGCCCAC |
| | | TACGTGAACCATCACCCTAATCAAGTTTTTTGGGGTCG |
| | | AGGTGCCGTAAAGCACTAAATCGGAACCCTAAAGGGA |
| | | GCCCCCGATTTAGAGCTTGACGGGGAAAGCCGGCGAA |
| | | CGTGGCGAGAAAGGAAGGGAAGAAAGCGAAAGGAGC |
| | | GGGCGCTAGGGCGCTGGCAAGTGTAGCGGTCACGCTG |
| | | CGCGTAACCACCACACCCGCCGCGCTTAATGCGCCGCT |
| | | ACAGGGCGCGTCCCATTCGCCATTCAGGCTGCGCAACT |
| | | GTTGGGAAGGGCGATCGGTGCGGGCCTCTTCGCTATTA |
| | | CGCCAGCTGGCGAAAGGGGGATGTGCTGCAAGGCGAT |
| | | TAAGTTGGGTAACGCCAGGGTTTTCCCAGTCACGACGT |
| | | TGTAAAACGACGGCCAGTGAATTGTAATACGACTCACT |
| | | ATAGGGCGAATTGGAGCTCCACCGCGGTGGCGGCCGC |
| | | TCTAGAACTAGTGGATCCCCCGGGCTGCAGGAATTCGA |
| | | TATCAAGCTTATCGATACCGTCGACCTCGAGGGGGGGC |
| | | CCGGTACCCAGCTTTTGTTCCCTTTAGTGAGGGTTAATT |
| | | TCGAGCTTGGCGTAATCATGGTCATAGCTGTTTCCTGT |
| | | GTGAAATTGTTATCCGCTCACAATTCCACACAACATAC |
| | | GAGCCGGAAGCATAAAGTGTAAAGCCTGGGGTGCCTA |
| | | ATGAGTGAGCTAACTCACATTAATTGCGTTGCGCTCAC |
| | | TGCCCGCTTTCCAGTCGGGAAACCTGTCGTGCCAGCTG |
| | | CATTAATGAATCGGCCAACGCGCGGGGAGAGGCGGTT |
| | | TGCGTATTGGGCGCTCTTCCGCTTCCTCGCTCACTGACT |
| | | CGCTGCGCTCGGTCGTTCGGCTGCGGCGAGCGGTATCA |
| | | GCTCACTCAAAGGCGGTAATACGGTTATCCACAGAATC |
| | | AGGGGATAACGCAGGAAAGAACATGTGAGCAAAAGGC |
| | | CAGCAAAAGGCCAGGAACCGTAAAAAGGCCGCGTTGC |
| | | TGGCGTTTTTCCATAGGCTCCGCCCCCCTGACGAGCAT |
| | | CACAAAAATCGACGCTCAAGTCAGAGGTGGCGAAACC |
| | | CGACAGGACTATAAAGATACCAGGCGTTTCCCCCTGGA |
| | | AGCTCCCTCGTGCGCTCTCCTGTTCCGACCCTGCCGCTT |
| | | ACCGGATACCTGTCCGCCTTTCTCCCTTCGGGAAGCGT |
| | | GGCGCTTTCTCATAGCTCACGCTGTAGGTATCTCAGTTC |
| | | GGTGTAGGTCGTTCGCTCCAAGCTGGGCTGTGTGCACG |
| | | AACCCCCCGTTCAGCCCGACCGCTGCGCCTTATCCGGT |
| | | AACTATCGTCTTGAGTCCAACCCGGTAAGACACGACTT |
| | | ATCGCCACTGGCAGCAGCCACTGGTAACAGGATTAGC |
| | | AGAGCGAGGTATGTAGGCGGTGCTACAGAGTTCTTGA |
| | | AGTGGTGGCCTAACTACGGCTACACTAGAAGGACAGT |
| | | ATTTGGTATCTGCGCTCTGCTGAAGCCAGTTACCTTCG |
| | | GAAAAAGAGTTGGTAGCTCTTGATCCGGCAAACAAAC |
| | | CACCGCTGGTAGCGGTGGTTTTTTTGTTTGCAAGCAGC |
| | | AGATTACGCGCAGAAAAAAAGGATCTCAAGAAGATCC |
| | | TTTGATCTTTTCTACGGGGTCTGACGCTCAGTGGAACG |
| | | AAAACTCACGTTAAGGGATTTTGGTCATGAGATTATCA |
| | | AAAAGGATCTTCACCTAGATCCTTTTAAATTAAAAATG |
| | | AAGTTTTAAATCAATCTAAAGTATATATGAGTAAACTT |
| | | GGTCTGACAGTTACCAATGCTTAATCAGTGAGGCACCT |
| | | ATCTCAGCGATCTGTCTATTTCGTTCATCCATAGTTGCC |
| | | TGACTCCCCGTCGTGTAGATAACTACGATACGGGAGGG |
| | | CTTACCATCTGGCCCCAGTGCTGCAATGATACCGCGAG |
| | | ACCCACGCTCACCGGCTCCAGATTTATCAGCAATAAAC |
| | | CAGCCAGCCGGAAGGGCCGAGCGCAGAAGTGGTCCTG |
| | | CAACTTTATCCGCCTCCATCCAGTCTATTAATTGTTGCC |
| | | GGGAAGCTAGAGTAAGTAGTTCGCCAGTTAATAGTTTG |
| | | CGCAACGTTGTTGCCATTGCTACAGGCATCGTGGTGTC |
| | | ACGCTCGTCGTTTGGTATGGCTTCATTCAGCTCCGGTTC |
| | | CCAACGATCAAGGCGAGTTACATGATCCCCCATGTTGT |
| | | GCAAAAAAGCGGTTAGCTCCTTCGGTCCTCCGATCGTT |
| | | GTCAGAAGTAAGTTGGCCGCAGTGTTATCACTCATGGT |
| | | TATGGCAGCACTGCATAATTCTCTTACTGTCATGCCATC |
| | | CGTAAGATGCTTTTCTGTGACTGGTGAGTACTCAACCA |
| | | AGTCATTCTGAGAATAGTGTATGCGGCGACCGAGTTGC |
| | | TCTTGCCCGGCGTCAATACGGGATAATACCGCGCCACA |
| | | TAGCAGAACTTTAAAAGTGCTCATCATTGGAAAACGTT |
| | | CTTCGGGGCGAAAACTCTCAAGGATCTTACCGCTGTTG |
| | | AGATCCAGTTCGATGTAACCCACTCGTGCACCCAACTG |
| | | ATCTTCAGCATCTTTTACTTTCACCAGCGTTTCTGGGTG |
| | | AGCAAAAACAGGAAGGCAAAATGCCGCAAAAAAGGG |
| | | AATAAGGGCGACACGGAAATGTTGAATACTCATACTCT |
| | | TCCTTTTTCAATATTATTGAAGCATTTATCAGGGTTATT |
| | | GTCTCATGAGCGGATACATATTTGAATGTATTTAGAAA |
| | | AATAAACAAATAGGGGTTCCGCGCACATTTCCCCGAAA |
| | | AGTGC |
| 2 | pBluescript II | CTAAATTGTAAGCGTTAATATTTTGTTAAAATTCGCGTT |
| | KS (+) | AAATTTTTGTTAAATCAGCTCATTTTTTAACCAATAGGC |
| | (GenBank | CGAAATCGGCAAAATCCCTTATAAATCAAAAGAATAG |
| | X52327.1) | ACCGAGATAGGGTTGAGTGTTGTTCCAGTTTGGAACAA |
| | | GAGTCCACTATTAAAGAACGTGGACTCCAACGTCAAA |
| | | GGGCGAAAAACCGTCTATCAGGGCGATGGCCCACTAC |
| | | GTGAACCATCACCCTAATCAAGTTTTTTGGGGTCGAGG |
| | | TGCCGTAAAGCACTAAATCGGAACCCTAAAGGGAGCC |
| | | CCCGATTTAGAGCTTGACGGGGAAAGCCGGCGAACGT |
| | | GGCGAGAAAGGAAGGGAAGAAAGCGAAAGGAGCGGG |
| | | CGCTAGGGCGCTGGCAAGTGTAGCGGTCACGCTGCGC |
| | | GTAACCACCACACCCGCCGCGCTTAATGCGCCGCTACA |
| | | GGGCGCGTCCCATTCGCCATTCAGGCTGCGCAACTGTT |
| | | GGGAAGGGCGATCGGTGCGGGCCTCTTCGCTATTACGC |
| | | CAGCTGGCGAAAGGGGGATGTGCTGCAAGGCGATTAA |
| | | GTTGGGTAACGCCAGGGTTTTCCCAGTCACGACGTTGT |
| | | AAAACGACGGCCAGTGAGCGCGCGTAATACGACTCAC |
| | | TATAGGGCGAATTGGAGCTCCACCGCGGTGGCGGCCG |
| | | CTCTAGAACTAGTGGATCCCCCGGGCTGCAGGAATTCG |
| | | ATATCAAGCTTATCGATACCGTCGACCTCGAGGGGGGG |
| | | CCCGGTACCCAGCTTTTGTTCCCTTTAGTGAGGGTTAAT |
| | | TGCGCGCTTGGCGTAATCATGGTCATAGCTGTTTCCTGT |
| | | GTGAAATTGTTATCCGCTCACAATTCCACACAACATAC |
| | | GAGCCGGAAGCATAAAGTGTAAAGCCTGGGGTGCCTA |
| | | ATGAGTGAGCTAACTCACATTAATTGCGTTGCGCTCAC |
| | | TGCCCGCTTTCCAGTCGGGAAACCTGTCGTGCCAGCTG |
| | | CATTAATGAATCGGCCAACGCGCGGGGAGAGGCGGTT |
| | | TGCGTATTGGGCGCTCTTCCGCTTCCTCGCTCACTGACT |
| | | CGCTGCGCTCGGTCGTTCGGCTGCGGCGAGCGGTATCA |
| | | GCTCACTCAAAGGCGGTAATACGGTTATCCACAGAATC |
| | | AGGGGATAACGCAGGAAAGAACATGTGAGCAAAAGGC |
| | | CAGCAAAAGGCCAGGAACCGTAAAAAGGCCGCGTTGC |
| | | TGGCGTTTTTCCATAGGCTCCGCCCCCCTGACGAGCAT |
| | | CACAAAAATCGACGCTCAAGTCAGAGGTGGCGAAACC |
| | | CGACAGGACTATAAAGATACCAGGCGTTTCCCCCTGGA |
| | | AGCTCCCTCGTGCGCTCTCCTGTTCCGACCCTGCCGCTT |
| | | ACCGGATACCTGTCCGCCTTTCTCCCTTCGGGAAGCGT |
| | | GGCGCTTTCTCATAGCTCACGCTGTAGGTATCTCAGTTC |
| | | GGTGTAGGTCGTTCGCTCCAAGCTGGGCTGTGTGCACG |
| | | AACCCCCCGTTCAGCCCGACCGCTGCGCCTTATCCGGT |
| | | AACTATCGTCTTGAGTCCAACCCGGTAAGACACGACTT |
| | | ATCGCCACTGGCAGCAGCCACTGGTAACAGGATTAGC |
| | | AGAGCGAGGTATGTAGGCGGTGCTACAGAGTTCTTGA |
| | | AGTGGTGGCCTAACTACGGCTACACTAGAAGGACAGT |
| | | ATTTGGTATCTGCGCTCTGCTGAAGCCAGTTACCTTCG |
| | | GAAAAAGAGTTGGTAGCTCTTGATCCGGCAAACAAAC |
| | | CACCGCTGGTAGCGGTGGTTTTTTTGTTTGCAAGCAGC |
| | | AGATTACGCGCAGAAAAAAAGGATCTCAAGAAGATCC |
| | | TTTGATCTTTTCTACGGGGTCTGACGCTCAGTGGAACG |
| | | AAAACTCACGTTAAGGGATTTTGGTCATGAGATTATCA |
| | | AAAAGGATCTTCACCTAGATCCTTTTAAATTAAAAATG |
| | | AAGTTTTAAATCAATCTAAAGTATATATGAGTAAACTT |
| | | GGTCTGACAGTTACCAATGCTTAATCAGTGAGGCACCT |
| | | ATCTCAGCGATCTGTCTATTTCGTTCATCCATAGTTGCC |
| | | TGACTCCCCGTCGTGTAGATAACTACGATACGGGAGGG |
| | | CTTACCATCTGGCCCCAGTGCTGCAATGATACCGCGAG |
| | | ACCCACGCTCACCGGCTCCAGATTTATCAGCAATAAAC |
| | | CAGCCAGCCGGAAGGGCCGAGCGCAGAAGTGGTCCTG |
| | | CAACTTTATCCGCCTCCATCCAGTCTATTAATTGTTGCC |
| | | GGGAAGCTAGAGTAAGTAGTTCGCCAGTTAATAGTTTG |
| | | CGCAACGTTGTTGCCATTGCTACAGGCATCGTGGTGTC |
| | | ACGCTCGTCGTTTGGTATGGCTTCATTCAGCTCCGGTTC |
| | | CCAACGATCAAGGCGAGTTACATGATCCCCCATGTTGT |
| | | GCAAAAAAGCGGTTAGCTCCTTCGGTCCTCCGATCGTT |
| | | GTCAGAAGTAAGTTGGCCGCAGTGTTATCACTCATGGT |
| | | TATGGCAGCACTGCATAATTCTCTTACTGTCATGCCATC |
| | | CGTAAGATGCTTTTCTGTGACTGGTGAGTACTCAACCA |
| | | AGTCATTCTGAGAATAGTGTATGCGGCGACCGAGTTGC |
| | | TCTTGCCCGGCGTCAATACGGGATAATACCGCGCCACA |
| | | TAGCAGAACTTTAAAAGTGCTCATCATTGGAAAACGTT |
| | | CTTCGGGGCGAAAACTCTCAAGGATCTTACCGCTGTTG |
| | | AGATCCAGTTCGATGTAACCCACTCGTGCACCCAACTG |
| | | ATCTTCAGCATCTTTTACTTTCACCAGCGTTTCTGGGTG |
| | | AGCAAAAACAGGAAGGCAAAATGCCGCAAAAAAGGG |
| | | AATAAGGGCGACACGGAAATGTTGAATACTCATACTCT |
| | | TCCTTTTTCAATATTATTGAAGCATTTATCAGGGTTATT |
| | | GTCTCATGAGCGGATACATATTTGAATGTATTTAGAAA |
| | | AATAAACAAATAGGGGTTCCGCGCACATTTCCCCGAAA |
| | | AGTGCCAC |
| 3 | pTM-MVSchw | GCGGCCGCTAATACGACTCACTATAGGGCCAACTTTGT |
| | T7 promoter: nt | TTGGTCTGATGAGTCCGTGAGGACGAAACCCGGAGTCC |
| | 9-28 | CGGGTCACCAAACAAAGTTGGGTAAGGATAGTTCAAT |
| | Hammerhead | CAATGATCATCTTCTAGTGCACTTAGGATTCAAGATCC |
| | ribozyme: nt | TATTATCAGGGACAAGAGCAGGATTAGGGATATCCGA |
| | 29-82 | GATGGCCACACTTTTAAGGAGCTTAGCATTGTTCAAAA |
| | N gene: nt | GAAACAAGGACAAACCACCCATTACATCAGGATCCGG |
| | 1138-,767 | TGGAGCCATCAGAGGAATCAAACACATTATTATAGTAC |
| | P gene: nt | CAATCCCTGGAGATTCCTCAATTACCACTCGATCCAGA |
| | 1,830-3,484 | CTTCTGGACCGGTTGGTGAGGTTAATTGGAAACCCGGA |
| | M gene: nt | TGTGAGCGGGCCCAAACTAACAGGGGCACTAATAGGT |
| | 3,488-4,953 | ATATTATCCTTATTTGTGGAGTCTCCAGGTCAATTGATT |
| | F gene: nt | CAGAGGATCACCGATGACCCTGACGTTAGCATAAGGCT |
| | 4,957-7,329 | GTTAGAGGTTGTCCAGAGTGACCAGTCACAATCTGGCC |
| | H gene: nt | TTACCTTCGCATCAAGAGGTACCAACATGGAGGATGAG |
| | 7,333-9,290 | GCGGACCAATACTTTTCACATGATGATCCAATTAGTAG |
| | L gene: nt | TGATCAATCCAGGTTCGGATGGTTCGGGAACAAGGAA |
| | 9,294-15,936 | ATCTCAGATATTGAAGTGCAAGACCCTGAGGGATTCAA |
| | Ribozyme & T7 | CATGATTCTGGGTACCATCCTAGCCCAAATTTGGGTCT |
| | terminator: nt | TGCTCGCAAAGGCGGTTACGGCCCCAGACACGGCAGC |
| | 15,977-16,202 | TGATTCGGAGCTAAGAAGGTGGATAAAGTACACCCAA |
| | | CAAAGAAGGGTAGTTGGTGAATTTAGATTGGAGAGAA |
| | | AATGGTTGGATGTGGTGAGGAACAGGATTGCCGAGGA |
| | | CCTCTCCTTACGCCGATTCATGGTCGCTCTAATCCTGGA |
| | | TATCAAGAGAACACCCGGAAACAAACCCAGGATTGCT |
| | | GAAATGATATGTGACATTGATACATATATCGTAGAGGC |
| | | AGGATTAGCCAGTTTTATCCTGACTATTAAGTTTGGGA |
| | | TAGAAACTATGTATCCTGCTCTTGGACTGCATGAATTT |
| | | GCTGGTGAGTTATCCACACTTGAGTCCTTGATGAACCT |
| | | TTACCAGCAAATGGGGGAAACTGCACCCTACATGGTA |
| | | ATCCTGGAGAACTCAATTCAGAACAAGTTCAGTGCAGG |
| | | ATCATACCCTCTGCTCTGGAGCTATGCCATGGGAGTAG |
| | | GAGTGGAACTTGAAAACTCCATGGGAGGTTTGAACTTT |
| | | GGCCGATCTTACTTTGATCCAGCATATTTTAGATTAGG |
| | | GCAAGAGATGGTAAGGAGGTCAGCTGGAAAGGTCAGT |
| | | TCCACATTGGCATCTGAACTCGGTATCACTGCCGAGGA |
| | | TGCAAGGCTTGTTTCAGAGATTGCAATGCATACTACTG |
| | | AGGACAAGATCAGTAGAGCGGTTGGACCCAGACAAGC |
| | | CCAAGTATCATTTCTACACGGTGATCAAAGTGAGAATG |
| | | AGCTACCGAGATTGGGGGGCAAGGAAGATAGGAGGGT |
| | | CAAACAGAGTCGAGGAGAAGCCAGGGAGAGCTACAGA |
| | | GAAACCGGGCCCAGCAGAGCAAGTGATGCGAGAGCTG |
| | | CCCATCTTCCAACCGGCACACCCCTAGACATTGACACT |
| | | GCAACGGAGTCCAGCCAAGATCCGCAGGACAGTCGAA |
| | | GGTCAGCTGACGCCCTGCTTAGGCTGCAAGCCATGGCA |
| | | GGAATCTCGGAAGAACAAGGCTCAGACACGGACACCC |
| | | CTATAGTGTACAATGACAGAAATCTTCTAGACTAGGTG |
| | | CGAGAGGCCGAGGGCCAGAACAACATCCGCCTACCAT |
| | | CCATCATTGTTATAAAAAACTTAGGAACCAGGTCCACA |
| | | CAGCCGCCAGCCCATCAACCATCCACTCCCACGATTGG |
| | | AGCCAATGGCAGAAGAGCAGGCACGCCATGTCAAAAA |
| | | CGGACTGGAATGCATCCGGGCTCTCAAGGCCGAGCCC |
| | | ATCGGCTCACTGGCCATCGAGGAAGCTATGGCAGCATG |
| | | GTCAGAAATATCAGACAACCCAGGACAGGAGCGAGCC |
| | | ACCTGCAGGGAAGAGAAGGCAGGCAGTTCGGGTCTCA |
| | | GCAAACCATGCCTCTCAGCAATTGGATCAACTGAAGGC |
| | | GGTGCACCTCGCATCCGCGGTCAGGGACCTGGAGAGA |
| | | GCGATGACGACGCTGAAACTTTGGGAATCCCCCCAAG |
| | | AAATCTCCAGGCATCAAGCACTGGGTTACAGTGTTATT |
| | | ACGTTTATGATCACAGCGGTGAAGCGGTTAAGGGAATC |
| | | CAAGATGCTGACTCTATCATGGTTCAATCAGGCCTTGA |
| | | TGGTGATAGCACCCTCTCAGGAGGAGACAATGAATCTG |
| | | AAAACAGCGATGTGGATATTGGCGAACCTGATACCGA |
| | | GGGATATGCTATCACTGACCGGGGATCTGCTCCCATCT |
| | | CTATGGGGTTCAGGGCTTCTGATGTTGAAACTGCAGAA |
| | | GGAGGGGAGATCCACGAGCTCCTGAGACTCCAATCCA |
| | | GAGGCAACAACTTTCCGAAGCTTGGGAAAACTCTCAAT |
| | | GTTCCTCCGCCCCCGGACCCCGGTAGGGCCAGCACTTC |
| | | CGGGACACCCATTAAAAAGGGCACAGACGCGAGATTA |
| | | GCCTCATTTGGAACGGAGATCGCGTCTTTATTGACAGG |
| | | TGGTGCAACCCAATGTGCTCGAAAGTCACCCTCGGAAC |
| | | CATCAGGGCCAGGTGCACCTGCGGGGAATGTCCCCGA |
| | | GTGTGTGAGCAATGCCGCACTGATACAGGAGTGGACA |
| | | CCCGAATCTGGTACCACAATCTCCCCGAGATCCCAGAA |
| | | TAATGAAGAAGGGGGAGACTATTATGATGATGAGCTG |
| | | TTCTCTGATGTCCAAGATATTAAAACAGCCTTGGCCAA |
| | | AATACACGAGGATAATCAGAAGATAATCTCCAAGCTA |
| | | GAATCACTGCTGTTATTGAAGGGAGAAGTTGAGTCAAT |
| | | TAAGAAGCAGATCAACAGGCAAAATATCAGCATATCC |
| | | ACCCTGGAAGGACACCTCTCAAGCATCATGATCGCCAT |
| | | TCCTGGACTTGGGAAGGATCCCAACGACCCCACTGCAG |
| | | ATGTCGAAATCAATCCCGACTTGAAACCCATCATAGGC |
| | | AGAGATTCAGGCCGAGCACTGGCCGAAGTTCTCAAGA |
| | | AACCCGTTGCCAGCCGACAACTCCAAGGAATGACAAA |
| | | TGGACGGACCAGTTCCAGAGGACAGCTGCTGAAGGAA |
| | | TTTCAGCTAAAGCCGATCGGGAAAAAGATGAGCTCAG |
| | | CCGTCGGGTTTGTTCCTGACACCGGCCCTGCATCACGC |
| | | AGTGTAATCCGCTCCATTATAAAATCCAGCCGGCTAGA |
| | | GGAGGATCGGAAGCGTTACCTGATGACTCTCCTTGATG |
| | | ATATCAAAGGAGCCAATGATCTTGCCAAGTTCCACCAG |
| | | ATGCTGATGAAGATAATAATGAAGTAGCTACAGCTCA |
| | | ACTTACCTGCCAACCCCATGCCAGTCGACCCAACTAGT |
| | | ACAACCTAAATCCATTATAAAAAACTTAGGAGCAAAG |
| | | TGATTGCCTCCCAAGGTCCACAATGACAGAGACCTACG |
| | | ACTTCGACAAGTCGGCATGGGACATCAAAGGGTCGAT |
| | | CGCTCCGATACAACCCACCACCTACAGTGATGGCAGGC |
| | | TGGTGCCCCAGGTCAGAGTCATAGATCCTGGTCTAGGC |
| | | GACAGGAAGGATGAATGCTTTATGTACATGTTTCTGCT |
| | | GGGGGTTGTTGAGGACAGCGATTCCCTAGGGCCTCCAA |
| | | TCGGGCGAGCATTTGGGTTCCTGCCCTTAGGTGTTGGC |
| | | AGATCCACAGCAAAGCCCGAAAAACTCCTCAAAGAGG |
| | | CCACTGAGCTTGACATAGTTGTTAGACGTACAGCAGGG |
| | | CTCAATGAAAAACTGGTGTTCTACAACAACACCCCACT |
| | | AACTCTCCTCACACCTTGGAGAAAGGTCCTAACAACAG |
| | | GGAGTGTCTTCAACGCAAACCAAGTGTGCAATGCGGTT |
| | | AATCTGATACCGCTCGATACCCCGCAGAGGTTCCGTGT |
| | | TGTTTATATGAGCATCACCCGTCTTTCGGATAACGGGT |
| | | ATTACACCGTTCCTAGAAGAATGCTGGAATTCAGATCG |
| | | GTCAATGCAGTGGCCTTCAACCTGCTGGTGACCCTTAG |
| | | GATTGACAAGGCGATAGGCCCTGGGAAGATCATCGAC |
| | | AATACAGAGCAACTTCCTGAGGCAACATTTATGGTCCA |
| | | CATCGGGAACTTCAGGAGAAAGAAGAGTGAAGTCTAC |
| | | TCTGCCGATTATTGCAAAATGAAAATCGAAAAGATGG |
| | | GCCTGGTTTTTGCACTTGGTGGGATAGGGGGCACCAGT |
| | | CTTCACATTAGAAGCACAGGCAAAATGAGCAAGACTC |
| | | TCCATGCACAACTCGGGTTCAAGAAGACCTTATGTTAC |
| | | CCGCTGATGGATATCAATGAAGACCTTAATCGATTACT |
| | | CTGGAGGAGCAGATGCAAGATAGTAAGAATCCAGGCA |
| | | GTTTTGCAGCCATCAGTTCCTCAAGAATTCCGCATTTAC |
| | | GACGACGTGATCATAAATGATGACCAAGGACTATTCA |
| | | AAGTTCTGTAGACCGTAGTGCCCAGCAATGCCCGAAAA |
| | | CGACCCCCCTCACAATGACAGCCAGAAGGCCCGGACA |
| | | AAAAAGCCCCCTCCGAAAGACTCCACGGACCAAGCGA |
| | | GAGGCCAGCCAGCAGCCGACGGCAAGCGCGAACACCA |
| | | GGCGGCCCCAGCACAGAACAGCCCTGACACAAGGCCA |
| | | CCACCAGCCACCCCAATCTGCATCCTCCTCGTGGGACC |
| | | CCCGAGGACCAACCCCCAAGGCTGCCCCCGATCCAAA |
| | | CCACCAACCGCATCCCCACCACCCCCGGGAAAGAAAC |
| | | CCCCAGCAATTGGAAGGCCCCTCCCCCTCTTCCTCAAC |
| | | ACAAGAACTCCACAACCGAACCGCACAAGCGACCGAG |
| | | GTGACCCAACCGCAGGCATCCGACTCCCTAGACAGATC |
| | | CTCTCTCCCCGGCAAACTAAACAAAACTTAGGGCCAAG |
| | | GAACATACACACCCAACAGAACCCAGACCCCGGCCCA |
| | | CGGCGCCGCGCCCCCAACCCCCGACAACCAGAGGGAG |
| | | CCCCCAACCAATCCCGCCGGCTCCCCCGGTGCCCACAG |
| | | GCAGGGACACCAACCCCCGAACAGACCCAGCACCCAA |
| | | CCATCGACAATCCAAGACGGGGGGGCCCCCCCAAAAA |
| | | AAGGCCCCCAGGGGCCGACAGCCAGCACCGCGAGGAA |
| | | GCCCACCCACCCCACACACGACCACGGCAACCAAACC |
| | | AGAACCCAGACCACCCTGGGCCACCAGCTCCCAGACTC |
| | | GGCCATCACCCCGCAGAAAGGAAAGGCCACAACCCGC |
| | | GCACCCCAGCCCCGATCCGGCGGGGAGCCACCCAACC |
| | | CGAACCAGCACCCAAGAGCGATCCCCGAAGGACCCCC |
| | | GAACCGCAAAGGACATCAGTATCCCACAGCCTCTCCAA |
| | | GTCCCCCGGTCTCCTCCTCTTCTCGAAGGGACCAAAAG |
| | | ATCAATCCACCACACCCGACGACACTCAACTCCCCACC |
| | | CCTAAAGGAGACACCGGGAATCCCAGAATCAAGACTC |
| | | ATCCAATGTCCATCATGGGTCTCAAGGTGAACGTCTCT |
| | | GCCATATTCATGGCAGTACTGTTAACTCTCCAAACACC |
| | | CACCGGTCAAATCCATTGGGGCAATCTCTCTAAGATAG |
| | | GGGTGGTAGGAATAGGAAGTGCAAGCTACAAAGTTAT |
| | | GACTCGTTCCAGCCATCAATCATTAGTCATAAAATTAA |
| | | TGCCCAATATAACTCTCCTCAATAACTGCACGAGGGTA |
| | | GAGATTGCAGAATACAGGAGACTACTGAGAACAGTTT |
| | | TGGAACCAATTAGAGATGCACTTAATGCAATGACCCAG |
| | | AATATAAGACCGGTTCAGAGTGTAGCTTCAAGTAGGA |
| | | GACACAAGAGATTTGCGGGAGTAGTCCTGGCAGGTGC |
| | | GGCCCTAGGCGTTGCCACAGCTGCTCAGATAACAGCCG |
| | | GCATTGCACTTCACCAGTCCATGCTGAACTCTCAAGCC |
| | | ATCGACAATCTGAGAGCGAGCCTGGAAACTACTAATC |
| | | AGGCAATTGAGACAATCAGACAAGCAGGGCAGGAGAT |
| | | GATATTGGCTGTTCAGGGTGTCCAAGACTACATCAATA |
| | | ATGAGCTGATACCGTCTATGAACCAACTATCTTGTGAT |
| | | TTAATCGGCCAGAAGCTCGGGCTCAAATTGCTCAGATA |
| | | CTATACAGAAATCCTGTCATTATTTGGCCCCAGTTTAC |
| | | GGGACCCCATATCTGCGGAGATATCTATCCAGGCTTTG |
| | | AGCTATGCGCTTGGAGGAGACATCAATAAGGTGTTAG |
| | | AAAAGCTCGGATACAGTGGAGGTGATTTACTGGGCATC |
| | | TTAGAGAGCGGAGGAATAAAGGCCCGGATAACTCACG |
| | | TCGACACAGAGTCCTACTTCATTGTCCTCAGTATAGCC |
| | | TATCCGACGCTGTCCGAGATTAAGGGGGTGATTGTCCA |
| | | CCGGCTAGAGGGGGTCTCGTACAACATAGGCTCTCAAG |
| | | AGTGGTATACCACTGTGCCCAAGTATGTTGCAACCCAA |
| | | GGGTACCTTATCTCGAATTTTGATGAGTCATCGTGTACT |
| | | TTCATGCCAGAGGGGACTGTGTGCAGCCAAAATGCCTT |
| | | GTACCCGATGAGTCCTCTGCTCCAAGAATGCCTCCGGG |
| | | GGTACACCAAGTCCTGTGCTCGTACACTCGTATCCGGG |
| | | TCTTTTGGGAACCGGTTCATTTTATCACAAGGGAACCT |
| | | AATAGCCAATTGTGCATCAATCCTTTGCAAGTGTTACA |
| | | CAACAGGAACGATCATTAATCAAGACCCTGACAAGAT |
| | | CCTAACATACATTGCTGCCGATCACTGCCCGGTAGTCG |
| | | AGGTGAACGGCGTGACCATCCAAGTCGGGAGCAGGAG |
| | | GTATCCAGACGCTGTGTACTTGCACAGAATTGACCTCG |
| | | GTCCTCCCATATCATTGGAGAGGTTGGACGTAGGGACA |
| | | AATCTGGGGAATGCAATTGCTAAGTTGGAGGATGCCA |
| | | AGGAATTGTTGGAGTCATCGGACCAGATATTGAGGAGT |
| | | ATGAAAGGTTTATCGAGCACTAGCATAGTCTACATCCT |
| | | GATTGCAGTGTGTCTTGGAGGGTTGATAGGGATCCCCG |
| | | CTTTAATATGTTGCTGCAGGGGGCGTTGTAACAAAAAG |
| | | GGAGAACAAGTTGGTATGTCAAGACCAGGCCTAAAGC |
| | | CTGATCTTACGGGAACATCAAAATCCTATGTAAGGTCG |
| | | CTCTGATCCTCTACAACTCTTGAAACACAAATGTCCCA |
| | | CAAGTCTCCTCTTCGTCATCAAGCAACCACCGCACCCA |
| | | GCATCAAGCCCACCTGAAATTATCTCCGGCTTCCCTCT |
| | | GGCCGAACAATATCGGTAGTTAATCAAAACTTAGGGTG |
| | | CAAGATCATCCACAATGTCACCACAACGAGACCGGAT |
| | | AAATGCCTTCTACAAAGATAACCCCCATCCCAAGGGAA |
| | | GTAGGATAGTCATTAACAGAGAACATCTTATGATTGAT |
| | | AGACCTTATGTTTTGCTGGCTGTTCTGTTTGTCATGTTT |
| | | CTGAGCTTGATCGGGTTGCTAGCCATTGCAGGCATTAG |
| | | ACTTCATCGGGCAGCCATCTACACCGCAGAGATCCATA |
| | | AAAGCCTCAGCACCAATCTAGATGTAACTAACTCAATC |
| | | GAGCATCAGGTCAAGGACGTGCTGACACCACTCTTCAA |
| | | AATCATCGGTGATGAAGTGGGCCTGAGGACACCTCAG |
| | | AGATTCACTGACCTAGTGAAATTAATCTCTGACAAGAT |
| | | TAAATTCCTTAATCCGGATAGGGAGTACGACTTCAGAG |
| | | ATCTCACTTGGTGTATCAACCCGCCAGAGAGAATCAAA |
| | | TTGGATTATGATCAATACTGTGCAGATGTGGCTGCTGA |
| | | AGAGCTCATGAATGCATTGGTGAACTCAACTCTACTGG |
| | | AGACCAGAACAACCAATCAGTTCCTAGCTGTCTCAAAG |
| | | GGAAACTGCTCAGGGCCCACTACAATCAGAGGTCAATT |
| | | CTCAAACATGTCGCTGTCCCTGTTAGACTTGTATTTAGG |
| | | TCGAGGTTACAATGTGTCATCTATAGTCACTATGACAT |
| | | CCCAGGGAATGTATGGGGGAACTTACCTAGTGGAAAA |
| | | GCCTAATCTGAGCAGCAAAAGGTCAGAGTTGTCACAA |
| | | CTGAGCATGTACCGAGTGTTTGAAGTAGGTGTTATCAG |
| | | AAATCCGGGTTTGGGGGCTCCGGTGTTCCATATGACAA |
| | | ACTATCTTGAGCAACCAGTCAGTAATGATCTCAGCAAC |
| | | TGTATGGTGGCTTTGGGGGAGCTCAAACTCGCAGCCCT |
| | | TTGTCACGGGGAAGATTCTATCACAATTCCCTATCAGG |
| | | GATCAGGGAAAGGTGTCAGCTTCCAGCTCGTCAAGCTA |
| | | GGTGTCTGGAAATCCCCAACCGACATGCAATCCTGGGT |
| | | CCCCTTATCAACGGATGATCCAGTGATAGACAGGCTTT |
| | | ACCTCTCATCTCACAGAGGTGTTATCGCTGACAATCAA |
| | | GCAAAATGGGCTGTCCCGACAACACGAACAGATGACA |
| | | AGTTGCGAATGGAGACATGCTTCCAACAGGCGTGTAA |
| | | GGGTAAAATCCAAGCACTCTGCGAGAATCCCGAGTGG |
| | | GCACCATTGAAGGATAACAGGATTCCTTCATACGGGGT |
| | | CTTGTCTGTTGATCTGAGTCTGACAGTTGAGCTTAAAA |
| | | TCAAAATTGCTTCGGGATTCGGGCCATTGATCACACAC |
| | | GGTTCAGGGATGGACCTATACAAATCCAACCACAACA |
| | | ATGTGTATTGGCTGACTATCCCGCCAATGAAGAACCTA |
| | | GCCTTAGGTGTAATCAACACATTGGAGTGGATACCGAG |
| | | ATTCAAGGTTAGTCCCTACCTCTTCACTGTCCCAATTAA |
| | | GGAAGCAGGCGAAGACTGCCATGCCCCAACATACCTA |
| | | CCTGCGGAGGTGGATGGTGATGTCAAACTCAGTTCCAA |
| | | TCTGGTGATTCTACCTGGTCAAGATCTCCAATATGTTTT |
| | | GGCAACCTACGATACTTCCAGGGTTGAACATGCTGTGG |
| | | TTTATTACGTTTACAGCCCAAGCCGCTCATTTTCTTACT |
| | | TTTATCCTTTTAGGTTGCCTATAAAGGGGGTCCCCATCG |
| | | AATTACAAGTGGAATGCTTCACATGGGACCAAAAACTC |
| | | TGGTGCCGTCACTTCTGTGTGCTTGCGGACTCAGAATC |
| | | TGGTGGACATATCACTCACTCTGGGATGGTGGGCATGG |
| | | GAGTCAGCTGCACAGTCACCCGGGAAGATGGAACCAA |
| | | TCGCAGATAGGGCTGCTAGTGAACCAATCACATGATGT |
| | | CACCCAGACATCAGGCATACCCACTAGTGTGAAATAG |
| | | ACATCAGAATTAAGAAAAACGTAGGGTCCAAGTGGTT |
| | | CCCCGTTATGGACTCGCTATCTGTCAACCAGATCTTAT |
| | | ACCCTGAAGTTCACCTAGATAGCCCGATAGTTACCAAT |
| | | AAGATAGTAGCCATCCTGGAGTATGCTCGAGTCCCTCA |
| | | CGCTTACAGCCTGGAGGACCCTACACTGTGTCAGAACA |
| | | TCAAGCACCGCCTAAAAAACGGATTTTCCAACCAAATG |
| | | ATTATAAACAATGTGGAAGTTGGGAATGTCATCAAGTC |
| | | CAAGCTTAGGAGTTATCCGGCCCACTCTCATATTCCAT |
| | | ATCCAAATTGTAATCAGGATTTATTTAACATAGAAGAC |
| | | AAAGAGTCAACGAGGAAGATCCGTGAACTCCTCAAAA |
| | | AGGGGAATTCGCTGTACTCCAAAGTCAGTGATAAGGTT |
| | | TTCCAATGCTTAAGGGACACTAACTCACGGCTTGGCCT |
| | | AGGCTCCGAATTGAGGGAGGACATCAAGGAGAAAGTT |
| | | ATTAACTTGGGAGTTTACATGCACAGCTCCCAGTGGTT |
| | | TGAGCCCTTTCTGTTTTGGTTTACAGTCAAGACTGAGAT |
| | | GAGGTCAGTGATTAAATCACAAACCCATACTTGCCATA |
| | | GGAGGAGACACACACCTGTATTCTTCACTGGTAGTTCA |
| | | GTTGAGTTGCTAATCTCTCGTGACCTTGTTGCTATAATC |
| | | AGTAAAGAGTCTCAACATGTATATTACCTGACATTTGA |
| | | ACTGGTTTTGATGTATTGTGATGTCATAGAGGGGAGGT |
| | | TAATGACAGAGACCGCTATGACTATTGATGCTAGGTAT |
| | | ACAGAGCTTCTAGGAAGAGTCAGATACATGTGGAAAC |
| | | TGATAGATGGTTTCTTCCCTGCACTCGGGAATCCAACT |
| | | TATCAAATTGTAGCCATGCTGGAGCCTCTTTCACTTGCT |
| | | TACCTGCAGCTGAGGGATATAACAGTAGAACTCAGAG |
| | | GTGCTTTCCTTAACCACTGCTTTACTGAAATACATGATG |
| | | TTCTTGACCAAAACGGGTTTTCTGATGAAGGTACTTAT |
| | | CATGAGTTAACTGAAGCTCTAGATTACATTTTCATAAC |
| | | TGATGACATACATCTGACAGGGGAGATTTTCTCATTTT |
| | | TCAGAAGTTTCGGCCACCCCAGACTTGAAGCAGTAACG |
| | | GCTGCTGAAAATGTTAGGAAATACATGAATCAGCCTAA |
| | | AGTCATTGTGTATGAGACTCTGATGAAAGGTCATGCCA |
| | | TATTTTGTGGAATCATAATCAACGGCTATCGTGACAGG |
| | | CACGGAGGCAGTTGGCCACCGCTGACCCTCCCCCTGCA |
| | | TGCTGCAGACACAATCCGGAATGCTCAAGCTTCAGGTG |
| | | AAGGGTTAACACATGAGCAGTGCGTTGATAACTGGAA |
| | | ATCTTTTGCTGGAGTGAAATTTGGCTGCTTTATGCCTCT |
| | | TAGCCTGGATAGTGATCTGACAATGTACCTAAAGGACA |
| | | AGGCACTTGCTGCTCTCCAAAGGGAATGGGATTCAGTT |
| | | TACCCGAAAGAGTTCCTGCGTTACGACCCTCCCAAGGG |
| | | AACCGGGTCACGGAGGCTTGTAGATGTTTTCCTTAATG |
| | | ATTCGAGCTTTGACCCATATGATGTGATAATGTATGTT |
| | | GTAAGTGGAGCTTACCTCCATGACCCTGAGTTCAACCT |
| | | GTCTTACAGCCTGAAAGAAAAGGAGATCAAGGAAACA |
| | | GGTAGACTTTTTGCTAAAATGACTTACAAAATGAGGGC |
| | | ATGCCAAGTGATTGCTGAAAATCTAATCTCAAACGGGA |
| | | TTGGCAAATATTTTAAGGACAATGGGATGGCCAAGGAT |
| | | GAGCACGATTTGACTAAGGCACTCCACACTCTAGCTGT |
| | | CTCAGGAGTCCCCAAAGATCTCAAAGAAAGTCACAGG |
| | | GGGGGGCCAGTCTTAAAAACCTACTCCCGAAGCCCAGT |
| | | CCACACAAGTACCAGGAACGTGAGAGCAGCAAAAGGG |
| | | TTTATAGGGTTCCCTCAAGTAATTCGGCAGGACCAAGA |
| | | CACTGATCATCCGGAGAATATGGAAGCTTACGAGACA |
| | | GTCAGTGCATTTATCACGACTGATCTCAAGAAGTACTG |
| | | CCTTAATTGGAGATATGAGACCATCAGCTTGTTTGCAC |
| | | AGAGGCTAAATGAGATTTACGGATTGCCCTCATTTTTC |
| | | CAGTGGCTGCATAAGAGGCTTGAGACCTCTGTCCTGTA |
| | | TGTAAGTGACCCTCATTGCCCCCCCGACCTTGACGCCC |
| | | ATATCCCGTTATATAAAGTCCCCAATGATCAAATCTTC |
| | | ATTAAGTACCCTATGGGAGGTATAGAAGGGTATTGTCA |
| | | GAAGCTGTGGACCATCAGCACCATTCCCTATCTATACC |
| | | TGGCTGCTTATGAGAGCGGAGTAAGGATTGCTTCGTTA |
| | | GTGCAAGGGGACAATCAGACCATAGCCGTAACAAAAA |
| | | GGGTACCCAGCACATGGCCCTACAACCTTAAGAAACG |
| | | GGAAGCTGCTAGAGTAACTAGAGATTACTTTGTAATTC |
| | | TTAGGCAAAGGCTACATGATATTGGCCATCACCTCAAG |
| | | GCAAATGAGACAATTGTTTCATCACATTTTTTTGTCTAT |
| | | TCAAAAGGAATATATTATGATGGGCTACTTGTGTCCCA |
| | | ATCACTCAAGAGCATCGCAAGATGTGTATTCTGGTCAG |
| | | AGACTATAGTTGATGAAACAAGGGCAGCATGCAGTAA |
| | | TATTGCTACAACAATGGCTAAAAGCATCGAGAGAGGTT |
| | | ATGACCGTTACCTTGCATATTCCCTGAACGTCCTAAAA |
| | | GTGATACAGCAAATTCTGATCTCTCTTGGCTTCACAAT |
| | | CAATTCAACCATGACCCGGGATGTAGTCATACCCCTCC |
| | | TCACAAACAACGACCTCTTAATAAGGATGGCACTGTTG |
| | | CCCGCTCCTATTGGGGGGATGAATTATCTGAATATGAG |
| | | CAGGCTGTTTGTCAGAAACATCGGTGATCCAGTAACAT |
| | | CATCAATTGCTGATCTCAAGAGAATGATTCTCGCCTCA |
| | | CTAATGCCTGAAGAGACCCTCCATCAAGTAATGACACA |
| | | ACAACCGGGGGACTCTTCATTCCTAGACTGGGCTAGCG |
| | | ACCCTTACTCAGCAAATCTTGTATGTGTCCAGAGCATC |
| | | ACTAGACTCCTCAAGAACATAACTGCAAGGTTTGTCCT |
| | | GATCCATAGTCCAAACCCAATGTTAAAAGGATTATTCC |
| | | ATGATGACAGTAAAGAAGAGGACGAGGGACTGGCGGC |
| | | ATTCCTCATGGACAGGCATATTATAGTACCTAGGGCAG |
| | | CTCATGAAATCCTGGATCATAGTGTCACAGGGGCAAGA |
| | | GAGTCTATTGCAGGCATGCTGGATACCACAAAAGGCTT |
| | | GATTCGAGCCAGCATGAGGAAGGGGGGGTTAACCTCT |
| | | CGAGTGATAACCAGATTGTCCAATTATGACTATGAACA |
| | | ATTCAGAGCAGGGATGGTGCTATTGACAGGAAGAAAG |
| | | AGAAATGTCCTCATTGACAAAGAGTCATGTTCAGTGCA |
| | | GCTGGCGAGAGCTCTAAGAAGCCATATGTGGGCGAGG |
| | | CTAGCTCGAGGACGGCCTATTTACGGCCTTGAGGTCCC |
| | | TGATGTACTAGAATCTATGCGAGGCCACCTTATTCGGC |
| | | GTCATGAGACATGTGTCATCTGCGAGTGTGGATCAGTC |
| | | AACTACGGATGGTTTTTTGTCCCCTCGGGTTGCCAACT |
| | | GGATGATATTGACAAGGAAACATCATCCTTGAGAGTCC |
| | | CATATATTGGTTCTACCACTGATGAGAGAACAGACATG |
| | | AAGCTTGCCTTCGTAAGAGCCCCAAGTCGATCCTTGCG |
| | | ATCTGCTGTTAGAATAGCAACAGTGTACTCATGGGCTT |
| | | ACGGTGATGATGATAGCTCTTGGAACGAAGCCTGGTTG |
| | | TTGGCTAGGCAAAGGGCCAATGTGAGCCTGGAGGAGC |
| | | TAAGGGTGATCACTCCCATCTCAACTTCGACTAATTTA |
| | | GCGCATAGGTTGAGGGATCGTAGCACTCAAGTGAAAT |
| | | ACTCAGGTACATCCCTTGTCCGAGTGGCGAGGTATACC |
| | | ACAATCTCCAACGACAATCTCTCATTTGTCATATCAGA |
| | | TAAGAAGGTTGATACTAACTTTATATACCAACAAGGAA |
| | | TGCTTCTAGGGTTGGGTGTTTTAGAAACATTGTTTCGAC |
| | | TCGAGAAAGATACCGGATCATCTAACACGGTATTACAT |
| | | CTTCACGTCGAAACAGATTGTTGCGTGATCCCGATGAT |
| | | AGATCATCCCAGGATACCCAGCTCCCGCAAGCTAGAGC |
| | | TGAGGGCAGAGCTATGTACCAACCCATTGATATATGAT |
| | | AATGCACCTTTAATTGACAGAGATGCAACAAGGCTATA |
| | | CACCCAGAGCCATAGGAGGCACCTTGTGGAATTTGTTA |
| | | CATGGTCCACACCCCAACTATATCACATTTTAGCTAAG |
| | | TCCACAGCACTATCTATGATTGACCTGGTAACAAAATT |
| | | TGAGAAGGACCATATGAATGAAATTTCAGCTCTCATAG |
| | | GGGATGACGATATCAATAGTTTCATAACTGAGTTTCTG |
| | | CTCATAGAGCCAAGATTATTCACTATCTACTTGGGCCA |
| | | GTGTGCGGCCATCAATTGGGCATTTGATGTACATTATC |
| | | ATAGACCATCAGGGAAATATCAGATGGGTGAGCTGTT |
| | | GTCATCGTTCCTTTCTAGAATGAGCAAAGGAGTGTTTA |
| | | AGGTGCTTGTCAATGCTCTAAGCCACCCAAAGATCTAC |
| | | AAGAAATTCTGGCATTGTGGTATTATAGAGCCTATCCA |
| | | TGGTCCTTCACTTGATGCTCAAAACTTGCACACAACTG |
| | | TGTGCAACATGGTTTACACATGCTATATGACCTACCTC |
| | | GACCTGTTGTTGAATGAAGAGTTAGAAGAGTTCACATT |
| | | TCTCTTGTGTGAAAGCGACGAGGATGTAGTACCGGACA |
| | | GATTCGACAACATCCAGGCAAAACACTTATGTGTTCTG |
| | | GCAGATTTGTACTGTCAACCAGGGACCTGCCCACCAAT |
| | | TCGAGGTCTAAGACCGGTAGAGAAATGTGCAGTTCTAA |
| | | CCGACCATATCAAGGCAGAGGCTATGTTATCTCCAGCA |
| | | GGATCTTCGTGGAACATAAATCCAATTATTGTAGACCA |
| | | TTACTCATGCTCTCTGACTTATCTCCGGCGAGGATCGAT |
| | | CAAACAGATAAGATTGAGAGTTGATCCAGGATTCATTT |
| | | TCGACGCCCTCGCTGAGGTAAATGTCAGTCAGCCAAAG |
| | | ATCGGCAGCAACAACATCTCAAATATGAGCATCAAGG |
| | | CTTTCAGACCCCCACACGATGATGTTGCAAAATTGCTC |
| | | AAAGATATCAACACAAGCAAGCACAATCTTCCCATTTC |
| | | AGGGGGCAATCTCGCCAATTATGAAATCCATGCTTTCC |
| | | GCAGAATCGGGTTGAACTCATCTGCTTGCTACAAAGCT |
| | | GTTGAGATATCAACATTAATTAGGAGATGCCTTGAGCC |
| | | AGGGGAGGACGGCTTGTTCTTGGGTGAGGGATCGGGTT |
| | | CTATGTTGATCACTTATAAAGAGATACTTAAACTAAAC |
| | | AAGTGCTTCTATAATAGTGGGGTTTCCGCCAATTCTAG |
| | | ATCTGGTCAAAGGGAATTAGCACCCTATCCCTCCGAAG |
| | | TTGGCCTTGTCGAACACAGAATGGGAGTAGGTAATATT |
| | | GTCAAAGTGCTCTTTAACGGGAGGCCCGAAGTCACGTG |
| | | GGTAGGCAGTGTAGATTGCTTCAATTTCATAGTTAGTA |
| | | ATATCCCTACCTCTAGTGTGGGGTTTATCCATTCAGATA |
| | | TAGAGACCTTGCCTGACAAAGATACTATAGAGAAGCT |
| | | AGAGGAATTGGCAGCCATCTTATCGATGGCTCTGCTCC |
| | | TGGGCAAAATAGGATCAATACTGGTGATTAAGCTTATG |
| | | CCTTTCAGCGGGGATTTTGTTCAGGGATTTATAAGTTAT |
| | | GTAGGGTCTCATTATAGAGAAGTGAACCTTGTATACCC |
| | | TAGATACAGCAACTTCATCTCTACTGAATCTTATTTGGT |
| | | TATGACAGATCTCAAGGCTAACCGGCTAATGAATCCTG |
| | | AAAAGATTAAGCAGCAGATAATTGAATCATCTGTGAG |
| | | GACTTCACCTGGACTTATAGGTCACATCCTATCCATTA |
| | | AGCAACTAAGCTGCATACAAGCAATTGTGGGAGACGC |
| | | AGTTAGTAGAGGTGATATCAATCCTACTCTGAAAAAAC |
| | | TTACACCTATAGAGCAGGTGCTGATCAATTGCGGGTTG |
| | | GCAATTAACGGACCTAAGCTGTGCAAAGAATTGATCCA |
| | | CCATGATGTTGCCTCAGGGCAAGATGGATTGCTTAATT |
| | | CTATACTCATCCTCTACAGGGAGTTGGCAAGATTCAAA |
| | | GACAACCAAAGAAGTCAACAAGGGATGTTCCACGCTT |
| | | ACCCCGTATTGGTAAGTAGCAGGCAACGAGAACTTATA |
| | | TCTAGGATCACCCGCAAATTCTGGGGGCACATTCTTCT |
| | | TTACTCCGGGAACAAAAAGTTGATAAATAAGTTTATCC |
| | | AGAATCTCAAGTCCGGCTATCTGATACTAGACTTACAC |
| | | CAGAATATCTTCGTTAAGAATCTATCCAAGTCAGAGAA |
| | | ACAGATTATTATGACGGGGGGTTTGAAACGTGAGTGG |
| | | GTTTTTAAGGTAACAGTCAAGGAGACCAAAGAATGGT |
| | | ATAAGTTAGTCGGATACAGTGCCCTGATTAAGGACTAA |
| | | TTGGTTGAACTCCGGAACCCTAATCCTGCCCTAGGTGG |
| | | TTAGGCATTATTTGCAATATATTAAAGAAAACTTTGAA |
| | | AATACGAAGTTTCTATTCCCAGCTTTGTCTGGTGGCCG |
| | | GCATGGTCCCAGCCTCCTCGCTGGCGCCGGCTGGGCAA |
| | | CATTCCGAGGGGACCGTCCCCTCGGTAATGGCGAATGG |
| | | GACGCGGCCGATCCGGCTGCTAACAAAGCCCGAAAGG |
| | | AAGCTGAGTTGGCTGCTGCCACCGCTGAGCAATAACTA |
| | | GCATAACCCCTTGGGGCCTCTAAACGGGTCTTGAGGGG |
| | | TTTTTTGCTGAAAGGAGGAACTATATCCGGATGCGGCC |
| | | GCGGGCCCTATGGTACCCAGCTTTTGTTCCCTTTAGTGA |
| | | GGGTTAATTCCGAGCTTGGCGTAATCATGGTCATAGCT |
| | | GTTTCCTGTGTGAAATTGTTATCCGCTCACAATTCCACA |
| | | CAACATAGGAGCCGGAAGCATAAAGTGTAAAGCCTGG |
| | | GGTGCCTAATGAGTGAGGTAACTCACATTAATTGCGTT |
| | | GCGCTCACTGCCCGCTTTCCAGTCGGGAAACCTGTCGT |
| | | GCCAGCTGCATTAATGAATCGGCCAACGCGCGGGGAG |
| | | AGGCGGTTTGCGTATTGGGCGCTCTTCCGCTTCCTCGCT |
| | | CACTGACTCGCTGCGCTCGGTCGTTCGGCTGCGGCGAG |
| | | CGGTATCAGCTCACTCAAAGGCGGTAATACGGTTATCC |
| | | ACAGAATCAGGGGATAACGCAGGAAAGAACATGTGAG |
| | | CAAAAGGCCAGCAAAAGGCCAGGAACCGTAAAAAGGC |
| | | CGCGTTGCTGGCGTTTTTCCATAGGCTCGGCCCCCCTG |
| | | ACGAGCATCACAAAAATCGACGCTCAAGTCAGAGGTG |
| | | GCGAAACCCGACAGGACTATAAAGATACCAGGCGTTC |
| | | CCCCCTGGAAGCTCCCTCGTGCGCTCTCCTGTTCCGACC |
| | | CTGCCGCTTACCGGATACCTGTCCGCCTTTCTCCCTTCG |
| | | GGAAGCGTGGCGCTTTCTCAATGCTCACGCTGTAGGTA |
| | | TCTCAGTTCGGTGTAGGTCGTTCGCTCCAAGCTGGGCT |
| | | GTGTGCACGAACCCCCCGTTCAGCCCGACCGCTGCGCC |
| | | TTATCCGGTAACTATCGTCTTGAGTCCAACCCGGTAAG |
| | | ACACGACTTATCGCCACTGGCAGCAGCCACTGGTAACA |
| | | GGATTAGCAGAGCGAGGTATGTAGGCGGTGCTACAGA |
| | | GTTCTTGAAGTGGTGGCCTAACTACGGCTACACTAGAA |
| | | GGACAGTATTTGGTATCTGCGCTCTGCTGAAGCCAGTT |
| | | ACCTTCGGAAAAAGAGTTGGTAGCTCTTGATCCGGCAA |
| | | ACAAACCACCGCTGGTAGCGGTGGTTTTTTTGTTTGCA |
| | | AGCAGCAGATTACGCGCAGAAAAAAAGGATCTCAAGA |
| | | AGATCCTTTGATCTTTTCTACGGGGTCTGACGCTCAGTG |
| | | GAACGAAAACTCACGTTAAGGGATTTTGGTCATGAGAT |
| | | TATCAAAAAGGATCTTCACCTAGATCCTTTTAAATTAA |
| | | AAATGAAGTTTTAAATCAATCTAAAGTATATATGAGTA |
| | | AACTTGGTCTGACAGTTACCAATGCTTAATCAGTGAGG |
| | | CACCTATCTCAGCGATCTGTCTATTTCGTTCATCCATAG |
| | | TTGCCTGACTGCCCGTCGTGTAGATAACTACGATACGG |
| | | GAGGGCTTACCATCTGGCCCCAGTGCTGCAATGATACC |
| | | GCGAGACCCACGCTCACCGGCTCCAGATTTATCAGCAA |
| | | TAAACCAGCCAGCCGGAAGGGCCGAGCGCAGAAGTGG |
| | | TCCTGCAACTTTATCCGCCTCCATCCAGTCTATTAATTG |
| | | TTGCCGGGAAGCTAGAGTAAGTAGTTCGCCAGTTAATA |
| | | GTTTGCGCAACGTTGTTGCCATTGCTACAGGCATCGTG |
| | | GTGTCACGCTCGTCGTTTGGTATGGCTTCATTCAGCTCC |
| | | GGTTCCCAACGATCAAGGCGAGTTACATGATCCCCCAT |
| | | GTTGTGAAAAAAAGCGGTTAGCTCCTTCGGTCCTCCGA |
| | | TCGTTGTCAGAAGTAAGTTGGCCGCAGTGTTATCACTC |
| | | ATGCTTATGGCAGCACTGCATAATTCTCTTACTGTCATG |
| | | CCATCCGTAAGATGCTTTTCTGTGACTGGTGAGTACTC |
| | | AACCAAGTCATTCTGAGAATAGTGTATGCGGCGACCGA |
| | | GTTGCTCTTGCCCGGCGTCAATACGGGATAATACCGCG |
| | | CCACATAGCAGAACTTTAAAAGTGCTCATCATTGGAAA |
| | | ACGTTCTTCGGGGCGAAAACTCTCAAGGATCTTACCGC |
| | | TGTTGAGATCCAGTTCGATGTAACCCACTCGTGCACCC |
| | | AACTGATCTTCAGCATCTTTTACTTTCACCAGCGTTTCT |
| | | GGGTGAGCAAAAACAGGAAGGCAAAATGCCGCAAAA |
| | | AAGGGAATAAGGGCGACACGGAAATGTTGAATACTCA |
| | | TACTCTTCCTTTTTCAATATTATTGAAGCATTTATCAGG |
| | | GTTATTGTCTCATGAGCGGATACATATTTGAATGTATTT |
| | | AGAAAAATAAACAAATAGGGGTTCCGCGCACATTTCC |
| | | CCGAAAAGTGCCACCTGAAATTGTAAACGTTAATATTT |
| | | TGTTAAAATTCGCGTTAAATTTTTGTTAAATCAGCTCAT |
| | | TTTTTAACCAATAGGCCGAAATCGGCAAAATCCCTTAT |
| | | AAATCAAAAGAATAGACCGAGATAGGGTTGAGTGTTG |
| | | TTCCAGTTTGGAACAAGAGTCCACTATTAAAGAACGTG |
| | | GACTCCAACGTCAAAGGGCGAAAAACCGTCTATCAGG |
| | | GCGATGGCCCACTACGTGAACCATCACCCTAATCAAGT |
| | | TTTTTGGGGTCGAGGTGCCGTAAAGCACTAAATCGGAA |
| | | CCCTAAAGGGAGCCCCCGATTTAGAGCTTGACGGGGA |
| | | AAGCCGGCGAACGTGGCGAGAAAGGAAGGGAAGAAA |
| | | GCGAAAGGAGCGGGCGCTAGGGCGCTGGCAAGTGTAG |
| | | CGGTCACGCTGCGCGTAACCACCACACCCGCCGCGCTT |
| | | AATGCGCCGCTACAGGGCGCGTCCCATTCGCCATTCAG |
| | | GCTGCGCAACTGTTGGGAAGGGCGATCGGTGCGGGCC |
| | | TCTTCGCTATTACGCCAGCCACCGCGGTG |
| 4 | T2A sequence | GSGEGRGSLLTCGDVEENPGP |
| | with GSG | |
| | linker at N- | |
| | terminus | |
| 5 | P2A sequence | GSGATNFSLLKQAGDVEENPGP |
| | with GSG | |
| | linker at N- | |
| | terminus | |
| 6 | E2A sequence | GSGQCTNYALLKLAGDVESNPGP |
| | with GSG | |
| | linker at N- | |
| | terminus | |
| 7 | F2A sequence | GSGVKQTLNFDLLKLAGDVESNPGP |
| | with GSG | |
| | linker at N- | |
| | terminus | |
| 8 | T2A sequence | EGRGSLLTCGDVEENPGP |
| | without GSG | |
| | linker at N- | |
| | terminus | |
| 9 | P2A sequence | ATNFSLLKQAGDVEENPGP |
| | without GSG | |
| | linker at N- | |
| | terminus | |
| 10 | E2A sequence | QCTNYALLKLAGDVESNPGP |
| | without GSG | |
| | linker at N- | |
| | terminus | |
| 11 | F2A sequence | VKQTLNFDLLKLAGDVESNPGP |
| | without GSG | |
| | linker at N- | |
| | terminus | |
| 12 | Furin cleavage | RXRR |
| | consensus | |
| | sequence | |
| 13 | Furin cleavage | RXKR |
| | consensus | |
| | sequence | |
| 14 | Furin cleavage | RGRR |
| | sequence | |
| 15 | Furin cleavage | RARR |
| | sequence | |
| 16 | Furin cleavage | RLRR |
| | sequence | |
| 17 | Furin cleavage | RMRR |
| | sequence | |
| 18 | Furin cleavage | RFRR |
| | sequence | |
| 19 | Furin cleavage | RWRR |
| | sequence | |
| 20 | Furin cleavage | RKRR |
| | sequence | |
| 21 | Furin cleavage | RQRR |
| | sequence | |
| 22 | Furin cleavage | RERR |
| | sequence | |
| 23 | Furin cleavage | RSRR |
| | sequence | |
| 24 | Furin cleavage | RPRR |
| | sequence | |
| 25 | Furin cleavage | RVRR |
| | sequence | |
| 26 | Furin cleavage | RIRR |
| | sequence | |
| 27 | Furin cleavage | RCRR |
| | sequence | |
| 28 | Furin cleavage | RYRR |
| | sequence | |
| 29 | Furin cleavage | RHRR |
| | sequence | |
| 30 | Furin cleavage | RRRR |
| | sequence | |
| 31 | Furin cleavage | RNRR |
| | sequence | |
| 32 | Furin cleavage | RDRR |
| | sequence | |
| 33 | Furin cleavage | RTRR |
| | sequence | |
| 34 | Furin cleavage | RGKR |
| | sequence | |
| 35 | Furin cleavage | RAKR |
| | sequence | |
| 36 | Furin cleavage | RLKR |
| | sequence | |
| 37 | Furin cleavage | RMKR |
| | sequence | |
| 38 | Furin cleavage | RFKR |
| | sequence | |
| 39 | Furin cleavage | RWKR |
| | sequence | |
| 40 | Furin cleavage | RKKR |
| | sequence | |
| 41 | Furin cleavage | RQKR |
| | sequence | |
| 42 | Furin cleavage | REKR |
| | sequence | |
| 43 | Furin cleavage | RSKR |
| | sequence | |
| 44 | Furin cleavage | RPKR |
| | sequence | |
| 45 | Furin cleavage | RVKR |
| | sequence | |
| 46 | Furin cleavage | RIKR |
| | sequence | |
| 47 | Furin cleavage | RCKR |
| | sequence | |
| 48 | Furin cleavage | RYKR |
| | sequence | |
| 49 | Furin cleavage | RHKR |
| | sequence | |
| 50 | Furin cleavage | RRKR |
| | sequence | |
| 51 | Furin cleavage | RNKR |
| | sequence | |
| 52 | Furin cleavage | RDKR |
| | sequence | |
| 53 | Furin cleavage | RTKR |
| | sequence | |
| 54 | EBV gp350 | MEAALLVCQYTIQSLIHLTGEDPGFFNVEIPEFPFYPTCNV |
| | | CTADVNVTINFDVGGKKHQLDLDFGQLTPHTKAVYQPR |
| | | GAFGGSENATNLFLLELLGAGELALTMRSKKLPINVTTGE |
| | | EQQVSLESVDVYFQDVFGTMWCHHAEMQNPVYLIPETV |
| | | PYIKWDNCNSTNITAVVRAQGLDVTLPLSLPTSAQDSNFS |
| | | VKTEMLGNEIDIECIMEDGEISQVLPGDNKFNITCSGYESH |
| | | VPSGGILTSTSPVATPIPGTGYAYSLRLTPRPVSRFLGNNSI |
| | | LYVFYSGNGPKASGGDYCIQSNIVFSDEIPASQDMPTNTT |
| | | DITYVGDNATYSVPMVTSEDANSPNVTVTAFWAWPNNT |
| | | ETDFKCKWTLTSGTPSGCENISGAFASNRTFDITVSGLGT |
| | | APKTLIITRTATNATTTTHKVIFSKAPESTTTSPTLNTTGFA |
| | | DPNTTTGLPSSTHVPTNLTAPASTGPTVSTADVTSPTPAGT |
| | | TSGASPVTPSPSPWDNGTESKAPDMTSSTSPVTTPTPNATS |
| | | PTPAVTTPTPNATSPTPAVTTPTPNATSPTLGKTSPTSAVT |
| | | TPTPNATSPTLGKTSPTSAVTTPTPNATSPTLGKTSPTSAV |
| | | TTPTPNATGPTVGETSPQANATNHTLGGTSPTPVVTSQPK |
| | | NATSAVTTGQHNITSSSTSSMSLRPSSNPETLSPSTSDNSTS |
| | | HMPLLTSAHPTGGENITQVTPASISTHHVSTSSPAPRPGTT |
| | | SQASGPGNSSTSTKPGEVNVTKGTPPQNATSPQAPSGQKT |
| | | AVPTVTSTGGKANSTTGGKHTTGHGARTSTEPTTDYGGD |
| | | STTPRPRYNATTYLPPSTSSKLRPRWTFTSPPVTTAQATVP |
| | | VPPTSQPRFSNLSMLVLQWASLAVLTLLLLLVMADCAFR |
| | | RNLSTSHTYTTPPYDDAETYV |
| 55 | EBV LMP2 | MGRSRERARGRGRGRGEKRPRSPSSQSSSSGSPPRRPPPG |
| | | RRPFFHPVGDADYFEYLQEGGPDGEPDVPPGAIEQGPTDD |
| | | PGEGPSTGPRGQGDGGRRKKGGWFGKHRGQGGSNPKFE |
| | | NIAEGLRVLLARSHVERTTEEGNWVAGVFVYGGSKTSLY |
| | | NLRRGIALAVPQCRITPLSRLPFGMAPGPGPQPGPLRESIV |
| | | CYFMVFLQTHIFAEVLKDAIKDLVMTKPAPTCNIKVTVCS |
| | | FDDGVDLPPWFPPMVEGAAAEGDDGDDGDEGGDGDEG |
| | | EEGQEMGSLEMVPMGAGPPSPGGDPDGDDGGNNSQYPS |
| | | ASGSSGNTPTPPNDEERESNEEPPPPYEDPYWGNGDRHSD |
| | | FQPLGTQDQSLYLGLQHDGNDGLPPPPYSPRDDSSQHIFE |
| | | EAGRGSMNPVCLPVIVAPYLFWLAAIAASCFTASVSTVVT |
| | | ATGLALSLLLLAAVASSYAAAQRKLLTPVTVLTAVVTFF |
| | | AICLTWRIEDPPFNSLLFALLAAAGGLOGIYVLVMLVLLIL |
| | | AYRRRWRRLTVCGGIMFLACVLVLIVDAVLQLSPLLGAV |
| | | TVVSMTLLLLAFVLWLSSPGGLGTLGAALLTLAAALALL |
| | | ASLILGTLNLTTMFLLMLLWTLVVLLICSSCSSCPLSKILL |
| | | ARLFLYALALLLLASALIAGGSILQTNFKSLSSTEFIPNLFC |
| | | MLLLIVAGILFILAILTEWGSGNRTYGPVFMCLGGLLTMV |
| | | AGAVWLTVMTNTLLSAWILTAGFLIFLIGFALFGVIRCCR |
| | | YCCYYCLTLESEERPPTPYRNTV |
| 56 | EBV gH | MQLLCVFCLVLLWEVGAASLSEVKLHLDIEGHASHYTIP |
| | | WTELMAKVPGLSPEALWREANVTEDLASMLNRYKLIYK |
| | | TSGTLGIALAEPVDIPAVSEGSMQVDASKVHPGVISGLNS |
| | | PACMLSAPLEKQLFYYIGTMLPNTRPHSYVFYQLRCHLSY |
| | | VALSINGDKFQYTGAMTSKFLMGTYKRVTEKGDEHVLSL |
| | | VFGKTKDLPDLRGPFSYPSLTSAQSGDYSLVIVTTFVHYA |
| | | NFHNYFVPNLKDMFSRAVTMTAASYARYVLQKLVLLEM |
| | | KGGCREPELDTETLTTMFEVSVAFFKVGHAVGETGNGCV |
| | | DLRWLAKSFFELTVLKDIIGICYGATVKGMQSYGLERLA |
| | | AMLMATVKMEELGHLTTEKQEYALRLATVGYPKAGVYS |
| | | GLIGGATSVLLSAYNRHPLFQPLHTVMRETLFIGSHVVLR |
| | | ELRLNVTTQGPNLALYQLLSTALCSALEIGEVLRGLALGT |
| | | ESGLFSPCYLSLRFDLTRDKLLSMAPQEATLDQAAVSNA |
| | | VDGFLGRLSLEREDRDAWHLPAYKCVDRLDKVLMIIPLI |
| | | NVTFIISSDREVRGSALYEASTTYLSSSLFLSPVIMNKCSQ |
| | | GAVAGEPRQIPKIQNFTRTQKSCIFCGFALLSYDEKEGLET |
| | | TTYITSQEVQNSILSSNYFDFDNLHVHYLLLTTNGTVMEI |
| | | AGLYEERAHVVLAIILYFIAFALGIFLVHKIVMFFL |
| 57 | EBV gL | MRAVGVFLAICLVTIFVLPTWGNWAYPCCHVTQLRAQH |
| | | LLALENISDIYLVSNQTCDGFSLASLNSPKNGSNQLVISRC |
| | | ANGLNVVSFFISILKRSSSALTGHLRELLTTLETLYGSFSVE |
| | | DLFGANLNRYAWHRGG |
| 58 | EBV gp42 | MVSFKQVRVPLFTAIALVIVLLLAYFLPPRVRGGGRVAAA |
| | | AITWVPKPNVEVWPVDPPPPVNFNKTAEQEYGDKEVKLP |
| | | HWTPTLHTFQVPQNYTKANCTYCNTREYTFSYKGCCFYF |
| | | TKKKHTWNGCFQACAELYPCTYFYGPTPDILPVVTRNLN |
| | | AIESLWVGVYRVGEGNWTSLDGGTFKVYQIFGSHCTYVS |
| | | KFSTVPVSHHECSFLKPCLCVSQRSNS |
| 59 | M-01 (gp350 at | ACCAAACAAAGTTGGGTAAGGATAGTTCAATCAATGA |
| | ATU3) | TCATCTTCTAGTGCACTTAGGATTCAAGATCCTATTATC |
| | MV leader: nt | AGGGACAAGAGCAGGATTAGGGATATCCGAGATGGCC |
| | 1-55 | ACACTTTTAAGGAGCTTAGCATTGTTCAAAAGAAACAA |
| | MV N ORF: nt | GGACAAACCACCCATTACATCAGGATCCGGTGGAGCC |
| | 108-1,685 | ATCAGAGGAATCAAACACATTATTATAGTACCAATCCC |
| | MV P ORF: nt | TGGAGATTCCTCAATTACCACTCGATCCAGACTTCTGG |
| | 1,807-3,330 | ACCGGTTGGTGAGGTTAATTGGAAACCCGGATGTGAGC |
| | MV M ORF: nt | GGGCCCAAACTAACAGGGGCACTAATAGGTATATTATC |
| | 3,438-4,445 | CTTATTTGTGGAGTCTCCAGGTCAATTGATTCAGAGGA |
| | MV F ORF: nt | TCACCGATGACCCTGACGTTAGCATAAGGCTGTTAGAG |
| | 5,449-7,110 | GTTGTCCAGAGTGACCAGTCACAATCTGGCCTTACCTT |
| | MV H ORF: nt | CGCATCAAGAGGTACCAACATGGAGGATGAGGCGGAC |
| | 7,271-9,124 | CAATACTTTTCACATGATGATCCAATTAGTAGTGATCA |
| | ATU upstream | ATCCAGGTTCGGATGGTTCGGGAACAAGGAAATCTCA |
| | motif: nt 9,180- | GATATTGAAGTGCAAGACCCTGAGGGATTCAACATGAT |
| | 9,257 | TCTGGGTACCATCCTAGCCCAAATTTGGGTCTTGCTCG |
| | GE of N gene: | CAAAGGCGGTTACGGCCCCAGACACGGCAGCTGATTC |
| | nt 9,196-9,206 | GGAGCTAAGAAGGTGGATAAAGTACACCCAACAAAGA |
| | Conserved | AGGGTAGTTGGTGAATTTAGATTGGAGAGAAAATGGTT |
| | trinucleotide | GGATGTGGTGAGGAACAGGATTGCCGAGGACCTCTCCT |
| | motif: 9,207- | TACGCCGATTCATGGTCGCTCTAATCCTGGATATCAAG |
| | 9,209 | AGAACACCCGGAAACAAACCCAGGATTGCTGAAATGA |
| | GS of P gene: | TATGTGACATTGATACATATATCGTAGAGGCAGGATTA |
| | nt 9,210-9,226 | GCCAGTTTTATCCTGACTATTAAGTTTGGGATAGAAAC |
| | BsiWI | TATGTATCCTGCTCTTGGACTGCATGAATTTGCTGGTGA |
| | restriction site: | GTTATCCACACTTGAGTCCTTGATGAACCTTTACCAGC |
| | nt 9,246-9,251 | AAATGGGGGAAACTGCACCCTACATGGTAATCCTGGA |
| | Kozak | GAACTCAATTCAGAACAAGTTCAGTGCAGGATCATACC |
| | sequence: nt | CTCTGCTCTGGAGCTATGCCATGGGAGTAGGAGTGGAA |
| | 9,252-9,260 | CTTGAAAACTCCATGGGAGGTTTGAACTTTGGCCGATC |
| | EBV gp350 | TTACTTTGATCCAGCATATTTTAGATTAGGGCAAGAGA |
| | ORF: nt 9,258- | TGGTAAGGAGGTCAGCTGGAAAGGTCAGTTCCACATTG |
| | 11,981 | GCATCTGAACTCGGTATCACTGCCGAGGATGCAAGGCT |
| | BssHII | TGTTTCAGAGATTGCAATGCATACTACTGAGGACAAGA |
| | restriction site: | TCAGTAGAGCGGTTGGACCCAGACAAGCCCAAGTATC |
| | nt 11,982- | ATTTCTACACGGTGATCAAAGTGAGAATGAGCTACCGA |
| | 11,987 | GATTGGGGGGCAAGGAAGATAGGAGGGTCAAACAGAG |
| | ATU | TCGAGGAGAAGCCAGGGAGAGCTACAGAGAAACCGGG |
| | downstream | CCCAGCAGAGCAAGTGATGCGAGAGCTGCCCATCTTCC |
| | motif: nt | AACCGGCACACCCCTAGACATTGACACTGCAACGGAG |
| | 11,982-12,017 | TCCAGCCAAGATCCGCAGGACAGTCGAAGGTCAGCTG |
| | MV L ORF: nt | ACGCCCTGCTTAGGCTGCAAGCCATGGCAGGAATCTCG |
| | 12,072-18,623 | GAAGAACAAGGCTCAGACACGGACACCCCTATAGTGT |
| | MV trailer: nt | ACAATGACAGAAATCTTCTAGACTAGGTGCGAGAGGC |
| | 18,696-18,732 | CGAGGGCCAGAACAACATCCGCCTACCATCCATCATTG |
| | | TTATAAAAAACTTAGGAACCAGGTCCACACAGCCGCC |
| | | AGCCCATCAACCATCCACTCCCACGATTGGAGCCAATG |
| | | GCAGAAGAGCAGGCACGCCATGTCAAAAACGGACTGG |
| | | AATGCATCCGGGCTCTCAAGGCCGAGCCCATCGGCTCA |
| | | CTGGCCATCGAGGAAGCTATGGCAGCATGGTCAGAAA |
| | | TATCAGACAACCCAGGACAGGAGCGAGCCACCTGCAG |
| | | GGAAGAGAAGGCAGGCAGTTCGGGTCTCAGCAAACCA |
| | | TGCCTCTCAGCAATTGGATCAACTGAAGGCGGTGCACC |
| | | TCGCATCCGCGGTCAGGGACCTGGAGAGAGCGATGAC |
| | | GACGCTGAAACTTTGGGAATCCCCCCAAGAAATCTCCA |
| | | GGCATCAAGCACTGGGTTACAGTGTTATTACGTTTATG |
| | | ATCACAGCGGTGAAGCGGTTAAGGGAATCCAAGATGC |
| | | TGACTCTATCATGGTTCAATCAGGCCTTGATGGTGATA |
| | | GCACCCTCTCAGGAGGAGACAATGAATCTGAAAACAG |
| | | CGATGTGGATATTGGCGAACCTGATACCGAGGGATATG |
| | | CTATCACTGACCGGGGATCTGCTCCCATCTCTATGGGG |
| | | TTCAGGGCTTCTGATGTTGAAACTGCAGAAGGAGGGG |
| | | AGATCCACGAGCTCCTGAGACTCCAATCCAGAGGCAA |
| | | CAACTTTCCGAAGCTTGGGAAAACTCTCAATGTTCCTC |
| | | CGCCCCCGGACCCCGGTAGGGCCAGCACTTCCGGGAC |
| | | ACCCATTAAAAAGGGCACAGACGCGAGATTAGCCTCA |
| | | TTTGGAACGGAGATCGCGTCTTTATTGACAGGTGGTGC |
| | | AACCCAATGTGCTCGAAAGTCACCCTCGGAACCATCAG |
| | | GGCCAGGTGCACCTGCGGGGAATGTCCCCGAGTGTGTG |
| | | AGCAATGCCGCACTGATACAGGAGTGGACACCCGAAT |
| | | CTGGTACCACAATCTCCCCGAGATCCCAGAATAATGAA |
| | | GAAGGGGGAGACTATTATGATGATGAGCTGTTCTCTGA |
| | | TGTCCAAGATATTAAAACAGCCTTGGCCAAAATACACG |
| | | AGGATAATCAGAAGATAATCTCCAAGCTAGAATCACT |
| | | GCTGTTATTGAAGGGAGAAGTTGAGTCAATTAAGAAG |
| | | CAGATCAACAGGCAAAATATCAGCATATCCACCCTGG |
| | | AAGGACACCTCTCAAGCATCATGATCGCCATTCCTGGA |
| | | CTTGGGAAGGATCCCAACGACCCCACTGCAGATGTCGA |
| | | AATCAATCCCGACTTGAAACCCATCATAGGCAGAGATT |
| | | CAGGCCGAGCACTGGCCGAAGTTCTCAAGAAACCCGTT |
| | | GCCAGCCGACAACTCCAAGGAATGACAAATGGACGGA |
| | | CCAGTTCCAGAGGACAGCTGCTGAAGGAATTTCAGCTA |
| | | AAGCCGATCGGGAAAAAGATGAGCTCAGCCGTCGGGT |
| | | TTGTTCCTGACACCGGCCCTGCATCACGCAGTGTAATC |
| | | CGCTCCATTATAAAATCCAGCCGGCTAGAGGAGGATCG |
| | | GAAGCGTTACCTGATGACTCTCCTTGATGATATCAAAG |
| | | GAGCCAATGATCTTGCCAAGTTCCACCAGATGCTGATG |
| | | AAGATAATAATGAAGTAGCTACAGCTCAACTTACCTGC |
| | | CAACCCCATGCCAGTCGACCCAACTAGTACAACCTAAA |
| | | TCCATTATAAAAAACTTAGGAGCAAAGTGATTGCCTCC |
| | | CAAGGTCCACAATGACAGAGACCTACGACTTCGACAA |
| | | GTCGGCATGGGACATCAAAGGGTCGATCGCTCCGATAC |
| | | AACCCACCACCTACAGTGATGGCAGGCTGGTGCCCCAG |
| | | GTCAGAGTCATAGATCCTGGTCTAGGCGACAGGAAGG |
| | | ATGAATGCTTTATGTACATGTTTCTGCTGGGGGTTGTTG |
| | | AGGACAGCGATTCCCTAGGGCCTCCAATCGGGCGAGC |
| | | ATTTGGGTTCCTGCCCTTAGGTGTTGGCAGATCCACAG |
| | | CAAAGCCCGAAAAACTCCTCAAAGAGGCCACTGAGCT |
| | | TGACATAGTTGTTAGACGTACAGCAGGGCTCAATGAAA |
| | | AACTGGTGTTCTACAACAACACCCCACTAACTCTCCTC |
| | | ACACCTTGGAGAAAGGTCCTAACAACAGGGAGTGTCTT |
| | | CAACGCAAACCAAGTGTGCAATGCGGTTAATCTGATAC |
| | | CGCTCGATACCCCGCAGAGGTTCCGTGTTGTTTATATG |
| | | AGCATCACCCGTCTTTCGGATAACGGGTATTACACCGT |
| | | TCCTAGAAGAATGCTGGAATTCAGATCGGTCAATGCAG |
| | | TGGCCTTCAACCTGCTGGTGACCCTTAGGATTGACAAG |
| | | GCGATAGGCCCTGGGAAGATCATCGACAATACAGAGC |
| | | AACTTCCTGAGGCAACATTTATGGTCCACATCGGGAAC |
| | | TTCAGGAGAAAGAAGAGTGAAGTCTACTCTGCCGATTA |
| | | TTGCAAAATGAAAATCGAAAAGATGGGCCTGGTTTTTG |
| | | CACTTGGTGGGATAGGGGGCACCAGTCTTCACATTAGA |
| | | AGCACAGGCAAAATGAGCAAGACTCTCCATGCACAAC |
| | | TCGGGTTCAAGAAGACCTTATGTTACCCGCTGATGGAT |
| | | ATCAATGAAGACCTTAATCGATTACTCTGGAGGAGCAG |
| | | ATGCAAGATAGTAAGAATCCAGGCAGTTTTGCAGCCAT |
| | | CAGTTCCTCAAGAATTCCGCATTTACGACGACGTGATC |
| | | ATAAATGATGACCAAGGACTATTCAAAGTTCTGTAGAC |
| | | CGTAGTGCCCAGCAATGCCCGAAAACGACCCCCCTCAC |
| | | AATGACAGCCAGAAGGCCCGGACAAAAAAGCCCCCTC |
| | | CGAAAGACTCCACGGACCAAGCGAGAGGCCAGCCAGC |
| | | AGCCGACGGCAAGCGCGAACACCAGGCGGCCCCAGCA |
| | | CAGAACAGCCCTGACACAAGGCCACCACCAGCCACCC |
| | | CAATCTGCATCCTCCTCGTGGGACCCCCGAGGACCAAC |
| | | CCCCAAGGCTGCCCCCGATCCAAACCACCAACCGCATC |
| | | CCCACCACCCCCGGGAAAGAAACCCCCAGCAATTGGA |
| | | AGGCCCCTCCCCCTCTTCCTCAACACAAGAACTCCACA |
| | | ACCGAACCGCACAAGCGACCGAGGTGACCCAACCGCA |
| | | GGCATCCGACTCCCTAGACAGATCCTCTCTCCCCGGCA |
| | | AACTAAACAAAACTTAGGGCCAAGGAACATACACACC |
| | | CAACAGAACCCAGACCCCGGCCCACGGCGCCGCGCCC |
| | | CCAACCCCCGACAACCAGAGGGAGCCCCCAACCAATC |
| | | CCGCCGGCTCCCCCGGTGCCCACAGGCAGGGACACCA |
| | | ACCCCCGAACAGACCCAGCACCCAACCATCGACAATC |
| | | CAAGACGGGGGGGCCCCCCCAAAAAAAGGCCCCCAGG |
| | | GGCCGACAGCCAGCACCGCGAGGAAGCCCACCCACCC |
| | | CACACACGACCACGGCAACCAAACCAGAACCCAGACC |
| | | ACCCTGGGCCACCAGCTCCCAGACTCGGCCATCACCCC |
| | | GCAGAAAGGAAAGGCCACAACCCGCGCACCCCAGCCC |
| | | CGATCCGGCGGGGAGCCACCCAACCCGAACCAGCACC |
| | | CAAGAGCGATCCCCGAAGGACCCCCGAACCGCAAAGG |
| | | ACATCAGTATCCCACAGCCTCTCCAAGTCCCCCGGTCT |
| | | CCTCCTCTTCTCGAAGGGACCAAAAGATCAATCCACCA |
| | | CACCCGACGACACTCAACTCCCCACCCCTAAAGGAGAC |
| | | ACCGGGAATCCCAGAATCAAGACTCATCCAATGTCCAT |
| | | CATGGGTCTCAAGGTGAACGTCTCTGCCATATTCATGG |
| | | CAGTACTGTTAACTCTCCAAACACCCACCGGTCAAATC |
| | | CATTGGGGCAATCTCTCTAAGATAGGGGTGGTAGGAAT |
| | | AGGAAGTGCAAGCTACAAAGTTATGACTCGTTCCAGCC |
| | | ATCAATCATTAGTCATAAAATTAATGCCCAATATAACT |
| | | CTCCTCAATAACTGCACGAGGGTAGAGATTGCAGAATA |
| | | CAGGAGACTACTGAGAACAGTTTTGGAACCAATTAGA |
| | | GATGCACTTAATGCAATGACCCAGAATATAAGACCGGT |
| | | TCAGAGTGTAGCTTCAAGTAGGAGACACAAGAGATTT |
| | | GCGGGAGTAGTCCTGGCAGGTGCGGCCCTAGGCGTTGC |
| | | CACAGCTGCTCAGATAACAGCCGGCATTGCACTTCACC |
| | | AGTCCATGCTGAACTCTCAAGCCATCGACAATCTGAGA |
| | | GCGAGCCTGGAAACTACTAATCAGGCAATTGAGACAA |
| | | TCAGACAAGCAGGGCAGGAGATGATATTGGCTGTTCA |
| | | GGGTGTCCAAGACTACATCAATAATGAGCTGATACCGT |
| | | CTATGAACCAACTATCTTGTGATTTAATCGGCCAGAAG |
| | | CTCGGGCTCAAATTGCTCAGATACTATACAGAAATCCT |
| | | GTCATTATTTGGCCCCAGTTTACGGGACCCCATATCTG |
| | | CGGAGATATCTATCCAGGCTTTGAGCTATGCGCTTGGA |
| | | GGAGACATCAATAAGGTGTTAGAAAAGCTCGGATACA |
| | | GTGGAGGTGATTTACTGGGCATCTTAGAGAGCGGAGG |
| | | AATAAAGGCCCGGATAACTCACGTCGACACAGAGTCC |
| | | TACTTCATTGTCCTCAGTATAGCCTATCCGACGCTGTCC |
| | | GAGATTAAGGGGGTGATTGTCCACCGGCTAGAGGGGG |
| | | TCTCGTACAACATAGGCTCTCAAGAGTGGTATACCACT |
| | | GTGCCCAAGTATGTTGCAACCCAAGGGTACCTTATCTC |
| | | GAATTTTGATGAGTCATCGTGTACTTTCATGCCAGAGG |
| | | GGACTGTGTGCAGCCAAAATGCCTTGTACCCGATGAGT |
| | | CCTCTGCTCCAAGAATGCCTCCGGGGGTACACCAAGTC |
| | | CTGTGCTCGTACACTCGTATCCGGGTCTTTTGGGAACC |
| | | GGTTCATTTTATCACAAGGGAACCTAATAGCCAATTGT |
| | | GCATCAATCCTTTGCAAGTGTTACACAACAGGAACGAT |
| | | CATTAATCAAGACCCTGACAAGATCCTAACATACATTG |
| | | CTGCCGATCACTGCCCGGTAGTCGAGGTGAACGGCGTG |
| | | ACCATCCAAGTCGGGAGCAGGAGGTATCCAGACGCTG |
| | | TGTACTTGCACAGAATTGACCTCGGTCCTCCCATATCA |
| | | TTGGAGAGGTTGGACGTAGGGACAAATCTGGGGAATG |
| | | CAATTGCTAAGTTGGAGGATGCCAAGGAATTGTTGGAG |
| | | TCATCGGACCAGATATTGAGGAGTATGAAAGGTTTATC |
| | | GAGCACTAGCATAGTCTACATCCTGATTGCAGTGTGTC |
| | | TTGGAGGGTTGATAGGGATCCCCGCTTTAATATGTTGC |
| | | TGCAGGGGGCGTTGTAACAAAAAGGGAGAACAAGTTG |
| | | GTATGTCAAGACCAGGCCTAAAGCCTGATCTTACGGGA |
| | | ACATCAAAATCCTATGTAAGGTCGCTCTGATCCTCTAC |
| | | AACTCTTGAAACACAAATGTCCCACAAGTCTCCTCTTC |
| | | GTCATCAAGCAACCACCGCACCCAGCATCAAGCCCACC |
| | | TGAAATTATCTCCGGCTTCCCTCTGGCCGAACAATATC |
| | | GGTAGTTAATCAAAACTTAGGGTGCAAGATCATCCACA |
| | | ATGTCACCACAACGAGACCGGATAAATGCCTTCTACAA |
| | | AGATAACCCCCATCCCAAGGGAAGTAGGATAGTCATT |
| | | AACAGAGAACATCTTATGATTGATAGACCTTATGTTTT |
| | | GCTGGCTGTTCTGTTTGTCATGTTTCTGAGCTTGATCGG |
| | | GTTGCTAGCCATTGCAGGCATTAGACTTCATCGGGCAG |
| | | CCATCTACACCGCAGAGATCCATAAAAGCCTCAGCACC |
| | | AATCTAGATGTAACTAACTCAATCGAGCATCAGGTCAA |
| | | GGACGTGCTGACACCACTCTTCAAAATCATCGGTGATG |
| | | AAGTGGGCCTGAGGACACCTCAGAGATTCACTGACCTA |
| | | GTGAAATTAATCTCTGACAAGATTAAATTCCTTAATCC |
| | | GGATAGGGAGTACGACTTCAGAGATCTCACTTGGTGTA |
| | | TCAACCCGCCAGAGAGAATCAAATTGGATTATGATCAA |
| | | TACTGTGCAGATGTGGCTGCTGAAGAGCTCATGAATGC |
| | | ATTGGTGAACTCAACTCTACTGGAGACCAGAACAACCA |
| | | ATCAGTTCCTAGCTGTCTCAAAGGGAAACTGCTCAGGG |
| | | CCCACTACAATCAGAGGTCAATTCTCAAACATGTCGCT |
| | | GTCCCTGTTAGACTTGTATTTAGGTCGAGGTTACAATG |
| | | TGTCATCTATAGTCACTATGACATCCCAGGGAATGTAT |
| | | GGGGGAACTTACCTAGTGGAAAAGCCTAATCTGAGCA |
| | | GCAAAAGGTCAGAGTTGTCACAACTGAGCATGTACCG |
| | | AGTGTTTGAAGTAGGTGTTATCAGAAATCCGGGTTTGG |
| | | GGGCTCCGGTGTTCCATATGACAAACTATCTTGAGCAA |
| | | CCAGTCAGTAATGATCTCAGCAACTGTATGGTGGCTTT |
| | | GGGGGAGCTCAAACTCGCAGCCCTTTGTCACGGGGAA |
| | | GATTCTATCACAATTCCCTATCAGGGATCAGGGAAAGG |
| | | TGTCAGCTTCCAGCTCGTCAAGCTAGGTGTCTGGAAAT |
| | | CCCCAACCGACATGCAATCCTGGGTCCCCTTATCAACG |
| | | GATGATCCAGTGATAGACAGGCTTTACCTCTCATCTCA |
| | | CAGAGGTGTTATCGCTGACAATCAAGCAAAATGGGCT |
| | | GTCCCGACAACACGAACAGATGACAAGTTGCGAATGG |
| | | AGACATGCTTCCAACAGGCGTGTAAGGGTAAAATCCA |
| | | AGCACTCTGCGAGAATCCCGAGTGGGCACCATTGAAG |
| | | GATAACAGGATTCCTTCATACGGGGTCTTGTCTGTTGA |
| | | TCTGAGTCTGACAGTTGAGCTTAAAATCAAAATTGCTT |
| | | CGGGATTCGGGCCATTGATCACACACGGTTCAGGGATG |
| | | GACCTATACAAATCCAACCACAACAATGTGTATTGGCT |
| | | GACTATCCCGCCAATGAAGAACCTAGCCTTAGGTGTAA |
| | | TCAACACATTGGAGTGGATACCGAGATTCAAGGTTAGT |
| | | CCCTACCTCTTCACTGTCCCAATTAAGGAAGCAGGCGA |
| | | AGACTGCCATGCCCCAACATACCTACCTGCGGAGGTGG |
| | | ATGGTGATGTCAAACTCAGTTCCAATCTGGTGATTCTA |
| | | CCTGGTCAAGATCTCCAATATGTTTTGGCAACCTACGA |
| | | TACTTCCAGGGTTGAACATGCTGTGGTTTATTACGTTTA |
| | | CAGCCCAAGCCGCTCATTTTCTTACTTTTATCCTTTTAG |
| | | GTTGCCTATAAAGGGGGTCCCCATCGAATTACAAGTGG |
| | | AATGCTTCACATGGGACCAAAAACTCTGGTGCCGTCAC |
| | | TTCTGTGTGCTTGCGGACTCAGAATCTGGTGGACATAT |
| | | CACTCACTCTGGGATGGTGGGCATGGGAGTCAGCTGCA |
| | | CAGTCACCCGGGAAGATGGAACCAATCGCAGATAGGG |
| | | CTGCTAGTGAACCAATCACATGATGTCACCCAGACATC |
| | | AGGCATACCCACTAGTCTACCCTCCATCATTGTTATAA |
| | | AAAACTTAGGAACCAGGTCCACACAGCCGCCAGCCCA |
| | | TCAACGCGTACGGCCACCATGGAGGCCGCTTTGCTGGT |
| | | GTGTCAGTACACAATTCAGAGCCTGATCCACTTGACGG |
| | | GAGAGGACCCTGGCTTTTTCAACGTTGAGATCCCCGAA |
| | | TTCCCATTTTATCCAACCTGCAACGTGTGTACTGCAGA |
| | | CGTAAACGTTACTATTAATTTCGACGTGGGAGGCAAGA |
| | | AACACCAACTGGATCTCGACTTTGGACAGCTGACTCCC |
| | | CACACCAAAGCTGTTTACCAACCCAGAGGGGCTTTTGG |
| | | CGGCTCTGAAAACGCTACCAACCTCTTCCTGCTCGAGC |
| | | TTCTGGGAGCTGGCGAGCTTGCTCTGACTATGCGCTCT |
| | | AAAAAGCTCCCGATTAACGTCACAACAGGGGAGGAAC |
| | | AACAGGTGAGCTTGGAGTCCGTAGACGTGTACTTCCAG |
| | | GACGTGTTCGGAACAATGTGGTGTCATCACGCTGAGAT |
| | | GCAGAATCCCGTGTACCTCATTCCAGAGACGGTGCCTT |
| | | ATATAAAGTGGGATAACTGTAACTCAACGAATATCACA |
| | | GCAGTGGTTAGGGCGCAAGGTCTGGACGTCACTCTTCC |
| | | TCTCTCACTCCCAACTTCCGCTCAAGACAGCAATTTCTC |
| | | AGTGAAAACAGAAATGCTGGGCAACGAGATAGATATT |
| | | GAGTGTATAATGGAAGACGGGGAGATTAGTCAAGTGC |
| | | TGCCTGGCGACAACAAATTTAATATCACCTGTAGCGGT |
| | | TACGAGAGTCACGTCCCGTCCGGGGGGATTCTGACCAG |
| | | CACTAGCCCTGTCGCCACTCCCATTCCGGGAACTGGAT |
| | | ACGCCTATAGCCTTAGGCTTACACCCAGACCTGTGTCA |
| | | CGGTTTCTGGGCAACAACAGCATTCTGTACGTGTTCTA |
| | | CAGTGGCAACGGGCCTAAAGCCTCTGGTGGCGACTATT |
| | | GTATTCAAAGCAACATCGTCTTCTCCGACGAGATCCCT |
| | | GCAAGCCAGGACATGCCCACTAATACCACCGACATTAC |
| | | CTACGTAGGGGACAACGCTACTTACTCTGTCCCGATGG |
| | | TGACTAGCGAGGATGCTAATTCTCCAAACGTTACCGTA |
| | | ACCGCCTTTTGGGCTTGGCCCAATAATACAGAGACGGA |
| | | TTTCAAATGCAAGTGGACTTTGACTTCCGGCACCCCCT |
| | | CCGGCTGTGAAAATATTTCTGGCGCTTTCGCCAGCAAC |
| | | AGGACGTTCGACATCACAGTCTCTGGTCTTGGCACTGC |
| | | CCCAAAGACCCTGATCATCACCCGGACTGCCACTAACG |
| | | CGACAACTACAACTCATAAGGTTATTTTTAGCAAGGCA |
| | | CCAGAGAGCACCACTACATCTCCCACCCTGAACACTAC |
| | | GGGCTTTGCTGACCCGAATACCACCACGGGCCTTCCCT |
| | | CATCAACCCATGTCCCAACCAACTTGACAGCACCAGCA |
| | | TCAACGGGTCCCACCGTCTCCACAGCAGACGTAACCTC |
| | | TCCTACACCTGCCGGCACCACAAGTGGCGCTAGTCCTG |
| | | TGACCCCTTCCCCATCTCCTTGGGATAATGGCACAGAG |
| | | TCAAAAGCCCCTGATATGACTAGCTCCACCAGTCCTGT |
| | | GACGACACCAACCCCAAACGCTACCTCCCCCACTCCAG |
| | | CCGTTACAACTCCAACGCCAAACGCTACCTCCCCTACT |
| | | CCTGCCGTAACCACGCCTACCCCTAATGCCACCAGTCC |
| | | TACTTTGGGCAAGACCTCCCCGACAAGCGCTGTGACAA |
| | | CCCCTACGCCCAACGCAACTTCTCCTACACTCGGAAAG |
| | | ACCTCACCTACCAGTGCTGTCACCACACCTACACCAAA |
| | | CGCCACTAGCCCCACATTGGGTAAGACATCCCCCACTT |
| | | CCGCAGTTACGACTCCCACACCGAACGCAACCGGACCT |
| | | ACTGTCGGTGAGACATCCCCTCAGGCCAACGCCACAAA |
| | | TCACACACTGGGCGGCACCTCACCAACTCCAGTAGTGA |
| | | CCTCCCAGCCAAAAAACGCCACCTCTGCTGTGACCACC |
| | | GGTCAGCATAACATCACAAGTTCCTCCACTTCTAGCAT |
| | | GAGTTTGCGCCCCTCATCCAACCCAGAAACGCTGAGCC |
| | | CTTCAACATCTGACAACTCAACAAGCCATATGCCTCTG |
| | | CTCACTAGTGCTCACCCAACAGGGGGAGAAAACATTA |
| | | CACAGGTGACACCCGCCAGCATCTCTACACACCACGTG |
| | | AGCACAAGCTCTCCCGCTCCTCGGCCAGGTACAACCTC |
| | | CCAAGCCTCTGGGCCTGGAAATTCCTCAACCTCCACCA |
| | | AACCCGGTGAGGTCAACGTCACCAAGGGAACACCCCC |
| | | CCAGAACGCAACGTCACCTCAAGCACCCAGCGGACAG |
| | | AAGACCGCTGTGCCTACTGTAACCTCTACCGGAGGGAA |
| | | AGCTAATAGTACGACTGGAGGTAAGCACACTACGGGC |
| | | CACGGTGCCAGAACCAGTACAGAGCCTACCACTGATTA |
| | | CGGCGGAGATAGTACAACCCCCCGCCCTAGATACAAC |
| | | GCCACAACTTATTTGCCTCCCTCAACGTCCTCCAAGCT |
| | | GAGACCCAGGTGGACGTTTACGTCTCCCCCAGTCACAA |
| | | CTGCGCAGGCAACCGTGCCAGTTCCACCTACAAGTCAG |
| | | CCGCGCTTCAGCAATCTCTCAATGCTCGTGCTGCAGTG |
| | | GGCCTCTCTGGCAGTACTGACCCTCTTGCTTCTCTTGGT |
| | | GATGGCTGACTGTGCCTTTCGGAGAAACCTGAGCACCT |
| | | CACACACTTATACCACACCTCCCTATGACGATGCCGAG |
| | | ACATATGTATAAGCGCGCAGCGCTTAGACGTCTCGCGA |
| | | TCGATGCTAGTGTGAAATAGACATCAGAATTAAGAAA |
| | | AACGTAGGGTCCAAGTGGTTCCCCGTTATGGACTCGCT |
| | | ATCTGTCAACCAGATCTTATACCCTGAAGTTCACCTAG |
| | | ATAGCCCGATAGTTACCAATAAGATAGTAGCCATCCTG |
| | | GAGTATGCTCGAGTCCCTCACGCTTACAGCCTGGAGGA |
| | | CCCTACACTGTGTCAGAACATCAAGCACCGCCTAAAAA |
| | | ACGGATTTTCCAACCAAATGATTATAAACAATGTGGAA |
| | | GTTGGGAATGTCATCAAGTCCAAGCTTAGGAGTTATCC |
| | | GGCCCACTCTCATATTCCATATCCAAATTGTAATCAGG |
| | | ATTTATTTAACATAGAAGACAAAGAGTCAACGAGGAA |
| | | GATCCGTGAACTCCTCAAAAAGGGGAATTCGCTGTACT |
| | | CCAAAGTCAGTGATAAGGTTTTCCAATGCTTAAGGGAC |
| | | ACTAACTCACGGCTTGGCCTAGGCTCCGAATTGAGGGA |
| | | GGACATCAAGGAGAAAGTTATTAACTTGGGAGTTTACA |
| | | TGCACAGCTCCCAGTGGTTTGAGCCCTTTCTGTTTTGGT |
| | | TTACAGTCAAGACTGAGATGAGGTCAGTGATTAAATCA |
| | | CAAACCCATACTTGCCATAGGAGGAGACACACACCTGT |
| | | ATTCTTCACTGGTAGTTCAGTTGAGTTGCTAATCTCTCG |
| | | TGACCTTGTTGCTATAATCAGTAAAGAGTCTCAACATG |
| | | TATATTACCTGACATTTGAACTGGTTTTGATGTATTGTG |
| | | ATGTCATAGAGGGGAGGTTAATGACAGAGACCGCTAT |
| | | GACTATTGATGCTAGGTATACAGAGCTTCTAGGAAGAG |
| | | TCAGATACATGTGGAAACTGATAGATGGTTTCTTCCCT |
| | | GCACTCGGGAATCCAACTTATCAAATTGTAGCCATGCT |
| | | GGAGCCTCTTTCACTTGCTTACCTGCAGCTGAGGGATA |
| | | TAACAGTAGAACTCAGAGGTGCTTTCCTTAACCACTGC |
| | | TTTACTGAAATACATGATGTTCTTGACCAAAACGGGTT |
| | | TTCTGATGAAGGTACTTATCATGAGTTAACTGAAGCTC |
| | | TAGATTACATTTTCATAACTGATGACATACATCTGACA |
| | | GGGGAGATTTTCTCATTTTTCAGAAGTTTCGGCCACCC |
| | | CAGACTTGAAGCAGTAACGGCTGCTGAAAATGTTAGG |
| | | AAATACATGAATCAGCCTAAAGTCATTGTGTATGAGAC |
| | | TCTGATGAAAGGTCATGCCATATTTTGTGGAATCATAA |
| | | TCAACGGCTATCGTGACAGGCACGGAGGCAGTTGGCC |
| | | ACCGCTGACCCTCCCCCTGCATGCTGCAGACACAATCC |
| | | GGAATGCTCAAGCTTCAGGTGAAGGGTTAACACATGA |
| | | GCAGTGCGTTGATAACTGGAAATCTTTTGCTGGAGTGA |
| | | AATTTGGCTGCTTTATGCCTCTTAGCCTGGATAGTGATC |
| | | TGACAATGTACCTAAAGGACAAGGCACTTGCTGCTCTC |
| | | CAAAGGGAATGGGATTCAGTTTACCCGAAAGAGTTCCT |
| | | GCGTTACGACCCTCCCAAGGGAACCGGGTCACGGAGG |
| | | CTTGTAGATGTTTTCCTTAATGATTCGAGCTTTGACCCA |
| | | TATGATGTGATAATGTATGTTGTAAGTGGAGCTTACCT |
| | | CCATGACCCTGAGTTCAACCTGTCTTACAGCCTGAAAG |
| | | AAAAGGAGATCAAGGAAACAGGTAGACTTTTTGCTAA |
| | | AATGACTTACAAAATGAGGGCATGCCAAGTGATTGCTG |
| | | AAAATCTAATCTCAAACGGGATTGGCAAATATTTTAAG |
| | | GACAATGGGATGGCCAAGGATGAGCACGATTTGACTA |
| | | AGGCACTCCACACTCTAGCTGTCTCAGGAGTCCCCAAA |
| | | GATCTCAAAGAAAGTCACAGGGGGGGGCCAGTCTTAA |
| | | AAACCTACTCCCGAAGCCCAGTCCACACAAGTACCAG |
| | | GAACGTGAGAGCAGCAAAAGGGTTTATAGGGTTCCCT |
| | | CAAGTAATTCGGCAGGACCAAGACACTGATCATCCGG |
| | | AGAATATGGAAGCTTACGAGACAGTCAGTGCATTTATC |
| | | ACGACTGATCTCAAGAAGTACTGCCTTAATTGGAGATA |
| | | TGAGACCATCAGCTTGTTTGCACAGAGGCTAAATGAGA |
| | | TTTACGGATTGCCCTCATTTTTCCAGTGGCTGCATAAGA |
| | | GGCTTGAGACCTCTGTCCTGTATGTAAGTGACCCTCAT |
| | | TGCCCCCCCGACCTTGACGCCCATATCCCGTTATATAA |
| | | AGTCCCCAATGATCAAATCTTCATTAAGTACCCTATGG |
| | | GAGGTATAGAAGGGTATTGTCAGAAGCTGTGGACCAT |
| | | CAGCACCATTCCCTATCTATACCTGGCTGCTTATGAGA |
| | | GCGGAGTAAGGATTGCTTCGTTAGTGCAAGGGGACAA |
| | | TCAGACCATAGCCGTAACAAAAAGGGTACCCAGCACA |
| | | TGGCCCTACAACCTTAAGAAACGGGAAGCTGCTAGAG |
| | | TAACTAGAGATTACTTTGTAATTCTTAGGCAAAGGCTA |
| | | CATGATATTGGCCATCACCTCAAGGCAAATGAGACAAT |
| | | TGTTTCATCACATTTTTTTGTCTATTCAAAAGGAATATA |
| | | TTATGATGGGCTACTTGTGTCCCAATCACTCAAGAGCA |
| | | TCGCAAGATGTGTATTCTGGTCAGAGACTATAGTTGAT |
| | | GAAACAAGGGCAGCATGCAGTAATATTGCTACAACAA |
| | | TGGCTAAAAGCATCGAGAGAGGTTATGACCGTTACCTT |
| | | GCATATTCCCTGAACGTCCTAAAAGTGATACAGCAAAT |
| | | TCTGATCTCTCTTGGCTTCACAATCAATTCAACCATGAC |
| | | CCGGGATGTAGTCATACCCCTCCTCACAAACAACGACC |
| | | TCTTAATAAGGATGGCACTGTTGCCCGCTCCTATTGGG |
| | | GGGATGAATTATCTGAATATGAGCAGGCTGTTTGTCAG |
| | | AAACATCGGTGATCCAGTAACATCATCAATTGCTGATC |
| | | TCAAGAGAATGATTCTCGCCTCACTAATGCCTGAAGAG |
| | | ACCCTCCATCAAGTAATGACACAACAACCGGGGGACT |
| | | CTTCATTCCTAGACTGGGCTAGCGACCCTTACTCAGCA |
| | | AATCTTGTATGTGTCCAGAGCATCACTAGACTCCTCAA |
| | | GAACATAACTGCAAGGTTTGTCCTGATCCATAGTCCAA |
| | | ACCCAATGTTAAAAGGATTATTCCATGATGACAGTAAA |
| | | GAAGAGGACGAGGGACTGGCGGCATTCCTCATGGACA |
| | | GGCATATTATAGTACCTAGGGCAGCTCATGAAATCCTG |
| | | GATCATAGTGTCACAGGGGCAAGAGAGTCTATTGCAG |
| | | GCATGCTGGATACCACAAAAGGCTTGATTCGAGCCAGC |
| | | ATGAGGAAGGGGGGGTTAACCTCTCGAGTGATAACCA |
| | | GATTGTCCAATTATGACTATGAACAATTCAGAGCAGGG |
| | | ATGGTGCTATTGACAGGAAGAAAGAGAAATGTCCTCA |
| | | TTGACAAAGAGTCATGTTCAGTGCAGCTGGCGAGAGCT |
| | | CTAAGAAGCCATATGTGGGCGAGGCTAGCTCGAGGAC |
| | | GGCCTATTTACGGCCTTGAGGTCCCTGATGTACTAGAA |
| | | TCTATGCGAGGCCACCTTATTCGGCGTCATGAGACATG |
| | | TGTCATCTGCGAGTGTGGATCAGTCAACTACGGATGGT |
| | | TTTTTGTCCCCTCGGGTTGCCAACTGGATGATATTGACA |
| | | AGGAAACATCATCCTTGAGAGTCCCATATATTGGTTCT |
| | | ACCACTGATGAGAGAACAGACATGAAGCTTGCCTTCGT |
| | | AAGAGCCCCAAGTCGATCCTTGCGATCTGCTGTTAGAA |
| | | TAGCAACAGTGTACTCATGGGCTTACGGTGATGATGAT |
| | | AGCTCTTGGAACGAAGCCTGGTTGTTGGCTAGGCAAAG |
| | | GGCCAATGTGAGCCTGGAGGAGCTAAGGGTGATCACT |
| | | CCCATCTCAACTTCGACTAATTTAGCGCATAGGTTGAG |
| | | GGATCGTAGCACTCAAGTGAAATACTCAGGTACATCCC |
| | | TTGTCCGAGTGGCGAGGTATACCACAATCTCCAACGAC |
| | | AATCTCTCATTTGTCATATCAGATAAGAAGGTTGATAC |
| | | TAACTTTATATACCAACAAGGAATGCTTCTAGGGTTGG |
| | | GTGTTTTAGAAACATTGTTTCGACTCGAGAAAGATACC |
| | | GGATCATCTAACACGGTATTACATCTTCACGTCGAAAC |
| | | AGATTGTTGCGTGATCCCGATGATAGATCATCCCAGGA |
| | | TACCCAGCTCCCGCAAGCTAGAGCTGAGGGCAGAGCT |
| | | ATGTACCAACCCATTGATATATGATAATGCACCTTTAA |
| | | TTGACAGAGATGCAACAAGGCTATACACCCAGAGCCA |
| | | TAGGAGGCACCTTGTGGAATTTGTTACATGGTCCACAC |
| | | CCCAACTATATCACATTTTAGCTAAGTCCACAGCACTA |
| | | TCTATGATTGACCTGGTAACAAAATTTGAGAAGGACCA |
| | | TATGAATGAAATTTCAGCTCTCATAGGGGATGACGATA |
| | | TCAATAGTTTCATAACTGAGTTTCTGCTCATAGAGCCA |
| | | AGATTATTCACTATCTACTTGGGCCAGTGTGCGGCCAT |
| | | CAATTGGGCATTTGATGTACATTATCATAGACCATCAG |
| | | GGAAATATCAGATGGGTGAGCTGTTGTCATCGTTCCTT |
| | | TCTAGAATGAGCAAAGGAGTGTTTAAGGTGCTTGTCAA |
| | | TGCTCTAAGCCACCCAAAGATCTACAAGAAATTCTGGC |
| | | ATTGTGGTATTATAGAGCCTATCCATGGTCCTTCACTTG |
| | | ATGCTCAAAACTTGCACACAACTGTGTGCAACATGGTT |
| | | TACACATGCTATATGACCTACCTCGACCTGTTGTTGAA |
| | | TGAAGAGTTAGAAGAGTTCACATTTCTCTTGTGTGAAA |
| | | GCGACGAGGATGTAGTACCGGACAGATTCGACAACAT |
| | | CCAGGCAAAACACTTATGTGTTCTGGCAGATTTGTACT |
| | | GTCAACCAGGGACCTGCCCACCAATTCGAGGTCTAAGA |
| | | CCGGTAGAGAAATGTGCAGTTCTAACCGACCATATCAA |
| | | GGCAGAGGCTATGTTATCTCCAGCAGGATCTTCGTGGA |
| | | ACATAAATCCAATTATTGTAGACCATTACTCATGCTCT |
| | | CTGACTTATCTCCGGCGAGGATCGATCAAACAGATAAG |
| | | ATTGAGAGTTGATCCAGGATTCATTTTCGACGCCCTCG |
| | | CTGAGGTAAATGTCAGTCAGCCAAAGATCGGCAGCAA |
| | | CAACATCTCAAATATGAGCATCAAGGCTTTCAGACCCC |
| | | CACACGATGATGTTGCAAAATTGCTCAAAGATATCAAC |
| | | ACAAGCAAGCACAATCTTCCCATTTCAGGGGGCAATCT |
| | | CGCCAATTATGAAATCCATGCTTTCCGCAGAATCGGGT |
| | | TGAACTCATCTGCTTGCTACAAAGCTGTTGAGATATCA |
| | | ACATTAATTAGGAGATGCCTTGAGCCAGGGGAGGACG |
| | | GCTTGTTCTTGGGTGAGGGATCGGGTTCTATGTTGATC |
| | | ACTTATAAAGAGATACTTAAACTAAACAAGTGCTTCTA |
| | | TAATAGTGGGGTTTCCGCCAATTCTAGATCTGGTCAAA |
| | | GGGAATTAGCACCCTATCCCTCCGAAGTTGGCCTTGTC |
| | | GAACACAGAATGGGAGTAGGTAATATTGTCAAAGTGC |
| | | TCTTTAACGGGAGGCCCGAAGTCACGTGGGTAGGCAGT |
| | | GTAGATTGCTTCAATTTCATAGTTAGTAATATCCCTACC |
| | | TCTAGTGTGGGGTTTATCCATTCAGATATAGAGACCTT |
| | | GCCTGACAAAGATACTATAGAGAAGCTAGAGGAATTG |
| | | GCAGCCATCTTATCGATGGCTCTGCTCCTGGGCAAAAT |
| | | AGGATCAATACTGGTGATTAAGCTTATGCCTTTCAGCG |
| | | GGGATTTTGTTCAGGGATTTATAAGTTATGTAGGGTCT |
| | | CATTATAGAGAAGTGAACCTTGTATACCCTAGATACAG |
| | | CAACTTCATCTCTACTGAATCTTATTTGGTTATGACAGA |
| | | TCTCAAGGCTAACCGGCTAATGAATCCTGAAAAGATTA |
| | | AGCAGCAGATAATTGAATCATCTGTGAGGACTTCACCT |
| | | GGACTTATAGGTCACATCCTATCCATTAAGCAACTAAG |
| | | CTGCATACAAGCAATTGTGGGAGACGCAGTTAGTAGA |
| | | GGTGATATCAATCCTACTCTGAAAAAACTTACACCTAT |
| | | AGAGCAGGTGCTGATCAATTGCGGGTTGGCAATTAACG |
| | | GACCTAAGCTGTGCAAAGAATTGATCCACCATGATGTT |
| | | GCCTCAGGGCAAGATGGATTGCTTAATTCTATACTCAT |
| | | CCTCTACAGGGAGTTGGCAAGATTCAAAGACAACCAA |
| | | AGAAGTCAACAAGGGATGTTCCACGCTTACCCCGTATT |
| | | GGTAAGTAGCAGGCAACGAGAACTTATATCTAGGATC |
| | | ACCCGCAAATTCTGGGGGCACATTCTTCTTTACTCCGG |
| | | GAACAAAAAGTTGATAAATAAGTTTATCCAGAATCTCA |
| | | AGTCCGGCTATCTGATACTAGACTTACACCAGAATATC |
| | | TTCGTTAAGAATCTATCCAAGTCAGAGAAACAGATTAT |
| | | TATGACGGGGGGTTTGAAACGTGAGTGGGTTTTTAAGG |
| | | TAACAGTCAAGGAGACCAAAGAATGGTATAAGTTAGT |
| | | CGGATACAGTGCCCTGATTAAGGACTAATTGGTTGAAC |
| | | TCCGGAACCCTAATCCTGCCCTAGGTGGTTAGGCATTA |
| | | TTTGCAATATATTAAAGAAAACTTTGAAAATACGAAGT |
| | | TTCTATTCCCAGCTTTGTCTGGT |
| 60 | B-02 | ACCAAACAAAGTTGGGTAAGGATAGTTCAATCAATGA |
| | (gp350_ATUa_ | TCATCTTCTAGTGCACTTAGGATTCAAGATCCTATTATC |
| | LMP2 at | AGGGACAAGAGCAGGATTAGGGATATCCGAGATGGCC |
| | ATU3) | ACACTTTTAAGGAGCTTAGCATTGTTCAAAAGAAACAA |
| | MV leader: nt | GGACAAACCACCCATTACATCAGGATCCGGTGGAGCC |
| | 1-55 | ATCAGAGGAATCAAACACATTATTATAGTACCAATCCC |
| | MV N ORF: nt | TGGAGATTCCTCAATTACCACTCGATCCAGACTTCTGG |
| | 108-1,685 | ACCGGTTGGTGAGGTTAATTGGAAACCCGGATGTGAGC |
| | MV P ORF: nt | GGGCCCAAACTAACAGGGGCACTAATAGGTATATTATC |
| | 1,807-3,330 | CTTATTTGTGGAGTCTCCAGGTCAATTGATTCAGAGGA |
| | MV M ORF: nt | TCACCGATGACCCTGACGTTAGCATAAGGCTGTTAGAG |
| | 3,438-4,445 | GTTGTCCAGAGTGACCAGTCACAATCTGGCCTTACCTT |
| | MV F ORF: nt | CGCATCAAGAGGTACCAACATGGAGGATGAGGCGGAC |
| | 5,449-7,110 | CAATACTTTTCACATGATGATCCAATTAGTAGTGATCA |
| | MV H ORF: nt | ATCCAGGTTCGGATGGTTCGGGAACAAGGAAATCTCA |
| | 7,271-9,124 | GATATTGAAGTGCAAGACCCTGAGGGATTCAACATGAT |
| | ATU upstream | TCTGGGTACCATCCTAGCCCAAATTTGGGTCTTGCTCG |
| | motif: nt 9,180- | CAAAGGCGGTTACGGCCCCAGACACGGCAGCTGATTC |
| | 9,257 | GGAGCTAAGAAGGTGGATAAAGTACACCCAACAAAGA |
| | GE of N gene: | AGGGTAGTTGGTGAATTTAGATTGGAGAGAAAATGGTT |
| | nt 9,196-9,206 | GGATGTGGTGAGGAACAGGATTGCCGAGGACCTCTCCT |
| | Conserved | TACGCCGATTCATGGTCGCTCTAATCCTGGATATCAAG |
| | trinucleotide | AGAACACCCGGAAACAAACCCAGGATTGCTGAAATGA |
| | motif: 9,207- | TATGTGACATTGATACATATATCGTAGAGGCAGGATTA |
| | 9,209 | GCCAGTTTTATCCTGACTATTAAGTTTGGGATAGAAAC |
| | GS of P gene: | TATGTATCCTGCTCTTGGACTGCATGAATTTGCTGGTGA |
| | nt 9,210-9,226 | GTTATCCACACTTGAGTCCTTGATGAACCTTTACCAGC |
| | BsiWI | AAATGGGGGAAACTGCACCCTACATGGTAATCCTGGA |
| | restriction site: | GAACTCAATTCAGAACAAGTTCAGTGCAGGATCATACC |
| | nt 9,246-9,251 | CTCTGCTCTGGAGCTATGCCATGGGAGTAGGAGTGGAA |
| | Kozak | CTTGAAAACTCCATGGGAGGTTTGAACTTTGGCCGATC |
| | sequence: nt | TTACTTTGATCCAGCATATTTTAGATTAGGGCAAGAGA |
| | 9,252-9,260 | TGGTAAGGAGGTCAGCTGGAAAGGTCAGTTCCACATTG |
| | EBV gp350 | GCATCTGAACTCGGTATCACTGCCGAGGATGCAAGGCT |
| | ORF: nt 9,258- | TGTTTCAGAGATTGCAATGCATACTACTGAGGACAAGA |
| | 11,981 | TCAGTAGAGCGGTTGGACCCAGACAAGCCCAAGTATC |
| | ATUa motif: nt | ATTTCTACACGGTGATCAAAGTGAGAATGAGCTACCGA |
| | 11,982-12,125 | GATTGGGGGGCAAGGAAGATAGGAGGGTCAAACAGAG |
| | GE of P gene: | TCGAGGAGAAGCCAGGGAGAGCTACAGAGAAACCGGG |
| | nt 12,049- | CCCAGCAGAGCAAGTGATGCGAGAGCTGCCCATCTTCC |
| | 12,059 | AACCGGCACACCCCTAGACATTGACACTGCAACGGAG |
| | Conserved | TCCAGCCAAGATCCGCAGGACAGTCGAAGGTCAGCTG |
| | trinucleotide | ACGCCCTGCTTAGGCTGCAAGCCATGGCAGGAATCTCG |
| | motif: nt | GAAGAACAAGGCTCAGACACGGACACCCCTATAGTGT |
| | 12,060-12,062 3 | ACAATGACAGAAATCTTCTAGACTAGGTGCGAGAGGC |
| | GS of P gene: | CGAGGGCCAGAACAACATCCGCCTACCATCCATCATTG |
| | nt 12,063- | TTATAAAAAACTTAGGAACCAGGTCCACACAGCCGCC |
| | 12,079 | AGCCCATCAACCATCCACTCCCACGATTGGAGCCAATG |
| | EBV LMP2 | GCAGAAGAGCAGGCACGCCATGTCAAAAACGGACTGG |
| | ORF: nt | AATGCATCCGGGCTCTCAAGGCCGAGCCCATCGGCTCA |
| | 12,126-14,459 | CTGGCCATCGAGGAAGCTATGGCAGCATGGTCAGAAA |
| | ATU | TATCAGACAACCCAGGACAGGAGCGAGCCACCTGCAG |
| | downstream | GGAAGAGAAGGCAGGCAGTTCGGGTCTCAGCAAACCA |
| | motif: nt | TGCCTCTCAGCAATTGGATCAACTGAAGGCGGTGCACC |
| | 14,460-14,495 | TCGCATCCGCGGTCAGGGACCTGGAGAGAGCGATGAC |
| | MV L ORF: nt | GACGCTGAAACTTTGGGAATCCCCCCAAGAAATCTCCA |
| | 14,550-21,101 | GGCATCAAGCACTGGGTTACAGTGTTATTACGTTTATG |
| | MV trailer: nt | ATCACAGCGGTGAAGCGGTTAAGGGAATCCAAGATGC |
| | 21,174-21,210 | TGACTCTATCATGGTTCAATCAGGCCTTGATGGTGATA |
| | | GCACCCTCTCAGGAGGAGACAATGAATCTGAAAACAG |
| | | CGATGTGGATATTGGCGAACCTGATACCGAGGGATATG |
| | | CTATCACTGACCGGGGATCTGCTCCCATCTCTATGGGG |
| | | TTCAGGGCTTCTGATGTTGAAACTGCAGAAGGAGGGG |
| | | AGATCCACGAGCTCCTGAGACTCCAATCCAGAGGCAA |
| | | CAACTTTCCGAAGCTTGGGAAAACTCTCAATGTTCCTC |
| | | CGCCCCCGGACCCCGGTAGGGCCAGCACTTCCGGGAC |
| | | ACCCATTAAAAAGGGCACAGACGCGAGATTAGCCTCA |
| | | TTTGGAACGGAGATCGCGTCTTTATTGACAGGTGGTGC |
| | | AACCCAATGTGCTCGAAAGTCACCCTCGGAACCATCAG |
| | | GGCCAGGTGCACCTGCGGGGAATGTCCCCGAGTGTGTG |
| | | AGCAATGCCGCACTGATACAGGAGTGGACACCCGAAT |
| | | CTGGTACCACAATCTCCCCGAGATCCCAGAATAATGAA |
| | | GAAGGGGGAGACTATTATGATGATGAGCTGTTCTCTGA |
| | | TGTCCAAGATATTAAAACAGCCTTGGCCAAAATACACG |
| | | AGGATAATCAGAAGATAATCTCCAAGCTAGAATCACT |
| | | GCTGTTATTGAAGGGAGAAGTTGAGTCAATTAAGAAG |
| | | CAGATCAACAGGCAAAATATCAGCATATCCACCCTGG |
| | | AAGGACACCTCTCAAGCATCATGATCGCCATTCCTGGA |
| | | CTTGGGAAGGATCCCAACGACCCCACTGCAGATGTCGA |
| | | AATCAATCCCGACTTGAAACCCATCATAGGCAGAGATT |
| | | CAGGCCGAGCACTGGCCGAAGTTCTCAAGAAACCCGTT |
| | | GCCAGCCGACAACTCCAAGGAATGACAAATGGACGGA |
| | | CCAGTTCCAGAGGACAGCTGCTGAAGGAATTTCAGCTA |
| | | AAGCCGATCGGGAAAAAGATGAGCTCAGCCGTCGGGT |
| | | TTGTTCCTGACACCGGCCCTGCATCACGCAGTGTAATC |
| | | CGCTCCATTATAAAATCCAGCCGGCTAGAGGAGGATCG |
| | | GAAGCGTTACCTGATGACTCTCCTTGATGATATCAAAG |
| | | GAGCCAATGATCTTGCCAAGTTCCACCAGATGCTGATG |
| | | AAGATAATAATGAAGTAGCTACAGCTCAACTTACCTGC |
| | | CAACCCCATGCCAGTCGACCCAACTAGTACAACCTAAA |
| | | TCCATTATAAAAAACTTAGGAGCAAAGTGATTGCCTCC |
| | | CAAGGTCCACAATGACAGAGACCTACGACTTCGACAA |
| | | GTCGGCATGGGACATCAAAGGGTCGATCGCTCCGATAC |
| | | AACCCACCACCTACAGTGATGGCAGGCTGGTGCCCCAG |
| | | GTCAGAGTCATAGATCCTGGTCTAGGCGACAGGAAGG |
| | | ATGAATGCTTTATGTACATGTTTCTGCTGGGGGTTGTTG |
| | | AGGACAGCGATTCCCTAGGGCCTCCAATCGGGCGAGC |
| | | ATTTGGGTTCCTGCCCTTAGGTGTTGGCAGATCCACAG |
| | | CAAAGCCCGAAAAACTCCTCAAAGAGGCCACTGAGCT |
| | | TGACATAGTTGTTAGACGTACAGCAGGGCTCAATGAAA |
| | | AACTGGTGTTCTACAACAACACCCCACTAACTCTCCTC |
| | | ACACCTTGGAGAAAGGTCCTAACAACAGGGAGTGTCTT |
| | | CAACGCAAACCAAGTGTGCAATGCGGTTAATCTGATAC |
| | | CGCTCGATACCCCGCAGAGGTTCCGTGTTGTTTATATG |
| | | AGCATCACCCGTCTTTCGGATAACGGGTATTACACCGT |
| | | TCCTAGAAGAATGCTGGAATTCAGATCGGTCAATGCAG |
| | | TGGCCTTCAACCTGCTGGTGACCCTTAGGATTGACAAG |
| | | GCGATAGGCCCTGGGAAGATCATCGACAATACAGAGC |
| | | AACTTCCTGAGGCAACATTTATGGTCCACATCGGGAAC |
| | | TTCAGGAGAAAGAAGAGTGAAGTCTACTCTGCCGATTA |
| | | TTGCAAAATGAAAATCGAAAAGATGGGCCTGGTTTTTG |
| | | CACTTGGTGGGATAGGGGGCACCAGTCTTCACATTAGA |
| | | AGCACAGGCAAAATGAGCAAGACTCTCCATGCACAAC |
| | | TCGGGTTCAAGAAGACCTTATGTTACCCGCTGATGGAT |
| | | ATCAATGAAGACCTTAATCGATTACTCTGGAGGAGCAG |
| | | ATGCAAGATAGTAAGAATCCAGGCAGTTTTGCAGCCAT |
| | | CAGTTCCTCAAGAATTCCGCATTTACGACGACGTGATC |
| | | ATAAATGATGACCAAGGACTATTCAAAGTTCTGTAGAC |
| | | CGTAGTGCCCAGCAATGCCCGAAAACGACCCCCCTCAC |
| | | AATGACAGCCAGAAGGCCCGGACAAAAAAGCCCCCTC |
| | | CGAAAGACTCCACGGACCAAGCGAGAGGCCAGCCAGC |
| | | AGCCGACGGCAAGCGCGAACACCAGGCGGCCCCAGCA |
| | | CAGAACAGCCCTGACACAAGGCCACCACCAGCCACCC |
| | | CAATCTGCATCCTCCTCGTGGGACCCCCGAGGACCAAC |
| | | CCCCAAGGCTGCCCCCGATCCAAACCACCAACCGCATC |
| | | CCCACCACCCCCGGGAAAGAAACCCCCAGCAATTGGA |
| | | AGGCCCCTCCCCCTCTTCCTCAACACAAGAACTCCACA |
| | | ACCGAACCGCACAAGCGACCGAGGTGACCCAACCGCA |
| | | GGCATCCGACTCCCTAGACAGATCCTCTCTCCCCGGCA |
| | | AACTAAACAAAACTTAGGGCCAAGGAACATACACACC |
| | | CAACAGAACCCAGACCCCGGCCCACGGCGCCGCGCCC |
| | | CCAACCCCCGACAACCAGAGGGAGCCCCCAACCAATC |
| | | CCGCCGGCTCCCCCGGTGCCCACAGGCAGGGACACCA |
| | | ACCCCCGAACAGACCCAGCACCCAACCATCGACAATC |
| | | CAAGACGGGGGGGCCCCCCCAAAAAAAGGCCCCCAGG |
| | | GGCCGACAGCCAGCACCGCGAGGAAGCCCACCCACCC |
| | | CACACACGACCACGGCAACCAAACCAGAACCCAGACC |
| | | ACCCTGGGCCACCAGCTCCCAGACTCGGCCATCACCCC |
| | | GCAGAAAGGAAAGGCCACAACCCGCGCACCCCAGCCC |
| | | CGATCCGGCGGGGAGCCACCCAACCCGAACCAGCACC |
| | | CAAGAGCGATCCCCGAAGGACCCCCGAACCGCAAAGG |
| | | ACATCAGTATCCCACAGCCTCTCCAAGTCCCCCGGTCT |
| | | CCTCCTCTTCTCGAAGGGACCAAAAGATCAATCCACCA |
| | | CACCCGACGACACTCAACTCCCCACCCCTAAAGGAGAC |
| | | ACCGGGAATCCCAGAATCAAGACTCATCCAATGTCCAT |
| | | CATGGGTCTCAAGGTGAACGTCTCTGCCATATTCATGG |
| | | CAGTACTGTTAACTCTCCAAACACCCACCGGTCAAATC |
| | | CATTGGGGCAATCTCTCTAAGATAGGGGTGGTAGGAAT |
| | | AGGAAGTGCAAGCTACAAAGTTATGACTCGTTCCAGCC |
| | | ATCAATCATTAGTCATAAAATTAATGCCCAATATAACT |
| | | CTCCTCAATAACTGCACGAGGGTAGAGATTGCAGAATA |
| | | CAGGAGACTACTGAGAACAGTTTTGGAACCAATTAGA |
| | | GATGCACTTAATGCAATGACCCAGAATATAAGACCGGT |
| | | TCAGAGTGTAGCTTCAAGTAGGAGACACAAGAGATTT |
| | | GCGGGAGTAGTCCTGGCAGGTGCGGCCCTAGGCGTTGC |
| | | CACAGCTGCTCAGATAACAGCCGGCATTGCACTTCACC |
| | | AGTCCATGCTGAACTCTCAAGCCATCGACAATCTGAGA |
| | | GCGAGCCTGGAAACTACTAATCAGGCAATTGAGACAA |
| | | TCAGACAAGCAGGGCAGGAGATGATATTGGCTGTTCA |
| | | GGGTGTCCAAGACTACATCAATAATGAGCTGATACCGT |
| | | CTATGAACCAACTATCTTGTGATTTAATCGGCCAGAAG |
| | | CTCGGGCTCAAATTGCTCAGATACTATACAGAAATCCT |
| | | GTCATTATTTGGCCCCAGTTTACGGGACCCCATATCTG |
| | | CGGAGATATCTATCCAGGCTTTGAGCTATGCGCTTGGA |
| | | GGAGACATCAATAAGGTGTTAGAAAAGCTCGGATACA |
| | | GTGGAGGTGATTTACTGGGCATCTTAGAGAGCGGAGG |
| | | AATAAAGGCCCGGATAACTCACGTCGACACAGAGTCC |
| | | TACTTCATTGTCCTCAGTATAGCCTATCCGACGCTGTCC |
| | | GAGATTAAGGGGGTGATTGTCCACCGGCTAGAGGGGG |
| | | TCTCGTACAACATAGGCTCTCAAGAGTGGTATACCACT |
| | | GTGCCCAAGTATGTTGCAACCCAAGGGTACCTTATCTC |
| | | GAATTTTGATGAGTCATCGTGTACTTTCATGCCAGAGG |
| | | GGACTGTGTGCAGCCAAAATGCCTTGTACCCGATGAGT |
| | | CCTCTGCTCCAAGAATGCCTCCGGGGGTACACCAAGTC |
| | | CTGTGCTCGTACACTCGTATCCGGGTCTTTTGGGAACC |
| | | GGTTCATTTTATCACAAGGGAACCTAATAGCCAATTGT |
| | | GCATCAATCCTTTGCAAGTGTTACACAACAGGAACGAT |
| | | CATTAATCAAGACCCTGACAAGATCCTAACATACATTG |
| | | CTGCCGATCACTGCCCGGTAGTCGAGGTGAACGGCGTG |
| | | ACCATCCAAGTCGGGAGCAGGAGGTATCCAGACGCTG |
| | | TGTACTTGCACAGAATTGACCTCGGTCCTCCCATATCA |
| | | TTGGAGAGGTTGGACGTAGGGACAAATCTGGGGAATG |
| | | CAATTGCTAAGTTGGAGGATGCCAAGGAATTGTTGGAG |
| | | TCATCGGACCAGATATTGAGGAGTATGAAAGGTTTATC |
| | | GAGCACTAGCATAGTCTACATCCTGATTGCAGTGTGTC |
| | | TTGGAGGGTTGATAGGGATCCCCGCTTTAATATGTTGC |
| | | TGCAGGGGGCGTTGTAACAAAAAGGGAGAACAAGTTG |
| | | GTATGTCAAGACCAGGCCTAAAGCCTGATCTTACGGGA |
| | | ACATCAAAATCCTATGTAAGGTCGCTCTGATCCTCTAC |
| | | AACTCTTGAAACACAAATGTCCCACAAGTCTCCTCTTC |
| | | GTCATCAAGCAACCACCGCACCCAGCATCAAGCCCACC |
| | | TGAAATTATCTCCGGCTTCCCTCTGGCCGAACAATATC |
| | | GGTAGTTAATCAAAACTTAGGGTGCAAGATCATCCACA |
| | | ATGTCACCACAACGAGACCGGATAAATGCCTTCTACAA |
| | | AGATAACCCCCATCCCAAGGGAAGTAGGATAGTCATT |
| | | AACAGAGAACATCTTATGATTGATAGACCTTATGTTTT |
| | | GCTGGCTGTTCTGTTTGTCATGTTTCTGAGCTTGATCGG |
| | | GTTGCTAGCCATTGCAGGCATTAGACTTCATCGGGCAG |
| | | CCATCTACACCGCAGAGATCCATAAAAGCCTCAGCACC |
| | | AATCTAGATGTAACTAACTCAATCGAGCATCAGGTCAA |
| | | GGACGTGCTGACACCACTCTTCAAAATCATCGGTGATG |
| | | AAGTGGGCCTGAGGACACCTCAGAGATTCACTGACCTA |
| | | GTGAAATTAATCTCTGACAAGATTAAATTCCTTAATCC |
| | | GGATAGGGAGTACGACTTCAGAGATCTCACTTGGTGTA |
| | | TCAACCCGCCAGAGAGAATCAAATTGGATTATGATCAA |
| | | TACTGTGCAGATGTGGCTGCTGAAGAGCTCATGAATGC |
| | | ATTGGTGAACTCAACTCTACTGGAGACCAGAACAACCA |
| | | ATCAGTTCCTAGCTGTCTCAAAGGGAAACTGCTCAGGG |
| | | CCCACTACAATCAGAGGTCAATTCTCAAACATGTCGCT |
| | | GTCCCTGTTAGACTTGTATTTAGGTCGAGGTTACAATG |
| | | TGTCATCTATAGTCACTATGACATCCCAGGGAATGTAT |
| | | GGGGGAACTTACCTAGTGGAAAAGCCTAATCTGAGCA |
| | | GCAAAAGGTCAGAGTTGTCACAACTGAGCATGTACCG |
| | | AGTGTTTGAAGTAGGTGTTATCAGAAATCCGGGTTTGG |
| | | GGGCTCCGGTGTTCCATATGACAAACTATCTTGAGCAA |
| | | CCAGTCAGTAATGATCTCAGCAACTGTATGGTGGCTTT |
| | | GGGGGAGCTCAAACTCGCAGCCCTTTGTCACGGGGAA |
| | | GATTCTATCACAATTCCCTATCAGGGATCAGGGAAAGG |
| | | TGTCAGCTTCCAGCTCGTCAAGCTAGGTGTCTGGAAAT |
| | | CCCCAACCGACATGCAATCCTGGGTCCCCTTATCAACG |
| | | GATGATCCAGTGATAGACAGGCTTTACCTCTCATCTCA |
| | | CAGAGGTGTTATCGCTGACAATCAAGCAAAATGGGCT |
| | | GTCCCGACAACACGAACAGATGACAAGTTGCGAATGG |
| | | AGACATGCTTCCAACAGGCGTGTAAGGGTAAAATCCA |
| | | AGCACTCTGCGAGAATCCCGAGTGGGCACCATTGAAG |
| | | GATAACAGGATTCCTTCATACGGGGTCTTGTCTGTTGA |
| | | TCTGAGTCTGACAGTTGAGCTTAAAATCAAAATTGCTT |
| | | CGGGATTCGGGCCATTGATCACACACGGTTCAGGGATG |
| | | GACCTATACAAATCCAACCACAACAATGTGTATTGGCT |
| | | GACTATCCCGCCAATGAAGAACCTAGCCTTAGGTGTAA |
| | | TCAACACATTGGAGTGGATACCGAGATTCAAGGTTAGT |
| | | CCCTACCTCTTCACTGTCCCAATTAAGGAAGCAGGCGA |
| | | AGACTGCCATGCCCCAACATACCTACCTGCGGAGGTGG |
| | | ATGGTGATGTCAAACTCAGTTCCAATCTGGTGATTCTA |
| | | CCTGGTCAAGATCTCCAATATGTTTTGGCAACCTACGA |
| | | TACTTCCAGGGTTGAACATGCTGTGGTTTATTACGTTTA |
| | | CAGCCCAAGCCGCTCATTTTCTTACTTTTATCCTTTTAG |
| | | GTTGCCTATAAAGGGGGTCCCCATCGAATTACAAGTGG |
| | | AATGCTTCACATGGGACCAAAAACTCTGGTGCCGTCAC |
| | | TTCTGTGTGCTTGCGGACTCAGAATCTGGTGGACATAT |
| | | CACTCACTCTGGGATGGTGGGCATGGGAGTCAGCTGCA |
| | | CAGTCACCCGGGAAGATGGAACCAATCGCAGATAGGG |
| | | CTGCTAGTGAACCAATCACATGATGTCACCCAGACATC |
| | | AGGCATACCCACTAGTCTACCCTCCATCATTGTTATAA |
| | | AAAACTTAGGAACCAGGTCCACACAGCCGCCAGCCCA |
| | | TCAACGCGTACGGCCACCATGGAGGCCGCTTTGCTGGT |
| | | GTGTCAGTACACAATTCAGAGCCTGATCCACTTGACGG |
| | | GAGAGGACCCTGGCTTTTTCAACGTTGAGATCCCCGAA |
| | | TTCCCATTTTATCCAACCTGCAACGTGTGTACTGCAGA |
| | | CGTAAACGTTACTATTAATTTCGACGTGGGAGGCAAGA |
| | | AACACCAACTGGATCTCGACTTTGGACAGCTGACTCCC |
| | | CACACCAAAGCTGTTTACCAACCCAGAGGGGCTTTTGG |
| | | CGGCTCTGAAAACGCTACCAACCTCTTCCTGCTCGAGC |
| | | TTCTGGGAGCTGGCGAGCTTGCTCTGACTATGCGCTCT |
| | | AAAAAGCTCCCGATTAACGTCACAACAGGGGAGGAAC |
| | | AACAGGTGAGCTTGGAGTCCGTAGACGTGTACTTCCAG |
| | | GACGTGTTCGGAACAATGTGGTGTCATCACGCTGAGAT |
| | | GCAGAATCCCGTGTACCTCATTCCAGAGACGGTGCCTT |
| | | ATATAAAGTGGGATAACTGTAACTCAACGAATATCACA |
| | | GCAGTGGTTAGGGCGCAAGGTCTGGACGTCACTCTTCC |
| | | TCTCTCACTCCCAACTTCCGCTCAAGACAGCAATTTCTC |
| | | AGTGAAAACAGAAATGCTGGGCAACGAGATAGATATT |
| | | GAGTGTATAATGGAAGACGGGGAGATTAGTCAAGTGC |
| | | TGCCTGGCGACAACAAATTTAATATCACCTGTAGCGGT |
| | | TACGAGAGTCACGTCCCGTCCGGGGGGATTCTGACCAG |
| | | CACTAGCCCTGTCGCCACTCCCATTCCGGGAACTGGAT |
| | | ACGCCTATAGCCTTAGGCTTACACCCAGACCTGTGTCA |
| | | CGGTTTCTGGGCAACAACAGCATTCTGTACGTGTTCTA |
| | | CAGTGGCAACGGGCCTAAAGCCTCTGGTGGCGACTATT |
| | | GTATTCAAAGCAACATCGTCTTCTCCGACGAGATCCCT |
| | | GCAAGCCAGGACATGCCCACTAATACCACCGACATTAC |
| | | CTACGTAGGGGACAACGCTACTTACTCTGTCCCGATGG |
| | | TGACTAGCGAGGATGCTAATTCTCCAAACGTTACCGTA |
| | | ACCGCCTTTTGGGCTTGGCCCAATAATACAGAGACGGA |
| | | TTTCAAATGCAAGTGGACTTTGACTTCCGGCACCCCCT |
| | | CCGGCTGTGAAAATATTTCTGGCGCTTTCGCCAGCAAC |
| | | AGGACGTTCGACATCACAGTCTCTGGTCTTGGCACTGC |
| | | CCCAAAGACCCTGATCATCACCCGGACTGCCACTAACG |
| | | CGACAACTACAACTCATAAGGTTATTTTTAGCAAGGCA |
| | | CCAGAGAGCACCACTACATCTCCCACCCTGAACACTAC |
| | | GGGCTTTGCTGACCCGAATACCACCACGGGCCTTCCCT |
| | | CATCAACCCATGTCCCAACCAACTTGACAGCACCAGCA |
| | | TCAACGGGTCCCACCGTCTCCACAGCAGACGTAACCTC |
| | | TCCTACACCTGCCGGCACCACAAGTGGCGCTAGTCCTG |
| | | TGACCCCTTCCCCATCTCCTTGGGATAATGGCACAGAG |
| | | TCAAAAGCCCCTGATATGACTAGCTCCACCAGTCCTGT |
| | | GACGACACCAACCCCAAACGCTACCTCCCCCACTCCAG |
| | | CCGTTACAACTCCAACGCCAAACGCTACCTCCCCTACT |
| | | CCTGCCGTAACCACGCCTACCCCTAATGCCACCAGTCC |
| | | TACTTTGGGCAAGACCTCCCCGACAAGCGCTGTGACAA |
| | | CCCCTACGCCCAACGCAACTTCTCCTACACTCGGAAAG |
| | | ACCTCACCTACCAGTGCTGTCACCACACCTACACCAAA |
| | | CGCCACTAGCCCCACATTGGGTAAGACATCCCCCACTT |
| | | CCGCAGTTACGACTCCCACACCGAACGCAACCGGACCT |
| | | ACTGTCGGTGAGACATCCCCTCAGGCCAACGCCACAAA |
| | | TCACACACTGGGCGGCACCTCACCAACTCCAGTAGTGA |
| | | CCTCCCAGCCAAAAAACGCCACCTCTGCTGTGACCACC |
| | | GGTCAGCATAACATCACAAGTTCCTCCACTTCTAGCAT |
| | | GAGTTTGCGCCCCTCATCCAACCCAGAAACGCTGAGCC |
| | | CTTCAACATCTGACAACTCAACAAGCCATATGCCTCTG |
| | | CTCACTAGTGCTCACCCAACAGGGGGAGAAAACATTA |
| | | CACAGGTGACACCCGCCAGCATCTCTACACACCACGTG |
| | | AGCACAAGCTCTCCCGCTCCTCGGCCAGGTACAACCTC |
| | | CCAAGCCTCTGGGCCTGGAAATTCCTCAACCTCCACCA |
| | | AACCCGGTGAGGTCAACGTCACCAAGGGAACACCCCC |
| | | CCAGAACGCAACGTCACCTCAAGCACCCAGCGGACAG |
| | | AAGACCGCTGTGCCTACTGTAACCTCTACCGGAGGGAA |
| | | AGCTAATAGTACGACTGGAGGTAAGCACACTACGGGC |
| | | CACGGTGCCAGAACCAGTACAGAGCCTACCACTGATTA |
| | | CGGCGGAGATAGTACAACCCCCCGCCCTAGATACAAC |
| | | GCCACAACTTATTTGCCTCCCTCAACGTCCTCCAAGCT |
| | | GAGACCCAGGTGGACGTTTACGTCTCCCCCAGTCACAA |
| | | CTGCGCAGGCAACCGTGCCAGTTCCACCTACAAGTCAG |
| | | CCGCGCTTCAGCAATCTCTCAATGCTCGTGCTGCAGTG |
| | | GGCCTCTCTGGCAGTACTGACCCTCTTGCTTCTCTTGGT |
| | | GATGGCTGACTGTGCCTTTCGGAGAAACCTGAGCACCT |
| | | CACACACTTATACCACACCTCCCTACGACGACGCCGAG |
| | | ACATACGTATAATTCGAACTACAGCTCAACTTACCTGC |
| | | CAACCCCATGCCAGTCGACCCAACTAGTACAACCTAAA |
| | | TCCATTATAAAAAACTTAGGAACCAGGTCCACACAGCC |
| | | GCCAGCCCATCAACCATCCACTCCCACGATTGGAGGCC |
| | | GGCCATGGGTAGATCAAGAGAAAGAGCCAGAGGACGG |
| | | GGTAGAGGCAGAGGAGAGAAGAGACCTCGCAGCCCTA |
| | | GCAGCCAGAGTTCTAGCTCAGGTTCTCCTCCAAGAAGA |
| | | CCTCCTCCAGGGAGGAGACCCTTCTTTCATCCGGTCGG |
| | | AGACGCCGATTACTTTGAGTACTTGCAGGAAGGCGGTC |
| | | CAGACGGAGAACCAGACGTCCCACCGGGAGCTATCGA |
| | | GCAAGGACCTACCGACGACCCAGGAGAAGGACCAAGC |
| | | ACGGGTCCAAGAGGTCAGGGAGACGGAGGACGGAGA |
| | | AAGAAGGGCGGCTGGTTTGGAAAGCATCGCGGGCAAG |
| | | GCGGATCAAACCCTAAATTCGAGAACATTGCCGAGGG |
| | | ACTGAGAGTGCTCCTGGCCAGATCACACGTGGAGAGG |
| | | ACTACTGAGGAAGGTAACTGGGTAGCCGGGGTATTCGT |
| | | TTACGGCGGGAGCAAAACATCACTCTACAATCTCAGAC |
| | | GAGGAATTGCTCTCGCGGTACCACAGTGCAGGATTACG |
| | | CCTCTGTCTCGGTTGCCATTTGGCATGGCACCTGGTCCT |
| | | GGACCTCAACCTGGTCCTCTCAGGGAGTCCATAGTGTG |
| | | TTATTTCATGGTGTTTCTCCAGACGCATATCTTCGCGGA |
| | | AGTATTGAAGGACGCTATAAAGGACCTGGTGATGACT |
| | | AAGCCAGCCCCCACATGTAACATAAAGGTGACAGTGT |
| | | GCAGTTTTGACGACGGCGTTGATCTGCCTCCTTGGTTCC |
| | | CTCCAATGGTAGAAGGAGCGGCGGCAGAAGGAGACGA |
| | | CGGCGACGACGGGGACGAGGGTGGAGACGGTGACGAA |
| | | GGTGAAGAAGGGCAGGAAATGGGCTCCCTGGAAATGG |
| | | TGCCTATGGGTGCTGGGCCACCAAGTCCGGGAGGTGAT |
| | | CCAGACGGAGACGACGGGGGTAACAATAGTCAGTACC |
| | | CCTCCGCCTCAGGCAGTAGTGGTAACACCCCGACCCCC |
| | | CCAAACGACGAAGAAAGGGAGTCCAACGAAGAGCCGC |
| | | CTCCTCCATACGAGGACCCGTATTGGGGCAACGGAGAT |
| | | CGCCATTCTGACTTCCAGCCTCTGGGAACGCAGGACCA |
| | | ATCTCTGTACCTTGGACTGCAGCACGATGGTAACGACG |
| | | GCCTTCCACCTCCACCATATTCACCTAGGGACGATAGT |
| | | AGCCAGCATATTTTCGAAGAAGCGGGCAGAGGGTCCA |
| | | TGAATCCAGTTTGTCTGCCGGTGATTGTGGCTCCGTATC |
| | | TCTTCTGGCTCGCGGCAATAGCTGCCTCCTGTTTCACCG |
| | | CTTCTGTGTCAACCGTTGTGACAGCAACTGGACTCGCG |
| | | CTGAGCCTTCTGCTCCTCGCTGCAGTCGCCTCAAGTTAC |
| | | GCAGCGGCCCAACGCAAACTTCTCACGCCAGTCACCGT |
| | | GCTGACTGCAGTGGTCACATTCTTTGCCATATGTTTGAC |
| | | TTGGAGGATTGAGGACCCACCCTTTAATTCACTGCTGT |
| | | TTGCCCTCCTTGCAGCGGCCGGAGGACTTCAAGGAATT |
| | | TACGTCCTGGTGATGCTGGTTCTCCTGATCCTCGCGTAC |
| | | AGACGAAGGTGGCGGAGGCTTACAGTATGTGGAGGGA |
| | | TTATGTTTCTGGCCTGTGTGCTGGTCTTGATCGTTGACG |
| | | CCGTTCTCCAATTGAGTCCCCTGTTGGGTGCTGTCACCG |
| | | TCGTATCTATGACACTGCTGCTTTTGGCTTTCGTCCTCT |
| | | GGTTGTCCAGCCCTGGAGGATTGGGTACCCTCGGAGCA |
| | | GCTTTGCTGACCTTGGCAGCCGCACTGGCTCTCCTGGC |
| | | TAGTCTCATCCTGGGCACGCTCAACCTCACTACAATGT |
| | | TCCTGCTGATGCTGCTGTGGACCCTTGTGGTGCTTCTCA |
| | | TTTGCAGCTCTTGTAGCTCTTGCCCCCTGTCAAAGATCC |
| | | TTCTCGCCAGATTGTTCCTGTACGCACTCGCTCTCTTGT |
| | | TGCTGGCCAGCGCTCTCATCGCAGGTGGAAGTATACTC |
| | | CAGACGAATTTCAAAAGTCTGAGTAGCACAGAGTTCAT |
| | | CCCAAACCTGTTCTGTATGTTGTTGCTGATCGTAGCTGG |
| | | AATCCTCTTCATCTTGGCAATCTTGACAGAGTGGGGAT |
| | | CCGGGAACCGGACATACGGACCTGTATTCATGTGTCTG |
| | | GGAGGCCTTCTTACAATGGTTGCGGGAGCTGTTTGGCT |
| | | TACCGTGATGACTAACACCCTGCTGAGTGCCTGGATTC |
| | | TGACTGCTGGATTTTTGATTTTTTTGATCGGCTTTGCTC |
| | | TCTTCGGCGTCATCAGGTGCTGTAGGTATTGCTGTTATT |
| | | ATTGCCTGACATTGGAAAGCGAGGAAAGACCACCCAC |
| | | ACCTTATAGAAATACGGTGTGAGCGCGCAGCGCTTAGA |
| | | CGTCTCGCGATCGATGCTAGTGTGAAATAGACATCAGA |
| | | ATTAAGAAAAACGTAGGGTCCAAGTGGTTCCCCGTTAT |
| | | GGACTCGCTATCTGTCAACCAGATCTTATACCCTGAAG |
| | | TTCACCTAGATAGCCCGATAGTTACCAATAAGATAGTA |
| | | GCCATCCTGGAGTATGCTCGAGTCCCTCACGCTTACAG |
| | | CCTGGAGGACCCTACACTGTGTCAGAACATCAAGCACC |
| | | GCCTAAAAAACGGATTTTCCAACCAAATGATTATAAAC |
| | | AATGTGGAAGTTGGGAATGTCATCAAGTCCAAGCTTAG |
| | | GAGTTATCCGGCCCACTCTCATATTCCATATCCAAATT |
| | | GTAATCAGGATTTATTTAACATAGAAGACAAAGAGTCA |
| | | ACGAGGAAGATCCGTGAACTCCTCAAAAAGGGGAATT |
| | | CGCTGTACTCCAAAGTCAGTGATAAGGTTTTCCAATGC |
| | | TTAAGGGACACTAACTCACGGCTTGGCCTAGGCTCCGA |
| | | ATTGAGGGAGGACATCAAGGAGAAAGTTATTAACTTG |
| | | GGAGTTTACATGCACAGCTCCCAGTGGTTTGAGCCCTT |
| | | TCTGTTTTGGTTTACAGTCAAGACTGAGATGAGGTCAG |
| | | TGATTAAATCACAAACCCATACTTGCCATAGGAGGAGA |
| | | CACACACCTGTATTCTTCACTGGTAGTTCAGTTGAGTTG |
| | | CTAATCTCTCGTGACCTTGTTGCTATAATCAGTAAAGA |
| | | GTCTCAACATGTATATTACCTGACATTTGAACTGGTTTT |
| | | GATGTATTGTGATGTCATAGAGGGGAGGTTAATGACAG |
| | | AGACCGCTATGACTATTGATGCTAGGTATACAGAGCTT |
| | | CTAGGAAGAGTCAGATACATGTGGAAACTGATAGATG |
| | | GTTTCTTCCCTGCACTCGGGAATCCAACTTATCAAATTG |
| | | TAGCCATGCTGGAGCCTCTTTCACTTGCTTACCTGCAGC |
| | | TGAGGGATATAACAGTAGAACTCAGAGGTGCTTTCCTT |
| | | AACCACTGCTTTACTGAAATACATGATGTTCTTGACCA |
| | | AAACGGGTTTTCTGATGAAGGTACTTATCATGAGTTAA |
| | | CTGAAGCTCTAGATTACATTTTCATAACTGATGACATA |
| | | CATCTGACAGGGGAGATTTTCTCATTTTTCAGAAGTTTC |
| | | GGCCACCCCAGACTTGAAGCAGTAACGGCTGCTGAAA |
| | | ATGTTAGGAAATACATGAATCAGCCTAAAGTCATTGTG |
| | | TATGAGACTCTGATGAAAGGTCATGCCATATTTTGTGG |
| | | AATCATAATCAACGGCTATCGTGACAGGCACGGAGGC |
| | | AGTTGGCCACCGCTGACCCTCCCCCTGCATGCTGCAGA |
| | | CACAATCCGGAATGCTCAAGCTTCAGGTGAAGGGTTAA |
| | | CACATGAGCAGTGCGTTGATAACTGGAAATCTTTTGCT |
| | | GGAGTGAAATTTGGCTGCTTTATGCCTCTTAGCCTGGA |
| | | TAGTGATCTGACAATGTACCTAAAGGACAAGGCACTTG |
| | | CTGCTCTCCAAAGGGAATGGGATTCAGTTTACCCGAAA |
| | | GAGTTCCTGCGTTACGACCCTCCCAAGGGAACCGGGTC |
| | | ACGGAGGCTTGTAGATGTTTTCCTTAATGATTCGAGCT |
| | | TTGACCCATATGATGTGATAATGTATGTTGTAAGTGGA |
| | | GCTTACCTCCATGACCCTGAGTTCAACCTGTCTTACAG |
| | | CCTGAAAGAAAAGGAGATCAAGGAAACAGGTAGACTT |
| | | TTTGCTAAAATGACTTACAAAATGAGGGCATGCCAAGT |
| | | GATTGCTGAAAATCTAATCTCAAACGGGATTGGCAAAT |
| | | ATTTTAAGGACAATGGGATGGCCAAGGATGAGCACGA |
| | | TTTGACTAAGGCACTCCACACTCTAGCTGTCTCAGGAG |
| | | TCCCCAAAGATCTCAAAGAAAGTCACAGGGGGGGGCC |
| | | AGTCTTAAAAACCTACTCCCGAAGCCCAGTCCACACAA |
| | | GTACCAGGAACGTGAGAGCAGCAAAAGGGTTTATAGG |
| | | GTTCCCTCAAGTAATTCGGCAGGACCAAGACACTGATC |
| | | ATCCGGAGAATATGGAAGCTTACGAGACAGTCAGTGC |
| | | ATTTATCACGACTGATCTCAAGAAGTACTGCCTTAATT |
| | | GGAGATATGAGACCATCAGCTTGTTTGCACAGAGGCTA |
| | | AATGAGATTTACGGATTGCCCTCATTTTTCCAGTGGCT |
| | | GCATAAGAGGCTTGAGACCTCTGTCCTGTATGTAAGTG |
| | | ACCCTCATTGCCCCCCCGACCTTGACGCCCATATCCCG |
| | | TTATATAAAGTCCCCAATGATCAAATCTTCATTAAGTA |
| | | CCCTATGGGAGGTATAGAAGGGTATTGTCAGAAGCTGT |
| | | GGACCATCAGCACCATTCCCTATCTATACCTGGCTGCT |
| | | TATGAGAGCGGAGTAAGGATTGCTTCGTTAGTGCAAGG |
| | | GGACAATCAGACCATAGCCGTAACAAAAAGGGTACCC |
| | | AGCACATGGCCCTACAACCTTAAGAAACGGGAAGCTG |
| | | CTAGAGTAACTAGAGATTACTTTGTAATTCTTAGGCAA |
| | | AGGCTACATGATATTGGCCATCACCTCAAGGCAAATGA |
| | | GACAATTGTTTCATCACATTTTTTTGTCTATTCAAAAGG |
| | | AATATATTATGATGGGCTACTTGTGTCCCAATCACTCA |
| | | AGAGCATCGCAAGATGTGTATTCTGGTCAGAGACTATA |
| | | GTTGATGAAACAAGGGCAGCATGCAGTAATATTGCTAC |
| | | AACAATGGCTAAAAGCATCGAGAGAGGTTATGACCGT |
| | | TACCTTGCATATTCCCTGAACGTCCTAAAAGTGATACA |
| | | GCAAATTCTGATCTCTCTTGGCTTCACAATCAATTCAAC |
| | | CATGACCCGGGATGTAGTCATACCCCTCCTCACAAACA |
| | | ACGACCTCTTAATAAGGATGGCACTGTTGCCCGCTCCT |
| | | ATTGGGGGGATGAATTATCTGAATATGAGCAGGCTGTT |
| | | TGTCAGAAACATCGGTGATCCAGTAACATCATCAATTG |
| | | CTGATCTCAAGAGAATGATTCTCGCCTCACTAATGCCT |
| | | GAAGAGACCCTCCATCAAGTAATGACACAACAACCGG |
| | | GGGACTCTTCATTCCTAGACTGGGCTAGCGACCCTTAC |
| | | TCAGCAAATCTTGTATGTGTCCAGAGCATCACTAGACT |
| | | CCTCAAGAACATAACTGCAAGGTTTGTCCTGATCCATA |
| | | GTCCAAACCCAATGTTAAAAGGATTATTCCATGATGAC |
| | | AGTAAAGAAGAGGACGAGGGACTGGCGGCATTCCTCA |
| | | TGGACAGGCATATTATAGTACCTAGGGCAGCTCATGAA |
| | | ATCCTGGATCATAGTGTCACAGGGGCAAGAGAGTCTAT |
| | | TGCAGGCATGCTGGATACCACAAAAGGCTTGATTCGAG |
| | | CCAGCATGAGGAAGGGGGGGTTAACCTCTCGAGTGAT |
| | | AACCAGATTGTCCAATTATGACTATGAACAATTCAGAG |
| | | CAGGGATGGTGCTATTGACAGGAAGAAAGAGAAATGT |
| | | CCTCATTGACAAAGAGTCATGTTCAGTGCAGCTGGCGA |
| | | GAGCTCTAAGAAGCCATATGTGGGCGAGGCTAGCTCG |
| | | AGGACGGCCTATTTACGGCCTTGAGGTCCCTGATGTAC |
| | | TAGAATCTATGCGAGGCCACCTTATTCGGCGTCATGAG |
| | | ACATGTGTCATCTGCGAGTGTGGATCAGTCAACTACGG |
| | | ATGGTTTTTTGTCCCCTCGGGTTGCCAACTGGATGATAT |
| | | TGACAAGGAAACATCATCCTTGAGAGTCCCATATATTG |
| | | GTTCTACCACTGATGAGAGAACAGACATGAAGCTTGCC |
| | | TTCGTAAGAGCCCCAAGTCGATCCTTGCGATCTGCTGT |
| | | TAGAATAGCAACAGTGTACTCATGGGCTTACGGTGATG |
| | | ATGATAGCTCTTGGAACGAAGCCTGGTTGTTGGCTAGG |
| | | CAAAGGGCCAATGTGAGCCTGGAGGAGCTAAGGGTGA |
| | | TCACTCCCATCTCAACTTCGACTAATTTAGCGCATAGG |
| | | TTGAGGGATCGTAGCACTCAAGTGAAATACTCAGGTAC |
| | | ATCCCTTGTCCGAGTGGCGAGGTATACCACAATCTCCA |
| | | ACGACAATCTCTCATTTGTCATATCAGATAAGAAGGTT |
| | | GATACTAACTTTATATACCAACAAGGAATGCTTCTAGG |
| | | GTTGGGTGTTTTAGAAACATTGTTTCGACTCGAGAAAG |
| | | ATACCGGATCATCTAACACGGTATTACATCTTCACGTC |
| | | GAAACAGATTGTTGCGTGATCCCGATGATAGATCATCC |
| | | CAGGATACCCAGCTCCCGCAAGCTAGAGCTGAGGGCA |
| | | GAGCTATGTACCAACCCATTGATATATGATAATGCACC |
| | | TTTAATTGACAGAGATGCAACAAGGCTATACACCCAGA |
| | | GCCATAGGAGGCACCTTGTGGAATTTGTTACATGGTCC |
| | | ACACCCCAACTATATCACATTTTAGCTAAGTCCACAGC |
| | | ACTATCTATGATTGACCTGGTAACAAAATTTGAGAAGG |
| | | ACCATATGAATGAAATTTCAGCTCTCATAGGGGATGAC |
| | | GATATCAATAGTTTCATAACTGAGTTTCTGCTCATAGA |
| | | GCCAAGATTATTCACTATCTACTTGGGCCAGTGTGCGG |
| | | CCATCAATTGGGCATTTGATGTACATTATCATAGACCA |
| | | TCAGGGAAATATCAGATGGGTGAGCTGTTGTCATCGTT |
| | | CCTTTCTAGAATGAGCAAAGGAGTGTTTAAGGTGCTTG |
| | | TCAATGCTCTAAGCCACCCAAAGATCTACAAGAAATTC |
| | | TGGCATTGTGGTATTATAGAGCCTATCCATGGTCCTTC |
| | | ACTTGATGCTCAAAACTTGCACACAACTGTGTGCAACA |
| | | TGGTTTACACATGCTATATGACCTACCTCGACCTGTTGT |
| | | TGAATGAAGAGTTAGAAGAGTTCACATTTCTCTTGTGT |
| | | GAAAGCGACGAGGATGTAGTACCGGACAGATTCGACA |
| | | ACATCCAGGCAAAACACTTATGTGTTCTGGCAGATTTG |
| | | TACTGTCAACCAGGGACCTGCCCACCAATTCGAGGTCT |
| | | AAGACCGGTAGAGAAATGTGCAGTTCTAACCGACCAT |
| | | ATCAAGGCAGAGGCTATGTTATCTCCAGCAGGATCTTC |
| | | GTGGAACATAAATCCAATTATTGTAGACCATTACTCAT |
| | | GCTCTCTGACTTATCTCCGGCGAGGATCGATCAAACAG |
| | | ATAAGATTGAGAGTTGATCCAGGATTCATTTTCGACGC |
| | | CCTCGCTGAGGTAAATGTCAGTCAGCCAAAGATCGGCA |
| | | GCAACAACATCTCAAATATGAGCATCAAGGCTTTCAGA |
| | | CCCCCACACGATGATGTTGCAAAATTGCTCAAAGATAT |
| | | CAACACAAGCAAGCACAATCTTCCCATTTCAGGGGGCA |
| | | ATCTCGCCAATTATGAAATCCATGCTTTCCGCAGAATC |
| | | GGGTTGAACTCATCTGCTTGCTACAAAGCTGTTGAGAT |
| | | ATCAACATTAATTAGGAGATGCCTTGAGCCAGGGGAG |
| | | GACGGCTTGTTCTTGGGTGAGGGATCGGGTTCTATGTT |
| | | GATCACTTATAAAGAGATACTTAAACTAAACAAGTGCT |
| | | TCTATAATAGTGGGGTTTCCGCCAATTCTAGATCTGGT |
| | | CAAAGGGAATTAGCACCCTATCCCTCCGAAGTTGGCCT |
| | | TGTCGAACACAGAATGGGAGTAGGTAATATTGTCAAA |
| | | GTGCTCTTTAACGGGAGGCCCGAAGTCACGTGGGTAGG |
| | | CAGTGTAGATTGCTTCAATTTCATAGTTAGTAATATCCC |
| | | TACCTCTAGTGTGGGGTTTATCCATTCAGATATAGAGA |
| | | CCTTGCCTGACAAAGATACTATAGAGAAGCTAGAGGA |
| | | ATTGGCAGCCATCTTATCGATGGCTCTGCTCCTGGGCA |
| | | AAATAGGATCAATACTGGTGATTAAGCTTATGCCTTTC |
| | | AGCGGGGATTTTGTTCAGGGATTTATAAGTTATGTAGG |
| | | GTCTCATTATAGAGAAGTGAACCTTGTATACCCTAGAT |
| | | ACAGCAACTTCATCTCTACTGAATCTTATTTGGTTATGA |
| | | CAGATCTCAAGGCTAACCGGCTAATGAATCCTGAAAA |
| | | GATTAAGCAGCAGATAATTGAATCATCTGTGAGGACTT |
| | | CACCTGGACTTATAGGTCACATCCTATCCATTAAGCAA |
| | | CTAAGCTGCATACAAGCAATTGTGGGAGACGCAGTTA |
| | | GTAGAGGTGATATCAATCCTACTCTGAAAAAACTTACA |
| | | CCTATAGAGCAGGTGCTGATCAATTGCGGGTTGGCAAT |
| | | TAACGGACCTAAGCTGTGCAAAGAATTGATCCACCATG |
| | | ATGTTGCCTCAGGGCAAGATGGATTGCTTAATTCTATA |
| | | CTCATCCTCTACAGGGAGTTGGCAAGATTCAAAGACAA |
| | | CCAAAGAAGTCAACAAGGGATGTTCCACGCTTACCCCG |
| | | TATTGGTAAGTAGCAGGCAACGAGAACTTATATCTAGG |
| | | ATCACCCGCAAATTCTGGGGGCACATTCTTCTTTACTCC |
| | | GGGAACAAAAAGTTGATAAATAAGTTTATCCAGAATCT |
| | | CAAGTCCGGCTATCTGATACTAGACTTACACCAGAATA |
| | | TCTTCGTTAAGAATCTATCCAAGTCAGAGAAACAGATT |
| | | ATTATGACGGGGGGTTTGAAACGTGAGTGGGTTTTTAA |
| | | GGTAACAGTCAAGGAGACCAAAGAATGGTATAAGTTA |
| | | GTCGGATACAGTGCCCTGATTAAGGACTAATTGGTTGA |
| | | ACTCCGGAACCCTAATCCTGCCCTAGGTGGTTAGGCAT |
| | | TATTTGCAATATATTAAAGAAAACTTTGAAAATACGAA |
| | | GTTTCTATTCCCAGCTTTGTCTGGT |
| 61 | T-03 | ACCAAACAAAGTTGGGTAAGGATAGTTCAATCAATGA |
| | (gH_ATUa_gL_ | TCATCTTCTAGTGCACTTAGGATTCAAGATCCTATTATC |
| | ATUb_gp42 at | AGGGACAAGAGCAGGATTAGGGATATCCGAGATGGCC |
| | ATU3) | ACACTTTTAAGGAGCTTAGCATTGTTCAAAAGAAACAA |
| | MV leader: nt | GGACAAACCACCCATTACATCAGGATCCGGTGGAGCC |
| | 1-55 | ATCAGAGGAATCAAACACATTATTATAGTACCAATCCC |
| | MV N ORF: nt | TGGAGATTCCTCAATTACCACTCGATCCAGACTTCTGG |
| | 108-1,685 | ACCGGTTGGTGAGGTTAATTGGAAACCCGGATGTGAGC |
| | MV P ORF: nt | GGGCCCAAACTAACAGGGGCACTAATAGGTATATTATC |
| | 1,807-3,330 | CTTATTTGTGGAGTCTCCAGGTCAATTGATTCAGAGGA |
| | MV M ORF: nt | TCACCGATGACCCTGACGTTAGCATAAGGCTGTTAGAG |
| | 3,438-4,445 | GTTGTCCAGAGTGACCAGTCACAATCTGGCCTTACCTT |
| | MV F ORF: nt | CGCATCAAGAGGTACCAACATGGAGGATGAGGCGGAC |
| | 5,449-7,110 | CAATACTTTTCACATGATGATCCAATTAGTAGTGATCA |
| | MV H ORF: nt | ATCCAGGTTCGGATGGTTCGGGAACAAGGAAATCTCA |
| | 7,271-9,124 | GATATTGAAGTGCAAGACCCTGAGGGATTCAACATGAT |
| | ATU upstream | TCTGGGTACCATCCTAGCCCAAATTTGGGTCTTGCTCG |
| | motif: nt 9,180- | CAAAGGCGGTTACGGCCCCAGACACGGCAGCTGATTC |
| | 9,257 | GGAGCTAAGAAGGTGGATAAAGTACACCCAACAAAGA |
| | GE of N gene: | AGGGTAGTTGGTGAATTTAGATTGGAGAGAAAATGGTT |
| | nt 9,196-9,206 | GGATGTGGTGAGGAACAGGATTGCCGAGGACCTCTCCT |
| | Conserved | TACGCCGATTCATGGTCGCTCTAATCCTGGATATCAAG |
| | trinucleotide | AGAACACCCGGAAACAAACCCAGGATTGCTGAAATGA |
| | motif: nt 9,207- | TATGTGACATTGATACATATATCGTAGAGGCAGGATTA |
| | 9,209 | GCCAGTTTTATCCTGACTATTAAGTTTGGGATAGAAAC |
| | GS of P gene: | TATGTATCCTGCTCTTGGACTGCATGAATTTGCTGGTGA |
| | nt 9,210-9,226 | GTTATCCACACTTGAGTCCTTGATGAACCTTTACCAGC |
| | Kozak | AAATGGGGGAAACTGCACCCTACATGGTAATCCTGGA |
| | sequence: nt | GAACTCAATTCAGAACAAGTTCAGTGCAGGATCATACC |
| | 9,252-9,260 | CTCTGCTCTGGAGCTATGCCATGGGAGTAGGAGTGGAA |
| | EBV gH ORF: | CTTGAAAACTCCATGGGAGGTTTGAACTTTGGCCGATC |
| | nt 9,258-11,381 | TTACTTTGATCCAGCATATTTTAGATTAGGGCAAGAGA |
| | ATUa motif: nt | TGGTAAGGAGGTCAGCTGGAAAGGTCAGTTCCACATTG |
| | 11,382-11,525 | GCATCTGAACTCGGTATCACTGCCGAGGATGCAAGGCT |
| | GE of P gene: | TGTTTCAGAGATTGCAATGCATACTACTGAGGACAAGA |
| | nt 11,449- | TCAGTAGAGCGGTTGGACCCAGACAAGCCCAAGTATC |
| | 11,459 | ATTTCTACACGGTGATCAAAGTGAGAATGAGCTACCGA |
| | Conserved | GATTGGGGGGCAAGGAAGATAGGAGGGTCAAACAGAG |
| | trinucleotide | TCGAGGAGAAGCCAGGGAGAGCTACAGAGAAACCGGG |
| | motif: nt | CCCAGCAGAGCAAGTGATGCGAGAGCTGCCCATCTTCC |
| | 11,460-11,462 | AACCGGCACACCCCTAGACATTGACACTGCAACGGAG |
| | GS of P gene: | TCCAGCCAAGATCCGCAGGACAGTCGAAGGTCAGCTG |
| | nt 11,463- | ACGCCCTGCTTAGGCTGCAAGCCATGGCAGGAATCTCG |
| | 11,479 | GAAGAACAAGGCTCAGACACGGACACCCCTATAGTGT |
| | EBV gp42 | ACAATGACAGAAATCTTCTAGACTAGGTGCGAGAGGC |
| | ORF: nt | CGAGGGCCAGAACAACATCCGCCTACCATCCATCATTG |
| | 11,526-12,197 | TTATAAAAAACTTAGGAACCAGGTCCACACAGCCGCC |
| | ATUb motif: nt | AGCCCATCAACCATCCACTCCCACGATTGGAGCCAATG |
| | 12,198-12,299 | GCAGAAGAGCAGGCACGCCATGTCAAAAACGGACTGG |
| | GE of N gene: | AATGCATCCGGGCTCTCAAGGCCGAGCCCATCGGCTCA |
| | nt 12,249- | CTGGCCATCGAGGAAGCTATGGCAGCATGGTCAGAAA |
| | 12,259 | TATCAGACAACCCAGGACAGGAGCGAGCCACCTGCAG |
| | Conserved | GGAAGAGAAGGCAGGCAGTTCGGGTCTCAGCAAACCA |
| | trinucleotide | TGCCTCTCAGCAATTGGATCAACTGAAGGCGGTGCACC |
| | motif: nt | TCGCATCCGCGGTCAGGGACCTGGAGAGAGCGATGAC |
| | 12,260-12,262 3 | GACGCTGAAACTTTGGGAATCCCCCCAAGAAATCTCCA |
| | Hybrid GS | GGCATCAAGCACTGGGTTACAGTGTTATTACGTTTATG |
| | (combination of | ATCACAGCGGTGAAGCGGTTAAGGGAATCCAAGATGC |
| | GS of P gene | TGACTCTATCATGGTTCAATCAGGCCTTGATGGTGATA |
| | and GS of M | GCACCCTCTCAGGAGGAGACAATGAATCTGAAAACAG |
| | gene): nt | CGATGTGGATATTGGCGAACCTGATACCGAGGGATATG |
| | 12,263-12,279 | CTATCACTGACCGGGGATCTGCTCCCATCTCTATGGGG |
| | EBV gL ORF: | TTCAGGGCTTCTGATGTTGAAACTGCAGAAGGAGGGG |
| | nt 12,300- | AGATCCACGAGCTCCTGAGACTCCAATCCAGAGGCAA |
| | 12,713 | CAACTTTCCGAAGCTTGGGAAAACTCTCAATGTTCCTC |
| | ATU | CGCCCCCGGACCCCGGTAGGGCCAGCACTTCCGGGAC |
| | downstream | ACCCATTAAAAAGGGCACAGACGCGAGATTAGCCTCA |
| | motif: nt | TTTGGAACGGAGATCGCGTCTTTATTGACAGGTGGTGC |
| | 12,714-12,749 | AACCCAATGTGCTCGAAAGTCACCCTCGGAACCATCAG |
| | MV L ORF: nt | GGCCAGGTGCACCTGCGGGGAATGTCCCCGAGTGTGTG |
| | 12,804-19,355 | AGCAATGCCGCACTGATACAGGAGTGGACACCCGAAT |
| | MV trailer: nt | CTGGTACCACAATCTCCCCGAGATCCCAGAATAATGAA |
| | 19,428-19,464 | GAAGGGGGAGACTATTATGATGATGAGCTGTTCTCTGA |
| | | TGTCCAAGATATTAAAACAGCCTTGGCCAAAATACACG |
| | | AGGATAATCAGAAGATAATCTCCAAGCTAGAATCACT |
| | | GCTGTTATTGAAGGGAGAAGTTGAGTCAATTAAGAAG |
| | | CAGATCAACAGGCAAAATATCAGCATATCCACCCTGG |
| | | AAGGACACCTCTCAAGCATCATGATCGCCATTCCTGGA |
| | | CTTGGGAAGGATCCCAACGACCCCACTGCAGATGTCGA |
| | | AATCAATCCCGACTTGAAACCCATCATAGGCAGAGATT |
| | | CAGGCCGAGCACTGGCCGAAGTTCTCAAGAAACCCGTT |
| | | GCCAGCCGACAACTCCAAGGAATGACAAATGGACGGA |
| | | CCAGTTCCAGAGGACAGCTGCTGAAGGAATTTCAGCTA |
| | | AAGCCGATCGGGAAAAAGATGAGCTCAGCCGTCGGGT |
| | | TTGTTCCTGACACCGGCCCTGCATCACGCAGTGTAATC |
| | | CGCTCCATTATAAAATCCAGCCGGCTAGAGGAGGATCG |
| | | GAAGCGTTACCTGATGACTCTCCTTGATGATATCAAAG |
| | | GAGCCAATGATCTTGCCAAGTTCCACCAGATGCTGATG |
| | | AAGATAATAATGAAGTAGCTACAGCTCAACTTACCTGC |
| | | CAACCCCATGCCAGTCGACCCAACTAGTACAACCTAAA |
| | | TCCATTATAAAAAACTTAGGAGCAAAGTGATTGCCTCC |
| | | CAAGGTCCACAATGACAGAGACCTACGACTTCGACAA |
| | | GTCGGCATGGGACATCAAAGGGTCGATCGCTCCGATAC |
| | | AACCCACCACCTACAGTGATGGCAGGCTGGTGCCCCAG |
| | | GTCAGAGTCATAGATCCTGGTCTAGGCGACAGGAAGG |
| | | ATGAATGCTTTATGTACATGTTTCTGCTGGGGGTTGTTG |
| | | AGGACAGCGATTCCCTAGGGCCTCCAATCGGGCGAGC |
| | | ATTTGGGTTCCTGCCCTTAGGTGTTGGCAGATCCACAG |
| | | CAAAGCCCGAAAAACTCCTCAAAGAGGCCACTGAGCT |
| | | TGACATAGTTGTTAGACGTACAGCAGGGCTCAATGAAA |
| | | AACTGGTGTTCTACAACAACACCCCACTAACTCTCCTC |
| | | ACACCTTGGAGAAAGGTCCTAACAACAGGGAGTGTCTT |
| | | CAACGCAAACCAAGTGTGCAATGCGGTTAATCTGATAC |
| | | CGCTCGATACCCCGCAGAGGTTCCGTGTTGTTTATATG |
| | | AGCATCACCCGTCTTTCGGATAACGGGTATTACACCGT |
| | | TCCTAGAAGAATGCTGGAATTCAGATCGGTCAATGCAG |
| | | TGGCCTTCAACCTGCTGGTGACCCTTAGGATTGACAAG |
| | | GCGATAGGCCCTGGGAAGATCATCGACAATACAGAGC |
| | | AACTTCCTGAGGCAACATTTATGGTCCACATCGGGAAC |
| | | TTCAGGAGAAAGAAGAGTGAAGTCTACTCTGCCGATTA |
| | | TTGCAAAATGAAAATCGAAAAGATGGGCCTGGTTTTTG |
| | | CACTTGGTGGGATAGGGGGCACCAGTCTTCACATTAGA |
| | | AGCACAGGCAAAATGAGCAAGACTCTCCATGCACAAC |
| | | TCGGGTTCAAGAAGACCTTATGTTACCCGCTGATGGAT |
| | | ATCAATGAAGACCTTAATCGATTACTCTGGAGGAGCAG |
| | | ATGCAAGATAGTAAGAATCCAGGCAGTTTTGCAGCCAT |
| | | CAGTTCCTCAAGAATTCCGCATTTACGACGACGTGATC |
| | | ATAAATGATGACCAAGGACTATTCAAAGTTCTGTAGAC |
| | | CGTAGTGCCCAGCAATGCCCGAAAACGACCCCCCTCAC |
| | | AATGACAGCCAGAAGGCCCGGACAAAAAAGCCCCCTC |
| | | CGAAAGACTCCACGGACCAAGCGAGAGGCCAGCCAGC |
| | | AGCCGACGGCAAGCGCGAACACCAGGCGGCCCCAGCA |
| | | CAGAACAGCCCTGACACAAGGCCACCACCAGCCACCC |
| | | CAATCTGCATCCTCCTCGTGGGACCCCCGAGGACCAAC |
| | | CCCCAAGGCTGCCCCCGATCCAAACCACCAACCGCATC |
| | | CCCACCACCCCCGGGAAAGAAACCCCCAGCAATTGGA |
| | | AGGCCCCTCCCCCTCTTCCTCAACACAAGAACTCCACA |
| | | ACCGAACCGCACAAGCGACCGAGGTGACCCAACCGCA |
| | | GGCATCCGACTCCCTAGACAGATCCTCTCTCCCCGGCA |
| | | AACTAAACAAAACTTAGGGCCAAGGAACATACACACC |
| | | CAACAGAACCCAGACCCCGGCCCACGGCGCCGCGCCC |
| | | CCAACCCCCGACAACCAGAGGGAGCCCCCAACCAATC |
| | | CCGCCGGCTCCCCCGGTGCCCACAGGCAGGGACACCA |
| | | ACCCCCGAACAGACCCAGCACCCAACCATCGACAATC |
| | | CAAGACGGGGGGGCCCCCCCAAAAAAAGGCCCCCAGG |
| | | GGCCGACAGCCAGCACCGCGAGGAAGCCCACCCACCC |
| | | CACACACGACCACGGCAACCAAACCAGAACCCAGACC |
| | | ACCCTGGGCCACCAGCTCCCAGACTCGGCCATCACCCC |
| | | GCAGAAAGGAAAGGCCACAACCCGCGCACCCCAGCCC |
| | | CGATCCGGCGGGGAGCCACCCAACCCGAACCAGCACC |
| | | CAAGAGCGATCCCCGAAGGACCCCCGAACCGCAAAGG |
| | | ACATCAGTATCCCACAGCCTCTCCAAGTCCCCCGGTCT |
| | | CCTCCTCTTCTCGAAGGGACCAAAAGATCAATCCACCA |
| | | CACCCGACGACACTCAACTCCCCACCCCTAAAGGAGAC |
| | | ACCGGGAATCCCAGAATCAAGACTCATCCAATGTCCAT |
| | | CATGGGTCTCAAGGTGAACGTCTCTGCCATATTCATGG |
| | | CAGTACTGTTAACTCTCCAAACACCCACCGGTCAAATC |
| | | CATTGGGGCAATCTCTCTAAGATAGGGGTGGTAGGAAT |
| | | AGGAAGTGCAAGCTACAAAGTTATGACTCGTTCCAGCC |
| | | ATCAATCATTAGTCATAAAATTAATGCCCAATATAACT |
| | | CTCCTCAATAACTGCACGAGGGTAGAGATTGCAGAATA |
| | | CAGGAGACTACTGAGAACAGTTTTGGAACCAATTAGA |
| | | GATGCACTTAATGCAATGACCCAGAATATAAGACCGGT |
| | | TCAGAGTGTAGCTTCAAGTAGGAGACACAAGAGATTT |
| | | GCGGGAGTAGTCCTGGCAGGTGCGGCCCTAGGCGTTGC |
| | | CACAGCTGCTCAGATAACAGCCGGCATTGCACTTCACC |
| | | AGTCCATGCTGAACTCTCAAGCCATCGACAATCTGAGA |
| | | GCGAGCCTGGAAACTACTAATCAGGCAATTGAGACAA |
| | | TCAGACAAGCAGGGCAGGAGATGATATTGGCTGTTCA |
| | | GGGTGTCCAAGACTACATCAATAATGAGCTGATACCGT |
| | | CTATGAACCAACTATCTTGTGATTTAATCGGCCAGAAG |
| | | CTCGGGCTCAAATTGCTCAGATACTATACAGAAATCCT |
| | | GTCATTATTTGGCCCCAGTTTACGGGACCCCATATCTG |
| | | CGGAGATATCTATCCAGGCTTTGAGCTATGCGCTTGGA |
| | | GGAGACATCAATAAGGTGTTAGAAAAGCTCGGATACA |
| | | GTGGAGGTGATTTACTGGGCATCTTAGAGAGCGGAGG |
| | | AATAAAGGCCCGGATAACTCACGTCGACACAGAGTCC |
| | | TACTTCATTGTCCTCAGTATAGCCTATCCGACGCTGTCC |
| | | GAGATTAAGGGGGTGATTGTCCACCGGCTAGAGGGGG |
| | | TCTCGTACAACATAGGCTCTCAAGAGTGGTATACCACT |
| | | GTGCCCAAGTATGTTGCAACCCAAGGGTACCTTATCTC |
| | | GAATTTTGATGAGTCATCGTGTACTTTCATGCCAGAGG |
| | | GGACTGTGTGCAGCCAAAATGCCTTGTACCCGATGAGT |
| | | CCTCTGCTCCAAGAATGCCTCCGGGGGTACACCAAGTC |
| | | CTGTGCTCGTACACTCGTATCCGGGTCTTTTGGGAACC |
| | | GGTTCATTTTATCACAAGGGAACCTAATAGCCAATTGT |
| | | GCATCAATCCTTTGCAAGTGTTACACAACAGGAACGAT |
| | | CATTAATCAAGACCCTGACAAGATCCTAACATACATTG |
| | | CTGCCGATCACTGCCCGGTAGTCGAGGTGAACGGCGTG |
| | | ACCATCCAAGTCGGGAGCAGGAGGTATCCAGACGCTG |
| | | TGTACTTGCACAGAATTGACCTCGGTCCTCCCATATCA |
| | | TTGGAGAGGTTGGACGTAGGGACAAATCTGGGGAATG |
| | | CAATTGCTAAGTTGGAGGATGCCAAGGAATTGTTGGAG |
| | | TCATCGGACCAGATATTGAGGAGTATGAAAGGTTTATC |
| | | GAGCACTAGCATAGTCTACATCCTGATTGCAGTGTGTC |
| | | TTGGAGGGTTGATAGGGATCCCCGCTTTAATATGTTGC |
| | | TGCAGGGGGCGTTGTAACAAAAAGGGAGAACAAGTTG |
| | | GTATGTCAAGACCAGGCCTAAAGCCTGATCTTACGGGA |
| | | ACATCAAAATCCTATGTAAGGTCGCTCTGATCCTCTAC |
| | | AACTCTTGAAACACAAATGTCCCACAAGTCTCCTCTTC |
| | | GTCATCAAGCAACCACCGCACCCAGCATCAAGCCCACC |
| | | TGAAATTATCTCCGGCTTCCCTCTGGCCGAACAATATC |
| | | GGTAGTTAATCAAAACTTAGGGTGCAAGATCATCCACA |
| | | ATGTCACCACAACGAGACCGGATAAATGCCTTCTACAA |
| | | AGATAACCCCCATCCCAAGGGAAGTAGGATAGTCATT |
| | | AACAGAGAACATCTTATGATTGATAGACCTTATGTTTT |
| | | GCTGGCTGTTCTGTTTGTCATGTTTCTGAGCTTGATCGG |
| | | GTTGCTAGCCATTGCAGGCATTAGACTTCATCGGGCAG |
| | | CCATCTACACCGCAGAGATCCATAAAAGCCTCAGCACC |
| | | AATCTAGATGTAACTAACTCAATCGAGCATCAGGTCAA |
| | | GGACGTGCTGACACCACTCTTCAAAATCATCGGTGATG |
| | | AAGTGGGCCTGAGGACACCTCAGAGATTCACTGACCTA |
| | | GTGAAATTAATCTCTGACAAGATTAAATTCCTTAATCC |
| | | GGATAGGGAGTACGACTTCAGAGATCTCACTTGGTGTA |
| | | TCAACCCGCCAGAGAGAATCAAATTGGATTATGATCAA |
| | | TACTGTGCAGATGTGGCTGCTGAAGAGCTCATGAATGC |
| | | ATTGGTGAACTCAACTCTACTGGAGACCAGAACAACCA |
| | | ATCAGTTCCTAGCTGTCTCAAAGGGAAACTGCTCAGGG |
| | | CCCACTACAATCAGAGGTCAATTCTCAAACATGTCGCT |
| | | GTCCCTGTTAGACTTGTATTTAGGTCGAGGTTACAATG |
| | | TGTCATCTATAGTCACTATGACATCCCAGGGAATGTAT |
| | | GGGGGAACTTACCTAGTGGAAAAGCCTAATCTGAGCA |
| | | GCAAAAGGTCAGAGTTGTCACAACTGAGCATGTACCG |
| | | AGTGTTTGAAGTAGGTGTTATCAGAAATCCGGGTTTGG |
| | | GGGCTCCGGTGTTCCATATGACAAACTATCTTGAGCAA |
| | | CCAGTCAGTAATGATCTCAGCAACTGTATGGTGGCTTT |
| | | GGGGGAGCTCAAACTCGCAGCCCTTTGTCACGGGGAA |
| | | GATTCTATCACAATTCCCTATCAGGGATCAGGGAAAGG |
| | | TGTCAGCTTCCAGCTCGTCAAGCTAGGTGTCTGGAAAT |
| | | CCCCAACCGACATGCAATCCTGGGTCCCCTTATCAACG |
| | | GATGATCCAGTGATAGACAGGCTTTACCTCTCATCTCA |
| | | CAGAGGTGTTATCGCTGACAATCAAGCAAAATGGGCT |
| | | GTCCCGACAACACGAACAGATGACAAGTTGCGAATGG |
| | | AGACATGCTTCCAACAGGCGTGTAAGGGTAAAATCCA |
| | | AGCACTCTGCGAGAATCCCGAGTGGGCACCATTGAAG |
| | | GATAACAGGATTCCTTCATACGGGGTCTTGTCTGTTGA |
| | | TCTGAGTCTGACAGTTGAGCTTAAAATCAAAATTGCTT |
| | | CGGGATTCGGGCCATTGATCACACACGGTTCAGGGATG |
| | | GACCTATACAAATCCAACCACAACAATGTGTATTGGCT |
| | | GACTATCCCGCCAATGAAGAACCTAGCCTTAGGTGTAA |
| | | TCAACACATTGGAGTGGATACCGAGATTCAAGGTTAGT |
| | | CCCTACCTCTTCACTGTCCCAATTAAGGAAGCAGGCGA |
| | | AGACTGCCATGCCCCAACATACCTACCTGCGGAGGTGG |
| | | ATGGTGATGTCAAACTCAGTTCCAATCTGGTGATTCTA |
| | | CCTGGTCAAGATCTCCAATATGTTTTGGCAACCTACGA |
| | | TACTTCCAGGGTTGAACATGCTGTGGTTTATTACGTTTA |
| | | CAGCCCAAGCCGCTCATTTTCTTACTTTTATCCTTTTAG |
| | | GTTGCCTATAAAGGGGGTCCCCATCGAATTACAAGTGG |
| | | AATGCTTCACATGGGACCAAAAACTCTGGTGCCGTCAC |
| | | TTCTGTGTGCTTGCGGACTCAGAATCTGGTGGACATAT |
| | | CACTCACTCTGGGATGGTGGGCATGGGAGTCAGCTGCA |
| | | CAGTCACCCGGGAAGATGGAACCAATCGCAGATAGGG |
| | | CTGCTAGTGAACCAATCACATGATGTCACCCAGACATC |
| | | AGGCATACCCACTAGTCTACCCTCCATCATTGTTATAA |
| | | AAAACTTAGGAACCAGGTCCACACAGCCGCCAGCCCA |
| | | TCAACGCGTACGGCCACCATGCAACTCCTCTGCGTCTT |
| | | TTGCTTGGTGCTTTTGTGGGAGGTAGGAGCGGCCTCAT |
| | | TGTCAGAAGTTAAGCTGCATCTCGATATTGAAGGCCAC |
| | | GCTTCACATTACACTATCCCTTGGACCGAACTCATGGC |
| | | TAAGGTGCCGGGTTTGTCTCCGGAAGCACTTTGGAGAG |
| | | AGGCAAACGTTACCGAAGACCTGGCCTCAATGCTGAAT |
| | | CGCTACAAGCTGATATACAAGACTTCCGGAACCCTGGG |
| | | TATAGCACTCGCAGAGCCCGTCGATATCCCCGCTGTCA |
| | | GCGAAGGTAGCATGCAGGTGGACGCTAGTAAAGTTCA |
| | | CCCAGGAGTTATCTCAGGTCTGAATAGCCCAGCTTGTA |
| | | TGCTGAGCGCTCCACTGGAGAAACAGCTCTTCTACTAT |
| | | ATAGGCACAATGCTCCCTAACACTCGACCTCACAGCTA |
| | | CGTCTTCTATCAGCTGCGCTGTCACCTGTCATACGTAGC |
| | | CTTGTCCATTAATGGTGATAAGTTCCAGTACACAGGTG |
| | | CCATGACCTCCAAGTTCCTTATGGGGACCTATAAACGG |
| | | GTCACTGAGAAAGGAGACGAGCATGTCCTTAGTTTGGT |
| | | TTTTGGGAAGACAAAAGACCTGCCGGATCTGAGGGGC |
| | | CCGTTCTCTTACCCCTCACTCACATCTGCACAGAGTGG |
| | | GGACTATTCATTGGTGATTGTGACAACCTTCGTTCACT |
| | | ACGCCAATTTTCACAACTATTTTGTCCCGAACCTTAAA |
| | | GACATGTTTTCACGCGCCGTGACCATGACAGCTGCTTC |
| | | CTACGCTAGGTATGTGCTCCAAAAGCTGGTGCTGCTGG |
| | | AGATGAAGGGCGGATGCAGGGAACCTGAACTGGACAC |
| | | AGAGACTCTCACCACTATGTTCGAGGTTTCCGTGGCGT |
| | | TCTTCAAGGTGGGTCATGCTGTGGGGGAGACTGGGAAC |
| | | GGCTGCGTAGATCTTAGGTGGCTGGCTAAATCTTTCTT |
| | | CGAGCTGACCGTGTTGAAAGATATAATTGGAATTTGCT |
| | | ACGGGGCTACGGTTAAAGGAATGCAGTCTTACGGGCT |
| | | GGAAAGGCTGGCCGCTATGCTCATGGCTACTGTGAAGA |
| | | TGGAGGAACTCGGCCACCTCACCACAGAAAAACAGGA |
| | | GTACGCTTTGCGCCTGGCCACTGTTGGCTACCCTAAGG |
| | | CAGGCGTTTACTCTGGATTGATTGGGGGGGCCACATCT |
| | | GTGCTCTTGTCAGCCTACAATCGCCATCCCCTGTTCCAG |
| | | CCTCTGCACACGGTGATGCGAGAGACTTTGTTTATTGG |
| | | CAGCCACGTTGTGCTGAGAGAACTGCGCCTTAACGTTA |
| | | CCACTCAGGGACCTAACCTTGCGCTGTATCAGCTGCTT |
| | | AGCACCGCTTTGTGCTCCGCTCTTGAAATTGGAGAAGT |
| | | ACTCAGAGGGCTCGCACTGGGTACTGAGTCAGGGCTGT |
| | | TCTCCCCCTGCTATCTGTCTCTCCGGTTCGACCTCACTC |
| | | GGGATAAGCTGTTGTCAATGGCTCCGCAGGAAGCCAC |
| | | ATTGGACCAGGCCGCAGTGAGTAACGCAGTGGACGGG |
| | | TTCCTGGGTAGACTGTCTCTTGAGAGGGAAGACAGAGA |
| | | CGCTTGGCATCTGCCTGCGTATAAGTGTGTTGATAGAC |
| | | TGGACAAAGTACTGATGATCATTCCCCTGATTAATGTG |
| | | ACATTTATTATATCTAGTGACCGAGAGGTGAGAGGCTC |
| | | CGCTCTCTATGAAGCATCCACAACGTACTTGAGCAGTT |
| | | CCCTGTTTCTGTCACCGGTTATTATGAACAAATGTTCTC |
| | | AGGGAGCTGTTGCTGGTGAACCGCGACAGATTCCAAA |
| | | GATTCAGAACTTCACTAGGACACAAAAGAGCTGCATTT |
| | | TCTGCGGGTTCGCACTGCTGAGTTACGACGAAAAGGAG |
| | | GGACTGGAGACTACTACATATATTACTTCACAGGAGGT |
| | | GCAGAATAGCATCCTGAGCTCCAATTATTTTGACTTTG |
| | | ACAACCTCCATGTCCATTATTTGCTGTTGACCACAAAC |
| | | GGCACTGTGATGGAGATCGCTGGCCTGTACGAGGAGC |
| | | GAGCCCACGTAGTACTGGCCATTATTCTGTACTTCATC |
| | | GCTTTCGCACTTGGGATTTTCCTCGTCCACAAAATTGTT |
| | | ATGTTTTTCCTCTAATGATTCGAACTACAGCTCAACTTA |
| | | CCTGCCAACCCCATGCCAGTCGACCCAACTAGTACAAC |
| | | CTAAATCCATTATAAAAAACTTAGGAACCAGGTCCACA |
| | | CAGCCGCCAGCCCATCAACCATCCACTCCCACGATTGG |
| | | AGGCCGGCCATGGTGAGTTTTAAGCAGGTCCGGGTACC |
| | | TCTGTTCACCGCCATTGCGCTGGTGATTGTTCTGCTGTT |
| | | GGCCTACTTTCTTCCACCTAGAGTCAGAGGAGGTGGCA |
| | | GGGTTGCTGCAGCTGCTATTACCTGGGTACCGAAACCC |
| | | AACGTGGAGGTTTGGCCAGTGGATCCGCCCCCACCAGT |
| | | GAACTTTAATAAAACAGCCGAACAAGAATACGGGGAT |
| | | AAAGAAGTAAAGCTGCCACACTGGACCCCTACCCTCCA |
| | | CACTTTCCAGGTGCCTCAAAACTATACAAAGGCCAATT |
| | | GCACATACTGTAACACAAGAGAATACACTTTTTCTTAT |
| | | AAAGGGTGCTGCTTCTACTTCACTAAGAAGAAGCATAC |
| | | TTGGAACGGATGTTTCCAGGCATGCGCTGAGTTGTATC |
| | | CCTGTACCTATTTCTACGGTCCTACACCAGACATTCTGC |
| | | CGGTCGTGACTCGCAATCTGAACGCCATAGAATCTCTG |
| | | TGGGTCGGGGTGTATCGGGTTGGGGAAGGAAATTGGA |
| | | CTAGCCTGGACGGCGGTACTTTCAAGGTGTACCAAATA |
| | | TTCGGCAGTCACTGTACTTACGTGTCAAAATTCTCAAC |
| | | AGTTCCCGTATCCCACCACGAGTGTTCTTTTTTGAAGCC |
| | | TTGCCTGTGCGTGTCACAGAGGTCAAATTCCTAACTAT |
| | | TCGAACTACTCAGCTAGTCTACGCGCCGTAGCCTACCC |
| | | TCCATCATTGTTATAAAAAACTTAGGAGCAAAGTCCAC |
| | | ACAGCCGCCGATAAGGTCCACAATGAGAGCCGTAGGT |
| | | GTCTTTCTGGCTATTTGTCTGGTAACAATCTTCGTGCTG |
| | | CCCACTTGGGGCAACTGGGCCTATCCTTGCTGCCATGT |
| | | TACACAGTTGAGAGCGCAGCACCTCCTGGCGTTGGAGA |
| | | ACATTTCAGACATCTACCTGGTGAGTAACCAAACCTGC |
| | | GACGGGTTCTCTTTGGCCAGTCTCAATAGTCCAAAAAA |
| | | CGGAAGCAATCAGCTGGTAATTTCACGGTGTGCAAACG |
| | | GACTTAACGTGGTCTCTTTTTTTATATCAATCCTGAAAC |
| | | GCAGTTCATCTGCATTGACCGGCCACTTGAGGGAGCTC |
| | | TTGACCACTCTCGAGACACTTTACGGGTCATTCTCCGT |
| | | GGAGGATCTGTTCGGGGCAAATCTGAACCGGTACGCCT |
| | | GGCATAGAGGCGGGTAAGCGCGCAGCGCTTAGACGTC |
| | | TCGCGATCGATGCTAGTGTGAAATAGACATCAGAATTA |
| | | AGAAAAACGTAGGGTCCAAGTGGTTCCCCGTTATGGAC |
| | | TCGCTATCTGTCAACCAGATCTTATACCCTGAAGTTCA |
| | | CCTAGATAGCCCGATAGTTACCAATAAGATAGTAGCCA |
| | | TCCTGGAGTATGCTCGAGTCCCTCACGCTTACAGCCTG |
| | | GAGGACCCTACACTGTGTCAGAACATCAAGCACCGCCT |
| | | AAAAAACGGATTTTCCAACCAAATGATTATAAACAATG |
| | | TGGAAGTTGGGAATGTCATCAAGTCCAAGCTTAGGAGT |
| | | TATCCGGCCCACTCTCATATTCCATATCCAAATTGTAAT |
| | | CAGGATTTATTTAACATAGAAGACAAAGAGTCAACGA |
| | | GGAAGATCCGTGAACTCCTCAAAAAGGGGAATTCGCT |
| | | GTACTCCAAAGTCAGTGATAAGGTTTTCCAATGCTTAA |
| | | GGGACACTAACTCACGGCTTGGCCTAGGCTCCGAATTG |
| | | AGGGAGGACATCAAGGAGAAAGTTATTAACTTGGGAG |
| | | TTTACATGCACAGCTCCCAGTGGTTTGAGCCCTTTCTGT |
| | | TTTGGTTTACAGTCAAGACTGAGATGAGGTCAGTGATT |
| | | AAATCACAAACCCATACTTGCCATAGGAGGAGACACA |
| | | CACCTGTATTCTTCACTGGTAGTTCAGTTGAGTTGCTAA |
| | | TCTCTCGTGACCTTGTTGCTATAATCAGTAAAGAGTCTC |
| | | AACATGTATATTACCTGACATTTGAACTGGTTTTGATGT |
| | | ATTGTGATGTCATAGAGGGGAGGTTAATGACAGAGAC |
| | | CGCTATGACTATTGATGCTAGGTATACAGAGCTTCTAG |
| | | GAAGAGTCAGATACATGTGGAAACTGATAGATGGTTTC |
| | | TTCCCTGCACTCGGGAATCCAACTTATCAAATTGTAGC |
| | | CATGCTGGAGCCTCTTTCACTTGCTTACCTGCAGCTGA |
| | | GGGATATAACAGTAGAACTCAGAGGTGCTTTCCTTAAC |
| | | CACTGCTTTACTGAAATACATGATGTTCTTGACCAAAA |
| | | CGGGTTTTCTGATGAAGGTACTTATCATGAGTTAACTG |
| | | AAGCTCTAGATTACATTTTCATAACTGATGACATACAT |
| | | CTGACAGGGGAGATTTTCTCATTTTTCAGAAGTTTCGG |
| | | CCACCCCAGACTTGAAGCAGTAACGGCTGCTGAAAAT |
| | | GTTAGGAAATACATGAATCAGCCTAAAGTCATTGTGTA |
| | | TGAGACTCTGATGAAAGGTCATGCCATATTTTGTGGAA |
| | | TCATAATCAACGGCTATCGTGACAGGCACGGAGGCAG |
| | | TTGGCCACCGCTGACCCTCCCCCTGCATGCTGCAGACA |
| | | CAATCCGGAATGCTCAAGCTTCAGGTGAAGGGTTAACA |
| | | CATGAGCAGTGCGTTGATAACTGGAAATCTTTTGCTGG |
| | | AGTGAAATTTGGCTGCTTTATGCCTCTTAGCCTGGATA |
| | | GTGATCTGACAATGTACCTAAAGGACAAGGCACTTGCT |
| | | GCTCTCCAAAGGGAATGGGATTCAGTTTACCCGAAAGA |
| | | GTTCCTGCGTTACGACCCTCCCAAGGGAACCGGGTCAC |
| | | GGAGGCTTGTAGATGTTTTCCTTAATGATTCGAGCTTTG |
| | | ACCCATATGATGTGATAATGTATGTTGTAAGTGGAGCT |
| | | TACCTCCATGACCCTGAGTTCAACCTGTCTTACAGCCT |
| | | GAAAGAAAAGGAGATCAAGGAAACAGGTAGACTTTTT |
| | | GCTAAAATGACTTACAAAATGAGGGCATGCCAAGTGA |
| | | TTGCTGAAAATCTAATCTCAAACGGGATTGGCAAATAT |
| | | TTTAAGGACAATGGGATGGCCAAGGATGAGCACGATT |
| | | TGACTAAGGCACTCCACACTCTAGCTGTCTCAGGAGTC |
| | | CCCAAAGATCTCAAAGAAAGTCACAGGGGGGGGCCAG |
| | | TCTTAAAAACCTACTCCCGAAGCCCAGTCCACACAAGT |
| | | ACCAGGAACGTGAGAGCAGCAAAAGGGTTTATAGGGT |
| | | TCCCTCAAGTAATTCGGCAGGACCAAGACACTGATCAT |
| | | CCGGAGAATATGGAAGCTTACGAGACAGTCAGTGCAT |
| | | TTATCACGACTGATCTCAAGAAGTACTGCCTTAATTGG |
| | | AGATATGAGACCATCAGCTTGTTTGCACAGAGGCTAAA |
| | | TGAGATTTACGGATTGCCCTCATTTTTCCAGTGGCTGCA |
| | | TAAGAGGCTTGAGACCTCTGTCCTGTATGTAAGTGACC |
| | | CTCATTGCCCCCCCGACCTTGACGCCCATATCCCGTTAT |
| | | ATAAAGTCCCCAATGATCAAATCTTCATTAAGTACCCT |
| | | ATGGGAGGTATAGAAGGGTATTGTCAGAAGCTGTGGA |
| | | CCATCAGCACCATTCCCTATCTATACCTGGCTGCTTATG |
| | | AGAGCGGAGTAAGGATTGCTTCGTTAGTGCAAGGGGA |
| | | CAATCAGACCATAGCCGTAACAAAAAGGGTACCCAGC |
| | | ACATGGCCCTACAACCTTAAGAAACGGGAAGCTGCTA |
| | | GAGTAACTAGAGATTACTTTGTAATTCTTAGGCAAAGG |
| | | CTACATGATATTGGCCATCACCTCAAGGCAAATGAGAC |
| | | AATTGTTTCATCACATTTTTTTGTCTATTCAAAAGGAAT |
| | | ATATTATGATGGGCTACTTGTGTCCCAATCACTCAAGA |
| | | GCATCGCAAGATGTGTATTCTGGTCAGAGACTATAGTT |
| | | GATGAAACAAGGGCAGCATGCAGTAATATTGCTACAA |
| | | CAATGGCTAAAAGCATCGAGAGAGGTTATGACCGTTA |
| | | CCTTGCATATTCCCTGAACGTCCTAAAAGTGATACAGC |
| | | AAATTCTGATCTCTCTTGGCTTCACAATCAATTCAACCA |
| | | TGACCCGGGATGTAGTCATACCCCTCCTCACAAACAAC |
| | | GACCTCTTAATAAGGATGGCACTGTTGCCCGCTCCTAT |
| | | TGGGGGGATGAATTATCTGAATATGAGCAGGCTGTTTG |
| | | TCAGAAACATCGGTGATCCAGTAACATCATCAATTGCT |
| | | GATCTCAAGAGAATGATTCTCGCCTCACTAATGCCTGA |
| | | AGAGACCCTCCATCAAGTAATGACACAACAACCGGGG |
| | | GACTCTTCATTCCTAGACTGGGCTAGCGACCCTTACTC |
| | | AGCAAATCTTGTATGTGTCCAGAGCATCACTAGACTCC |
| | | TCAAGAACATAACTGCAAGGTTTGTCCTGATCCATAGT |
| | | CCAAACCCAATGTTAAAAGGATTATTCCATGATGACAG |
| | | TAAAGAAGAGGACGAGGGACTGGCGGCATTCCTCATG |
| | | GACAGGCATATTATAGTACCTAGGGCAGCTCATGAAAT |
| | | CCTGGATCATAGTGTCACAGGGGCAAGAGAGTCTATTG |
| | | CAGGCATGCTGGATACCACAAAAGGCTTGATTCGAGCC |
| | | AGCATGAGGAAGGGGGGGTTAACCTCTCGAGTGATAA |
| | | CCAGATTGTCCAATTATGACTATGAACAATTCAGAGCA |
| | | GGGATGGTGCTATTGACAGGAAGAAAGAGAAATGTCC |
| | | TCATTGACAAAGAGTCATGTTCAGTGCAGCTGGCGAGA |
| | | GCTCTAAGAAGCCATATGTGGGCGAGGCTAGCTCGAG |
| | | GACGGCCTATTTACGGCCTTGAGGTCCCTGATGTACTA |
| | | GAATCTATGCGAGGCCACCTTATTCGGCGTCATGAGAC |
| | | ATGTGTCATCTGCGAGTGTGGATCAGTCAACTACGGAT |
| | | GGTTTTTTGTCCCCTCGGGTTGCCAACTGGATGATATTG |
| | | ACAAGGAAACATCATCCTTGAGAGTCCCATATATTGGT |
| | | TCTACCACTGATGAGAGAACAGACATGAAGCTTGCCTT |
| | | CGTAAGAGCCCCAAGTCGATCCTTGCGATCTGCTGTTA |
| | | GAATAGCAACAGTGTACTCATGGGCTTACGGTGATGAT |
| | | GATAGCTCTTGGAACGAAGCCTGGTTGTTGGCTAGGCA |
| | | AAGGGCCAATGTGAGCCTGGAGGAGCTAAGGGTGATC |
| | | ACTCCCATCTCAACTTCGACTAATTTAGCGCATAGGTT |
| | | GAGGGATCGTAGCACTCAAGTGAAATACTCAGGTACA |
| | | TCCCTTGTCCGAGTGGCGAGGTATACCACAATCTCCAA |
| | | CGACAATCTCTCATTTGTCATATCAGATAAGAAGGTTG |
| | | ATACTAACTTTATATACCAACAAGGAATGCTTCTAGGG |
| | | TTGGGTGTTTTAGAAACATTGTTTCGACTCGAGAAAGA |
| | | TACCGGATCATCTAACACGGTATTACATCTTCACGTCG |
| | | AAACAGATTGTTGCGTGATCCCGATGATAGATCATCCC |
| | | AGGATACCCAGCTCCCGCAAGCTAGAGCTGAGGGCAG |
| | | AGCTATGTACCAACCCATTGATATATGATAATGCACCT |
| | | TTAATTGACAGAGATGCAACAAGGCTATACACCCAGA |
| | | GCCATAGGAGGCACCTTGTGGAATTTGTTACATGGTCC |
| | | ACACCCCAACTATATCACATTTTAGCTAAGTCCACAGC |
| | | ACTATCTATGATTGACCTGGTAACAAAATTTGAGAAGG |
| | | ACCATATGAATGAAATTTCAGCTCTCATAGGGGATGAC |
| | | GATATCAATAGTTTCATAACTGAGTTTCTGCTCATAGA |
| | | GCCAAGATTATTCACTATCTACTTGGGCCAGTGTGCGG |
| | | CCATCAATTGGGCATTTGATGTACATTATCATAGACCA |
| | | TCAGGGAAATATCAGATGGGTGAGCTGTTGTCATCGTT |
| | | CCTTTCTAGAATGAGCAAAGGAGTGTTTAAGGTGCTTG |
| | | TCAATGCTCTAAGCCACCCAAAGATCTACAAGAAATTC |
| | | TGGCATTGTGGTATTATAGAGCCTATCCATGGTCCTTC |
| | | ACTTGATGCTCAAAACTTGCACACAACTGTGTGCAACA |
| | | TGGTTTACACATGCTATATGACCTACCTCGACCTGTTGT |
| | | TGAATGAAGAGTTAGAAGAGTTCACATTTCTCTTGTGT |
| | | GAAAGCGACGAGGATGTAGTACCGGACAGATTCGACA |
| | | ACATCCAGGCAAAACACTTATGTGTTCTGGCAGATTTG |
| | | TACTGTCAACCAGGGACCTGCCCACCAATTCGAGGTCT |
| | | AAGACCGGTAGAGAAATGTGCAGTTCTAACCGACCAT |
| | | ATCAAGGCAGAGGCTATGTTATCTCCAGCAGGATCTTC |
| | | GTGGAACATAAATCCAATTATTGTAGACCATTACTCAT |
| | | GCTCTCTGACTTATCTCCGGCGAGGATCGATCAAACAG |
| | | ATAAGATTGAGAGTTGATCCAGGATTCATTTTCGACGC |
| | | CCTCGCTGAGGTAAATGTCAGTCAGCCAAAGATCGGCA |
| | | GCAACAACATCTCAAATATGAGCATCAAGGCTTTCAGA |
| | | CCCCCACACGATGATGTTGCAAAATTGCTCAAAGATAT |
| | | CAACACAAGCAAGCACAATCTTCCCATTTCAGGGGGCA |
| | | ATCTCGCCAATTATGAAATCCATGCTTTCCGCAGAATC |
| | | GGGTTGAACTCATCTGCTTGCTACAAAGCTGTTGAGAT |
| | | ATCAACATTAATTAGGAGATGCCTTGAGCCAGGGGAG |
| | | GACGGCTTGTTCTTGGGTGAGGGATCGGGTTCTATGTT |
| | | GATCACTTATAAAGAGATACTTAAACTAAACAAGTGCT |
| | | TCTATAATAGTGGGGTTTCCGCCAATTCTAGATCTGGT |
| | | CAAAGGGAATTAGCACCCTATCCCTCCGAAGTTGGCCT |
| | | TGTCGAACACAGAATGGGAGTAGGTAATATTGTCAAA |
| | | GTGCTCTTTAACGGGAGGCCCGAAGTCACGTGGGTAGG |
| | | CAGTGTAGATTGCTTCAATTTCATAGTTAGTAATATCCC |
| | | TACCTCTAGTGTGGGGTTTATCCATTCAGATATAGAGA |
| | | CCTTGCCTGACAAAGATACTATAGAGAAGCTAGAGGA |
| | | ATTGGCAGCCATCTTATCGATGGCTCTGCTCCTGGGCA |
| | | AAATAGGATCAATACTGGTGATTAAGCTTATGCCTTTC |
| | | AGCGGGGATTTTGTTCAGGGATTTATAAGTTATGTAGG |
| | | GTCTCATTATAGAGAAGTGAACCTTGTATACCCTAGAT |
| | | ACAGCAACTTCATCTCTACTGAATCTTATTTGGTTATGA |
| | | CAGATCTCAAGGCTAACCGGCTAATGAATCCTGAAAA |
| | | GATTAAGCAGCAGATAATTGAATCATCTGTGAGGACTT |
| | | CACCTGGACTTATAGGTCACATCCTATCCATTAAGCAA |
| | | CTAAGCTGCATACAAGCAATTGTGGGAGACGCAGTTA |
| | | GTAGAGGTGATATCAATCCTACTCTGAAAAAACTTACA |
| | | CCTATAGAGCAGGTGCTGATCAATTGCGGGTTGGCAAT |
| | | TAACGGACCTAAGCTGTGCAAAGAATTGATCCACCATG |
| | | ATGTTGCCTCAGGGCAAGATGGATTGCTTAATTCTATA |
| | | CTCATCCTCTACAGGGAGTTGGCAAGATTCAAAGACAA |
| | | CCAAAGAAGTCAACAAGGGATGTTCCACGCTTACCCCG |
| | | TATTGGTAAGTAGCAGGCAACGAGAACTTATATCTAGG |
| | | ATCACCCGCAAATTCTGGGGGCACATTCTTCTTTACTCC |
| | | GGGAACAAAAAGTTGATAAATAAGTTTATCCAGAATCT |
| | | CAAGTCCGGCTATCTGATACTAGACTTACACCAGAATA |
| | | TCTTCGTTAAGAATCTATCCAAGTCAGAGAAACAGATT |
| | | ATTATGACGGGGGGTTTGAAACGTGAGTGGGTTTTTAA |
| | | GGTAACAGTCAAGGAGACCAAAGAATGGTATAAGTTA |
| | | GTCGGATACAGTGCCCTGATTAAGGACTAATTGGTTGA |
| | | ACTCCGGAACCCTAATCCTGCCCTAGGTGGTTAGGCAT |
| | | TATTTGCAATATATTAAAGAAAACTTTGAAAATACGAA |
| | | GTTTCTATTCCCAGCTTTGTCTGGT |
| 62 | ATU3-PCR- | CGAAGACTGCCATGCCCCAACATAC |
| | 1F1 (forward | |
| | primer) | |
| 63 | ATU3-PCR- | TAGCGAGTCCATAACGGGGAACCAC |
| | 1R1 (reverse | |
| | primer) | |
| 64 | Seq_Pr_LMP2_ | TTCTTTCTCCGTCCTCCGTCTC |
| | 278 | |
| 65 | ATU1 motif | CTTCTAGTGCACTTAGGATTCAA |
| 66 | GE (gene end)- | CTTCTAGTGCA |
| | like sequence | |
| | upstream of N | |
| | ORF | |
| 67 | Conserved | GTT |
| | trinucleotide | |
| | sequence | |
| 68 | GS (gene start) | AGGATTCAA |
| | sequence of N | |
| | gene | |
| 69 | ATU upstream | GTTATAAAAAACTTAGGAACCAGGTCCACAC |
| | motif | |
| 70 | GE (gene end) | GTTATAAAAAA |
| | sequence of N | |
| | gene | |
| 71 | GS (gene start) | AGGAACCAGGTCCACAC |
| | sequence of P | |
| | gene | |
| 72 | ATU | ATTATAAAAAACTTAGGAGCAAAGTGATTGC |
| | downstream | |
| | motif | |
| 73 | GE sequence of | ATTATAAAAAA |
| | P gene | |
| 74 | GS sequence of | AGGAGCAAAGTGATTGC |
| | M gene | |
| 75 | ATUa- | ATTATAAAAAACTTAGGAACCAGGTCCACAC |
| | interstitial ATU | |
| 76 | Hybrid GS | AGGAGCAAAGTCCACAC |
| | sequence | |
| 77 | Consensus GS | AGGATCCAAGAGCATAC |
| | sequence | |
| 78 | ATUb- | GTTATAAAAAACTTAGGAGCAAAGTCCACAC |
| | interstitial ATU | |
| 79 | ATUc- | GTTATAAAAAACTTAGGATCCAAGAGCATAC |
| | interstitial ATU | |
| 80 | Kozak sequence | GCCGCCATG |
| 81 | Kozak sequence | GCCACCATG |
| 82 | M-04 | ACCAAACAAAGTTGGGTAAGGATAGTTCAATCAATGA |
| | EBV_gp350 at | TCATCTTCTAGTGCACTTAGGATTCAAGATCCTATTATC |
| | ATU2 | AGGGACAAGAGCAGGATTAGGGATATCCGAGATGGCC |
| | MV leader: nt | ACACTTTTAAGGAGCTTAGCATTGTTCAAAAGAAACAA |
| | 1-55 | GGACAAACCACCCATTACATCAGGATCCGGTGGAGCC |
| | MV N ORF: nt | ATCAGAGGAATCAAACACATTATTATAGTACCAATCCC |
| | 108-1,685 | TGGAGATTCCTCAATTACCACTCGATCCAGACTTCTGG |
| | MV P ORF: nt | ACCGGTTGGTGAGGTTAATTGGAAACCCGGATGTGAGC |
| | 1,807-3,330 | GGGCCCAAACTAACAGGGGCACTAATAGGTATATTATC |
| | ATU upstream | CTTATTTGTGGAGTCTCCAGGTCAATTGATTCAGAGGA |
| | motif: nt 3,378- | TCACCGATGACCCTGACGTTAGCATAAGGCTGTTAGAG |
| | 3,455 | GTTGTCCAGAGTGACCAGTCACAATCTGGCCTTACCTT |
| | GE of N gene: | CGCATCAAGAGGTACCAACATGGAGGATGAGGCGGAC |
| | nt 3,394-3,404 | CAATACTTTTCACATGATGATCCAATTAGTAGTGATCA |
| | Conserved | ATCCAGGTTCGGATGGTTCGGGAACAAGGAAATCTCA |
| | trinucleotide | GATATTGAAGTGCAAGACCCTGAGGGATTCAACATGAT |
| | motif: nt 3,405- | TCTGGGTACCATCCTAGCCCAAATTTGGGTCTTGCTCG |
| | 3,407 | CAAAGGCGGTTACGGCCCCAGACACGGCAGCTGATTC |
| | GS of P gene: | GGAGCTAAGAAGGTGGATAAAGTACACCCAACAAAGA |
| | nt 3,408-3,424 | AGGGTAGTTGGTGAATTTAGATTGGAGAGAAAATGGTT |
| | BsiWI | GGATGTGGTGAGGAACAGGATTGCCGAGGACCTCTCCT |
| | restriction site: | TACGCCGATTCATGGTCGCTCTAATCCTGGATATCAAG |
| | nt 3,444-3,449 | AGAACACCCGGAAACAAACCCAGGATTGCTGAAATGA |
| | Kozak | TATGTGACATTGATACATATATCGTAGAGGCAGGATTA |
| | sequence: nt | GCCAGTTTTATCCTGACTATTAAGTTTGGGATAGAAAC |
| | 3,450-3,458 | TATGTATCCTGCTCTTGGACTGCATGAATTTGCTGGTGA |
| | EBV gp350 | GTTATCCACACTTGAGTCCTTGATGAACCTTTACCAGC |
| | ORF: nt 3,456- | AAATGGGGGAAACTGCACCCTACATGGTAATCCTGGA |
| | 6,179-2,724 | GAACTCAATTCAGAACAAGTTCAGTGCAGGATCATACC |
| | BssHII | CTCTGCTCTGGAGCTATGCCATGGGAGTAGGAGTGGAA |
| | restriction site: | CTTGAAAACTCCATGGGAGGTTTGAACTTTGGCCGATC |
| | nt 6,180-6,185 | TTACTTTGATCCAGCATATTTTAGATTAGGGCAAGAGA |
| | ATU | TGGTAAGGAGGTCAGCTGGAAAGGTCAGTTCCACATTG |
| | downstream | GCATCTGAACTCGGTATCACTGCCGAGGATGCAAGGCT |
| | motif: nt 6,180- | TGTTTCAGAGATTGCAATGCATACTACTGAGGACAAGA |
| | 6,215 | TCAGTAGAGCGGTTGGACCCAGACAAGCCCAAGTATC |
| | GE of P gene: | ATTTCTACACGGTGATCAAAGTGAGAATGAGCTACCGA |
| | nt 6,230-6,240 | GATTGGGGGGCAAGGAAGATAGGAGGGTCAAACAGAG |
| | GS of M gene: | TCGAGGAGAAGCCAGGGAGAGCTACAGAGAAACCGGG |
| | nt 6,244-6,260 | CCCAGCAGAGCAAGTGATGCGAGAGCTGCCCATCTTCC |
| | MV M ORF: nt | AACCGGCACACCCCTAGACATTGACACTGCAACGGAG |
| | 6,276-7,283 | TCCAGCCAAGATCCGCAGGACAGTCGAAGGTCAGCTG |
| | MV F ORF: nt | ACGCCCTGCTTAGGCTGCAAGCCATGGCAGGAATCTCG |
| | 8,287-9,948 | GAAGAACAAGGCTCAGACACGGACACCCCTATAGTGT |
| | MV H ORF: nt | ACAATGACAGAAATCTTCTAGACTAGGTGCGAGAGGC |
| | 10,109-11,962 | CGAGGGCCAGAACAACATCCGCCTACCATCCATCATTG |
| | MV L ORF: nt | TTATAAAAAACTTAGGAACCAGGTCCACACAGCCGCC |
| | 12,072-18,623 | AGCCCATCAACCATCCACTCCCACGATTGGAGCCAATG |
| | MV trailer: nt | GCAGAAGAGCAGGCACGCCATGTCAAAAACGGACTGG |
| | 18,696-18,732 | AATGCATCCGGGCTCTCAAGGCCGAGCCCATCGGCTCA |
| | | CTGGCCATCGAGGAAGCTATGGCAGCATGGTCAGAAA |
| | | TATCAGACAACCCAGGACAGGAGCGAGCCACCTGCAG |
| | | GGAAGAGAAGGCAGGCAGTTCGGGTCTCAGCAAACCA |
| | | TGCCTCTCAGCAATTGGATCAACTGAAGGCGGTGCACC |
| | | TCGCATCCGCGGTCAGGGACCTGGAGAGAGCGATGAC |
| | | GACGCTGAAACTTTGGGAATCCCCCCAAGAAATCTCCA |
| | | GGCATCAAGCACTGGGTTACAGTGTTATTACGTTTATG |
| | | ATCACAGCGGTGAAGCGGTTAAGGGAATCCAAGATGC |
| | | TGACTCTATCATGGTTCAATCAGGCCTTGATGGTGATA |
| | | GCACCCTCTCAGGAGGAGACAATGAATCTGAAAACAG |
| | | CGATGTGGATATTGGCGAACCTGATACCGAGGGATATG |
| | | CTATCACTGACCGGGGATCTGCTCCCATCTCTATGGGG |
| | | TTCAGGGCTTCTGATGTTGAAACTGCAGAAGGAGGGG |
| | | AGATCCACGAGCTCCTGAGACTCCAATCCAGAGGCAA |
| | | CAACTTTCCGAAGCTTGGGAAAACTCTCAATGTTCCTC |
| | | CGCCCCCGGACCCCGGTAGGGCCAGCACTTCCGGGAC |
| | | ACCCATTAAAAAGGGCACAGACGCGAGATTAGCCTCA |
| | | TTTGGAACGGAGATCGCGTCTTTATTGACAGGTGGTGC |
| | | AACCCAATGTGCTCGAAAGTCACCCTCGGAACCATCAG |
| | | GGCCAGGTGCACCTGCGGGGAATGTCCCCGAGTGTGTG |
| | | AGCAATGCCGCACTGATACAGGAGTGGACACCCGAAT |
| | | CTGGTACCACAATCTCCCCGAGATCCCAGAATAATGAA |
| | | GAAGGGGGAGACTATTATGATGATGAGCTGTTCTCTGA |
| | | TGTCCAAGATATTAAAACAGCCTTGGCCAAAATACACG |
| | | AGGATAATCAGAAGATAATCTCCAAGCTAGAATCACT |
| | | GCTGTTATTGAAGGGAGAAGTTGAGTCAATTAAGAAG |
| | | CAGATCAACAGGCAAAATATCAGCATATCCACCCTGG |
| | | AAGGACACCTCTCAAGCATCATGATCGCCATTCCTGGA |
| | | CTTGGGAAGGATCCCAACGACCCCACTGCAGATGTCGA |
| | | AATCAATCCCGACTTGAAACCCATCATAGGCAGAGATT |
| | | CAGGCCGAGCACTGGCCGAAGTTCTCAAGAAACCCGTT |
| | | GCCAGCCGACAACTCCAAGGAATGACAAATGGACGGA |
| | | CCAGTTCCAGAGGACAGCTGCTGAAGGAATTTCAGCTA |
| | | AAGCCGATCGGGAAAAAGATGAGCTCAGCCGTCGGGT |
| | | TTGTTCCTGACACCGGCCCTGCATCACGCAGTGTAATC |
| | | CGCTCCATTATAAAATCCAGCCGGCTAGAGGAGGATCG |
| | | GAAGCGTTACCTGATGACTCTCCTTGATGATATCAAAG |
| | | GAGCCAATGATCTTGCCAAGTTCCACCAGATGCTGATG |
| | | AAGATAATAATGAAGTAGCTACAGCTCAACTTACCTGC |
| | | CAACCCCATGCCAGTCGACCCAACTAGCCTACCCTCCA |
| | | TCATTGTTATAAAAAACTTAGGAACCAGGTCCACACAG |
| | | CCGCCAGCCCATCAACGCGTACGGCCACCATGGAGGC |
| | | CGCTTTGCTGGTGTGTCAGTACACAATTCAGAGCCTGA |
| | | TCCACTTGACGGGAGAGGACCCTGGCTTTTTCAACGTT |
| | | GAGATCCCCGAATTCCCATTTTATCCAACCTGCAACGT |
| | | GTGTACTGCAGACGTAAACGTTACTATTAATTTCGACG |
| | | TGGGAGGCAAGAAACACCAACTGGATCTCGACTTTGG |
| | | ACAGCTGACTCCCCACACCAAAGCTGTTTACCAACCCA |
| | | GAGGGGCTTTTGGCGGCTCTGAAAACGCTACCAACCTC |
| | | TTCCTGCTCGAGCTTCTGGGAGCTGGCGAGCTTGCTCT |
| | | GACTATGCGCTCTAAAAAGCTCCCGATTAACGTCACAA |
| | | CAGGGGAGGAACAACAGGTGAGCTTGGAGTCCGTAGA |
| | | CGTGTACTTCCAGGACGTGTTCGGAACAATGTGGTGTC |
| | | ATCACGCTGAGATGCAGAATCCCGTGTACCTCATTCCA |
| | | GAGACGGTGCCTTATATAAAGTGGGATAACTGTAACTC |
| | | AACGAATATCACAGCAGTGGTTAGGGCGCAAGGTCTG |
| | | GACGTCACTCTTCCTCTCTCACTCCCAACTTCCGCTCAA |
| | | GACAGCAATTTCTCAGTGAAAACAGAAATGCTGGGCA |
| | | ACGAGATAGATATTGAGTGTATAATGGAAGACGGGGA |
| | | GATTAGTCAAGTGCTGCCTGGCGACAACAAATTTAATA |
| | | TCACCTGTAGCGGTTACGAGAGTCACGTCCCGTCCGGG |
| | | GGGATTCTGACCAGCACTAGCCCTGTCGCCACTCCCAT |
| | | TCCGGGAACTGGATACGCCTATAGCCTTAGGCTTACAC |
| | | CCAGACCTGTGTCACGGTTTCTGGGCAACAACAGCATT |
| | | CTGTACGTGTTCTACAGTGGCAACGGGCCTAAAGCCTC |
| | | TGGTGGCGACTATTGTATTCAAAGCAACATCGTCTTCT |
| | | CCGACGAGATCCCTGCAAGCCAGGACATGCCCACTAAT |
| | | ACCACCGACATTACCTACGTAGGGGACAACGCTACTTA |
| | | CTCTGTCCCGATGGTGACTAGCGAGGATGCTAATTCTC |
| | | CAAACGTTACCGTAACCGCCTTTTGGGCTTGGCCCAAT |
| | | AATACAGAGACGGATTTCAAATGCAAGTGGACTTTGAC |
| | | TTCCGGCACCCCCTCCGGCTGTGAAAATATTTCTGGCG |
| | | CTTTCGCCAGCAACAGGACGTTCGACATCACAGTCTCT |
| | | GGTCTTGGCACTGCCCCAAAGACCCTGATCATCACCCG |
| | | GACTGCCACTAACGCGACAACTACAACTCATAAGGTTA |
| | | TTTTTAGCAAGGCACCAGAGAGCACCACTACATCTCCC |
| | | ACCCTGAACACTACGGGCTTTGCTGACCCGAATACCAC |
| | | CACGGGCCTTCCCTCATCAACCCATGTCCCAACCAACT |
| | | TGACAGCACCAGCATCAACGGGTCCCACCGTCTCCACA |
| | | GCAGACGTAACCTCTCCTACACCTGCCGGCACCACAAG |
| | | TGGCGCTAGTCCTGTGACCCCTTCCCCATCTCCTTGGGA |
| | | TAATGGCACAGAGTCAAAAGCCCCTGATATGACTAGCT |
| | | CCACCAGTCCTGTGACGACACCAACCCCAAACGCTACC |
| | | TCCCCCACTCCAGCCGTTACAACTCCAACGCCAAACGC |
| | | TACCTCCCCTACTCCTGCCGTAACCACGCCTACCCCTA |
| | | ATGCCACCAGTCCTACTTTGGGCAAGACCTCCCCGACA |
| | | AGCGCTGTGACAACCCCTACGCCCAACGCAACTTCTCC |
| | | TACACTCGGAAAGACCTCACCTACCAGTGCTGTCACCA |
| | | CACCTACACCAAACGCCACTAGCCCCACATTGGGTAAG |
| | | ACATCCCCCACTTCCGCAGTTACGACTCCCACACCGAA |
| | | CGCAACCGGACCTACTGTCGGTGAGACATCCCCTCAGG |
| | | CCAACGCCACAAATCACACACTGGGCGGCACCTCACC |
| | | AACTCCAGTAGTGACCTCCCAGCCAAAAAACGCCACCT |
| | | CTGCTGTGACCACCGGTCAGCATAACATCACAAGTTCC |
| | | TCCACTTCTAGCATGAGTTTGCGCCCCTCATCCAACCC |
| | | AGAAACGCTGAGCCCTTCAACATCTGACAACTCAACAA |
| | | GCCATATGCCTCTGCTCACTAGTGCTCACCCAACAGGG |
| | | GGAGAAAACATTACACAGGTGACACCCGCCAGCATCT |
| | | CTACACACCACGTGAGCACAAGCTCTCCCGCTCCTCGG |
| | | CCAGGTACAACCTCCCAAGCCTCTGGGCCTGGAAATTC |
| | | CTCAACCTCCACCAAACCCGGTGAGGTCAACGTCACCA |
| | | AGGGAACACCCCCCCAGAACGCAACGTCACCTCAAGC |
| | | ACCCAGCGGACAGAAGACCGCTGTGCCTACTGTAACCT |
| | | CTACCGGAGGGAAAGCTAATAGTACGACTGGAGGTAA |
| | | GCACACTACGGGCCACGGTGCCAGAACCAGTACAGAG |
| | | CCTACCACTGATTACGGCGGAGATAGTACAACCCCCCG |
| | | CCCTAGATACAACGCCACAACTTATTTGCCTCCCTCAA |
| | | CGTCCTCCAAGCTGAGACCCAGGTGGACGTTTACGTCT |
| | | CCCCCAGTCACAACTGCGCAGGCAACCGTGCCAGTTCC |
| | | ACCTACAAGTCAGCCGCGCTTCAGCAATCTCTCAATGC |
| | | TCGTGCTGCAGTGGGCCTCTCTGGCAGTACTGACCCTC |
| | | TTGCTTCTCTTGGTGATGGCTGACTGTGCCTTTCGGAGA |
| | | AACCTGAGCACCTCACACACTTATACCACACCTCCCTA |
| | | TGACGATGCCGAGACATATGTATAAGCGCGCAGCGCTT |
| | | AGACGTCTCGCGATCGATACTAGTACAACCTAAATCCA |
| | | TTATAAAAAACTTAGGAGCAAAGTGATTGCCTCCCAAG |
| | | GTCCACAATGACAGAGACCTACGACTTCGACAAGTCG |
| | | GCATGGGACATCAAAGGGTCGATCGCTCCGATACAAC |
| | | CCACCACCTACAGTGATGGCAGGCTGGTGCCCCAGGTC |
| | | AGAGTCATAGATCCTGGTCTAGGCGACAGGAAGGATG |
| | | AATGCTTTATGTACATGTTTCTGCTGGGGGTTGTTGAG |
| | | GACAGCGATTCCCTAGGGCCTCCAATCGGGCGAGCATT |
| | | TGGGTTCCTGCCCTTAGGTGTTGGCAGATCCACAGCAA |
| | | AGCCCGAAAAACTCCTCAAAGAGGCCACTGAGCTTGA |
| | | CATAGTTGTTAGACGTACAGCAGGGCTCAATGAAAAA |
| | | CTGGTGTTCTACAACAACACCCCACTAACTCTCCTCAC |
| | | ACCTTGGAGAAAGGTCCTAACAACAGGGAGTGTCTTCA |
| | | ACGCAAACCAAGTGTGCAATGCGGTTAATCTGATACCG |
| | | CTCGATACCCCGCAGAGGTTCCGTGTTGTTTATATGAG |
| | | CATCACCCGTCTTTCGGATAACGGGTATTACACCGTTC |
| | | CTAGAAGAATGCTGGAATTCAGATCGGTCAATGCAGTG |
| | | GCCTTCAACCTGCTGGTGACCCTTAGGATTGACAAGGC |
| | | GATAGGCCCTGGGAAGATCATCGACAATACAGAGCAA |
| | | CTTCCTGAGGCAACATTTATGGTCCACATCGGGAACTT |
| | | CAGGAGAAAGAAGAGTGAAGTCTACTCTGCCGATTATT |
| | | GCAAAATGAAAATCGAAAAGATGGGCCTGGTTTTTGC |
| | | ACTTGGTGGGATAGGGGGCACCAGTCTTCACATTAGAA |
| | | GCACAGGCAAAATGAGCAAGACTCTCCATGCACAACT |
| | | CGGGTTCAAGAAGACCTTATGTTACCCGCTGATGGATA |
| | | TCAATGAAGACCTTAATCGATTACTCTGGAGGAGCAGA |
| | | TGCAAGATAGTAAGAATCCAGGCAGTTTTGCAGCCATC |
| | | AGTTCCTCAAGAATTCCGCATTTACGACGACGTGATCA |
| | | TAAATGATGACCAAGGACTATTCAAAGTTCTGTAGACC |
| | | GTAGTGCCCAGCAATGCCCGAAAACGACCCCCCTCACA |
| | | ATGACAGCCAGAAGGCCCGGACAAAAAAGCCCCCTCC |
| | | GAAAGACTCCACGGACCAAGCGAGAGGCCAGCCAGCA |
| | | GCCGACGGCAAGCGCGAACACCAGGCGGCCCCAGCAC |
| | | AGAACAGCCCTGACACAAGGCCACCACCAGCCACCCC |
| | | AATCTGCATCCTCCTCGTGGGACCCCCGAGGACCAACC |
| | | CCCAAGGCTGCCCCCGATCCAAACCACCAACCGCATCC |
| | | CCACCACCCCCGGGAAAGAAACCCCCAGCAATTGGAA |
| | | GGCCCCTCCCCCTCTTCCTCAACACAAGAACTCCACAA |
| | | CCGAACCGCACAAGCGACCGAGGTGACCCAACCGCAG |
| | | GCATCCGACTCCCTAGACAGATCCTCTCTCCCCGGCAA |
| | | ACTAAACAAAACTTAGGGCCAAGGAACATACACACCC |
| | | AACAGAACCCAGACCCCGGCCCACGGCGCCGCGCCCC |
| | | CAACCCCCGACAACCAGAGGGAGCCCCCAACCAATCC |
| | | CGCCGGCTCCCCCGGTGCCCACAGGCAGGGACACCAA |
| | | CCCCCGAACAGACCCAGCACCCAACCATCGACAATCC |
| | | AAGACGGGGGGGCCCCCCCAAAAAAAGGCCCCCAGGG |
| | | GCCGACAGCCAGCACCGCGAGGAAGCCCACCCACCCC |
| | | ACACACGACCACGGCAACCAAACCAGAACCCAGACCA |
| | | CCCTGGGCCACCAGCTCCCAGACTCGGCCATCACCCCG |
| | | CAGAAAGGAAAGGCCACAACCCGCGCACCCCAGCCCC |
| | | GATCCGGCGGGGAGCCACCCAACCCGAACCAGCACCC |
| | | AAGAGCGATCCCCGAAGGACCCCCGAACCGCAAAGGA |
| | | CATCAGTATCCCACAGCCTCTCCAAGTCCCCCGGTCTC |
| | | CTCCTCTTCTCGAAGGGACCAAAAGATCAATCCACCAC |
| | | ACCCGACGACACTCAACTCCCCACCCCTAAAGGAGAC |
| | | ACCGGGAATCCCAGAATCAAGACTCATCCAATGTCCAT |
| | | CATGGGTCTCAAGGTGAACGTCTCTGCCATATTCATGG |
| | | CAGTACTGTTAACTCTCCAAACACCCACCGGTCAAATC |
| | | CATTGGGGCAATCTCTCTAAGATAGGGGTGGTAGGAAT |
| | | AGGAAGTGCAAGCTACAAAGTTATGACTCGTTCCAGCC |
| | | ATCAATCATTAGTCATAAAATTAATGCCCAATATAACT |
| | | CTCCTCAATAACTGCACGAGGGTAGAGATTGCAGAATA |
| | | CAGGAGACTACTGAGAACAGTTTTGGAACCAATTAGA |
| | | GATGCACTTAATGCAATGACCCAGAATATAAGACCGGT |
| | | TCAGAGTGTAGCTTCAAGTAGGAGACACAAGAGATTT |
| | | GCGGGAGTAGTCCTGGCAGGTGCGGCCCTAGGCGTTGC |
| | | CACAGCTGCTCAGATAACAGCCGGCATTGCACTTCACC |
| | | AGTCCATGCTGAACTCTCAAGCCATCGACAATCTGAGA |
| | | GCGAGCCTGGAAACTACTAATCAGGCAATTGAGACAA |
| | | TCAGACAAGCAGGGCAGGAGATGATATTGGCTGTTCA |
| | | GGGTGTCCAAGACTACATCAATAATGAGCTGATACCGT |
| | | CTATGAACCAACTATCTTGTGATTTAATCGGCCAGAAG |
| | | CTCGGGCTCAAATTGCTCAGATACTATACAGAAATCCT |
| | | GTCATTATTTGGCCCCAGTTTACGGGACCCCATATCTG |
| | | CGGAGATATCTATCCAGGCTTTGAGCTATGCGCTTGGA |
| | | GGAGACATCAATAAGGTGTTAGAAAAGCTCGGATACA |
| | | GTGGAGGTGATTTACTGGGCATCTTAGAGAGCGGAGG |
| | | AATAAAGGCCCGGATAACTCACGTCGACACAGAGTCC |
| | | TACTTCATTGTCCTCAGTATAGCCTATCCGACGCTGTCC |
| | | GAGATTAAGGGGGTGATTGTCCACCGGCTAGAGGGGG |
| | | TCTCGTACAACATAGGCTCTCAAGAGTGGTATACCACT |
| | | GTGCCCAAGTATGTTGCAACCCAAGGGTACCTTATCTC |
| | | GAATTTTGATGAGTCATCGTGTACTTTCATGCCAGAGG |
| | | GGACTGTGTGCAGCCAAAATGCCTTGTACCCGATGAGT |
| | | CCTCTGCTCCAAGAATGCCTCCGGGGGTACACCAAGTC |
| | | CTGTGCTCGTACACTCGTATCCGGGTCTTTTGGGAACC |
| | | GGTTCATTTTATCACAAGGGAACCTAATAGCCAATTGT |
| | | GCATCAATCCTTTGCAAGTGTTACACAACAGGAACGAT |
| | | CATTAATCAAGACCCTGACAAGATCCTAACATACATTG |
| | | CTGCCGATCACTGCCCGGTAGTCGAGGTGAACGGCGTG |
| | | ACCATCCAAGTCGGGAGCAGGAGGTATCCAGACGCTG |
| | | TGTACTTGCACAGAATTGACCTCGGTCCTCCCATATCA |
| | | TTGGAGAGGTTGGACGTAGGGACAAATCTGGGGAATG |
| | | CAATTGCTAAGTTGGAGGATGCCAAGGAATTGTTGGAG |
| | | TCATCGGACCAGATATTGAGGAGTATGAAAGGTTTATC |
| | | GAGCACTAGCATAGTCTACATCCTGATTGCAGTGTGTC |
| | | TTGGAGGGTTGATAGGGATCCCCGCTTTAATATGTTGC |
| | | TGCAGGGGGCGTTGTAACAAAAAGGGAGAACAAGTTG |
| | | GTATGTCAAGACCAGGCCTAAAGCCTGATCTTACGGGA |
| | | ACATCAAAATCCTATGTAAGGTCGCTCTGATCCTCTAC |
| | | AACTCTTGAAACACAAATGTCCCACAAGTCTCCTCTTC |
| | | GTCATCAAGCAACCACCGCACCCAGCATCAAGCCCACC |
| | | TGAAATTATCTCCGGCTTCCCTCTGGCCGAACAATATC |
| | | GGTAGTTAATCAAAACTTAGGGTGCAAGATCATCCACA |
| | | ATGTCACCACAACGAGACCGGATAAATGCCTTCTACAA |
| | | AGATAACCCCCATCCCAAGGGAAGTAGGATAGTCATT |
| | | AACAGAGAACATCTTATGATTGATAGACCTTATGTTTT |
| | | GCTGGCTGTTCTGTTTGTCATGTTTCTGAGCTTGATCGG |
| | | GTTGCTAGCCATTGCAGGCATTAGACTTCATCGGGCAG |
| | | CCATCTACACCGCAGAGATCCATAAAAGCCTCAGCACC |
| | | AATCTAGATGTAACTAACTCAATCGAGCATCAGGTCAA |
| | | GGACGTGCTGACACCACTCTTCAAAATCATCGGTGATG |
| | | AAGTGGGCCTGAGGACACCTCAGAGATTCACTGACCTA |
| | | GTGAAATTAATCTCTGACAAGATTAAATTCCTTAATCC |
| | | GGATAGGGAGTACGACTTCAGAGATCTCACTTGGTGTA |
| | | TCAACCCGCCAGAGAGAATCAAATTGGATTATGATCAA |
| | | TACTGTGCAGATGTGGCTGCTGAAGAGCTCATGAATGC |
| | | ATTGGTGAACTCAACTCTACTGGAGACCAGAACAACCA |
| | | ATCAGTTCCTAGCTGTCTCAAAGGGAAACTGCTCAGGG |
| | | CCCACTACAATCAGAGGTCAATTCTCAAACATGTCGCT |
| | | GTCCCTGTTAGACTTGTATTTAGGTCGAGGTTACAATG |
| | | TGTCATCTATAGTCACTATGACATCCCAGGGAATGTAT |
| | | GGGGGAACTTACCTAGTGGAAAAGCCTAATCTGAGCA |
| | | GCAAAAGGTCAGAGTTGTCACAACTGAGCATGTACCG |
| | | AGTGTTTGAAGTAGGTGTTATCAGAAATCCGGGTTTGG |
| | | GGGCTCCGGTGTTCCATATGACAAACTATCTTGAGCAA |
| | | CCAGTCAGTAATGATCTCAGCAACTGTATGGTGGCTTT |
| | | GGGGGAGCTCAAACTCGCAGCCCTTTGTCACGGGGAA |
| | | GATTCTATCACAATTCCCTATCAGGGATCAGGGAAAGG |
| | | TGTCAGCTTCCAGCTCGTCAAGCTAGGTGTCTGGAAAT |
| | | CCCCAACCGACATGCAATCCTGGGTCCCCTTATCAACG |
| | | GATGATCCAGTGATAGACAGGCTTTACCTCTCATCTCA |
| | | CAGAGGTGTTATCGCTGACAATCAAGCAAAATGGGCT |
| | | GTCCCGACAACACGAACAGATGACAAGTTGCGAATGG |
| | | AGACATGCTTCCAACAGGCGTGTAAGGGTAAAATCCA |
| | | AGCACTCTGCGAGAATCCCGAGTGGGCACCATTGAAG |
| | | GATAACAGGATTCCTTCATACGGGGTCTTGTCTGTTGA |
| | | TCTGAGTCTGACAGTTGAGCTTAAAATCAAAATTGCTT |
| | | CGGGATTCGGGCCATTGATCACACACGGTTCAGGGATG |
| | | GACCTATACAAATCCAACCACAACAATGTGTATTGGCT |
| | | GACTATCCCGCCAATGAAGAACCTAGCCTTAGGTGTAA |
| | | TCAACACATTGGAGTGGATACCGAGATTCAAGGTTAGT |
| | | CCCTACCTCTTCACTGTCCCAATTAAGGAAGCAGGCGA |
| | | AGACTGCCATGCCCCAACATACCTACCTGCGGAGGTGG |
| | | ATGGTGATGTCAAACTCAGTTCCAATCTGGTGATTCTA |
| | | CCTGGTCAAGATCTCCAATATGTTTTGGCAACCTACGA |
| | | TACTTCCAGGGTTGAACATGCTGTGGTTTATTACGTTTA |
| | | CAGCCCAAGCCGCTCATTTTCTTACTTTTATCCTTTTAG |
| | | GTTGCCTATAAAGGGGGTCCCCATCGAATTACAAGTGG |
| | | AATGCTTCACATGGGACCAAAAACTCTGGTGCCGTCAC |
| | | TTCTGTGTGCTTGCGGACTCAGAATCTGGTGGACATAT |
| | | CACTCACTCTGGGATGGTGGGCATGGGAGTCAGCTGCA |
| | | CAGTCACCCGGGAAGATGGAACCAATCGCAGATAGGG |
| | | CTGCTAGTGAACCAATCACATGATGTCACCCAGACATC |
| | | AGGCATACCCACTAGTGTGAAATAGACATCAGAATTA |
| | | AGAAAAACGTAGGGTCCAAGTGGTTCCCCGTTATGGAC |
| | | TCGCTATCTGTCAACCAGATCTTATACCCTGAAGTTCA |
| | | CCTAGATAGCCCGATAGTTACCAATAAGATAGTAGCCA |
| | | TCCTGGAGTATGCTCGAGTCCCTCACGCTTACAGCCTG |
| | | GAGGACCCTACACTGTGTCAGAACATCAAGCACCGCCT |
| | | AAAAAACGGATTTTCCAACCAAATGATTATAAACAATG |
| | | TGGAAGTTGGGAATGTCATCAAGTCCAAGCTTAGGAGT |
| | | TATCCGGCCCACTCTCATATTCCATATCCAAATTGTAAT |
| | | CAGGATTTATTTAACATAGAAGACAAAGAGTCAACGA |
| | | GGAAGATCCGTGAACTCCTCAAAAAGGGGAATTCGCT |
| | | GTACTCCAAAGTCAGTGATAAGGTTTTCCAATGCTTAA |
| | | GGGACACTAACTCACGGCTTGGCCTAGGCTCCGAATTG |
| | | AGGGAGGACATCAAGGAGAAAGTTATTAACTTGGGAG |
| | | TTTACATGCACAGCTCCCAGTGGTTTGAGCCCTTTCTGT |
| | | TTTGGTTTACAGTCAAGACTGAGATGAGGTCAGTGATT |
| | | AAATCACAAACCCATACTTGCCATAGGAGGAGACACA |
| | | CACCTGTATTCTTCACTGGTAGTTCAGTTGAGTTGCTAA |
| | | TCTCTCGTGACCTTGTTGCTATAATCAGTAAAGAGTCTC |
| | | AACATGTATATTACCTGACATTTGAACTGGTTTTGATGT |
| | | ATTGTGATGTCATAGAGGGGAGGTTAATGACAGAGAC |
| | | CGCTATGACTATTGATGCTAGGTATACAGAGCTTCTAG |
| | | GAAGAGTCAGATACATGTGGAAACTGATAGATGGTTTC |
| | | TTCCCTGCACTCGGGAATCCAACTTATCAAATTGTAGC |
| | | CATGCTGGAGCCTCTTTCACTTGCTTACCTGCAGCTGA |
| | | GGGATATAACAGTAGAACTCAGAGGTGCTTTCCTTAAC |
| | | CACTGCTTTACTGAAATACATGATGTTCTTGACCAAAA |
| | | CGGGTTTTCTGATGAAGGTACTTATCATGAGTTAACTG |
| | | AAGCTCTAGATTACATTTTCATAACTGATGACATACAT |
| | | CTGACAGGGGAGATTTTCTCATTTTTCAGAAGTTTCGG |
| | | CCACCCCAGACTTGAAGCAGTAACGGCTGCTGAAAAT |
| | | GTTAGGAAATACATGAATCAGCCTAAAGTCATTGTGTA |
| | | TGAGACTCTGATGAAAGGTCATGCCATATTTTGTGGAA |
| | | TCATAATCAACGGCTATCGTGACAGGCACGGAGGCAG |
| | | TTGGCCACCGCTGACCCTCCCCCTGCATGCTGCAGACA |
| | | CAATCCGGAATGCTCAAGCTTCAGGTGAAGGGTTAACA |
| | | CATGAGCAGTGCGTTGATAACTGGAAATCTTTTGCTGG |
| | | AGTGAAATTTGGCTGCTTTATGCCTCTTAGCCTGGATA |
| | | GTGATCTGACAATGTACCTAAAGGACAAGGCACTTGCT |
| | | GCTCTCCAAAGGGAATGGGATTCAGTTTACCCGAAAGA |
| | | GTTCCTGCGTTACGACCCTCCCAAGGGAACCGGGTCAC |
| | | GGAGGCTTGTAGATGTTTTCCTTAATGATTCGAGCTTTG |
| | | ACCCATATGATGTGATAATGTATGTTGTAAGTGGAGCT |
| | | TACCTCCATGACCCTGAGTTCAACCTGTCTTACAGCCT |
| | | GAAAGAAAAGGAGATCAAGGAAACAGGTAGACTTTTT |
| | | GCTAAAATGACTTACAAAATGAGGGCATGCCAAGTGA |
| | | TTGCTGAAAATCTAATCTCAAACGGGATTGGCAAATAT |
| | | TTTAAGGACAATGGGATGGCCAAGGATGAGCACGATT |
| | | TGACTAAGGCACTCCACACTCTAGCTGTCTCAGGAGTC |
| | | CCCAAAGATCTCAAAGAAAGTCACAGGGGGGGGCCAG |
| | | TCTTAAAAACCTACTCCCGAAGCCCAGTCCACACAAGT |
| | | ACCAGGAACGTGAGAGCAGCAAAAGGGTTTATAGGGT |
| | | TCCCTCAAGTAATTCGGCAGGACCAAGACACTGATCAT |
| | | CCGGAGAATATGGAAGCTTACGAGACAGTCAGTGCAT |
| | | TTATCACGACTGATCTCAAGAAGTACTGCCTTAATTGG |
| | | AGATATGAGACCATCAGCTTGTTTGCACAGAGGCTAAA |
| | | TGAGATTTACGGATTGCCCTCATTTTTCCAGTGGCTGCA |
| | | TAAGAGGCTTGAGACCTCTGTCCTGTATGTAAGTGACC |
| | | CTCATTGCCCCCCCGACCTTGACGCCCATATCCCGTTAT |
| | | ATAAAGTCCCCAATGATCAAATCTTCATTAAGTACCCT |
| | | ATGGGAGGTATAGAAGGGTATTGTCAGAAGCTGTGGA |
| | | CCATCAGCACCATTCCCTATCTATACCTGGCTGCTTATG |
| | | AGAGCGGAGTAAGGATTGCTTCGTTAGTGCAAGGGGA |
| | | CAATCAGACCATAGCCGTAACAAAAAGGGTACCCAGC |
| | | ACATGGCCCTACAACCTTAAGAAACGGGAAGCTGCTA |
| | | GAGTAACTAGAGATTACTTTGTAATTCTTAGGCAAAGG |
| | | CTACATGATATTGGCCATCACCTCAAGGCAAATGAGAC |
| | | AATTGTTTCATCACATTTTTTTGTCTATTCAAAAGGAAT |
| | | ATATTATGATGGGCTACTTGTGTCCCAATCACTCAAGA |
| | | GCATCGCAAGATGTGTATTCTGGTCAGAGACTATAGTT |
| | | GATGAAACAAGGGCAGCATGCAGTAATATTGCTACAA |
| | | CAATGGCTAAAAGCATCGAGAGAGGTTATGACCGTTA |
| | | CCTTGCATATTCCCTGAACGTCCTAAAAGTGATACAGC |
| | | AAATTCTGATCTCTCTTGGCTTCACAATCAATTCAACCA |
| | | TGACCCGGGATGTAGTCATACCCCTCCTCACAAACAAC |
| | | GACCTCTTAATAAGGATGGCACTGTTGCCCGCTCCTAT |
| | | TGGGGGGATGAATTATCTGAATATGAGCAGGCTGTTTG |
| | | TCAGAAACATCGGTGATCCAGTAACATCATCAATTGCT |
| | | GATCTCAAGAGAATGATTCTCGCCTCACTAATGCCTGA |
| | | AGAGACCCTCCATCAAGTAATGACACAACAACCGGGG |
| | | GACTCTTCATTCCTAGACTGGGCTAGCGACCCTTACTC |
| | | AGCAAATCTTGTATGTGTCCAGAGCATCACTAGACTCC |
| | | TCAAGAACATAACTGCAAGGTTTGTCCTGATCCATAGT |
| | | CCAAACCCAATGTTAAAAGGATTATTCCATGATGACAG |
| | | TAAAGAAGAGGACGAGGGACTGGCGGCATTCCTCATG |
| | | GACAGGCATATTATAGTACCTAGGGCAGCTCATGAAAT |
| | | CCTGGATCATAGTGTCACAGGGGCAAGAGAGTCTATTG |
| | | CAGGCATGCTGGATACCACAAAAGGCTTGATTCGAGCC |
| | | AGCATGAGGAAGGGGGGGTTAACCTCTCGAGTGATAA |
| | | CCAGATTGTCCAATTATGACTATGAACAATTCAGAGCA |
| | | GGGATGGTGCTATTGACAGGAAGAAAGAGAAATGTCC |
| | | TCATTGACAAAGAGTCATGTTCAGTGCAGCTGGCGAGA |
| | | GCTCTAAGAAGCCATATGTGGGCGAGGCTAGCTCGAG |
| | | GACGGCCTATTTACGGCCTTGAGGTCCCTGATGTACTA |
| | | GAATCTATGCGAGGCCACCTTATTCGGCGTCATGAGAC |
| | | ATGTGTCATCTGCGAGTGTGGATCAGTCAACTACGGAT |
| | | GGTTTTTTGTCCCCTCGGGTTGCCAACTGGATGATATTG |
| | | ACAAGGAAACATCATCCTTGAGAGTCCCATATATTGGT |
| | | TCTACCACTGATGAGAGAACAGACATGAAGCTTGCCTT |
| | | CGTAAGAGCCCCAAGTCGATCCTTGCGATCTGCTGTTA |
| | | GAATAGCAACAGTGTACTCATGGGCTTACGGTGATGAT |
| | | GATAGCTCTTGGAACGAAGCCTGGTTGTTGGCTAGGCA |
| | | AAGGGCCAATGTGAGCCTGGAGGAGCTAAGGGTGATC |
| | | ACTCCCATCTCAACTTCGACTAATTTAGCGCATAGGTT |
| | | GAGGGATCGTAGCACTCAAGTGAAATACTCAGGTACA |
| | | TCCCTTGTCCGAGTGGCGAGGTATACCACAATCTCCAA |
| | | CGACAATCTCTCATTTGTCATATCAGATAAGAAGGTTG |
| | | ATACTAACTTTATATACCAACAAGGAATGCTTCTAGGG |
| | | TTGGGTGTTTTAGAAACATTGTTTCGACTCGAGAAAGA |
| | | TACCGGATCATCTAACACGGTATTACATCTTCACGTCG |
| | | AAACAGATTGTTGCGTGATCCCGATGATAGATCATCCC |
| | | AGGATACCCAGCTCCCGCAAGCTAGAGCTGAGGGCAG |
| | | AGCTATGTACCAACCCATTGATATATGATAATGCACCT |
| | | TTAATTGACAGAGATGCAACAAGGCTATACACCCAGA |
| | | GCCATAGGAGGCACCTTGTGGAATTTGTTACATGGTCC |
| | | ACACCCCAACTATATCACATTTTAGCTAAGTCCACAGC |
| | | ACTATCTATGATTGACCTGGTAACAAAATTTGAGAAGG |
| | | ACCATATGAATGAAATTTCAGCTCTCATAGGGGATGAC |
| | | GATATCAATAGTTTCATAACTGAGTTTCTGCTCATAGA |
| | | GCCAAGATTATTCACTATCTACTTGGGCCAGTGTGCGG |
| | | CCATCAATTGGGCATTTGATGTACATTATCATAGACCA |
| | | TCAGGGAAATATCAGATGGGTGAGCTGTTGTCATCGTT |
| | | CCTTTCTAGAATGAGCAAAGGAGTGTTTAAGGTGCTTG |
| | | TCAATGCTCTAAGCCACCCAAAGATCTACAAGAAATTC |
| | | TGGCATTGTGGTATTATAGAGCCTATCCATGGTCCTTC |
| | | ACTTGATGCTCAAAACTTGCACACAACTGTGTGCAACA |
| | | TGGTTTACACATGCTATATGACCTACCTCGACCTGTTGT |
| | | TGAATGAAGAGTTAGAAGAGTTCACATTTCTCTTGTGT |
| | | GAAAGCGACGAGGATGTAGTACCGGACAGATTCGACA |
| | | ACATCCAGGCAAAACACTTATGTGTTCTGGCAGATTTG |
| | | TACTGTCAACCAGGGACCTGCCCACCAATTCGAGGTCT |
| | | AAGACCGGTAGAGAAATGTGCAGTTCTAACCGACCAT |
| | | ATCAAGGCAGAGGCTATGTTATCTCCAGCAGGATCTTC |
| | | GTGGAACATAAATCCAATTATTGTAGACCATTACTCAT |
| | | GCTCTCTGACTTATCTCCGGCGAGGATCGATCAAACAG |
| | | ATAAGATTGAGAGTTGATCCAGGATTCATTTTCGACGC |
| | | CCTCGCTGAGGTAAATGTCAGTCAGCCAAAGATCGGCA |
| | | GCAACAACATCTCAAATATGAGCATCAAGGCTTTCAGA |
| | | CCCCCACACGATGATGTTGCAAAATTGCTCAAAGATAT |
| | | CAACACAAGCAAGCACAATCTTCCCATTTCAGGGGGCA |
| | | ATCTCGCCAATTATGAAATCCATGCTTTCCGCAGAATC |
| | | GGGTTGAACTCATCTGCTTGCTACAAAGCTGTTGAGAT |
| | | ATCAACATTAATTAGGAGATGCCTTGAGCCAGGGGAG |
| | | GACGGCTTGTTCTTGGGTGAGGGATCGGGTTCTATGTT |
| | | GATCACTTATAAAGAGATACTTAAACTAAACAAGTGCT |
| | | TCTATAATAGTGGGGTTTCCGCCAATTCTAGATCTGGT |
| | | CAAAGGGAATTAGCACCCTATCCCTCCGAAGTTGGCCT |
| | | TGTCGAACACAGAATGGGAGTAGGTAATATTGTCAAA |
| | | GTGCTCTTTAACGGGAGGCCCGAAGTCACGTGGGTAGG |
| | | CAGTGTAGATTGCTTCAATTTCATAGTTAGTAATATCCC |
| | | TACCTCTAGTGTGGGGTTTATCCATTCAGATATAGAGA |
| | | CCTTGCCTGACAAAGATACTATAGAGAAGCTAGAGGA |
| | | ATTGGCAGCCATCTTATCGATGGCTCTGCTCCTGGGCA |
| | | AAATAGGATCAATACTGGTGATTAAGCTTATGCCTTTC |
| | | AGCGGGGATTTTGTTCAGGGATTTATAAGTTATGTAGG |
| | | GTCTCATTATAGAGAAGTGAACCTTGTATACCCTAGAT |
| | | ACAGCAACTTCATCTCTACTGAATCTTATTTGGTTATGA |
| | | CAGATCTCAAGGCTAACCGGCTAATGAATCCTGAAAA |
| | | GATTAAGCAGCAGATAATTGAATCATCTGTGAGGACTT |
| | | CACCTGGACTTATAGGTCACATCCTATCCATTAAGCAA |
| | | CTAAGCTGCATACAAGCAATTGTGGGAGACGCAGTTA |
| | | GTAGAGGTGATATCAATCCTACTCTGAAAAAACTTACA |
| | | CCTATAGAGCAGGTGCTGATCAATTGCGGGTTGGCAAT |
| | | TAACGGACCTAAGCTGTGCAAAGAATTGATCCACCATG |
| | | ATGTTGCCTCAGGGCAAGATGGATTGCTTAATTCTATA |
| | | CTCATCCTCTACAGGGAGTTGGCAAGATTCAAAGACAA |
| | | CCAAAGAAGTCAACAAGGGATGTTCCACGCTTACCCCG |
| | | TATTGGTAAGTAGCAGGCAACGAGAACTTATATCTAGG |
| | | ATCACCCGCAAATTCTGGGGGCACATTCTTCTTTACTCC |
| | | GGGAACAAAAAGTTGATAAATAAGTTTATCCAGAATCT |
| | | CAAGTCCGGCTATCTGATACTAGACTTACACCAGAATA |
| | | TCTTCGTTAAGAATCTATCCAAGTCAGAGAAACAGATT |
| | | ATTATGACGGGGGGTTTGAAACGTGAGTGGGTTTTTAA |
| | | GGTAACAGTCAAGGAGACCAAAGAATGGTATAAGTTA |
| | | GTCGGATACAGTGCCCTGATTAAGGACTAATTGGTTGA |
| | | ACTCCGGAACCCTAATCCTGCCCTAGGTGGTTAGGCAT |
| | | TATTTGCAATATATTAAAGAAAACTTTGAAAATACGAA |
| | | GTTTCTATTCCCAGCTTTGTCTGGT |
| 83 | T-05 | ACCAAACAAAGTTGGGTAAGGATAGTTCAATCAATGA |
| | EBV_gH ATUa_ | TCATCTTCTAGTGCACTTAGGATTCAAGATCCTATTATC |
| | gL_ATUb_gp42 | AGGGACAAGAGCAGGATTAGGGATATCCGAGATGGCC |
| | at ATU2 | ACACTTTTAAGGAGCTTAGCATTGTTCAAAAGAAACAA |
| | MV leader: nt | GGACAAACCACCCATTACATCAGGATCCGGTGGAGCC |
| | 1-55 | ATCAGAGGAATCAAACACATTATTATAGTACCAATCCC |
| | MV N ORF: nt | TGGAGATTCCTCAATTACCACTCGATCCAGACTTCTGG |
| | 108-1,685 | ACCGGTTGGTGAGGTTAATTGGAAACCCGGATGTGAGC |
| | MV P ORF: nt | GGGCCCAAACTAACAGGGGCACTAATAGGTATATTATC |
| | 1,807-3,330 | CTTATTTGTGGAGTCTCCAGGTCAATTGATTCAGAGGA |
| | ATU upstream | TCACCGATGACCCTGACGTTAGCATAAGGCTGTTAGAG |
| | motif: nt 3,378- | GTTGTCCAGAGTGACCAGTCACAATCTGGCCTTACCTT |
| | 3,455 | CGCATCAAGAGGTACCAACATGGAGGATGAGGCGGAC |
| | GE of N gene: | CAATACTTTTCACATGATGATCCAATTAGTAGTGATCA |
| | nt 3,394-3,404 | ATCCAGGTTCGGATGGTTCGGGAACAAGGAAATCTCA |
| | Conserved | GATATTGAAGTGCAAGACCCTGAGGGATTCAACATGAT |
| | trinucleotide | TCTGGGTACCATCCTAGCCCAAATTTGGGTCTTGCTCG |
| | motif: nt 3,405- | CAAAGGCGGTTACGGCCCCAGACACGGCAGCTGATTC |
| | 3,407 | GGAGCTAAGAAGGTGGATAAAGTACACCCAACAAAGA |
| | GS of P gene: | AGGGTAGTTGGTGAATTTAGATTGGAGAGAAAATGGTT |
| | nt 3,408-3,424 | GGATGTGGTGAGGAACAGGATTGCCGAGGACCTCTCCT |
| | Kozak | TACGCCGATTCATGGTCGCTCTAATCCTGGATATCAAG |
| | sequence: nt | AGAACACCCGGAAACAAACCCAGGATTGCTGAAATGA |
| | 3,450-3,458 | TATGTGACATTGATACATATATCGTAGAGGCAGGATTA |
| | EBV gH ORF: | GCCAGTTTTATCCTGACTATTAAGTTTGGGATAGAAAC |
| | nt 3,456-5,579 | TATGTATCCTGCTCTTGGACTGCATGAATTTGCTGGTGA |
| | ATUa motif: nt | GTTATCCACACTTGAGTCCTTGATGAACCTTTACCAGC |
| | 5,580-5,723 | AAATGGGGGAAACTGCACCCTACATGGTAATCCTGGA |
| | GE of P gene: | GAACTCAATTCAGAACAAGTTCAGTGCAGGATCATACC |
| | nt 5,647-5,657 | CTCTGCTCTGGAGCTATGCCATGGGAGTAGGAGTGGAA |
| | Conserved | CTTGAAAACTCCATGGGAGGTTTGAACTTTGGCCGATC |
| | trinucleotide | TTACTTTGATCCAGCATATTTTAGATTAGGGCAAGAGA |
| | motif: nt 5,658- | TGGTAAGGAGGTCAGCTGGAAAGGTCAGTTCCACATTG |
| | 5,660 | GCATCTGAACTCGGTATCACTGCCGAGGATGCAAGGCT |
| | GS of P gene: | TGTTTCAGAGATTGCAATGCATACTACTGAGGACAAGA |
| | nt 5,661-5,677 | TCAGTAGAGCGGTTGGACCCAGACAAGCCCAAGTATC |
| | EBV gp42 | ATTTCTACACGGTGATCAAAGTGAGAATGAGCTACCGA |
| | ORF: nt 5,724- | GATTGGGGGGCAAGGAAGATAGGAGGGTCAAACAGAG |
| | 6,395 | TCGAGGAGAAGCCAGGGAGAGCTACAGAGAAACCGGG |
| | ATUb motif: nt | CCCAGCAGAGCAAGTGATGCGAGAGCTGCCCATCTTCC |
| | 6,396-6,497 | AACCGGCACACCCCTAGACATTGACACTGCAACGGAG |
| | GE of N gene: | TCCAGCCAAGATCCGCAGGACAGTCGAAGGTCAGCTG |
| | nt 6,447-6,457 | ACGCCCTGCTTAGGCTGCAAGCCATGGCAGGAATCTCG |
| | Conserved | GAAGAACAAGGCTCAGACACGGACACCCCTATAGTGT |
| | trinucleotide | ACAATGACAGAAATCTTCTAGACTAGGTGCGAGAGGC |
| | motif: nt 6,458- | CGAGGGCCAGAACAACATCCGCCTACCATCCATCATTG |
| | 6,460 | TTATAAAAAACTTAGGAACCAGGTCCACACAGCCGCC |
| | Hybrid GS | AGCCCATCAACCATCCACTCCCACGATTGGAGCCAATG |
| | (combination of | GCAGAAGAGCAGGCACGCCATGTCAAAAACGGACTGG |
| | GS of P gene | AATGCATCCGGGCTCTCAAGGCCGAGCCCATCGGCTCA |
| | and GS of M | CTGGCCATCGAGGAAGCTATGGCAGCATGGTCAGAAA |
| | gene): nt 6,461- | TATCAGACAACCCAGGACAGGAGCGAGCCACCTGCAG |
| | 6,477 | GGAAGAGAAGGCAGGCAGTTCGGGTCTCAGCAAACCA |
| | EBV gL ORF: | TGCCTCTCAGCAATTGGATCAACTGAAGGCGGTGCACC |
| | nt 6,498-6,911 | TCGCATCCGCGGTCAGGGACCTGGAGAGAGCGATGAC |
| | ATU | GACGCTGAAACTTTGGGAATCCCCCCAAGAAATCTCCA |
| | downstream | GGCATCAAGCACTGGGTTACAGTGTTATTACGTTTATG |
| | motif: nt 6,912- | ATCACAGCGGTGAAGCGGTTAAGGGAATCCAAGATGC |
| | 6,947 | TGACTCTATCATGGTTCAATCAGGCCTTGATGGTGATA |
| | MV M ORF: nt | GCACCCTCTCAGGAGGAGACAATGAATCTGAAAACAG |
| | 7,008-8,015 | CGATGTGGATATTGGCGAACCTGATACCGAGGGATATG |
| | MV F ORF: nt | CTATCACTGACCGGGGATCTGCTCCCATCTCTATGGGG |
| | 9,019-10,680 | TTCAGGGCTTCTGATGTTGAAACTGCAGAAGGAGGGG |
| | MV H ORF: nt | AGATCCACGAGCTCCTGAGACTCCAATCCAGAGGCAA |
| | 10,841-12,694 | CAACTTTCCGAAGCTTGGGAAAACTCTCAATGTTCCTC |
| | MV L ORF: nt | CGCCCCCGGACCCCGGTAGGGCCAGCACTTCCGGGAC |
| | 12,804-19,355 | ACCCATTAAAAAGGGCACAGACGCGAGATTAGCCTCA |
| | MV trailer: nt | TTTGGAACGGAGATCGCGTCTTTATTGACAGGTGGTGC |
| | 19,428-19,464 | AACCCAATGTGCTCGAAAGTCACCCTCGGAACCATCAG |
| | | GGCCAGGTGCACCTGCGGGGAATGTCCCCGAGTGTGTG |
| | | AGCAATGCCGCACTGATACAGGAGTGGACACCCGAAT |
| | | CTGGTACCACAATCTCCCCGAGATCCCAGAATAATGAA |
| | | GAAGGGGGAGACTATTATGATGATGAGCTGTTCTCTGA |
| | | TGTCCAAGATATTAAAACAGCCTTGGCCAAAATACACG |
| | | AGGATAATCAGAAGATAATCTCCAAGCTAGAATCACT |
| | | GCTGTTATTGAAGGGAGAAGTTGAGTCAATTAAGAAG |
| | | CAGATCAACAGGCAAAATATCAGCATATCCACCCTGG |
| | | AAGGACACCTCTCAAGCATCATGATCGCCATTCCTGGA |
| | | CTTGGGAAGGATCCCAACGACCCCACTGCAGATGTCGA |
| | | AATCAATCCCGACTTGAAACCCATCATAGGCAGAGATT |
| | | CAGGCCGAGCACTGGCCGAAGTTCTCAAGAAACCCGTT |
| | | GCCAGCCGACAACTCCAAGGAATGACAAATGGACGGA |
| | | CCAGTTCCAGAGGACAGCTGCTGAAGGAATTTCAGCTA |
| | | AAGCCGATCGGGAAAAAGATGAGCTCAGCCGTCGGGT |
| | | TTGTTCCTGACACCGGCCCTGCATCACGCAGTGTAATC |
| | | CGCTCCATTATAAAATCCAGCCGGCTAGAGGAGGATCG |
| | | GAAGCGTTACCTGATGACTCTCCTTGATGATATCAAAG |
| | | GAGCCAATGATCTTGCCAAGTTCCACCAGATGCTGATG |
| | | AAGATAATAATGAAGTAGCTACAGCTCAACTTACCTGC |
| | | CAACCCCATGCCAGTCGACCCAACTAGCCTACCCTCCA |
| | | TCATTGTTATAAAAAACTTAGGAACCAGGTCCACACAG |
| | | CCGCCAGCCCATCAACGCGTACGGCCACCATGCAACTC |
| | | CTCTGCGTCTTTTGCTTGGTGCTTTTGTGGGAGGTAGGA |
| | | GCGGCCTCATTGTCAGAAGTTAAGCTGCATCTCGATAT |
| | | TGAAGGCCACGCTTCACATTACACTATCCCTTGGACCG |
| | | AACTCATGGCTAAGGTGCCGGGTTTGTCTCCGGAAGCA |
| | | CTTTGGAGAGAGGCAAACGTTACCGAAGACCTGGCCTC |
| | | AATGCTGAATCGCTACAAGCTGATATACAAGACTTCCG |
| | | GAACCCTGGGTATAGCACTCGCAGAGCCCGTCGATATC |
| | | CCCGCTGTCAGCGAAGGTAGCATGCAGGTGGACGCTA |
| | | GTAAAGTTCACCCAGGAGTTATCTCAGGTCTGAATAGC |
| | | CCAGCTTGTATGCTGAGCGCTCCACTGGAGAAACAGCT |
| | | CTTCTACTATATAGGCACAATGCTCCCTAACACTCGAC |
| | | CTCACAGCTACGTCTTCTATCAGCTGCGCTGTCACCTGT |
| | | CATACGTAGCCTTGTCCATTAATGGTGATAAGTTCCAG |
| | | TACACAGGTGCCATGACCTCCAAGTTCCTTATGGGGAC |
| | | CTATAAACGGGTCACTGAGAAAGGAGACGAGCATGTC |
| | | CTTAGTTTGGTTTTTGGGAAGACAAAAGACCTGCCGGA |
| | | TCTGAGGGGCCCGTTCTCTTACCCCTCACTCACATCTGC |
| | | ACAGAGTGGGGACTATTCATTGGTGATTGTGACAACCT |
| | | TCGTTCACTACGCCAATTTTCACAACTATTTTGTCCCGA |
| | | ACCTTAAAGACATGTTTTCACGCGCCGTGACCATGACA |
| | | GCTGCTTCCTACGCTAGGTATGTGCTCCAAAAGCTGGT |
| | | GCTGCTGGAGATGAAGGGCGGATGCAGGGAACCTGAA |
| | | CTGGACACAGAGACTCTCACCACTATGTTCGAGGTTTC |
| | | CGTGGCGTTCTTCAAGGTGGGTCATGCTGTGGGGGAGA |
| | | CTGGGAACGGCTGCGTAGATCTTAGGTGGCTGGCTAAA |
| | | TCTTTCTTCGAGCTGACCGTGTTGAAAGATATAATTGG |
| | | AATTTGCTACGGGGCTACGGTTAAAGGAATGCAGTCTT |
| | | ACGGGCTGGAAAGGCTGGCCGCTATGCTCATGGCTACT |
| | | GTGAAGATGGAGGAACTCGGCCACCTCACCACAGAAA |
| | | AACAGGAGTACGCTTTGCGCCTGGCCACTGTTGGCTAC |
| | | CCTAAGGCAGGCGTTTACTCTGGATTGATTGGGGGGGC |
| | | CACATCTGTGCTCTTGTCAGCCTACAATCGCCATCCCCT |
| | | GTTCCAGCCTCTGCACACGGTGATGCGAGAGACTTTGT |
| | | TTATTGGCAGCCACGTTGTGCTGAGAGAACTGCGCCTT |
| | | AACGTTACCACTCAGGGACCTAACCTTGCGCTGTATCA |
| | | GCTGCTTAGCACCGCTTTGTGCTCCGCTCTTGAAATTGG |
| | | AGAAGTACTCAGAGGGCTCGCACTGGGTACTGAGTCA |
| | | GGGCTGTTCTCCCCCTGCTATCTGTCTCTCCGGTTCGAC |
| | | CTCACTCGGGATAAGCTGTTGTCAATGGCTCCGCAGGA |
| | | AGCCACATTGGACCAGGCCGCAGTGAGTAACGCAGTG |
| | | GACGGGTTCCTGGGTAGACTGTCTCTTGAGAGGGAAGA |
| | | CAGAGACGCTTGGCATCTGCCTGCGTATAAGTGTGTTG |
| | | ATAGACTGGACAAAGTACTGATGATCATTCCCCTGATT |
| | | AATGTGACATTTATTATATCTAGTGACCGAGAGGTGAG |
| | | AGGCTCCGCTCTCTATGAAGCATCCACAACGTACTTGA |
| | | GCAGTTCCCTGTTTCTGTCACCGGTTATTATGAACAAAT |
| | | GTTCTCAGGGAGCTGTTGCTGGTGAACCGCGACAGATT |
| | | CCAAAGATTCAGAACTTCACTAGGACACAAAAGAGCT |
| | | GCATTTTCTGCGGGTTCGCACTGCTGAGTTACGACGAA |
| | | AAGGAGGGACTGGAGACTACTACATATATTACTTCACA |
| | | GGAGGTGCAGAATAGCATCCTGAGCTCCAATTATTTTG |
| | | ACTTTGACAACCTCCATGTCCATTATTTGCTGTTGACCA |
| | | CAAACGGCACTGTGATGGAGATCGCTGGCCTGTACGA |
| | | GGAGCGAGCCCACGTAGTACTGGCCATTATTCTGTACT |
| | | TCATCGCTTTCGCACTTGGGATTTTCCTCGTCCACAAAA |
| | | TTGTTATGTTTTTCCTCTAATGATTCGAACTACAGCTCA |
| | | ACTTACCTGCCAACCCCATGCCAGTCGACCCAACTAGT |
| | | ACAACCTAAATCCATTATAAAAAACTTAGGAACCAGGT |
| | | CCACACAGCCGCCAGCCCATCAACCATCCACTCCCACG |
| | | ATTGGAGGCCGGCCATGGTGAGTTTTAAGCAGGTCCGG |
| | | GTACCTCTGTTCACCGCCATTGCGCTGGTGATTGTTCTG |
| | | CTGTTGGCCTACTTTCTTCCACCTAGAGTCAGAGGAGG |
| | | TGGCAGGGTTGCTGCAGCTGCTATTACCTGGGTACCGA |
| | | AACCCAACGTGGAGGTTTGGCCAGTGGATCCGCCCCCA |
| | | CCAGTGAACTTTAATAAAACAGCCGAACAAGAATACG |
| | | GGGATAAAGAAGTAAAGCTGCCACACTGGACCCCTAC |
| | | CCTCCACACTTTCCAGGTGCCTCAAAACTATACAAAGG |
| | | CCAATTGCACATACTGTAACACAAGAGAATACACTTTT |
| | | TCTTATAAAGGGTGCTGCTTCTACTTCACTAAGAAGAA |
| | | GCATACTTGGAACGGATGTTTCCAGGCATGCGCTGAGT |
| | | TGTATCCCTGTACCTATTTCTACGGTCCTACACCAGACA |
| | | TTCTGCCGGTCGTGACTCGCAATCTGAACGCCATAGAA |
| | | TCTCTGTGGGTCGGGGTGTATCGGGTTGGGGAAGGAAA |
| | | TTGGACTAGCCTGGACGGCGGTACTTTCAAGGTGTACC |
| | | AAATATTCGGCAGTCACTGTACTTACGTGTCAAAATTC |
| | | TCAACAGTTCCCGTATCCCACCACGAGTGTTCTTTTTTG |
| | | AAGCCTTGCCTGTGCGTGTCACAGAGGTCAAATTCCTA |
| | | ACTATTCGAACTACTCAGCTAGTCTACGCGCCGTAGCC |
| | | TACCCTCCATCATTGTTATAAAAAACTTAGGAGCAAAG |
| | | TCCACACAGCCGCCGATAAGGTCCACAATGAGAGCCG |
| | | TAGGTGTCTTTCTGGCTATTTGTCTGGTAACAATCTTCG |
| | | TGCTGCCCACTTGGGGCAACTGGGCCTATCCTTGCTGC |
| | | CATGTTACACAGTTGAGAGCGCAGCACCTCCTGGCGTT |
| | | GGAGAACATTTCAGACATCTACCTGGTGAGTAACCAAA |
| | | CCTGCGACGGGTTCTCTTTGGCCAGTCTCAATAGTCCA |
| | | AAAAACGGAAGCAATCAGCTGGTAATTTCACGGTGTG |
| | | CAAACGGACTTAACGTGGTCTCTTTTTTTATATCAATCC |
| | | TGAAACGCAGTTCATCTGCATTGACCGGCCACTTGAGG |
| | | GAGCTCTTGACCACTCTCGAGACACTTTACGGGTCATT |
| | | CTCCGTGGAGGATCTGTTCGGGGCAAATCTGAACCGGT |
| | | ACGCCTGGCATAGAGGCGGGTAAGCGCGCAGCGCTTA |
| | | GACGTCTCGCGATCGATACTAGTACAACCTAAATCCAT |
| | | TATAAAAAACTTAGGAGCAAAGTGATTGCCTCCCAAG |
| | | GTCCACAATGACAGAGACCTACGACTTCGACAAGTCG |
| | | GCATGGGACATCAAAGGGTCGATCGCTCCGATACAAC |
| | | CCACCACCTACAGTGATGGCAGGCTGGTGCCCCAGGTC |
| | | AGAGTCATAGATCCTGGTCTAGGCGACAGGAAGGATG |
| | | AATGCTTTATGTACATGTTTCTGCTGGGGGTTGTTGAG |
| | | GACAGCGATTCCCTAGGGCCTCCAATCGGGCGAGCATT |
| | | TGGGTTCCTGCCCTTAGGTGTTGGCAGATCCACAGCAA |
| | | AGCCCGAAAAACTCCTCAAAGAGGCCACTGAGCTTGA |
| | | CATAGTTGTTAGACGTACAGCAGGGCTCAATGAAAAA |
| | | CTGGTGTTCTACAACAACACCCCACTAACTCTCCTCAC |
| | | ACCTTGGAGAAAGGTCCTAACAACAGGGAGTGTCTTCA |
| | | ACGCAAACCAAGTGTGCAATGCGGTTAATCTGATACCG |
| | | CTCGATACCCCGCAGAGGTTCCGTGTTGTTTATATGAG |
| | | CATCACCCGTCTTTCGGATAACGGGTATTACACCGTTC |
| | | CTAGAAGAATGCTGGAATTCAGATCGGTCAATGCAGTG |
| | | GCCTTCAACCTGCTGGTGACCCTTAGGATTGACAAGGC |
| | | GATAGGCCCTGGGAAGATCATCGACAATACAGAGCAA |
| | | CTTCCTGAGGCAACATTTATGGTCCACATCGGGAACTT |
| | | CAGGAGAAAGAAGAGTGAAGTCTACTCTGCCGATTATT |
| | | GCAAAATGAAAATCGAAAAGATGGGCCTGGTTTTTGC |
| | | ACTTGGTGGGATAGGGGGCACCAGTCTTCACATTAGAA |
| | | GCACAGGCAAAATGAGCAAGACTCTCCATGCACAACT |
| | | CGGGTTCAAGAAGACCTTATGTTACCCGCTGATGGATA |
| | | TCAATGAAGACCTTAATCGATTACTCTGGAGGAGCAGA |
| | | TGCAAGATAGTAAGAATCCAGGCAGTTTTGCAGCCATC |
| | | AGTTCCTCAAGAATTCCGCATTTACGACGACGTGATCA |
| | | TAAATGATGACCAAGGACTATTCAAAGTTCTGTAGACC |
| | | GTAGTGCCCAGCAATGCCCGAAAACGACCCCCCTCACA |
| | | ATGACAGCCAGAAGGCCCGGACAAAAAAGCCCCCTCC |
| | | GAAAGACTCCACGGACCAAGCGAGAGGCCAGCCAGCA |
| | | GCCGACGGCAAGCGCGAACACCAGGCGGCCCCAGCAC |
| | | AGAACAGCCCTGACACAAGGCCACCACCAGCCACCCC |
| | | AATCTGCATCCTCCTCGTGGGACCCCCGAGGACCAACC |
| | | CCCAAGGCTGCCCCCGATCCAAACCACCAACCGCATCC |
| | | CCACCACCCCCGGGAAAGAAACCCCCAGCAATTGGAA |
| | | GGCCCCTCCCCCTCTTCCTCAACACAAGAACTCCACAA |
| | | CCGAACCGCACAAGCGACCGAGGTGACCCAACCGCAG |
| | | GCATCCGACTCCCTAGACAGATCCTCTCTCCCCGGCAA |
| | | ACTAAACAAAACTTAGGGCCAAGGAACATACACACCC |
| | | AACAGAACCCAGACCCCGGCCCACGGCGCCGCGCCCC |
| | | CAACCCCCGACAACCAGAGGGAGCCCCCAACCAATCC |
| | | CGCCGGCTCCCCCGGTGCCCACAGGCAGGGACACCAA |
| | | CCCCCGAACAGACCCAGCACCCAACCATCGACAATCC |
| | | AAGACGGGGGGGCCCCCCCAAAAAAAGGCCCCCAGGG |
| | | GCCGACAGCCAGCACCGCGAGGAAGCCCACCCACCCC |
| | | ACACACGACCACGGCAACCAAACCAGAACCCAGACCA |
| | | CCCTGGGCCACCAGCTCCCAGACTCGGCCATCACCCCG |
| | | CAGAAAGGAAAGGCCACAACCCGCGCACCCCAGCCCC |
| | | GATCCGGCGGGGAGCCACCCAACCCGAACCAGCACCC |
| | | AAGAGCGATCCCCGAAGGACCCCCGAACCGCAAAGGA |
| | | CATCAGTATCCCACAGCCTCTCCAAGTCCCCCGGTCTC |
| | | CTCCTCTTCTCGAAGGGACCAAAAGATCAATCCACCAC |
| | | ACCCGACGACACTCAACTCCCCACCCCTAAAGGAGAC |
| | | ACCGGGAATCCCAGAATCAAGACTCATCCAATGTCCAT |
| | | CATGGGTCTCAAGGTGAACGTCTCTGCCATATTCATGG |
| | | CAGTACTGTTAACTCTCCAAACACCCACCGGTCAAATC |
| | | CATTGGGGCAATCTCTCTAAGATAGGGGTGGTAGGAAT |
| | | AGGAAGTGCAAGCTACAAAGTTATGACTCGTTCCAGCC |
| | | ATCAATCATTAGTCATAAAATTAATGCCCAATATAACT |
| | | CTCCTCAATAACTGCACGAGGGTAGAGATTGCAGAATA |
| | | CAGGAGACTACTGAGAACAGTTTTGGAACCAATTAGA |
| | | GATGCACTTAATGCAATGACCCAGAATATAAGACCGGT |
| | | TCAGAGTGTAGCTTCAAGTAGGAGACACAAGAGATTT |
| | | GCGGGAGTAGTCCTGGCAGGTGCGGCCCTAGGCGTTGC |
| | | CACAGCTGCTCAGATAACAGCCGGCATTGCACTTCACC |
| | | AGTCCATGCTGAACTCTCAAGCCATCGACAATCTGAGA |
| | | GCGAGCCTGGAAACTACTAATCAGGCAATTGAGACAA |
| | | TCAGACAAGCAGGGCAGGAGATGATATTGGCTGTTCA |
| | | GGGTGTCCAAGACTACATCAATAATGAGCTGATACCGT |
| | | CTATGAACCAACTATCTTGTGATTTAATCGGCCAGAAG |
| | | CTCGGGCTCAAATTGCTCAGATACTATACAGAAATCCT |
| | | GTCATTATTTGGCCCCAGTTTACGGGACCCCATATCTG |
| | | CGGAGATATCTATCCAGGCTTTGAGCTATGCGCTTGGA |
| | | GGAGACATCAATAAGGTGTTAGAAAAGCTCGGATACA |
| | | GTGGAGGTGATTTACTGGGCATCTTAGAGAGCGGAGG |
| | | AATAAAGGCCCGGATAACTCACGTCGACACAGAGTCC |
| | | TACTTCATTGTCCTCAGTATAGCCTATCCGACGCTGTCC |
| | | GAGATTAAGGGGGTGATTGTCCACCGGCTAGAGGGGG |
| | | TCTCGTACAACATAGGCTCTCAAGAGTGGTATACCACT |
| | | GTGCCCAAGTATGTTGCAACCCAAGGGTACCTTATCTC |
| | | GAATTTTGATGAGTCATCGTGTACTTTCATGCCAGAGG |
| | | GGACTGTGTGCAGCCAAAATGCCTTGTACCCGATGAGT |
| | | CCTCTGCTCCAAGAATGCCTCCGGGGGTACACCAAGTC |
| | | CTGTGCTCGTACACTCGTATCCGGGTCTTTTGGGAACC |
| | | GGTTCATTTTATCACAAGGGAACCTAATAGCCAATTGT |
| | | GCATCAATCCTTTGCAAGTGTTACACAACAGGAACGAT |
| | | CATTAATCAAGACCCTGACAAGATCCTAACATACATTG |
| | | CTGCCGATCACTGCCCGGTAGTCGAGGTGAACGGCGTG |
| | | ACCATCCAAGTCGGGAGCAGGAGGTATCCAGACGCTG |
| | | TGTACTTGCACAGAATTGACCTCGGTCCTCCCATATCA |
| | | TTGGAGAGGTTGGACGTAGGGACAAATCTGGGGAATG |
| | | CAATTGCTAAGTTGGAGGATGCCAAGGAATTGTTGGAG |
| | | TCATCGGACCAGATATTGAGGAGTATGAAAGGTTTATC |
| | | GAGCACTAGCATAGTCTACATCCTGATTGCAGTGTGTC |
| | | TTGGAGGGTTGATAGGGATCCCCGCTTTAATATGTTGC |
| | | TGCAGGGGGCGTTGTAACAAAAAGGGAGAACAAGTTG |
| | | GTATGTCAAGACCAGGCCTAAAGCCTGATCTTACGGGA |
| | | ACATCAAAATCCTATGTAAGGTCGCTCTGATCCTCTAC |
| | | AACTCTTGAAACACAAATGTCCCACAAGTCTCCTCTTC |
| | | GTCATCAAGCAACCACCGCACCCAGCATCAAGCCCACC |
| | | TGAAATTATCTCCGGCTTCCCTCTGGCCGAACAATATC |
| | | GGTAGTTAATCAAAACTTAGGGTGCAAGATCATCCACA |
| | | ATGTCACCACAACGAGACCGGATAAATGCCTTCTACAA |
| | | AGATAACCCCCATCCCAAGGGAAGTAGGATAGTCATT |
| | | AACAGAGAACATCTTATGATTGATAGACCTTATGTTTT |
| | | GCTGGCTGTTCTGTTTGTCATGTTTCTGAGCTTGATCGG |
| | | GTTGCTAGCCATTGCAGGCATTAGACTTCATCGGGCAG |
| | | CCATCTACACCGCAGAGATCCATAAAAGCCTCAGCACC |
| | | AATCTAGATGTAACTAACTCAATCGAGCATCAGGTCAA |
| | | GGACGTGCTGACACCACTCTTCAAAATCATCGGTGATG |
| | | AAGTGGGCCTGAGGACACCTCAGAGATTCACTGACCTA |
| | | GTGAAATTAATCTCTGACAAGATTAAATTCCTTAATCC |
| | | GGATAGGGAGTACGACTTCAGAGATCTCACTTGGTGTA |
| | | TCAACCCGCCAGAGAGAATCAAATTGGATTATGATCAA |
| | | TACTGTGCAGATGTGGCTGCTGAAGAGCTCATGAATGC |
| | | ATTGGTGAACTCAACTCTACTGGAGACCAGAACAACCA |
| | | ATCAGTTCCTAGCTGTCTCAAAGGGAAACTGCTCAGGG |
| | | CCCACTACAATCAGAGGTCAATTCTCAAACATGTCGCT |
| | | GTCCCTGTTAGACTTGTATTTAGGTCGAGGTTACAATG |
| | | TGTCATCTATAGTCACTATGACATCCCAGGGAATGTAT |
| | | GGGGGAACTTACCTAGTGGAAAAGCCTAATCTGAGCA |
| | | GCAAAAGGTCAGAGTTGTCACAACTGAGCATGTACCG |
| | | AGTGTTTGAAGTAGGTGTTATCAGAAATCCGGGTTTGG |
| | | GGGCTCCGGTGTTCCATATGACAAACTATCTTGAGCAA |
| | | CCAGTCAGTAATGATCTCAGCAACTGTATGGTGGCTTT |
| | | GGGGGAGCTCAAACTCGCAGCCCTTTGTCACGGGGAA |
| | | GATTCTATCACAATTCCCTATCAGGGATCAGGGAAAGG |
| | | TGTCAGCTTCCAGCTCGTCAAGCTAGGTGTCTGGAAAT |
| | | CCCCAACCGACATGCAATCCTGGGTCCCCTTATCAACG |
| | | GATGATCCAGTGATAGACAGGCTTTACCTCTCATCTCA |
| | | CAGAGGTGTTATCGCTGACAATCAAGCAAAATGGGCT |
| | | GTCCCGACAACACGAACAGATGACAAGTTGCGAATGG |
| | | AGACATGCTTCCAACAGGCGTGTAAGGGTAAAATCCA |
| | | AGCACTCTGCGAGAATCCCGAGTGGGCACCATTGAAG |
| | | GATAACAGGATTCCTTCATACGGGGTCTTGTCTGTTGA |
| | | TCTGAGTCTGACAGTTGAGCTTAAAATCAAAATTGCTT |
| | | CGGGATTCGGGCCATTGATCACACACGGTTCAGGGATG |
| | | GACCTATACAAATCCAACCACAACAATGTGTATTGGCT |
| | | GACTATCCCGCCAATGAAGAACCTAGCCTTAGGTGTAA |
| | | TCAACACATTGGAGTGGATACCGAGATTCAAGGTTAGT |
| | | CCCTACCTCTTCACTGTCCCAATTAAGGAAGCAGGCGA |
| | | AGACTGCCATGCCCCAACATACCTACCTGCGGAGGTGG |
| | | ATGGTGATGTCAAACTCAGTTCCAATCTGGTGATTCTA |
| | | CCTGGTCAAGATCTCCAATATGTTTTGGCAACCTACGA |
| | | TACTTCCAGGGTTGAACATGCTGTGGTTTATTACGTTTA |
| | | CAGCCCAAGCCGCTCATTTTCTTACTTTTATCCTTTTAG |
| | | GTTGCCTATAAAGGGGGTCCCCATCGAATTACAAGTGG |
| | | AATGCTTCACATGGGACCAAAAACTCTGGTGCCGTCAC |
| | | TTCTGTGTGCTTGCGGACTCAGAATCTGGTGGACATAT |
| | | CACTCACTCTGGGATGGTGGGCATGGGAGTCAGCTGCA |
| | | CAGTCACCCGGGAAGATGGAACCAATCGCAGATAGGG |
| | | CTGCTAGTGAACCAATCACATGATGTCACCCAGACATC |
| | | AGGCATACCCACTAGTGTGAAATAGACATCAGAATTA |
| | | AGAAAAACGTAGGGTCCAAGTGGTTCCCCGTTATGGAC |
| | | TCGCTATCTGTCAACCAGATCTTATACCCTGAAGTTCA |
| | | CCTAGATAGCCCGATAGTTACCAATAAGATAGTAGCCA |
| | | TCCTGGAGTATGCTCGAGTCCCTCACGCTTACAGCCTG |
| | | GAGGACCCTACACTGTGTCAGAACATCAAGCACCGCCT |
| | | AAAAAACGGATTTTCCAACCAAATGATTATAAACAATG |
| | | TGGAAGTTGGGAATGTCATCAAGTCCAAGCTTAGGAGT |
| | | TATCCGGCCCACTCTCATATTCCATATCCAAATTGTAAT |
| | | CAGGATTTATTTAACATAGAAGACAAAGAGTCAACGA |
| | | GGAAGATCCGTGAACTCCTCAAAAAGGGGAATTCGCT |
| | | GTACTCCAAAGTCAGTGATAAGGTTTTCCAATGCTTAA |
| | | GGGACACTAACTCACGGCTTGGCCTAGGCTCCGAATTG |
| | | AGGGAGGACATCAAGGAGAAAGTTATTAACTTGGGAG |
| | | TTTACATGCACAGCTCCCAGTGGTTTGAGCCCTTTCTGT |
| | | TTTGGTTTACAGTCAAGACTGAGATGAGGTCAGTGATT |
| | | AAATCACAAACCCATACTTGCCATAGGAGGAGACACA |
| | | CACCTGTATTCTTCACTGGTAGTTCAGTTGAGTTGCTAA |
| | | TCTCTCGTGACCTTGTTGCTATAATCAGTAAAGAGTCTC |
| | | AACATGTATATTACCTGACATTTGAACTGGTTTTGATGT |
| | | ATTGTGATGTCATAGAGGGGAGGTTAATGACAGAGAC |
| | | CGCTATGACTATTGATGCTAGGTATACAGAGCTTCTAG |
| | | GAAGAGTCAGATACATGTGGAAACTGATAGATGGTTTC |
| | | TTCCCTGCACTCGGGAATCCAACTTATCAAATTGTAGC |
| | | CATGCTGGAGCCTCTTTCACTTGCTTACCTGCAGCTGA |
| | | GGGATATAACAGTAGAACTCAGAGGTGCTTTCCTTAAC |
| | | CACTGCTTTACTGAAATACATGATGTTCTTGACCAAAA |
| | | CGGGTTTTCTGATGAAGGTACTTATCATGAGTTAACTG |
| | | AAGCTCTAGATTACATTTTCATAACTGATGACATACAT |
| | | CTGACAGGGGAGATTTTCTCATTTTTCAGAAGTTTCGG |
| | | CCACCCCAGACTTGAAGCAGTAACGGCTGCTGAAAAT |
| | | GTTAGGAAATACATGAATCAGCCTAAAGTCATTGTGTA |
| | | TGAGACTCTGATGAAAGGTCATGCCATATTTTGTGGAA |
| | | TCATAATCAACGGCTATCGTGACAGGCACGGAGGCAG |
| | | TTGGCCACCGCTGACCCTCCCCCTGCATGCTGCAGACA |
| | | CAATCCGGAATGCTCAAGCTTCAGGTGAAGGGTTAACA |
| | | CATGAGCAGTGCGTTGATAACTGGAAATCTTTTGCTGG |
| | | AGTGAAATTTGGCTGCTTTATGCCTCTTAGCCTGGATA |
| | | GTGATCTGACAATGTACCTAAAGGACAAGGCACTTGCT |
| | | GCTCTCCAAAGGGAATGGGATTCAGTTTACCCGAAAGA |
| | | GTTCCTGCGTTACGACCCTCCCAAGGGAACCGGGTCAC |
| | | GGAGGCTTGTAGATGTTTTCCTTAATGATTCGAGCTTTG |
| | | ACCCATATGATGTGATAATGTATGTTGTAAGTGGAGCT |
| | | TACCTCCATGACCCTGAGTTCAACCTGTCTTACAGCCT |
| | | GAAAGAAAAGGAGATCAAGGAAACAGGTAGACTTTTT |
| | | GCTAAAATGACTTACAAAATGAGGGCATGCCAAGTGA |
| | | TTGCTGAAAATCTAATCTCAAACGGGATTGGCAAATAT |
| | | TTTAAGGACAATGGGATGGCCAAGGATGAGCACGATT |
| | | TGACTAAGGCACTCCACACTCTAGCTGTCTCAGGAGTC |
| | | CCCAAAGATCTCAAAGAAAGTCACAGGGGGGGGCCAG |
| | | TCTTAAAAACCTACTCCCGAAGCCCAGTCCACACAAGT |
| | | ACCAGGAACGTGAGAGCAGCAAAAGGGTTTATAGGGT |
| | | TCCCTCAAGTAATTCGGCAGGACCAAGACACTGATCAT |
| | | CCGGAGAATATGGAAGCTTACGAGACAGTCAGTGCAT |
| | | TTATCACGACTGATCTCAAGAAGTACTGCCTTAATTGG |
| | | AGATATGAGACCATCAGCTTGTTTGCACAGAGGCTAAA |
| | | TGAGATTTACGGATTGCCCTCATTTTTCCAGTGGCTGCA |
| | | TAAGAGGCTTGAGACCTCTGTCCTGTATGTAAGTGACC |
| | | CTCATTGCCCCCCCGACCTTGACGCCCATATCCCGTTAT |
| | | ATAAAGTCCCCAATGATCAAATCTTCATTAAGTACCCT |
| | | ATGGGAGGTATAGAAGGGTATTGTCAGAAGCTGTGGA |
| | | CCATCAGCACCATTCCCTATCTATACCTGGCTGCTTATG |
| | | AGAGCGGAGTAAGGATTGCTTCGTTAGTGCAAGGGGA |
| | | CAATCAGACCATAGCCGTAACAAAAAGGGTACCCAGC |
| | | ACATGGCCCTACAACCTTAAGAAACGGGAAGCTGCTA |
| | | GAGTAACTAGAGATTACTTTGTAATTCTTAGGCAAAGG |
| | | CTACATGATATTGGCCATCACCTCAAGGCAAATGAGAC |
| | | AATTGTTTCATCACATTTTTTTGTCTATTCAAAAGGAAT |
| | | ATATTATGATGGGCTACTTGTGTCCCAATCACTCAAGA |
| | | GCATCGCAAGATGTGTATTCTGGTCAGAGACTATAGTT |
| | | GATGAAACAAGGGCAGCATGCAGTAATATTGCTACAA |
| | | CAATGGCTAAAAGCATCGAGAGAGGTTATGACCGTTA |
| | | CCTTGCATATTCCCTGAACGTCCTAAAAGTGATACAGC |
| | | AAATTCTGATCTCTCTTGGCTTCACAATCAATTCAACCA |
| | | TGACCCGGGATGTAGTCATACCCCTCCTCACAAACAAC |
| | | GACCTCTTAATAAGGATGGCACTGTTGCCCGCTCCTAT |
| | | TGGGGGGATGAATTATCTGAATATGAGCAGGCTGTTTG |
| | | TCAGAAACATCGGTGATCCAGTAACATCATCAATTGCT |
| | | GATCTCAAGAGAATGATTCTCGCCTCACTAATGCCTGA |
| | | AGAGACCCTCCATCAAGTAATGACACAACAACCGGGG |
| | | GACTCTTCATTCCTAGACTGGGCTAGCGACCCTTACTC |
| | | AGCAAATCTTGTATGTGTCCAGAGCATCACTAGACTCC |
| | | TCAAGAACATAACTGCAAGGTTTGTCCTGATCCATAGT |
| | | CCAAACCCAATGTTAAAAGGATTATTCCATGATGACAG |
| | | TAAAGAAGAGGACGAGGGACTGGCGGCATTCCTCATG |
| | | GACAGGCATATTATAGTACCTAGGGCAGCTCATGAAAT |
| | | CCTGGATCATAGTGTCACAGGGGCAAGAGAGTCTATTG |
| | | CAGGCATGCTGGATACCACAAAAGGCTTGATTCGAGCC |
| | | AGCATGAGGAAGGGGGGGTTAACCTCTCGAGTGATAA |
| | | CCAGATTGTCCAATTATGACTATGAACAATTCAGAGCA |
| | | GGGATGGTGCTATTGACAGGAAGAAAGAGAAATGTCC |
| | | TCATTGACAAAGAGTCATGTTCAGTGCAGCTGGCGAGA |
| | | GCTCTAAGAAGCCATATGTGGGCGAGGCTAGCTCGAG |
| | | GACGGCCTATTTACGGCCTTGAGGTCCCTGATGTACTA |
| | | GAATCTATGCGAGGCCACCTTATTCGGCGTCATGAGAC |
| | | ATGTGTCATCTGCGAGTGTGGATCAGTCAACTACGGAT |
| | | GGTTTTTTGTCCCCTCGGGTTGCCAACTGGATGATATTG |
| | | ACAAGGAAACATCATCCTTGAGAGTCCCATATATTGGT |
| | | TCTACCACTGATGAGAGAACAGACATGAAGCTTGCCTT |
| | | CGTAAGAGCCCCAAGTCGATCCTTGCGATCTGCTGTTA |
| | | GAATAGCAACAGTGTACTCATGGGCTTACGGTGATGAT |
| | | GATAGCTCTTGGAACGAAGCCTGGTTGTTGGCTAGGCA |
| | | AAGGGCCAATGTGAGCCTGGAGGAGCTAAGGGTGATC |
| | | ACTCCCATCTCAACTTCGACTAATTTAGCGCATAGGTT |
| | | GAGGGATCGTAGCACTCAAGTGAAATACTCAGGTACA |
| | | TCCCTTGTCCGAGTGGCGAGGTATACCACAATCTCCAA |
| | | CGACAATCTCTCATTTGTCATATCAGATAAGAAGGTTG |
| | | ATACTAACTTTATATACCAACAAGGAATGCTTCTAGGG |
| | | TTGGGTGTTTTAGAAACATTGTTTCGACTCGAGAAAGA |
| | | TACCGGATCATCTAACACGGTATTACATCTTCACGTCG |
| | | AAACAGATTGTTGCGTGATCCCGATGATAGATCATCCC |
| | | AGGATACCCAGCTCCCGCAAGCTAGAGCTGAGGGCAG |
| | | AGCTATGTACCAACCCATTGATATATGATAATGCACCT |
| | | TTAATTGACAGAGATGCAACAAGGCTATACACCCAGA |
| | | GCCATAGGAGGCACCTTGTGGAATTTGTTACATGGTCC |
| | | ACACCCCAACTATATCACATTTTAGCTAAGTCCACAGC |
| | | ACTATCTATGATTGACCTGGTAACAAAATTTGAGAAGG |
| | | ACCATATGAATGAAATTTCAGCTCTCATAGGGGATGAC |
| | | GATATCAATAGTTTCATAACTGAGTTTCTGCTCATAGA |
| | | GCCAAGATTATTCACTATCTACTTGGGCCAGTGTGCGG |
| | | CCATCAATTGGGCATTTGATGTACATTATCATAGACCA |
| | | TCAGGGAAATATCAGATGGGTGAGCTGTTGTCATCGTT |
| | | CCTTTCTAGAATGAGCAAAGGAGTGTTTAAGGTGCTTG |
| | | TCAATGCTCTAAGCCACCCAAAGATCTACAAGAAATTC |
| | | TGGCATTGTGGTATTATAGAGCCTATCCATGGTCCTTC |
| | | ACTTGATGCTCAAAACTTGCACACAACTGTGTGCAACA |
| | | TGGTTTACACATGCTATATGACCTACCTCGACCTGTTGT |
| | | TGAATGAAGAGTTAGAAGAGTTCACATTTCTCTTGTGT |
| | | GAAAGCGACGAGGATGTAGTACCGGACAGATTCGACA |
| | | ACATCCAGGCAAAACACTTATGTGTTCTGGCAGATTTG |
| | | TACTGTCAACCAGGGACCTGCCCACCAATTCGAGGTCT |
| | | AAGACCGGTAGAGAAATGTGCAGTTCTAACCGACCAT |
| | | ATCAAGGCAGAGGCTATGTTATCTCCAGCAGGATCTTC |
| | | GTGGAACATAAATCCAATTATTGTAGACCATTACTCAT |
| | | GCTCTCTGACTTATCTCCGGCGAGGATCGATCAAACAG |
| | | ATAAGATTGAGAGTTGATCCAGGATTCATTTTCGACGC |
| | | CCTCGCTGAGGTAAATGTCAGTCAGCCAAAGATCGGCA |
| | | GCAACAACATCTCAAATATGAGCATCAAGGCTTTCAGA |
| | | CCCCCACACGATGATGTTGCAAAATTGCTCAAAGATAT |
| | | CAACACAAGCAAGCACAATCTTCCCATTTCAGGGGGCA |
| | | ATCTCGCCAATTATGAAATCCATGCTTTCCGCAGAATC |
| | | GGGTTGAACTCATCTGCTTGCTACAAAGCTGTTGAGAT |
| | | ATCAACATTAATTAGGAGATGCCTTGAGCCAGGGGAG |
| | | GACGGCTTGTTCTTGGGTGAGGGATCGGGTTCTATGTT |
| | | GATCACTTATAAAGAGATACTTAAACTAAACAAGTGCT |
| | | TCTATAATAGTGGGGTTTCCGCCAATTCTAGATCTGGT |
| | | CAAAGGGAATTAGCACCCTATCCCTCCGAAGTTGGCCT |
| | | TGTCGAACACAGAATGGGAGTAGGTAATATTGTCAAA |
| | | GTGCTCTTTAACGGGAGGCCCGAAGTCACGTGGGTAGG |
| | | CAGTGTAGATTGCTTCAATTTCATAGTTAGTAATATCCC |
| | | TACCTCTAGTGTGGGGTTTATCCATTCAGATATAGAGA |
| | | CCTTGCCTGACAAAGATACTATAGAGAAGCTAGAGGA |
| | | ATTGGCAGCCATCTTATCGATGGCTCTGCTCCTGGGCA |
| | | AAATAGGATCAATACTGGTGATTAAGCTTATGCCTTTC |
| | | AGCGGGGATTTTGTTCAGGGATTTATAAGTTATGTAGG |
| | | GTCTCATTATAGAGAAGTGAACCTTGTATACCCTAGAT |
| | | ACAGCAACTTCATCTCTACTGAATCTTATTTGGTTATGA |
| | | CAGATCTCAAGGCTAACCGGCTAATGAATCCTGAAAA |
| | | GATTAAGCAGCAGATAATTGAATCATCTGTGAGGACTT |
| | | CACCTGGACTTATAGGTCACATCCTATCCATTAAGCAA |
| | | CTAAGCTGCATACAAGCAATTGTGGGAGACGCAGTTA |
| | | GTAGAGGTGATATCAATCCTACTCTGAAAAAACTTACA |
| | | CCTATAGAGCAGGTGCTGATCAATTGCGGGTTGGCAAT |
| | | TAACGGACCTAAGCTGTGCAAAGAATTGATCCACCATG |
| | | ATGTTGCCTCAGGGCAAGATGGATTGCTTAATTCTATA |
| | | CTCATCCTCTACAGGGAGTTGGCAAGATTCAAAGACAA |
| | | CCAAAGAAGTCAACAAGGGATGTTCCACGCTTACCCCG |
| | | TATTGGTAAGTAGCAGGCAACGAGAACTTATATCTAGG |
| | | ATCACCCGCAAATTCTGGGGGCACATTCTTCTTTACTCC |
| | | GGGAACAAAAAGTTGATAAATAAGTTTATCCAGAATCT |
| | | CAAGTCCGGCTATCTGATACTAGACTTACACCAGAATA |
| | | TCTTCGTTAAGAATCTATCCAAGTCAGAGAAACAGATT |
| | | ATTATGACGGGGGGTTTGAAACGTGAGTGGGTTTTTAA |
| | | GGTAACAGTCAAGGAGACCAAAGAATGGTATAAGTTA |
| | | GTCGGATACAGTGCCCTGATTAAGGACTAATTGGTTGA |
| | | ACTCCGGAACCCTAATCCTGCCCTAGGTGGTTAGGCAT |
| | | TATTTGCAATATATTAAAGAAAACTTTGAAAATACGAA |
| | | GTTTCTATTCCCAGCTTTGTCTGGT |
| 84 | T-06 | ACCAAACAAAGTTGGGTAAGGATAGTTCAATCAATGA |
| | EBV_gH_ATUa_ | TCATCTTCTAGTGCACTTAGGATTCAAGATCCTATTATC |
| | gL_ATUc_gp42 | AGGGACAAGAGCAGGATTAGGGATATCCGAGATGGCC |
| | at ATU3 | ACACTTTTAAGGAGCTTAGCATTGTTCAAAAGAAACAA |
| | MV leader: nt | GGACAAACCACCCATTACATCAGGATCCGGTGGAGCC |
| | 1-55 | ATCAGAGGAATCAAACACATTATTATAGTACCAATCCC |
| | MV N ORF: nt | TGGAGATTCCTCAATTACCACTCGATCCAGACTTCTGG |
| | 108-1,685 | ACCGGTTGGTGAGGTTAATTGGAAACCCGGATGTGAGC |
| | MV P ORF: nt | GGGCCCAAACTAACAGGGGCACTAATAGGTATATTATC |
| | 1,807-3,330 | CTTATTTGTGGAGTCTCCAGGTCAATTGATTCAGAGGA |
| | MV M ORF: nt | TCACCGATGACCCTGACGTTAGCATAAGGCTGTTAGAG |
| | 3,438-4,445 | GTTGTCCAGAGTGACCAGTCACAATCTGGCCTTACCTT |
| | MV F ORF: nt | CGCATCAAGAGGTACCAACATGGAGGATGAGGCGGAC |
| | 5,449-7,110 | CAATACTTTTCACATGATGATCCAATTAGTAGTGATCA |
| | MV H ORF: nt | ATCCAGGTTCGGATGGTTCGGGAACAAGGAAATCTCA |
| | 7,271-9,124 | GATATTGAAGTGCAAGACCCTGAGGGATTCAACATGAT |
| | ATU upstream | TCTGGGTACCATCCTAGCCCAAATTTGGGTCTTGCTCG |
| | motif: nt 9,180- | CAAAGGCGGTTACGGCCCCAGACACGGCAGCTGATTC |
| | 9,257 | GGAGCTAAGAAGGTGGATAAAGTACACCCAACAAAGA |
| | GE of N gene: | AGGGTAGTTGGTGAATTTAGATTGGAGAGAAAATGGTT |
| | nt 9,196-9,206 | GGATGTGGTGAGGAACAGGATTGCCGAGGACCTCTCCT |
| | Conserved | TACGCCGATTCATGGTCGCTCTAATCCTGGATATCAAG |
| | trinucleotide | AGAACACCCGGAAACAAACCCAGGATTGCTGAAATGA |
| | motif: nt 9,207- | TATGTGACATTGATACATATATCGTAGAGGCAGGATTA |
| | 9,209 | GCCAGTTTTATCCTGACTATTAAGTTTGGGATAGAAAC |
| | GS of P gene: | TATGTATCCTGCTCTTGGACTGCATGAATTTGCTGGTGA |
| | nt 9,210-9,226 | GTTATCCACACTTGAGTCCTTGATGAACCTTTACCAGC |
| | BsiWI | AAATGGGGGAAACTGCACCCTACATGGTAATCCTGGA |
| | restriction site: | GAACTCAATTCAGAACAAGTTCAGTGCAGGATCATACC |
| | nt 9,246-9,251 | CTCTGCTCTGGAGCTATGCCATGGGAGTAGGAGTGGAA |
| | EBV gH ORF: | CTTGAAAACTCCATGGGAGGTTTGAACTTTGGCCGATC |
| | nt 9,258-11,381 | TTACTTTGATCCAGCATATTTTAGATTAGGGCAAGAGA |
| | ATUa motif: nt | TGGTAAGGAGGTCAGCTGGAAAGGTCAGTTCCACATTG |
| | 11,382-11,525 | GCATCTGAACTCGGTATCACTGCCGAGGATGCAAGGCT |
| | GE of P gene: | TGTTTCAGAGATTGCAATGCATACTACTGAGGACAAGA |
| | nt 11,449- | TCAGTAGAGCGGTTGGACCCAGACAAGCCCAAGTATC |
| | 11,459 | ATTTCTACACGGTGATCAAAGTGAGAATGAGCTACCGA |
| | Conserved | GATTGGGGGGCAAGGAAGATAGGAGGGTCAAACAGAG |
| | trinucleotide | TCGAGGAGAAGCCAGGGAGAGCTACAGAGAAACCGGG |
| | motif: nt | CCCAGCAGAGCAAGTGATGCGAGAGCTGCCCATCTTCC |
| | 11,460-11,462 | AACCGGCACACCCCTAGACATTGACACTGCAACGGAG |
| | GS of P gene: | TCCAGCCAAGATCCGCAGGACAGTCGAAGGTCAGCTG |
| | nt 11,463- | ACGCCCTGCTTAGGCTGCAAGCCATGGCAGGAATCTCG |
| | 11,479 | GAAGAACAAGGCTCAGACACGGACACCCCTATAGTGT |
| | EBV gL ORF: | ACAATGACAGAAATCTTCTAGACTAGGTGCGAGAGGC |
| | nt 11,526- | CGAGGGCCAGAACAACATCCGCCTACCATCCATCATTG |
| | 11,939 | TTATAAAAAACTTAGGAACCAGGTCCACACAGCCGCC |
| | ATUc motif: nt | AGCCCATCAACCATCCACTCCCACGATTGGAGCCAATG |
| | 11,940-12,041 | GCAGAAGAGCAGGCACGCCATGTCAAAAACGGACTGG |
| | GE of N gene: | AATGCATCCGGGCTCTCAAGGCCGAGCCCATCGGCTCA |
| | nt 11,991- | CTGGCCATCGAGGAAGCTATGGCAGCATGGTCAGAAA |
| | 12,001 | TATCAGACAACCCAGGACAGGAGCGAGCCACCTGCAG |
| | Conserved | GGAAGAGAAGGCAGGCAGTTCGGGTCTCAGCAAACCA |
| | trinucleotide | TGCCTCTCAGCAATTGGATCAACTGAAGGCGGTGCACC |
| | motif: nt | TCGCATCCGCGGTCAGGGACCTGGAGAGAGCGATGAC |
| | 12,002-12,004 | GACGCTGAAACTTTGGGAATCCCCCCAAGAAATCTCCA |
| | Consensus GS | GGCATCAAGCACTGGGTTACAGTGTTATTACGTTTATG |
| | motif: nt | ATCACAGCGGTGAAGCGGTTAAGGGAATCCAAGATGC |
| | 12,005-12,021 | TGACTCTATCATGGTTCAATCAGGCCTTGATGGTGATA |
| | EBV gp42 | GCACCCTCTCAGGAGGAGACAATGAATCTGAAAACAG |
| | ORF: nt | CGATGTGGATATTGGCGAACCTGATACCGAGGGATATG |
| | 12,042-12,713 | CTATCACTGACCGGGGATCTGCTCCCATCTCTATGGGG |
| | BssHII | TTCAGGGCTTCTGATGTTGAAACTGCAGAAGGAGGGG |
| | restriction site: | AGATCCACGAGCTCCTGAGACTCCAATCCAGAGGCAA |
| | nt 12,714- | CAACTTTCCGAAGCTTGGGAAAACTCTCAATGTTCCTC |
| | 12,719 | CGCCCCCGGACCCCGGTAGGGCCAGCACTTCCGGGAC |
| | ATU | ACCCATTAAAAAGGGCACAGACGCGAGATTAGCCTCA |
| | downstream | TTTGGAACGGAGATCGCGTCTTTATTGACAGGTGGTGC |
| | motif: nt | AACCCAATGTGCTCGAAAGTCACCCTCGGAACCATCAG |
| | 12,714-12,749 | GGCCAGGTGCACCTGCGGGGAATGTCCCCGAGTGTGTG |
| | MV L ORF: nt | AGCAATGCCGCACTGATACAGGAGTGGACACCCGAAT |
| | 12,804-19,355 | CTGGTACCACAATCTCCCCGAGATCCCAGAATAATGAA |
| | MV trailer: nt | GAAGGGGGAGACTATTATGATGATGAGCTGTTCTCTGA |
| | 19,428-19,464 | TGTCCAAGATATTAAAACAGCCTTGGCCAAAATACACG |
| | | AGGATAATCAGAAGATAATCTCCAAGCTAGAATCACT |
| | | GCTGTTATTGAAGGGAGAAGTTGAGTCAATTAAGAAG |
| | | CAGATCAACAGGCAAAATATCAGCATATCCACCCTGG |
| | | AAGGACACCTCTCAAGCATCATGATCGCCATTCCTGGA |
| | | CTTGGGAAGGATCCCAACGACCCCACTGCAGATGTCGA |
| | | AATCAATCCCGACTTGAAACCCATCATAGGCAGAGATT |
| | | CAGGCCGAGCACTGGCCGAAGTTCTCAAGAAACCCGTT |
| | | GCCAGCCGACAACTCCAAGGAATGACAAATGGACGGA |
| | | CCAGTTCCAGAGGACAGCTGCTGAAGGAATTTCAGCTA |
| | | AAGCCGATCGGGAAAAAGATGAGCTCAGCCGTCGGGT |
| | | TTGTTCCTGACACCGGCCCTGCATCACGCAGTGTAATC |
| | | CGCTCCATTATAAAATCCAGCCGGCTAGAGGAGGATCG |
| | | GAAGCGTTACCTGATGACTCTCCTTGATGATATCAAAG |
| | | GAGCCAATGATCTTGCCAAGTTCCACCAGATGCTGATG |
| | | AAGATAATAATGAAGTAGCTACAGCTCAACTTACCTGC |
| | | CAACCCCATGCCAGTCGACCCAACTAGTACAACCTAAA |
| | | TCCATTATAAAAAACTTAGGAGCAAAGTGATTGCCTCC |
| | | CAAGGTCCACAATGACAGAGACCTACGACTTCGACAA |
| | | GTCGGCATGGGACATCAAAGGGTCGATCGCTCCGATAC |
| | | AACCCACCACCTACAGTGATGGCAGGCTGGTGCCCCAG |
| | | GTCAGAGTCATAGATCCTGGTCTAGGCGACAGGAAGG |
| | | ATGAATGCTTTATGTACATGTTTCTGCTGGGGGTTGTTG |
| | | AGGACAGCGATTCCCTAGGGCCTCCAATCGGGCGAGC |
| | | ATTTGGGTTCCTGCCCTTAGGTGTTGGCAGATCCACAG |
| | | CAAAGCCCGAAAAACTCCTCAAAGAGGCCACTGAGCT |
| | | TGACATAGTTGTTAGACGTACAGCAGGGCTCAATGAAA |
| | | AACTGGTGTTCTACAACAACACCCCACTAACTCTCCTC |
| | | ACACCTTGGAGAAAGGTCCTAACAACAGGGAGTGTCTT |
| | | CAACGCAAACCAAGTGTGCAATGCGGTTAATCTGATAC |
| | | CGCTCGATACCCCGCAGAGGTTCCGTGTTGTTTATATG |
| | | AGCATCACCCGTCTTTCGGATAACGGGTATTACACCGT |
| | | TCCTAGAAGAATGCTGGAATTCAGATCGGTCAATGCAG |
| | | TGGCCTTCAACCTGCTGGTGACCCTTAGGATTGACAAG |
| | | GCGATAGGCCCTGGGAAGATCATCGACAATACAGAGC |
| | | AACTTCCTGAGGCAACATTTATGGTCCACATCGGGAAC |
| | | TTCAGGAGAAAGAAGAGTGAAGTCTACTCTGCCGATTA |
| | | TTGCAAAATGAAAATCGAAAAGATGGGCCTGGTTTTTG |
| | | CACTTGGTGGGATAGGGGGCACCAGTCTTCACATTAGA |
| | | AGCACAGGCAAAATGAGCAAGACTCTCCATGCACAAC |
| | | TCGGGTTCAAGAAGACCTTATGTTACCCGCTGATGGAT |
| | | ATCAATGAAGACCTTAATCGATTACTCTGGAGGAGCAG |
| | | ATGCAAGATAGTAAGAATCCAGGCAGTTTTGCAGCCAT |
| | | CAGTTCCTCAAGAATTCCGCATTTACGACGACGTGATC |
| | | ATAAATGATGACCAAGGACTATTCAAAGTTCTGTAGAC |
| | | CGTAGTGCCCAGCAATGCCCGAAAACGACCCCCCTCAC |
| | | AATGACAGCCAGAAGGCCCGGACAAAAAAGCCCCCTC |
| | | CGAAAGACTCCACGGACCAAGCGAGAGGCCAGCCAGC |
| | | AGCCGACGGCAAGCGCGAACACCAGGCGGCCCCAGCA |
| | | CAGAACAGCCCTGACACAAGGCCACCACCAGCCACCC |
| | | CAATCTGCATCCTCCTCGTGGGACCCCCGAGGACCAAC |
| | | CCCCAAGGCTGCCCCCGATCCAAACCACCAACCGCATC |
| | | CCCACCACCCCCGGGAAAGAAACCCCCAGCAATTGGA |
| | | AGGCCCCTCCCCCTCTTCCTCAACACAAGAACTCCACA |
| | | ACCGAACCGCACAAGCGACCGAGGTGACCCAACCGCA |
| | | GGCATCCGACTCCCTAGACAGATCCTCTCTCCCCGGCA |
| | | AACTAAACAAAACTTAGGGCCAAGGAACATACACACC |
| | | CAACAGAACCCAGACCCCGGCCCACGGCGCCGCGCCC |
| | | CCAACCCCCGACAACCAGAGGGAGCCCCCAACCAATC |
| | | CCGCCGGCTCCCCCGGTGCCCACAGGCAGGGACACCA |
| | | ACCCCCGAACAGACCCAGCACCCAACCATCGACAATC |
| | | CAAGACGGGGGGGCCCCCCCAAAAAAAGGCCCCCAGG |
| | | GGCCGACAGCCAGCACCGCGAGGAAGCCCACCCACCC |
| | | CACACACGACCACGGCAACCAAACCAGAACCCAGACC |
| | | ACCCTGGGCCACCAGCTCCCAGACTCGGCCATCACCCC |
| | | GCAGAAAGGAAAGGCCACAACCCGCGCACCCCAGCCC |
| | | CGATCCGGCGGGGAGCCACCCAACCCGAACCAGCACC |
| | | CAAGAGCGATCCCCGAAGGACCCCCGAACCGCAAAGG |
| | | ACATCAGTATCCCACAGCCTCTCCAAGTCCCCCGGTCT |
| | | CCTCCTCTTCTCGAAGGGACCAAAAGATCAATCCACCA |
| | | CACCCGACGACACTCAACTCCCCACCCCTAAAGGAGAC |
| | | ACCGGGAATCCCAGAATCAAGACTCATCCAATGTCCAT |
| | | CATGGGTCTCAAGGTGAACGTCTCTGCCATATTCATGG |
| | | CAGTACTGTTAACTCTCCAAACACCCACCGGTCAAATC |
| | | CATTGGGGCAATCTCTCTAAGATAGGGGTGGTAGGAAT |
| | | AGGAAGTGCAAGCTACAAAGTTATGACTCGTTCCAGCC |
| | | ATCAATCATTAGTCATAAAATTAATGCCCAATATAACT |
| | | CTCCTCAATAACTGCACGAGGGTAGAGATTGCAGAATA |
| | | CAGGAGACTACTGAGAACAGTTTTGGAACCAATTAGA |
| | | GATGCACTTAATGCAATGACCCAGAATATAAGACCGGT |
| | | TCAGAGTGTAGCTTCAAGTAGGAGACACAAGAGATTT |
| | | GCGGGAGTAGTCCTGGCAGGTGCGGCCCTAGGCGTTGC |
| | | CACAGCTGCTCAGATAACAGCCGGCATTGCACTTCACC |
| | | AGTCCATGCTGAACTCTCAAGCCATCGACAATCTGAGA |
| | | GCGAGCCTGGAAACTACTAATCAGGCAATTGAGACAA |
| | | TCAGACAAGCAGGGCAGGAGATGATATTGGCTGTTCA |
| | | GGGTGTCCAAGACTACATCAATAATGAGCTGATACCGT |
| | | CTATGAACCAACTATCTTGTGATTTAATCGGCCAGAAG |
| | | CTCGGGCTCAAATTGCTCAGATACTATACAGAAATCCT |
| | | GTCATTATTTGGCCCCAGTTTACGGGACCCCATATCTG |
| | | CGGAGATATCTATCCAGGCTTTGAGCTATGCGCTTGGA |
| | | GGAGACATCAATAAGGTGTTAGAAAAGCTCGGATACA |
| | | GTGGAGGTGATTTACTGGGCATCTTAGAGAGCGGAGG |
| | | CATCAAGGCCCGGATAACTCACGTCGACACAGAGTCCT |
| | | ACTTCATTGTCCTCAGTATAGCCTATCCGACGCTGTCCG |
| | | AGATTAAGGGGGTGATTGTCCACCGGCTAGAGGGGGT |
| | | CTCGTACAACATAGGCTCTCAAGAGTGGTATACCACTG |
| | | TGCCCAAGTATGTTGCAACCCAAGGGTACCTTATCTCG |
| | | AATTTTGATGAGTCATCGTGTACTTTCATGCCAGAGGG |
| | | GACTGTGTGCAGCCAAAATGCCTTGTACCCGATGAGTC |
| | | CTCTGCTCCAAGAATGCCTCCGGGGGTACACCAAGTCC |
| | | TGTGCTCGTACACTCGTATCCGGGTCTTTTGGGAACCG |
| | | GTTCATTTTATCACAAGGGAACCTAATAGCCAATTGTG |
| | | CATCAATCCTTTGCAAGTGTTACACAACAGGAACGATC |
| | | ATTAATCAAGACCCTGACAAGATCCTAACATACATTGC |
| | | TGCCGATCACTGCCCGGTAGTCGAGGTGAACGGCGTGA |
| | | CCATCCAAGTCGGGAGCAGGAGGTATCCAGACGCTGT |
| | | GTACTTGCACAGAATTGACCTCGGTCCTCCCATATCAT |
| | | TGGAGAGGTTGGACGTAGGGACAAATCTGGGGAATGC |
| | | AATTGCTAAGTTGGAGGATGCCAAGGAATTGTTGGAGT |
| | | CATCGGACCAGATATTGAGGAGTATGAAAGGTTTATCG |
| | | AGCACTAGCATAGTCTACATCCTGATTGCAGTGTGTCT |
| | | TGGAGGGTTGATAGGGATCCCCGCTTTAATATGTTGCT |
| | | GCAGGGGGCGTTGTAACAAAAAGGGAGAACAAGTTGG |
| | | TATGTCAAGACCAGGCCTAAAGCCTGATCTTACGGGAA |
| | | CATCAAAATCCTATGTAAGGTCGCTCTGATCCTCTACA |
| | | ACTCTTGAAACACAAATGTCCCACAAGTCTCCTCTTCG |
| | | TCATCAAGCAACCACCGCACCCAGCATCAAGCCCACCT |
| | | GAAATTATCTCCGGCTTCCCTCTGGCCGAACAATATCG |
| | | GTAGTTAATCAAAACTTAGGGTGCAAGATCATCCACAA |
| | | TGTCACCACAACGAGACCGGATAAATGCCTTCTACAAA |
| | | GATAACCCCCATCCCAAGGGAAGTAGGATAGTCATTA |
| | | ACAGAGAACATCTTATGATTGATAGACCTTATGTTTTG |
| | | CTGGCTGTTCTGTTTGTCATGTTTCTGAGCTTGATCGGG |
| | | TTGCTAGCCATTGCAGGCATTAGACTTCATCGGGCAGC |
| | | CATCTACACCGCAGAGATCCATAAAAGCCTCAGCACCA |
| | | ATCTAGATGTAACTAACTCAATCGAGCATCAGGTCAAG |
| | | GACGTGCTGACACCACTCTTCAAAATCATCGGTGATGA |
| | | AGTGGGCCTGAGGACACCTCAGAGATTCACTGACCTAG |
| | | TGAAATTAATCTCTGACAAGATCAAGTTCCTTAATCCG |
| | | GATAGGGAGTACGACTTCAGAGATCTCACTTGGTGTAT |
| | | CAACCCGCCAGAGAGAATCAAATTGGATTATGATCAAT |
| | | ACTGTGCAGATGTGGCTGCTGAAGAGCTCATGAATGCA |
| | | TTGGTGAACTCAACTCTACTGGAGACCAGAACAACCAA |
| | | TCAGTTCCTAGCTGTCTCAAAGGGAAACTGCTCAGGGC |
| | | CCACTACAATCAGAGGTCAATTCTCAAACATGTCGCTG |
| | | TCCCTGTTAGACTTGTATTTAGGTCGAGGTTACAATGT |
| | | GTCATCTATAGTCACTATGACATCCCAGGGAATGTATG |
| | | GGGGAACTTACCTAGTGGAAAAGCCTAATCTGAGCAG |
| | | CAAAAGGTCAGAGTTGTCACAACTGAGCATGTACCGA |
| | | GTGTTTGAAGTAGGTGTTATCAGAAATCCGGGTTTGGG |
| | | GGCTCCGGTGTTCCATATGACAAACTATCTTGAGCAAC |
| | | CAGTCAGTAATGATCTCAGCAACTGTATGGTGGCTTTG |
| | | GGGGAGCTCAAACTCGCAGCCCTTTGTCACGGGGAAG |
| | | ATTCTATCACAATTCCCTATCAGGGATCAGGGAAAGGT |
| | | GTCAGCTTCCAGCTCGTCAAGCTAGGTGTCTGGAAATC |
| | | CCCAACCGACATGCAATCCTGGGTCCCCTTATCAACGG |
| | | ATGATCCAGTGATAGACAGGCTTTACCTCTCATCTCAC |
| | | AGAGGTGTTATCGCTGACAATCAAGCAAAATGGGCTGT |
| | | CCCGACAACACGAACAGATGACAAGTTGCGAATGGAG |
| | | ACATGCTTCCAACAGGCGTGTAAGGGTAAAATCCAAG |
| | | CACTCTGCGAGAATCCCGAGTGGGCACCATTGAAGGAT |
| | | AACAGGATTCCTTCATACGGGGTCTTGTCTGTTGATCT |
| | | GAGTCTGACAGTTGAGCTTAAAATCAAAATTGCTTCGG |
| | | GATTCGGGCCATTGATCACACACGGTTCAGGGATGGAC |
| | | CTATACAAATCCAACCACAACAATGTGTATTGGCTGAC |
| | | TATCCCGCCAATGAAGAACCTAGCCTTAGGTGTAATCA |
| | | ACACATTGGAGTGGATACCGAGATTCAAGGTTAGTCCC |
| | | TACCTCTTCACTGTCCCAATTAAGGAAGCAGGCGAAGA |
| | | CTGCCATGCCCCAACATACCTACCTGCGGAGGTGGATG |
| | | GTGATGTCAAACTCAGTTCCAATCTGGTGATTCTACCT |
| | | GGTCAAGATCTCCAATATGTTTTGGCAACCTACGATAC |
| | | TTCCAGGGTTGAACATGCTGTGGTTTATTACGTTTACA |
| | | GCCCAAGCCGCTCATTTTCTTACTTTTATCCTTTTAGGT |
| | | TGCCTATAAAGGGGGTCCCCATCGAATTACAAGTGGAA |
| | | TGCTTCACATGGGACCAAAAACTCTGGTGCCGTCACTT |
| | | CTGTGTGCTTGCGGACTCAGAATCTGGTGGACATATCA |
| | | CTCACTCTGGGATGGTGGGCATGGGAGTCAGCTGCACA |
| | | GTCACCCGGGAAGATGGAACCAATCGCAGATAGGGCT |
| | | GCTAGTGAACCAATCACATGATGTCACCCAGACATCAG |
| | | GCATACCCACTAGTCTACCCTCCATCATTGTTATAAAA |
| | | AACTTAGGAACCAGGTCCACACAGCCGCCAGCCCATC |
| | | AACGCGTACGGCCACCATGCAACTCCTCTGCGTCTTTT |
| | | GCTTGGTGCTTTTGTGGGAGGTAGGAGCGGCCTCATTG |
| | | TCAGAAGTTAAGCTGCATCTCGATATTGAAGGCCACGC |
| | | TTCACATTACACTATCCCTTGGACCGAACTCATGGCTA |
| | | AGGTGCCGGGTTTGTCTCCGGAAGCACTTTGGAGAGAG |
| | | GCAAACGTTACCGAAGACCTGGCCTCAATGCTGAATCG |
| | | CTACAAGCTGATATACAAGACTTCCGGAACCCTGGGTA |
| | | TAGCACTCGCAGAGCCCGTCGATATCCCCGCTGTCAGC |
| | | GAAGGTAGCATGCAGGTGGACGCTAGTAAAGTTCACC |
| | | CAGGAGTTATCTCAGGTCTGAATAGCCCAGCTTGTATG |
| | | CTGAGCGCTCCACTGGAGAAACAGCTCTTCTACTATAT |
| | | AGGCACAATGCTCCCTAACACTCGACCTCACAGCTACG |
| | | TCTTCTATCAGCTGCGCTGTCACCTGTCATACGTAGCCT |
| | | TGTCCATTAATGGTGATAAGTTCCAGTACACAGGTGCC |
| | | ATGACCTCCAAGTTCCTTATGGGGACCTATAAACGGGT |
| | | CACTGAGAAAGGAGACGAGCATGTCCTTAGTTTGGTTT |
| | | TTGGGAAGACAAAAGACCTGCCGGATCTGAGGGGCCC |
| | | GTTCTCTTACCCCTCACTCACATCTGCACAGAGTGGGG |
| | | ACTATTCATTGGTGATTGTGACAACCTTCGTTCACTACG |
| | | CCAATTTTCACAACTATTTTGTCCCGAACCTTAAAGAC |
| | | ATGTTTTCACGCGCCGTGACCATGACAGCTGCTTCCTA |
| | | CGCTAGGTATGTGCTCCAAAAGCTGGTGCTGCTGGAGA |
| | | TGAAGGGCGGATGCAGGGAACCTGAACTGGACACAGA |
| | | GACTCTCACCACTATGTTCGAGGTTTCCGTGGCGTTCTT |
| | | CAAGGTGGGTCATGCTGTGGGGGAGACTGGGAACGGC |
| | | TGCGTAGATCTTAGGTGGCTGGCTAAATCTTTCTTCGA |
| | | GCTGACCGTGTTGAAAGATATAATTGGAATTTGCTACG |
| | | GGGCTACGGTTAAAGGAATGCAGTCTTACGGGCTGGA |
| | | AAGGCTGGCCGCTATGCTCATGGCTACTGTGAAGATGG |
| | | AGGAACTCGGCCACCTCACCACAGAAAAACAGGAGTA |
| | | CGCTTTGCGCCTGGCCACTGTTGGCTACCCTAAGGCAG |
| | | GCGTTTACTCTGGATTGATTGGGGGGGCCACATCTGTG |
| | | CTCTTGTCAGCCTACAATCGCCATCCCCTGTTCCAGCCT |
| | | CTGCACACGGTGATGCGAGAGACTTTGTTTATTGGCAG |
| | | CCACGTTGTGCTGAGAGAACTGCGCCTTAACGTTACCA |
| | | CTCAGGGACCTAACCTTGCGCTGTATCAGCTGCTTAGC |
| | | ACCGCTTTGTGCTCCGCTCTTGAAATTGGAGAAGTACT |
| | | CAGAGGGCTCGCACTGGGTACTGAGTCAGGGCTGTTCT |
| | | CCCCCTGCTATCTGTCTCTCCGGTTCGACCTCACTCGGG |
| | | ATAAGCTGTTGTCAATGGCTCCGCAGGAAGCCACATTG |
| | | GACCAGGCCGCAGTGAGTAACGCAGTGGACGGGTTCC |
| | | TGGGTAGACTGTCTCTTGAGAGGGAAGACAGAGACGC |
| | | TTGGCATCTGCCTGCGTATAAGTGTGTTGATAGACTGG |
| | | ACAAAGTACTGATGATCATTCCCCTGATTAATGTGACA |
| | | TTTATTATATCTAGTGACCGAGAGGTGAGAGGCTCCGC |
| | | TCTCTATGAAGCATCCACAACGTACTTGAGCAGTTCCC |
| | | TGTTTCTGTCACCGGTTATTATGAACAAATGTTCTCAGG |
| | | GAGCTGTTGCTGGTGAACCGCGACAGATTCCAAAGATT |
| | | CAGAACTTCACTAGGACACAAAAGAGCTGCATTTTCTG |
| | | CGGGTTCGCACTGCTGAGTTACGACGAAAAGGAGGGA |
| | | CTGGAGACTACTACATATATTACTTCACAGGAGGTGCA |
| | | GAATAGCATCCTGAGCTCCAATTATTTTGACTTTGACA |
| | | ACCTCCATGTCCATTATTTGCTGTTGACCACAAACGGC |
| | | ACTGTGATGGAGATCGCTGGCCTGTACGAGGAGCGAG |
| | | CCCACGTAGTACTGGCCATTATTCTGTACTTCATCGCTT |
| | | TCGCACTTGGGATTTTCCTCGTCCACAAAATTGTTATGT |
| | | TTTTCCTCTAATGATTCGAACTACAGCTCAACTTACCTG |
| | | CCAACCCCATGCCAGTCGACCCAACTAGTACAACCTAA |
| | | ATCCATTATAAAAAACTTAGGAACCAGGTCCACACAGC |
| | | CGCCAGCCCATCAACCATCCACTCCCACGATTGGAGGC |
| | | CGGCCATGAGAGCCGTAGGTGTCTTTCTGGCTATTTGT |
| | | CTGGTAACAATCTTCGTGCTGCCCACTTGGGGCAACTG |
| | | GGCCTATCCTTGCTGCCATGTTACACAGTTGAGAGCGC |
| | | AGCACCTCCTGGCGTTGGAGAACATTTCAGACATCTAC |
| | | CTGGTGAGTAACCAAACCTGCGACGGGTTCTCTTTGGC |
| | | CAGTCTCAATAGTCCAAAAAACGGAAGCAATCAGCTG |
| | | GTAATTTCACGGTGTGCAAACGGACTTAACGTGGTCTC |
| | | TTTTTTTATATCAATCCTGAAACGCAGTTCATCTGCATT |
| | | GACCGGCCACTTGAGGGAGCTCTTGACCACTCTCGAGA |
| | | CACTTTACGGGTCATTCTCCGTGGAGGATCTGTTCGGG |
| | | GCAAATCTGAACCGGTACGCCTGGCATAGAGGCGGGT |
| | | AACTATTCGAACTACTCAGCTAGTCTACGCGCCGTAGC |
| | | CTACCCTCCATCATTGTTATAAAAAACTTAGGATCCAA |
| | | GAGCATACAGCCGCCGATAAGGTCCACCATGGTGAGTT |
| | | TTAAGCAGGTCCGGGTACCTCTGTTCACCGCCATTGCG |
| | | CTGGTGATTGTTCTGCTGTTGGCCTACTTTCTTCCACCT |
| | | AGAGTCAGAGGAGGTGGCAGGGTTGCTGCAGCTGCTA |
| | | TTACCTGGGTACCGAAACCCAACGTGGAGGTTTGGCCA |
| | | GTGGATCCGCCCCCACCAGTGAACTTTAATAAAACAGC |
| | | CGAACAAGAATACGGGGATAAAGAAGTAAAGCTGCCA |
| | | CACTGGACCCCTACCCTCCACACTTTCCAGGTGCCTCA |
| | | AAACTATACAAAGGCCAATTGCACATACTGTAACACA |
| | | AGAGAATACACTTTTTCTTATAAAGGGTGCTGCTTCTA |
| | | CTTCACTAAGAAGAAGCATACTTGGAACGGATGTTTCC |
| | | AGGCATGCGCTGAGTTGTATCCCTGTACCTATTTCTAC |
| | | GGTCCTACACCAGACATTCTGCCGGTCGTGACTCGCAA |
| | | TCTGAACGCCATAGAATCTCTGTGGGTCGGGGTGTATC |
| | | GGGTTGGGGAAGGAAATTGGACTAGCCTGGACGGCGG |
| | | TACTTTCAAGGTGTACCAAATATTCGGCAGTCACTGTA |
| | | CTTACGTGTCAAAATTCTCAACAGTTCCCGTATCCCAC |
| | | CACGAGTGTTCTTTTTTGAAGCCTTGCCTGTGCGTGTCA |
| | | CAGAGGTCAAATTCCTAAGCGCGCAGCGCTTAGACGTC |
| | | TCGCGATCGATGCTAGTGTGAAATAGACATCAGAATTA |
| | | AGAAAAACGTAGGGTCCAAGTGGTTCCCCGTTATGGAC |
| | | TCGCTATCTGTCAACCAGATCTTATACCCTGAAGTTCA |
| | | CCTAGATAGCCCGATAGTTACCAATAAGATAGTAGCCA |
| | | TCCTGGAGTATGCTCGAGTCCCTCACGCTTACAGCCTG |
| | | GAGGACCCTACACTGTGTCAGAACATCAAGCACCGCCT |
| | | AAAAAACGGATTTTCCAACCAAATGATTATAAACAATG |
| | | TGGAAGTTGGGAATGTCATCAAGTCCAAGCTTAGGAGT |
| | | TATCCGGCCCACTCTCATATTCCATATCCAAATTGTAAT |
| | | CAGGATTTATTTAACATAGAAGACAAAGAGTCAACGA |
| | | GGAAGATCCGTGAACTCCTCAAAAAGGGGAATTCGCT |
| | | GTACTCCAAAGTCAGTGATAAGGTTTTCCAATGCTTAA |
| | | GGGACACTAACTCACGGCTTGGCCTAGGCTCCGAATTG |
| | | AGGGAGGACATCAAGGAGAAAGTTATTAACTTGGGAG |
| | | TTTACATGCACAGCTCCCAGTGGTTTGAGCCCTTTCTGT |
| | | TTTGGTTTACAGTCAAGACTGAGATGAGGTCAGTGATT |
| | | AAATCACAAACCCATACTTGCCATAGGAGGAGACACA |
| | | CACCTGTATTCTTCACTGGTAGTTCAGTTGAGTTGCTAA |
| | | TCTCTCGTGACCTTGTTGCTATAATCAGTAAAGAGTCTC |
| | | AACATGTATATTACCTGACATTTGAACTGGTTTTGATGT |
| | | ATTGTGATGTCATAGAGGGGAGGTTAATGACAGAGAC |
| | | CGCTATGACTATTGATGCTAGGTATACAGAGCTTCTAG |
| | | GAAGAGTCAGATACATGTGGAAACTGATAGATGGTTTC |
| | | TTCCCTGCACTCGGGAATCCAACTTATCAAATTGTAGC |
| | | CATGCTGGAGCCTCTTTCACTTGCTTACCTGCAGCTGA |
| | | GGGATATAACAGTAGAACTCAGAGGTGCTTTCCTTAAC |
| | | CACTGCTTTACTGAAATACATGATGTTCTTGACCAAAA |
| | | CGGGTTTTCTGATGAAGGTACTTATCATGAGTTAACTG |
| | | AAGCTCTAGATTACATTTTCATAACTGATGACATACAT |
| | | CTGACAGGGGAGATTTTCTCATTTTTCAGAAGTTTCGG |
| | | CCACCCCAGACTTGAAGCAGTAACGGCTGCTGAAAAT |
| | | GTTAGGAAATACATGAATCAGCCTAAAGTCATTGTGTA |
| | | TGAGACTCTGATGAAAGGTCATGCCATATTTTGTGGAA |
| | | TCATAATCAACGGCTATCGTGACAGGCACGGAGGCAG |
| | | TTGGCCACCGCTGACCCTCCCCCTGCATGCTGCAGACA |
| | | CAATCCGGAATGCTCAAGCTTCAGGTGAAGGGTTAACA |
| | | CATGAGCAGTGCGTTGATAACTGGAAATCTTTTGCTGG |
| | | AGTGAAATTTGGCTGCTTTATGCCTCTTAGCCTGGATA |
| | | GTGATCTGACAATGTACCTAAAGGACAAGGCACTTGCT |
| | | GCTCTCCAAAGGGAATGGGATTCAGTTTACCCGAAAGA |
| | | GTTCCTGCGTTACGACCCTCCCAAGGGAACCGGGTCAC |
| | | GGAGGCTTGTAGATGTTTTCCTTAATGATTCGAGCTTTG |
| | | ACCCATATGATGTGATAATGTATGTTGTAAGTGGAGCT |
| | | TACCTCCATGACCCTGAGTTCAACCTGTCTTACAGCCT |
| | | GAAAGAAAAGGAGATCAAGGAAACAGGTAGACTTTTT |
| | | GCTAAAATGACTTACAAAATGAGGGCATGCCAAGTGA |
| | | TTGCTGAAAATCTAATCTCAAACGGGATTGGCAAATAT |
| | | TTTAAGGACAATGGGATGGCCAAGGATGAGCACGATT |
| | | TGACTAAGGCACTCCACACTCTAGCTGTCTCAGGAGTC |
| | | CCCAAAGATCTCAAAGAAAGTCACAGGGGGGGGCCAG |
| | | TCTTAAAAACCTACTCCCGAAGCCCAGTCCACACAAGT |
| | | ACCAGGAACGTGAGAGCAGCAAAAGGGTTTATAGGGT |
| | | TCCCTCAAGTAATTCGGCAGGACCAAGACACTGATCAT |
| | | CCGGAGAATATGGAAGCTTACGAGACAGTCAGTGCAT |
| | | TTATCACGACTGATCTCAAGAAGTACTGCCTTAATTGG |
| | | AGATATGAGACCATCAGCTTGTTTGCACAGAGGCTAAA |
| | | TGAGATTTACGGATTGCCCTCATTTTTCCAGTGGCTGCA |
| | | TAAGAGGCTTGAGACCTCTGTCCTGTATGTAAGTGACC |
| | | CTCATTGCCCCCCCGACCTTGACGCCCATATCCCGTTAT |
| | | ATAAAGTCCCCAATGATCAAATCTTCATTAAGTACCCT |
| | | ATGGGAGGTATAGAAGGGTATTGTCAGAAGCTGTGGA |
| | | CCATCAGCACCATTCCCTATCTATACCTGGCTGCTTATG |
| | | AGAGCGGAGTAAGGATTGCTTCGTTAGTGCAAGGGGA |
| | | CAATCAGACCATAGCCGTAACAAAAAGGGTACCCAGC |
| | | ACATGGCCCTACAACCTTAAGAAACGGGAAGCTGCTA |
| | | GAGTAACTAGAGATTACTTTGTAATTCTTAGGCAAAGG |
| | | CTACATGATATTGGCCATCACCTCAAGGCAAATGAGAC |
| | | AATTGTTTCATCACATTTTTTTGTCTATTCAAAAGGAAT |
| | | ATATTATGATGGGCTACTTGTGTCCCAATCACTCAAGA |
| | | GCATCGCAAGATGTGTATTCTGGTCAGAGACTATAGTT |
| | | GATGAAACAAGGGCAGCATGCAGTAATATTGCTACAA |
| | | CAATGGCTAAAAGCATCGAGAGAGGTTATGACCGTTA |
| | | CCTTGCATATTCCCTGAACGTCCTAAAAGTGATACAGC |
| | | AAATTCTGATCTCTCTTGGCTTCACAATCAATTCAACCA |
| | | TGACCCGGGATGTAGTCATACCCCTCCTCACAAACAAC |
| | | GACCTCTTAATAAGGATGGCACTGTTGCCCGCTCCTAT |
| | | TGGGGGGATGAATTATCTGAATATGAGCAGGCTGTTTG |
| | | TCAGAAACATCGGTGATCCAGTAACATCATCAATTGCT |
| | | GATCTCAAGAGAATGATTCTCGCCTCACTAATGCCTGA |
| | | AGAGACCCTCCATCAAGTAATGACACAACAACCGGGG |
| | | GACTCTTCATTCCTAGACTGGGCTAGCGACCCTTACTC |
| | | AGCAAATCTTGTATGTGTCCAGAGCATCACTAGACTCC |
| | | TCAAGAACATAACTGCAAGGTTTGTCCTGATCCATAGT |
| | | CCAAACCCAATGTTAAAAGGATTATTCCATGATGACAG |
| | | TAAAGAAGAGGACGAGGGACTGGCGGCATTCCTCATG |
| | | GACAGGCATATTATAGTACCTAGGGCAGCTCATGAAAT |
| | | CCTGGATCATAGTGTCACAGGGGCAAGAGAGTCTATTG |
| | | CAGGCATGCTGGATACCACAAAAGGCTTGATTCGAGCC |
| | | AGCATGAGGAAGGGGGGGTTAACCTCTCGAGTGATAA |
| | | CCAGATTGTCCAATTATGACTATGAACAATTCAGAGCA |
| | | GGGATGGTGCTATTGACAGGAAGAAAGAGAAATGTCC |
| | | TCATTGACAAAGAGTCATGTTCAGTGCAGCTGGCGAGA |
| | | GCTCTAAGAAGCCATATGTGGGCGAGGCTAGCTCGAG |
| | | GACGGCCTATTTACGGCCTTGAGGTCCCTGATGTACTA |
| | | GAATCTATGCGAGGCCACCTTATTCGGCGTCATGAGAC |
| | | ATGTGTCATCTGCGAGTGTGGATCAGTCAACTACGGAT |
| | | GGTTTTTTGTCCCCTCGGGTTGCCAACTGGATGATATTG |
| | | ACAAGGAAACATCATCCTTGAGAGTCCCATATATTGGT |
| | | TCTACCACTGATGAGAGAACAGACATGAAGCTTGCCTT |
| | | CGTAAGAGCCCCAAGTCGATCCTTGCGATCTGCTGTTA |
| | | GAATAGCAACAGTGTACTCATGGGCTTACGGTGATGAT |
| | | GATAGCTCTTGGAACGAAGCCTGGTTGTTGGCTAGGCA |
| | | AAGGGCCAATGTGAGCCTGGAGGAGCTAAGGGTGATC |
| | | ACTCCCATCTCAACTTCGACTAATTTAGCGCATAGGTT |
| | | GAGGGATCGTAGCACTCAAGTGAAATACTCAGGTACA |
| | | TCCCTTGTCCGAGTGGCGAGGTATACCACAATCTCCAA |
| | | CGACAATCTCTCATTTGTCATATCAGATAAGAAGGTTG |
| | | ATACTAACTTTATATACCAACAAGGAATGCTTCTAGGG |
| | | TTGGGTGTTTTAGAAACATTGTTTCGACTCGAGAAAGA |
| | | TACCGGATCATCTAACACGGTATTACATCTTCACGTCG |
| | | AAACAGATTGTTGCGTGATCCCGATGATAGATCATCCC |
| | | AGGATACCCAGCTCCCGCAAGCTAGAGCTGAGGGCAG |
| | | AGCTATGTACCAACCCATTGATATATGATAATGCACCT |
| | | TTAATTGACAGAGATGCAACAAGGCTATACACCCAGA |
| | | GCCATAGGAGGCACCTTGTGGAATTTGTTACATGGTCC |
| | | ACACCCCAACTATATCACATTTTAGCTAAGTCCACAGC |
| | | ACTATCTATGATTGACCTGGTAACAAAATTTGAGAAGG |
| | | ACCATATGAATGAAATTTCAGCTCTCATAGGGGATGAC |
| | | GATATCAATAGTTTCATAACTGAGTTTCTGCTCATAGA |
| | | GCCAAGATTATTCACTATCTACTTGGGCCAGTGTGCGG |
| | | CCATCAATTGGGCATTTGATGTACATTATCATAGACCA |
| | | TCAGGGAAATATCAGATGGGTGAGCTGTTGTCATCGTT |
| | | CCTTTCTAGAATGAGCAAAGGAGTGTTTAAGGTGCTTG |
| | | TCAATGCTCTAAGCCACCCAAAGATCTACAAGAAATTC |
| | | TGGCATTGTGGTATTATAGAGCCTATCCATGGTCCTTC |
| | | ACTTGATGCTCAAAACTTGCACACAACTGTGTGCAACA |
| | | TGGTTTACACATGCTATATGACCTACCTCGACCTGTTGT |
| | | TGAATGAAGAGTTAGAAGAGTTCACATTTCTCTTGTGT |
| | | GAAAGCGACGAGGATGTAGTACCGGACAGATTCGACA |
| | | ACATCCAGGCAAAACACTTATGTGTTCTGGCAGATTTG |
| | | TACTGTCAACCAGGGACCTGCCCACCAATTCGAGGTCT |
| | | AAGACCGGTAGAGAAATGTGCAGTTCTAACCGACCAT |
| | | ATCAAGGCAGAGGCTATGTTATCTCCAGCAGGATCTTC |
| | | GTGGAACATAAATCCAATTATTGTAGACCATTACTCAT |
| | | GCTCTCTGACTTATCTCCGGCGAGGATCGATCAAACAG |
| | | ATAAGATTGAGAGTTGATCCAGGATTCATTTTCGACGC |
| | | CCTCGCTGAGGTAAATGTCAGTCAGCCAAAGATCGGCA |
| | | GCAACAACATCTCAAATATGAGCATCAAGGCTTTCAGA |
| | | CCCCCACACGATGATGTTGCAAAATTGCTCAAAGATAT |
| | | CAACACAAGCAAGCACAATCTTCCCATTTCAGGGGGCA |
| | | ATCTCGCCAATTATGAAATCCATGCTTTCCGCAGAATC |
| | | GGGTTGAACTCATCTGCTTGCTACAAAGCTGTTGAGAT |
| | | ATCAACATTAATTAGGAGATGCCTTGAGCCAGGGGAG |
| | | GACGGCTTGTTCTTGGGTGAGGGATCGGGTTCTATGTT |
| | | GATCACTTATAAAGAGATACTTAAACTAAACAAGTGCT |
| | | TCTATAATAGTGGGGTTTCCGCCAATTCTAGATCTGGT |
| | | CAAAGGGAATTAGCACCCTATCCCTCCGAAGTTGGCCT |
| | | TGTCGAACACAGAATGGGAGTAGGTAATATTGTCAAA |
| | | GTGCTCTTTAACGGGAGGCCCGAAGTCACGTGGGTAGG |
| | | CAGTGTAGATTGCTTCAATTTCATAGTTAGTAATATCCC |
| | | TACCTCTAGTGTGGGGTTTATCCATTCAGATATAGAGA |
| | | CCTTGCCTGACAAAGATACTATAGAGAAGCTAGAGGA |
| | | ATTGGCAGCCATCTTATCGATGGCTCTGCTCCTGGGCA |
| | | AAATAGGATCAATACTGGTGATTAAGCTTATGCCTTTC |
| | | AGCGGGGATTTTGTTCAGGGATTTATAAGTTATGTAGG |
| | | GTCTCATTATAGAGAAGTGAACCTTGTATACCCTAGAT |
| | | ACAGCAACTTCATCTCTACTGAATCTTATTTGGTTATGA |
| | | CAGATCTCAAGGCTAACCGGCTAATGAATCCTGAAAA |
| | | GATTAAGCAGCAGATAATTGAATCATCTGTGAGGACTT |
| | | CACCTGGACTTATAGGTCACATCCTATCCATTAAGCAA |
| | | CTAAGCTGCATACAAGCAATTGTGGGAGACGCAGTTA |
| | | GTAGAGGTGATATCAATCCTACTCTGAAAAAACTTACA |
| | | CCTATAGAGCAGGTGCTGATCAATTGCGGGTTGGCAAT |
| | | TAACGGACCTAAGCTGTGCAAAGAATTGATCCACCATG |
| | | ATGTTGCCTCAGGGCAAGATGGATTGCTTAATTCTATA |
| | | CTCATCCTCTACAGGGAGTTGGCAAGATTCAAAGACAA |
| | | CCAAAGAAGTCAACAAGGGATGTTCCACGCTTACCCCG |
| | | TATTGGTAAGTAGCAGGCAACGAGAACTTATATCTAGG |
| | | ATCACCCGCAAATTCTGGGGGCACATTCTTCTTTACTCC |
| | | GGGAACAAAAAGTTGATAAATAAGTTTATCCAGAATCT |
| | | CAAGTCCGGCTATCTGATACTAGACTTACACCAGAATA |
| | | TCTTCGTTAAGAATCTATCCAAGTCAGAGAAACAGATT |
| | | ATTATGACGGGGGGTTTGAAACGTGAGTGGGTTTTTAA |
| | | GGTAACAGTCAAGGAGACCAAAGAATGGTATAAGTTA |
| | | GTCGGATACAGTGCCCTGATTAAGGACTAATTGGTTGA |
| | | ACTCCGGAACCCTAATCCTGCCCTAGGTGGTTAGGCAT |
| | | TATTTGCAATATATTAAAGAAAACTTTGAAAATACGAA |
| | | GTTTCTATTCCCAGCTTTGTCTGGT |
| 85 | T-07 | ACCAAACAAAGTTGGGTAAGGATAGTTCAATCAATGA |
| | EBV_gp42- | TCATCTTCTAGTGCACTTAGGATTCAAGATCCTATTATC |
| | ATUa_gH- | AGGGACAAGAGCAGGATTAGGGATATCCGAGATGGCC |
| | ATUc_gL at | ACACTTTTAAGGAGCTTAGCATTGTTCAAAAGAAACAA |
| | ATU3 | GGACAAACCACCCATTACATCAGGATCCGGTGGAGCC |
| | MV leader: nt | ATCAGAGGAATCAAACACATTATTATAGTACCAATCCC |
| | 1-55 55 | TGGAGATTCCTCAATTACCACTCGATCCAGACTTCTGG |
| | MV N ORF: nt | ACCGGTTGGTGAGGTTAATTGGAAACCCGGATGTGAGC |
| | 108-1,685 | GGGCCCAAACTAACAGGGGCACTAATAGGTATATTATC |
| | MV P ORF: nt | CTTATTTGTGGAGTCTCCAGGTCAATTGATTCAGAGGA |
| | 1,807-3,330 | TCACCGATGACCCTGACGTTAGCATAAGGCTGTTAGAG |
| | MV M ORF: nt | GTTGTCCAGAGTGACCAGTCACAATCTGGCCTTACCTT |
| | 3,438-4,445 | CGCATCAAGAGGTACCAACATGGAGGATGAGGCGGAC |
| | MV F ORF: nt | CAATACTTTTCACATGATGATCCAATTAGTAGTGATCA |
| | 5,449-7,110 | ATCCAGGTTCGGATGGTTCGGGAACAAGGAAATCTCA |
| | MV H ORF: nt | GATATTGAAGTGCAAGACCCTGAGGGATTCAACATGAT |
| | 7,271-9,124 | TCTGGGTACCATCCTAGCCCAAATTTGGGTCTTGCTCG |
| | ATU upstream | CAAAGGCGGTTACGGCCCCAGACACGGCAGCTGATTC |
| | motif: nt 9,180- | GGAGCTAAGAAGGTGGATAAAGTACACCCAACAAAGA |
| | 9,257 | AGGGTAGTTGGTGAATTTAGATTGGAGAGAAAATGGTT |
| | GE of N gene: | GGATGTGGTGAGGAACAGGATTGCCGAGGACCTCTCCT |
| | nt 9,196-9,206 | TACGCCGATTCATGGTCGCTCTAATCCTGGATATCAAG |
| | Conserved | AGAACACCCGGAAACAAACCCAGGATTGCTGAAATGA |
| | trinucleotide | TATGTGACATTGATACATATATCGTAGAGGCAGGATTA |
| | motif: nt 9,207- | GCCAGTTTTATCCTGACTATTAAGTTTGGGATAGAAAC |
| | 9,209 | TATGTATCCTGCTCTTGGACTGCATGAATTTGCTGGTGA |
| | GS of P gene: | GTTATCCACACTTGAGTCCTTGATGAACCTTTACCAGC |
| | nt 9,210-9,226 | AAATGGGGGAAACTGCACCCTACATGGTAATCCTGGA |
| | Kozak | GAACTCAATTCAGAACAAGTTCAGTGCAGGATCATACC |
| | sequence: nt | CTCTGCTCTGGAGCTATGCCATGGGAGTAGGAGTGGAA |
| | 9,252-9,260 | CTTGAAAACTCCATGGGAGGTTTGAACTTTGGCCGATC |
| | EBV gp42 | TTACTTTGATCCAGCATATTTTAGATTAGGGCAAGAGA |
| | ORF: nt 9,258- | TGGTAAGGAGGTCAGCTGGAAAGGTCAGTTCCACATTG |
| | 9,929 | GCATCTGAACTCGGTATCACTGCCGAGGATGCAAGGCT |
| | ATUa motif: nt | TGTTTCAGAGATTGCAATGCATACTACTGAGGACAAGA |
| | 9,930-10,073 | TCAGTAGAGCGGTTGGACCCAGACAAGCCCAAGTATC |
| | GE of P gene: | ATTTCTACACGGTGATCAAAGTGAGAATGAGCTACCGA |
| | nt 9,997-10,007 | GATTGGGGGGCAAGGAAGATAGGAGGGTCAAACAGAG |
| | Conserved | TCGAGGAGAAGCCAGGGAGAGCTACAGAGAAACCGGG |
| | trinucleotide | CCCAGCAGAGCAAGTGATGCGAGAGCTGCCCATCTTCC |
| | motif: nt | AACCGGCACACCCCTAGACATTGACACTGCAACGGAG |
| | 10,008-10,010 | TCCAGCCAAGATCCGCAGGACAGTCGAAGGTCAGCTG |
| | GS of P gene: | ACGCCCTGCTTAGGCTGCAAGCCATGGCAGGAATCTCG |
| | nt 10,011- | GAAGAACAAGGCTCAGACACGGACACCCCTATAGTGT |
| | 10,027 | ACAATGACAGAAATCTTCTAGACTAGGTGCGAGAGGC |
| | EBV gH ORF: | CGAGGGCCAGAACAACATCCGCCTACCATCCATCATTG |
| | nt 10,074- | TTATAAAAAACTTAGGAACCAGGTCCACACAGCCGCC |
| | 12,197 | AGCCCATCAACCATCCACTCCCACGATTGGAGCCAATG |
| | ATUc motif: nt | GCAGAAGAGCAGGCACGCCATGTCAAAAACGGACTGG |
| | 12,198-12,299 | AATGCATCCGGGCTCTCAAGGCCGAGCCCATCGGCTCA |
| | GE of N gene: | CTGGCCATCGAGGAAGCTATGGCAGCATGGTCAGAAA |
| | nt 12,249- | TATCAGACAACCCAGGACAGGAGCGAGCCACCTGCAG |
| | 12,259 | GGAAGAGAAGGCAGGCAGTTCGGGTCTCAGCAAACCA |
| | Conserved | TGCCTCTCAGCAATTGGATCAACTGAAGGCGGTGCACC |
| | trinucleotide | TCGCATCCGCGGTCAGGGACCTGGAGAGAGCGATGAC |
| | motif: nt | GACGCTGAAACTTTGGGAATCCCCCCAAGAAATCTCCA |
| | 12,260-12,262 | GGCATCAAGCACTGGGTTACAGTGTTATTACGTTTATG |
| | Consensus GS | ATCACAGCGGTGAAGCGGTTAAGGGAATCCAAGATGC |
| | motif: nt | TGACTCTATCATGGTTCAATCAGGCCTTGATGGTGATA |
| | 12,263-12,279 | GCACCCTCTCAGGAGGAGACAATGAATCTGAAAACAG |
| | EBV gL ORF: | CGATGTGGATATTGGCGAACCTGATACCGAGGGATATG |
| | nt 12,300- | CTATCACTGACCGGGGATCTGCTCCCATCTCTATGGGG |
| | 12,713 | TTCAGGGCTTCTGATGTTGAAACTGCAGAAGGAGGGG |
| | ATU | AGATCCACGAGCTCCTGAGACTCCAATCCAGAGGCAA |
| | downstream | CAACTTTCCGAAGCTTGGGAAAACTCTCAATGTTCCTC |
| | motif: nt | CGCCCCCGGACCCCGGTAGGGCCAGCACTTCCGGGAC |
| | 12,714-12,749 | ACCCATTAAAAAGGGCACAGACGCGAGATTAGCCTCA |
| | MV L ORF: nt | TTTGGAACGGAGATCGCGTCTTTATTGACAGGTGGTGC |
| | 12,804-19,355 | AACCCAATGTGCTCGAAAGTCACCCTCGGAACCATCAG |
| | MV trailer: nt | GGCCAGGTGCACCTGCGGGGAATGTCCCCGAGTGTGTG |
| | 19,428-19,464 | AGCAATGCCGCACTGATACAGGAGTGGACACCCGAAT |
| | | CTGGTACCACAATCTCCCCGAGATCCCAGAATAATGAA |
| | | GAAGGGGGAGACTATTATGATGATGAGCTGTTCTCTGA |
| | | TGTCCAAGATATTAAAACAGCCTTGGCCAAAATACACG |
| | | AGGATAATCAGAAGATAATCTCCAAGCTAGAATCACT |
| | | GCTGTTATTGAAGGGAGAAGTTGAGTCAATTAAGAAG |
| | | CAGATCAACAGGCAAAATATCAGCATATCCACCCTGG |
| | | AAGGACACCTCTCAAGCATCATGATCGCCATTCCTGGA |
| | | CTTGGGAAGGATCCCAACGACCCCACTGCAGATGTCGA |
| | | AATCAATCCCGACTTGAAACCCATCATAGGCAGAGATT |
| | | CAGGCCGAGCACTGGCCGAAGTTCTCAAGAAACCCGTT |
| | | GCCAGCCGACAACTCCAAGGAATGACAAATGGACGGA |
| | | CCAGTTCCAGAGGACAGCTGCTGAAGGAATTTCAGCTA |
| | | AAGCCGATCGGGAAAAAGATGAGCTCAGCCGTCGGGT |
| | | TTGTTCCTGACACCGGCCCTGCATCACGCAGTGTAATC |
| | | CGCTCCATTATAAAATCCAGCCGGCTAGAGGAGGATCG |
| | | GAAGCGTTACCTGATGACTCTCCTTGATGATATCAAAG |
| | | GAGCCAATGATCTTGCCAAGTTCCACCAGATGCTGATG |
| | | AAGATAATAATGAAGTAGCTACAGCTCAACTTACCTGC |
| | | CAACCCCATGCCAGTCGACCCAACTAGTACAACCTAAA |
| | | TCCATTATAAAAAACTTAGGAGCAAAGTGATTGCCTCC |
| | | CAAGGTCCACAATGACAGAGACCTACGACTTCGACAA |
| | | GTCGGCATGGGACATCAAAGGGTCGATCGCTCCGATAC |
| | | AACCCACCACCTACAGTGATGGCAGGCTGGTGCCCCAG |
| | | GTCAGAGTCATAGATCCTGGTCTAGGCGACAGGAAGG |
| | | ATGAATGCTTTATGTACATGTTTCTGCTGGGGGTTGTTG |
| | | AGGACAGCGATTCCCTAGGGCCTCCAATCGGGCGAGC |
| | | ATTTGGGTTCCTGCCCTTAGGTGTTGGCAGATCCACAG |
| | | CAAAGCCCGAAAAACTCCTCAAAGAGGCCACTGAGCT |
| | | TGACATAGTTGTTAGACGTACAGCAGGGCTCAATGAAA |
| | | AACTGGTGTTCTACAACAACACCCCACTAACTCTCCTC |
| | | ACACCTTGGAGAAAGGTCCTAACAACAGGGAGTGTCTT |
| | | CAACGCAAACCAAGTGTGCAATGCGGTTAATCTGATAC |
| | | CGCTCGATACCCCGCAGAGGTTCCGTGTTGTTTATATG |
| | | AGCATCACCCGTCTTTCGGATAACGGGTATTACACCGT |
| | | TCCTAGAAGAATGCTGGAATTCAGATCGGTCAATGCAG |
| | | TGGCCTTCAACCTGCTGGTGACCCTTAGGATTGACAAG |
| | | GCGATAGGCCCTGGGAAGATCATCGACAATACAGAGC |
| | | AACTTCCTGAGGCAACATTTATGGTCCACATCGGGAAC |
| | | TTCAGGAGAAAGAAGAGTGAAGTCTACTCTGCCGATTA |
| | | TTGCAAAATGAAAATCGAAAAGATGGGCCTGGTTTTTG |
| | | CACTTGGTGGGATAGGGGGCACCAGTCTTCACATTAGA |
| | | AGCACAGGCAAAATGAGCAAGACTCTCCATGCACAAC |
| | | TCGGGTTCAAGAAGACCTTATGTTACCCGCTGATGGAT |
| | | ATCAATGAAGACCTTAATCGATTACTCTGGAGGAGCAG |
| | | ATGCAAGATAGTAAGAATCCAGGCAGTTTTGCAGCCAT |
| | | CAGTTCCTCAAGAATTCCGCATTTACGACGACGTGATC |
| | | ATAAATGATGACCAAGGACTATTCAAAGTTCTGTAGAC |
| | | CGTAGTGCCCAGCAATGCCCGAAAACGACCCCCCTCAC |
| | | AATGACAGCCAGAAGGCCCGGACAAAAAAGCCCCCTC |
| | | CGAAAGACTCCACGGACCAAGCGAGAGGCCAGCCAGC |
| | | AGCCGACGGCAAGCGCGAACACCAGGCGGCCCCAGCA |
| | | CAGAACAGCCCTGACACAAGGCCACCACCAGCCACCC |
| | | CAATCTGCATCCTCCTCGTGGGACCCCCGAGGACCAAC |
| | | CCCCAAGGCTGCCCCCGATCCAAACCACCAACCGCATC |
| | | CCCACCACCCCCGGGAAAGAAACCCCCAGCAATTGGA |
| | | AGGCCCCTCCCCCTCTTCCTCAACACAAGAACTCCACA |
| | | ACCGAACCGCACAAGCGACCGAGGTGACCCAACCGCA |
| | | GGCATCCGACTCCCTAGACAGATCCTCTCTCCCCGGCA |
| | | AACTAAACAAAACTTAGGGCCAAGGAACATACACACC |
| | | CAACAGAACCCAGACCCCGGCCCACGGCGCCGCGCCC |
| | | CCAACCCCCGACAACCAGAGGGAGCCCCCAACCAATC |
| | | CCGCCGGCTCCCCCGGTGCCCACAGGCAGGGACACCA |
| | | ACCCCCGAACAGACCCAGCACCCAACCATCGACAATC |
| | | CAAGACGGGGGGGCCCCCCCAAAAAAAGGCCCCCAGG |
| | | GGCCGACAGCCAGCACCGCGAGGAAGCCCACCCACCC |
| | | CACACACGACCACGGCAACCAAACCAGAACCCAGACC |
| | | ACCCTGGGCCACCAGCTCCCAGACTCGGCCATCACCCC |
| | | GCAGAAAGGAAAGGCCACAACCCGCGCACCCCAGCCC |
| | | CGATCCGGCGGGGAGCCACCCAACCCGAACCAGCACC |
| | | CAAGAGCGATCCCCGAAGGACCCCCGAACCGCAAAGG |
| | | ACATCAGTATCCCACAGCCTCTCCAAGTCCCCCGGTCT |
| | | CCTCCTCTTCTCGAAGGGACCAAAAGATCAATCCACCA |
| | | CACCCGACGACACTCAACTCCCCACCCCTAAAGGAGAC |
| | | ACCGGGAATCCCAGAATCAAGACTCATCCAATGTCCAT |
| | | CATGGGTCTCAAGGTGAACGTCTCTGCCATATTCATGG |
| | | CAGTACTGTTAACTCTCCAAACACCCACCGGTCAAATC |
| | | CATTGGGGCAATCTCTCTAAGATAGGGGTGGTAGGAAT |
| | | AGGAAGTGCAAGCTACAAAGTTATGACTCGTTCCAGCC |
| | | ATCAATCATTAGTCATAAAATTAATGCCCAATATAACT |
| | | CTCCTCAATAACTGCACGAGGGTAGAGATTGCAGAATA |
| | | CAGGAGACTACTGAGAACAGTTTTGGAACCAATTAGA |
| | | GATGCACTTAATGCAATGACCCAGAATATAAGACCGGT |
| | | TCAGAGTGTAGCTTCAAGTAGGAGACACAAGAGATTT |
| | | GCGGGAGTAGTCCTGGCAGGTGCGGCCCTAGGCGTTGC |
| | | CACAGCTGCTCAGATAACAGCCGGCATTGCACTTCACC |
| | | AGTCCATGCTGAACTCTCAAGCCATCGACAATCTGAGA |
| | | GCGAGCCTGGAAACTACTAATCAGGCAATTGAGACAA |
| | | TCAGACAAGCAGGGCAGGAGATGATATTGGCTGTTCA |
| | | GGGTGTCCAAGACTACATCAATAATGAGCTGATACCGT |
| | | CTATGAACCAACTATCTTGTGATTTAATCGGCCAGAAG |
| | | CTCGGGCTCAAATTGCTCAGATACTATACAGAAATCCT |
| | | GTCATTATTTGGCCCCAGTTTACGGGACCCCATATCTG |
| | | CGGAGATATCTATCCAGGCTTTGAGCTATGCGCTTGGA |
| | | GGAGACATCAATAAGGTGTTAGAAAAGCTCGGATACA |
| | | GTGGAGGTGATTTACTGGGCATCTTAGAGAGCGGAGG |
| | | CATCAAGGCCCGGATAACTCACGTCGACACAGAGTCCT |
| | | ACTTCATTGTCCTCAGTATAGCCTATCCGACGCTGTCCG |
| | | AGATTAAGGGGGTGATTGTCCACCGGCTAGAGGGGGT |
| | | CTCGTACAACATAGGCTCTCAAGAGTGGTATACCACTG |
| | | TGCCCAAGTATGTTGCAACCCAAGGGTACCTTATCTCG |
| | | AATTTTGATGAGTCATCGTGTACTTTCATGCCAGAGGG |
| | | GACTGTGTGCAGCCAAAATGCCTTGTACCCGATGAGTC |
| | | CTCTGCTCCAAGAATGCCTCCGGGGGTACACCAAGTCC |
| | | TGTGCTCGTACACTCGTATCCGGGTCTTTTGGGAACCG |
| | | GTTCATTTTATCACAAGGGAACCTAATAGCCAATTGTG |
| | | CATCAATCCTTTGCAAGTGTTACACAACAGGAACGATC |
| | | ATTAATCAAGACCCTGACAAGATCCTAACATACATTGC |
| | | TGCCGATCACTGCCCGGTAGTCGAGGTGAACGGCGTGA |
| | | CCATCCAAGTCGGGAGCAGGAGGTATCCAGACGCTGT |
| | | GTACTTGCACAGAATTGACCTCGGTCCTCCCATATCAT |
| | | TGGAGAGGTTGGACGTAGGGACAAATCTGGGGAATGC |
| | | AATTGCTAAGTTGGAGGATGCCAAGGAATTGTTGGAGT |
| | | CATCGGACCAGATATTGAGGAGTATGAAAGGTTTATCG |
| | | AGCACTAGCATAGTCTACATCCTGATTGCAGTGTGTCT |
| | | TGGAGGGTTGATAGGGATCCCCGCTTTAATATGTTGCT |
| | | GCAGGGGGCGTTGTAACAAAAAGGGAGAACAAGTTGG |
| | | TATGTCAAGACCAGGCCTAAAGCCTGATCTTACGGGAA |
| | | CATCAAAATCCTATGTAAGGTCGCTCTGATCCTCTACA |
| | | ACTCTTGAAACACAAATGTCCCACAAGTCTCCTCTTCG |
| | | TCATCAAGCAACCACCGCACCCAGCATCAAGCCCACCT |
| | | GAAATTATCTCCGGCTTCCCTCTGGCCGAACAATATCG |
| | | GTAGTTAATCAAAACTTAGGGTGCAAGATCATCCACAA |
| | | TGTCACCACAACGAGACCGGATAAATGCCTTCTACAAA |
| | | GATAACCCCCATCCCAAGGGAAGTAGGATAGTCATTA |
| | | ACAGAGAACATCTTATGATTGATAGACCTTATGTTTTG |
| | | CTGGCTGTTCTGTTTGTCATGTTTCTGAGCTTGATCGGG |
| | | TTGCTAGCCATTGCAGGCATTAGACTTCATCGGGCAGC |
| | | CATCTACACCGCAGAGATCCATAAAAGCCTCAGCACCA |
| | | ATCTAGATGTAACTAACTCAATCGAGCATCAGGTCAAG |
| | | GACGTGCTGACACCACTCTTCAAAATCATCGGTGATGA |
| | | AGTGGGCCTGAGGACACCTCAGAGATTCACTGACCTAG |
| | | TGAAATTAATCTCTGACAAGATCAAGTTCCTTAATCCG |
| | | GATAGGGAGTACGACTTCAGAGATCTCACTTGGTGTAT |
| | | CAACCCGCCAGAGAGAATCAAATTGGATTATGATCAAT |
| | | ACTGTGCAGATGTGGCTGCTGAAGAGCTCATGAATGCA |
| | | TTGGTGAACTCAACTCTACTGGAGACCAGAACAACCAA |
| | | TCAGTTCCTAGCTGTCTCAAAGGGAAACTGCTCAGGGC |
| | | CCACTACAATCAGAGGTCAATTCTCAAACATGTCGCTG |
| | | TCCCTGTTAGACTTGTATTTAGGTCGAGGTTACAATGT |
| | | GTCATCTATAGTCACTATGACATCCCAGGGAATGTATG |
| | | GGGGAACTTACCTAGTGGAAAAGCCTAATCTGAGCAG |
| | | CAAAAGGTCAGAGTTGTCACAACTGAGCATGTACCGA |
| | | GTGTTTGAAGTAGGTGTTATCAGAAATCCGGGTTTGGG |
| | | GGCTCCGGTGTTCCATATGACAAACTATCTTGAGCAAC |
| | | CAGTCAGTAATGATCTCAGCAACTGTATGGTGGCTTTG |
| | | GGGGAGCTCAAACTCGCAGCCCTTTGTCACGGGGAAG |
| | | ATTCTATCACAATTCCCTATCAGGGATCAGGGAAAGGT |
| | | GTCAGCTTCCAGCTCGTCAAGCTAGGTGTCTGGAAATC |
| | | CCCAACCGACATGCAATCCTGGGTCCCCTTATCAACGG |
| | | ATGATCCAGTGATAGACAGGCTTTACCTCTCATCTCAC |
| | | AGAGGTGTTATCGCTGACAATCAAGCAAAATGGGCTGT |
| | | CCCGACAACACGAACAGATGACAAGTTGCGAATGGAG |
| | | ACATGCTTCCAACAGGCGTGTAAGGGTAAAATCCAAG |
| | | CACTCTGCGAGAATCCCGAGTGGGCACCATTGAAGGAT |
| | | AACAGGATTCCTTCATACGGGGTCTTGTCTGTTGATCT |
| | | GAGTCTGACAGTTGAGCTTAAAATCAAAATTGCTTCGG |
| | | GATTCGGGCCATTGATCACACACGGTTCAGGGATGGAC |
| | | CTATACAAATCCAACCACAACAATGTGTATTGGCTGAC |
| | | TATCCCGCCAATGAAGAACCTAGCCTTAGGTGTAATCA |
| | | ACACATTGGAGTGGATACCGAGATTCAAGGTTAGTCCC |
| | | TACCTCTTCACTGTCCCAATTAAGGAAGCAGGCGAAGA |
| | | CTGCCATGCCCCAACATACCTACCTGCGGAGGTGGATG |
| | | GTGATGTCAAACTCAGTTCCAATCTGGTGATTCTACCT |
| | | GGTCAAGATCTCCAATATGTTTTGGCAACCTACGATAC |
| | | TTCCAGGGTTGAACATGCTGTGGTTTATTACGTTTACA |
| | | GCCCAAGCCGCTCATTTTCTTACTTTTATCCTTTTAGGT |
| | | TGCCTATAAAGGGGGTCCCCATCGAATTACAAGTGGAA |
| | | TGCTTCACATGGGACCAAAAACTCTGGTGCCGTCACTT |
| | | CTGTGTGCTTGCGGACTCAGAATCTGGTGGACATATCA |
| | | CTCACTCTGGGATGGTGGGCATGGGAGTCAGCTGCACA |
| | | GTCACCCGGGAAGATGGAACCAATCGCAGATAGGGCT |
| | | GCTAGTGAACCAATCACATGATGTCACCCAGACATCAG |
| | | GCATACCCACTAGTCTACCCTCCATCATTGTTATAAAA |
| | | AACTTAGGAACCAGGTCCACACAGCCGCCAGCCCATC |
| | | AACGCGTACGGCCACCATGGTGAGTTTTAAGCAGGTCC |
| | | GGGTACCTCTGTTCACCGCCATTGCGCTGGTGATTGTTC |
| | | TGCTGTTGGCCTACTTTCTTCCACCTAGAGTCAGAGGA |
| | | GGTGGCAGGGTTGCTGCAGCTGCTATTACCTGGGTACC |
| | | GAAACCCAACGTGGAGGTTTGGCCAGTGGATCCGCCCC |
| | | CACCAGTGAACTTTAATAAAACAGCCGAACAAGAATA |
| | | CGGGGATAAAGAAGTAAAGCTGCCACACTGGACCCCT |
| | | ACCCTCCACACTTTCCAGGTGCCTCAAAACTATACAAA |
| | | GGCCAATTGCACATACTGTAACACAAGAGAATACACTT |
| | | TTTCTTATAAAGGGTGCTGCTTCTACTTCACTAAGAAG |
| | | AAGCATACTTGGAACGGATGTTTCCAGGCATGCGCTGA |
| | | GTTGTATCCCTGTACCTATTTCTACGGTCCTACACCAGA |
| | | CATTCTGCCGGTCGTGACTCGCAATCTGAACGCCATAG |
| | | AATCTCTGTGGGTCGGGGTGTATCGGGTTGGGGAAGGA |
| | | AATTGGACTAGCCTGGACGGCGGTACTTTCAAGGTGTA |
| | | CCAAATATTCGGCAGTCACTGTACTTACGTGTCAAAAT |
| | | TCTCAACAGTTCCCGTATCCCACCACGAGTGTTCTTTTT |
| | | TGAAGCCTTGCCTGTGCGTGTCACAGAGGTCAAATTCC |
| | | TAATTCGAACTACAGCTCAACTTACCTGCCAACCCCAT |
| | | GCCAGTCGACCCAACTAGTACAACCTAAATCCATTATA |
| | | AAAAACTTAGGAACCAGGTCCACACAGCCGCCAGCCC |
| | | ATCAACCATCCACTCCCACGATTGGAGGCCGGCCATGC |
| | | AACTCCTCTGCGTCTTTTGCTTGGTGCTTTTGTGGGAGG |
| | | TAGGAGCGGCCTCATTGTCAGAAGTTAAGCTGCATCTC |
| | | GATATTGAAGGCCACGCTTCACATTACACTATCCCTTG |
| | | GACCGAACTCATGGCTAAGGTGCCGGGTTTGTCTCCGG |
| | | AAGCACTTTGGAGAGAGGCAAACGTTACCGAAGACCT |
| | | GGCCTCAATGCTGAATCGCTACAAGCTGATATACAAGA |
| | | CTTCCGGAACCCTGGGTATAGCACTCGCAGAGCCCGTC |
| | | GATATCCCCGCTGTCAGCGAAGGTAGCATGCAGGTGG |
| | | ACGCTAGTAAAGTTCACCCAGGAGTTATCTCAGGTCTG |
| | | AATAGCCCAGCTTGTATGCTGAGCGCTCCACTGGAGAA |
| | | ACAGCTCTTCTACTATATAGGCACAATGCTCCCTAACA |
| | | CTCGACCTCACAGCTACGTCTTCTATCAGCTGCGCTGTC |
| | | ACCTGTCATACGTAGCCTTGTCCATTAATGGTGATAAG |
| | | TTCCAGTACACAGGTGCCATGACCTCCAAGTTCCTTAT |
| | | GGGGACCTATAAACGGGTCACTGAGAAAGGAGACGAG |
| | | CATGTCCTTAGTTTGGTTTTTGGGAAGACAAAAGACCT |
| | | GCCGGATCTGAGGGGCCCGTTCTCTTACCCCTCACTCA |
| | | CATCTGCACAGAGTGGGGACTATTCATTGGTGATTGTG |
| | | ACAACCTTCGTTCACTACGCCAATTTTCACAACTATTTT |
| | | GTCCCGAACCTTAAAGACATGTTTTCACGCGCCGTGAC |
| | | CATGACAGCTGCTTCCTACGCTAGGTATGTGCTCCAAA |
| | | AGCTGGTGCTGCTGGAGATGAAGGGCGGATGCAGGGA |
| | | ACCTGAACTGGACACAGAGACTCTCACCACTATGTTCG |
| | | AGGTTTCCGTGGCGTTCTTCAAGGTGGGTCATGCTGTG |
| | | GGGGAGACTGGGAACGGCTGCGTAGATCTTAGGTGGC |
| | | TGGCTAAATCTTTCTTCGAGCTGACCGTGTTGAAAGAT |
| | | ATAATTGGAATTTGCTACGGGGCTACGGTTAAAGGAAT |
| | | GCAGTCTTACGGGCTGGAAAGGCTGGCCGCTATGCTCA |
| | | TGGCTACTGTGAAGATGGAGGAACTCGGCCACCTCACC |
| | | ACAGAAAAACAGGAGTACGCTTTGCGCCTGGCCACTGT |
| | | TGGCTACCCTAAGGCAGGCGTTTACTCTGGATTGATTG |
| | | GGGGGGCCACATCTGTGCTCTTGTCAGCCTACAATCGC |
| | | CATCCCCTGTTCCAGCCTCTGCACACGGTGATGCGAGA |
| | | GACTTTGTTTATTGGCAGCCACGTTGTGCTGAGAGAAC |
| | | TGCGCCTTAACGTTACCACTCAGGGACCTAACCTTGCG |
| | | CTGTATCAGCTGCTTAGCACCGCTTTGTGCTCCGCTCTT |
| | | GAAATTGGAGAAGTACTCAGAGGGCTCGCACTGGGTA |
| | | CTGAGTCAGGGCTGTTCTCCCCCTGCTATCTGTCTCTCC |
| | | GGTTCGACCTCACTCGGGATAAGCTGTTGTCAATGGCT |
| | | CCGCAGGAAGCCACATTGGACCAGGCCGCAGTGAGTA |
| | | ACGCAGTGGACGGGTTCCTGGGTAGACTGTCTCTTGAG |
| | | AGGGAAGACAGAGACGCTTGGCATCTGCCTGCGTATA |
| | | AGTGTGTTGATAGACTGGACAAAGTACTGATGATCATT |
| | | CCCCTGATTAATGTGACATTTATTATATCTAGTGACCG |
| | | AGAGGTGAGAGGCTCCGCTCTCTATGAAGCATCCACAA |
| | | CGTACTTGAGCAGTTCCCTGTTTCTGTCACCGGTTATTA |
| | | TGAACAAATGTTCTCAGGGAGCTGTTGCTGGTGAACCG |
| | | CGACAGATTCCAAAGATTCAGAACTTCACTAGGACACA |
| | | AAAGAGCTGCATTTTCTGCGGGTTCGCACTGCTGAGTT |
| | | ACGACGAAAAGGAGGGACTGGAGACTACTACATATAT |
| | | TACTTCACAGGAGGTGCAGAATAGCATCCTGAGCTCCA |
| | | ATTATTTTGACTTTGACAACCTCCATGTCCATTATTTGC |
| | | TGTTGACCACAAACGGCACTGTGATGGAGATCGCTGGC |
| | | CTGTACGAGGAGCGAGCCCACGTAGTACTGGCCATTAT |
| | | TCTGTACTTCATCGCTTTCGCACTTGGGATTTTCCTCGT |
| | | CCACAAAATTGTTATGTTTTTCCTCTAATGACTATTCGA |
| | | ACTACTCAGCTAGTCTACGCGCCGTAGCCTACCCTCCA |
| | | TCATTGTTATAAAAAACTTAGGATCCAAGAGCATACAG |
| | | CCGCCGATAAGGTCCACAATGAGAGCCGTAGGTGTCTT |
| | | TCTGGCTATTTGTCTGGTAACAATCTTCGTGCTGCCCAC |
| | | TTGGGGCAACTGGGCCTATCCTTGCTGCCATGTTACAC |
| | | AGTTGAGAGCGCAGCACCTCCTGGCGTTGGAGAACATT |
| | | TCAGACATCTACCTGGTGAGTAACCAAACCTGCGACGG |
| | | GTTCTCTTTGGCCAGTCTCAATAGTCCAAAAAACGGAA |
| | | GCAATCAGCTGGTAATTTCACGGTGTGCAAACGGACTT |
| | | AACGTGGTCTCTTTTTTTATATCAATCCTGAAACGCAGT |
| | | TCATCTGCATTGACCGGCCACTTGAGGGAGCTCTTGAC |
| | | CACTCTCGAGACACTTTACGGGTCATTCTCCGTGGAGG |
| | | ATCTGTTCGGGGCAAATCTGAACCGGTACGCCTGGCAT |
| | | AGAGGCGGGTAAGCGCGCAGCGCTTAGACGTCTCGCG |
| | | ATCGATGCTAGTGTGAAATAGACATCAGAATTAAGAA |
| | | AAACGTAGGGTCCAAGTGGTTCCCCGTTATGGACTCGC |
| | | TATCTGTCAACCAGATCTTATACCCTGAAGTTCACCTA |
| | | GATAGCCCGATAGTTACCAATAAGATAGTAGCCATCCT |
| | | GGAGTATGCTCGAGTCCCTCACGCTTACAGCCTGGAGG |
| | | ACCCTACACTGTGTCAGAACATCAAGCACCGCCTAAAA |
| | | AACGGATTTTCCAACCAAATGATTATAAACAATGTGGA |
| | | AGTTGGGAATGTCATCAAGTCCAAGCTTAGGAGTTATC |
| | | CGGCCCACTCTCATATTCCATATCCAAATTGTAATCAG |
| | | GATTTATTTAACATAGAAGACAAAGAGTCAACGAGGA |
| | | AGATCCGTGAACTCCTCAAAAAGGGGAATTCGCTGTAC |
| | | TCCAAAGTCAGTGATAAGGTTTTCCAATGCTTAAGGGA |
| | | CACTAACTCACGGCTTGGCCTAGGCTCCGAATTGAGGG |
| | | AGGACATCAAGGAGAAAGTTATTAACTTGGGAGTTTAC |
| | | ATGCACAGCTCCCAGTGGTTTGAGCCCTTTCTGTTTTGG |
| | | TTTACAGTCAAGACTGAGATGAGGTCAGTGATTAAATC |
| | | ACAAACCCATACTTGCCATAGGAGGAGACACACACCT |
| | | GTATTCTTCACTGGTAGTTCAGTTGAGTTGCTAATCTCT |
| | | CGTGACCTTGTTGCTATAATCAGTAAAGAGTCTCAACA |
| | | TGTATATTACCTGACATTTGAACTGGTTTTGATGTATTG |
| | | TGATGTCATAGAGGGGAGGTTAATGACAGAGACCGCT |
| | | ATGACTATTGATGCTAGGTATACAGAGCTTCTAGGAAG |
| | | AGTCAGATACATGTGGAAACTGATAGATGGTTTCTTCC |
| | | CTGCACTCGGGAATCCAACTTATCAAATTGTAGCCATG |
| | | CTGGAGCCTCTTTCACTTGCTTACCTGCAGCTGAGGGA |
| | | TATAACAGTAGAACTCAGAGGTGCTTTCCTTAACCACT |
| | | GCTTTACTGAAATACATGATGTTCTTGACCAAAACGGG |
| | | TTTTCTGATGAAGGTACTTATCATGAGTTAACTGAAGC |
| | | TCTAGATTACATTTTCATAACTGATGACATACATCTGA |
| | | CAGGGGAGATTTTCTCATTTTTCAGAAGTTTCGGCCAC |
| | | CCCAGACTTGAAGCAGTAACGGCTGCTGAAAATGTTAG |
| | | GAAATACATGAATCAGCCTAAAGTCATTGTGTATGAGA |
| | | CTCTGATGAAAGGTCATGCCATATTTTGTGGAATCATA |
| | | ATCAACGGCTATCGTGACAGGCACGGAGGCAGTTGGC |
| | | CACCGCTGACCCTCCCCCTGCATGCTGCAGACACAATC |
| | | CGGAATGCTCAAGCTTCAGGTGAAGGGTTAACACATG |
| | | AGCAGTGCGTTGATAACTGGAAATCTTTTGCTGGAGTG |
| | | AAATTTGGCTGCTTTATGCCTCTTAGCCTGGATAGTGAT |
| | | CTGACAATGTACCTAAAGGACAAGGCACTTGCTGCTCT |
| | | CCAAAGGGAATGGGATTCAGTTTACCCGAAAGAGTTCC |
| | | TGCGTTACGACCCTCCCAAGGGAACCGGGTCACGGAG |
| | | GCTTGTAGATGTTTTCCTTAATGATTCGAGCTTTGACCC |
| | | ATATGATGTGATAATGTATGTTGTAAGTGGAGCTTACC |
| | | TCCATGACCCTGAGTTCAACCTGTCTTACAGCCTGAAA |
| | | GAAAAGGAGATCAAGGAAACAGGTAGACTTTTTGCTA |
| | | AAATGACTTACAAAATGAGGGCATGCCAAGTGATTGCT |
| | | GAAAATCTAATCTCAAACGGGATTGGCAAATATTTTAA |
| | | GGACAATGGGATGGCCAAGGATGAGCACGATTTGACT |
| | | AAGGCACTCCACACTCTAGCTGTCTCAGGAGTCCCCAA |
| | | AGATCTCAAAGAAAGTCACAGGGGGGGGCCAGTCTTA |
| | | AAAACCTACTCCCGAAGCCCAGTCCACACAAGTACCA |
| | | GGAACGTGAGAGCAGCAAAAGGGTTTATAGGGTTCCC |
| | | TCAAGTAATTCGGCAGGACCAAGACACTGATCATCCGG |
| | | AGAATATGGAAGCTTACGAGACAGTCAGTGCATTTATC |
| | | ACGACTGATCTCAAGAAGTACTGCCTTAATTGGAGATA |
| | | TGAGACCATCAGCTTGTTTGCACAGAGGCTAAATGAGA |
| | | TTTACGGATTGCCCTCATTTTTCCAGTGGCTGCATAAGA |
| | | GGCTTGAGACCTCTGTCCTGTATGTAAGTGACCCTCAT |
| | | TGCCCCCCCGACCTTGACGCCCATATCCCGTTATATAA |
| | | AGTCCCCAATGATCAAATCTTCATTAAGTACCCTATGG |
| | | GAGGTATAGAAGGGTATTGTCAGAAGCTGTGGACCAT |
| | | CAGCACCATTCCCTATCTATACCTGGCTGCTTATGAGA |
| | | GCGGAGTAAGGATTGCTTCGTTAGTGCAAGGGGACAA |
| | | TCAGACCATAGCCGTAACAAAAAGGGTACCCAGCACA |
| | | TGGCCCTACAACCTTAAGAAACGGGAAGCTGCTAGAG |
| | | TAACTAGAGATTACTTTGTAATTCTTAGGCAAAGGCTA |
| | | CATGATATTGGCCATCACCTCAAGGCAAATGAGACAAT |
| | | TGTTTCATCACATTTTTTTGTCTATTCAAAAGGAATATA |
| | | TTATGATGGGCTACTTGTGTCCCAATCACTCAAGAGCA |
| | | TCGCAAGATGTGTATTCTGGTCAGAGACTATAGTTGAT |
| | | GAAACAAGGGCAGCATGCAGTAATATTGCTACAACAA |
| | | TGGCTAAAAGCATCGAGAGAGGTTATGACCGTTACCTT |
| | | GCATATTCCCTGAACGTCCTAAAAGTGATACAGCAAAT |
| | | TCTGATCTCTCTTGGCTTCACAATCAATTCAACCATGAC |
| | | CCGGGATGTAGTCATACCCCTCCTCACAAACAACGACC |
| | | TCTTAATAAGGATGGCACTGTTGCCCGCTCCTATTGGG |
| | | GGGATGAATTATCTGAATATGAGCAGGCTGTTTGTCAG |
| | | AAACATCGGTGATCCAGTAACATCATCAATTGCTGATC |
| | | TCAAGAGAATGATTCTCGCCTCACTAATGCCTGAAGAG |
| | | ACCCTCCATCAAGTAATGACACAACAACCGGGGGACT |
| | | CTTCATTCCTAGACTGGGCTAGCGACCCTTACTCAGCA |
| | | AATCTTGTATGTGTCCAGAGCATCACTAGACTCCTCAA |
| | | GAACATAACTGCAAGGTTTGTCCTGATCCATAGTCCAA |
| | | ACCCAATGTTAAAAGGATTATTCCATGATGACAGTAAA |
| | | GAAGAGGACGAGGGACTGGCGGCATTCCTCATGGACA |
| | | GGCATATTATAGTACCTAGGGCAGCTCATGAAATCCTG |
| | | GATCATAGTGTCACAGGGGCAAGAGAGTCTATTGCAG |
| | | GCATGCTGGATACCACAAAAGGCTTGATTCGAGCCAGC |
| | | ATGAGGAAGGGGGGGTTAACCTCTCGAGTGATAACCA |
| | | GATTGTCCAATTATGACTATGAACAATTCAGAGCAGGG |
| | | ATGGTGCTATTGACAGGAAGAAAGAGAAATGTCCTCA |
| | | TTGACAAAGAGTCATGTTCAGTGCAGCTGGCGAGAGCT |
| | | CTAAGAAGCCATATGTGGGCGAGGCTAGCTCGAGGAC |
| | | GGCCTATTTACGGCCTTGAGGTCCCTGATGTACTAGAA |
| | | TCTATGCGAGGCCACCTTATTCGGCGTCATGAGACATG |
| | | TGTCATCTGCGAGTGTGGATCAGTCAACTACGGATGGT |
| | | TTTTTGTCCCCTCGGGTTGCCAACTGGATGATATTGACA |
| | | AGGAAACATCATCCTTGAGAGTCCCATATATTGGTTCT |
| | | ACCACTGATGAGAGAACAGACATGAAGCTTGCCTTCGT |
| | | AAGAGCCCCAAGTCGATCCTTGCGATCTGCTGTTAGAA |
| | | TAGCAACAGTGTACTCATGGGCTTACGGTGATGATGAT |
| | | AGCTCTTGGAACGAAGCCTGGTTGTTGGCTAGGCAAAG |
| | | GGCCAATGTGAGCCTGGAGGAGCTAAGGGTGATCACT |
| | | CCCATCTCAACTTCGACTAATTTAGCGCATAGGTTGAG |
| | | GGATCGTAGCACTCAAGTGAAATACTCAGGTACATCCC |
| | | TTGTCCGAGTGGCGAGGTATACCACAATCTCCAACGAC |
| | | AATCTCTCATTTGTCATATCAGATAAGAAGGTTGATAC |
| | | TAACTTTATATACCAACAAGGAATGCTTCTAGGGTTGG |
| | | GTGTTTTAGAAACATTGTTTCGACTCGAGAAAGATACC |
| | | GGATCATCTAACACGGTATTACATCTTCACGTCGAAAC |
| | | AGATTGTTGCGTGATCCCGATGATAGATCATCCCAGGA |
| | | TACCCAGCTCCCGCAAGCTAGAGCTGAGGGCAGAGCT |
| | | ATGTACCAACCCATTGATATATGATAATGCACCTTTAA |
| | | TTGACAGAGATGCAACAAGGCTATACACCCAGAGCCA |
| | | TAGGAGGCACCTTGTGGAATTTGTTACATGGTCCACAC |
| | | CCCAACTATATCACATTTTAGCTAAGTCCACAGCACTA |
| | | TCTATGATTGACCTGGTAACAAAATTTGAGAAGGACCA |
| | | TATGAATGAAATTTCAGCTCTCATAGGGGATGACGATA |
| | | TCAATAGTTTCATAACTGAGTTTCTGCTCATAGAGCCA |
| | | AGATTATTCACTATCTACTTGGGCCAGTGTGCGGCCAT |
| | | CAATTGGGCATTTGATGTACATTATCATAGACCATCAG |
| | | GGAAATATCAGATGGGTGAGCTGTTGTCATCGTTCCTT |
| | | TCTAGAATGAGCAAAGGAGTGTTTAAGGTGCTTGTCAA |
| | | TGCTCTAAGCCACCCAAAGATCTACAAGAAATTCTGGC |
| | | ATTGTGGTATTATAGAGCCTATCCATGGTCCTTCACTTG |
| | | ATGCTCAAAACTTGCACACAACTGTGTGCAACATGGTT |
| | | TACACATGCTATATGACCTACCTCGACCTGTTGTTGAA |
| | | TGAAGAGTTAGAAGAGTTCACATTTCTCTTGTGTGAAA |
| | | GCGACGAGGATGTAGTACCGGACAGATTCGACAACAT |
| | | CCAGGCAAAACACTTATGTGTTCTGGCAGATTTGTACT |
| | | GTCAACCAGGGACCTGCCCACCAATTCGAGGTCTAAGA |
| | | CCGGTAGAGAAATGTGCAGTTCTAACCGACCATATCAA |
| | | GGCAGAGGCTATGTTATCTCCAGCAGGATCTTCGTGGA |
| | | ACATAAATCCAATTATTGTAGACCATTACTCATGCTCT |
| | | CTGACTTATCTCCGGCGAGGATCGATCAAACAGATAAG |
| | | ATTGAGAGTTGATCCAGGATTCATTTTCGACGCCCTCG |
| | | CTGAGGTAAATGTCAGTCAGCCAAAGATCGGCAGCAA |
| | | CAACATCTCAAATATGAGCATCAAGGCTTTCAGACCCC |
| | | CACACGATGATGTTGCAAAATTGCTCAAAGATATCAAC |
| | | ACAAGCAAGCACAATCTTCCCATTTCAGGGGGCAATCT |
| | | CGCCAATTATGAAATCCATGCTTTCCGCAGAATCGGGT |
| | | TGAACTCATCTGCTTGCTACAAAGCTGTTGAGATATCA |
| | | ACATTAATTAGGAGATGCCTTGAGCCAGGGGAGGACG |
| | | GCTTGTTCTTGGGTGAGGGATCGGGTTCTATGTTGATC |
| | | ACTTATAAAGAGATACTTAAACTAAACAAGTGCTTCTA |
| | | TAATAGTGGGGTTTCCGCCAATTCTAGATCTGGTCAAA |
| | | GGGAATTAGCACCCTATCCCTCCGAAGTTGGCCTTGTC |
| | | GAACACAGAATGGGAGTAGGTAATATTGTCAAAGTGC |
| | | TCTTTAACGGGAGGCCCGAAGTCACGTGGGTAGGCAGT |
| | | GTAGATTGCTTCAATTTCATAGTTAGTAATATCCCTACC |
| | | TCTAGTGTGGGGTTTATCCATTCAGATATAGAGACCTT |
| | | GCCTGACAAAGATACTATAGAGAAGCTAGAGGAATTG |
| | | GCAGCCATCTTATCGATGGCTCTGCTCCTGGGCAAAAT |
| | | AGGATCAATACTGGTGATTAAGCTTATGCCTTTCAGCG |
| | | GGGATTTTGTTCAGGGATTTATAAGTTATGTAGGGTCT |
| | | CATTATAGAGAAGTGAACCTTGTATACCCTAGATACAG |
| | | CAACTTCATCTCTACTGAATCTTATTTGGTTATGACAGA |
| | | TCTCAAGGCTAACCGGCTAATGAATCCTGAAAAGATTA |
| | | AGCAGCAGATAATTGAATCATCTGTGAGGACTTCACCT |
| | | GGACTTATAGGTCACATCCTATCCATTAAGCAACTAAG |
| | | CTGCATACAAGCAATTGTGGGAGACGCAGTTAGTAGA |
| | | GGTGATATCAATCCTACTCTGAAAAAACTTACACCTAT |
| | | AGAGCAGGTGCTGATCAATTGCGGGTTGGCAATTAACG |
| | | GACCTAAGCTGTGCAAAGAATTGATCCACCATGATGTT |
| | | GCCTCAGGGCAAGATGGATTGCTTAATTCTATACTCAT |
| | | CCTCTACAGGGAGTTGGCAAGATTCAAAGACAACCAA |
| | | AGAAGTCAACAAGGGATGTTCCACGCTTACCCCGTATT |
| | | GGTAAGTAGCAGGCAACGAGAACTTATATCTAGGATC |
| | | ACCCGCAAATTCTGGGGGCACATTCTTCTTTACTCCGG |
| | | GAACAAAAAGTTGATAAATAAGTTTATCCAGAATCTCA |
| | | AGTCCGGCTATCTGATACTAGACTTACACCAGAATATC |
| | | TTCGTTAAGAATCTATCCAAGTCAGAGAAACAGATTAT |
| | | TATGACGGGGGGTTTGAAACGTGAGTGGGTTTTTAAGG |
| | | TAACAGTCAAGGAGACCAAAGAATGGTATAAGTTAGT |
| | | CGGATACAGTGCCCTGATTAAGGACTAATTGGTTGAAC |
| | | TCCGGAACCCTAATCCTGCCCTAGGTGGTTAGGCATTA |
| | | TTTGCAATATATTAAAGAAAACTTTGAAAATACGAAGT |
| | | TTCTATTCCCAGCTTTGTCTGGT |
| 86 | T-08 | ACCAAACAAAGTTGGGTAAGGATAGTTCAATCAATGA |
| | EBV_gH_ATUa_ | TCATCTTCTAGTGCACTTAGGATTCAAGATCCTATTATC |
| | gp42_ATUc_ | AGGGACAAGAGCAGGATTAGGGATATCCGAGATGGCC |
| | gL at ATU3 | ACACTTTTAAGGAGCTTAGCATTGTTCAAAAGAAACAA |
| | MV leader: nt | GGACAAACCACCCATTACATCAGGATCCGGTGGAGCC |
| | 1-55 | ATCAGAGGAATCAAACACATTATTATAGTACCAATCCC |
| | MV N ORF: nt | TGGAGATTCCTCAATTACCACTCGATCCAGACTTCTGG |
| | 108-1,685 | ACCGGTTGGTGAGGTTAATTGGAAACCCGGATGTGAGC |
| | MV P ORF: nt | GGGCCCAAACTAACAGGGGCACTAATAGGTATATTATC |
| | 1,807-3,330 | CTTATTTGTGGAGTCTCCAGGTCAATTGATTCAGAGGA |
| | MV M ORF: nt | TCACCGATGACCCTGACGTTAGCATAAGGCTGTTAGAG |
| | 3,438-4,445 | GTTGTCCAGAGTGACCAGTCACAATCTGGCCTTACCTT |
| | MV F ORF: nt | CGCATCAAGAGGTACCAACATGGAGGATGAGGCGGAC |
| | 5,449-7,110 | CAATACTTTTCACATGATGATCCAATTAGTAGTGATCA |
| | MV H ORF: nt | ATCCAGGTTCGGATGGTTCGGGAACAAGGAAATCTCA |
| | 7,271-9,124 | GATATTGAAGTGCAAGACCCTGAGGGATTCAACATGAT |
| | ATU upstream | TCTGGGTACCATCCTAGCCCAAATTTGGGTCTTGCTCG |
| | motif: nt 9,180- | CAAAGGCGGTTACGGCCCCAGACACGGCAGCTGATTC |
| | 9,257 | GGAGCTAAGAAGGTGGATAAAGTACACCCAACAAAGA |
| | GE of N gene: | AGGGTAGTTGGTGAATTTAGATTGGAGAGAAAATGGTT |
| | nt 9,196-9,206 | GGATGTGGTGAGGAACAGGATTGCCGAGGACCTCTCCT |
| | Conserved | TACGCCGATTCATGGTCGCTCTAATCCTGGATATCAAG |
| | trinucleotide | AGAACACCCGGAAACAAACCCAGGATTGCTGAAATGA |
| | motif: nt 9,207- | TATGTGACATTGATACATATATCGTAGAGGCAGGATTA |
| | 9,209 | GCCAGTTTTATCCTGACTATTAAGTTTGGGATAGAAAC |
| | GS of P gene: | TATGTATCCTGCTCTTGGACTGCATGAATTTGCTGGTGA |
| | nt 9,210-9,226 | GTTATCCACACTTGAGTCCTTGATGAACCTTTACCAGC |
| | Kozak | AAATGGGGGAAACTGCACCCTACATGGTAATCCTGGA |
| | sequence: nt | GAACTCAATTCAGAACAAGTTCAGTGCAGGATCATACC |
| | 9,252-9,260 | CTCTGCTCTGGAGCTATGCCATGGGAGTAGGAGTGGAA |
| | EBV gH ORF: | CTTGAAAACTCCATGGGAGGTTTGAACTTTGGCCGATC |
| | nt 9,258-11,381 | TTACTTTGATCCAGCATATTTTAGATTAGGGCAAGAGA |
| | ATUa motif: nt | TGGTAAGGAGGTCAGCTGGAAAGGTCAGTTCCACATTG |
| | 11,382-11,525 | GCATCTGAACTCGGTATCACTGCCGAGGATGCAAGGCT |
| | GE of P gene: | TGTTTCAGAGATTGCAATGCATACTACTGAGGACAAGA |
| | nt 11,449- | TCAGTAGAGCGGTTGGACCCAGACAAGCCCAAGTATC |
| | 11,459 | ATTTCTACACGGTGATCAAAGTGAGAATGAGCTACCGA |
| | Conserved | GATTGGGGGGCAAGGAAGATAGGAGGGTCAAACAGAG |
| | trinucleotide | TCGAGGAGAAGCCAGGGAGAGCTACAGAGAAACCGGG |
| | motif: nt | CCCAGCAGAGCAAGTGATGCGAGAGCTGCCCATCTTCC |
| | 11,460-11,462 | AACCGGCACACCCCTAGACATTGACACTGCAACGGAG |
| | GS of P gene: | TCCAGCCAAGATCCGCAGGACAGTCGAAGGTCAGCTG |
| | nt 11,463- | ACGCCCTGCTTAGGCTGCAAGCCATGGCAGGAATCTCG |
| | 11,479 | GAAGAACAAGGCTCAGACACGGACACCCCTATAGTGT |
| | EBV gp42 | ACAATGACAGAAATCTTCTAGACTAGGTGCGAGAGGC |
| | ORF: nt | CGAGGGCCAGAACAACATCCGCCTACCATCCATCATTG |
| | 11,526-12,197 | TTATAAAAAACTTAGGAACCAGGTCCACACAGCCGCC |
| | ATUc motif: nt | AGCCCATCAACCATCCACTCCCACGATTGGAGCCAATG |
| | 12,198-12,299 | GCAGAAGAGCAGGCACGCCATGTCAAAAACGGACTGG |
| | GE of N gene: | AATGCATCCGGGCTCTCAAGGCCGAGCCCATCGGCTCA |
| | nt 12,249- | CTGGCCATCGAGGAAGCTATGGCAGCATGGTCAGAAA |
| | 12,259 | TATCAGACAACCCAGGACAGGAGCGAGCCACCTGCAG |
| | Conserved | GGAAGAGAAGGCAGGCAGTTCGGGTCTCAGCAAACCA |
| | trinucleotide | TGCCTCTCAGCAATTGGATCAACTGAAGGCGGTGCACC |
| | motif: nt | TCGCATCCGCGGTCAGGGACCTGGAGAGAGCGATGAC |
| | 12,260-12,262 | GACGCTGAAACTTTGGGAATCCCCCCAAGAAATCTCCA |
| | Consensus GS | GGCATCAAGCACTGGGTTACAGTGTTATTACGTTTATG |
| | motif: nt | ATCACAGCGGTGAAGCGGTTAAGGGAATCCAAGATGC |
| | 12,263-12,279 | TGACTCTATCATGGTTCAATCAGGCCTTGATGGTGATA |
| | EBV gL ORF: | GCACCCTCTCAGGAGGAGACAATGAATCTGAAAACAG |
| | nt 12,300- | CGATGTGGATATTGGCGAACCTGATACCGAGGGATATG |
| | 12,713 | CTATCACTGACCGGGGATCTGCTCCCATCTCTATGGGG |
| | ATU | TTCAGGGCTTCTGATGTTGAAACTGCAGAAGGAGGGG |
| | downstream | AGATCCACGAGCTCCTGAGACTCCAATCCAGAGGCAA |
| | motif: nt | CAACTTTCCGAAGCTTGGGAAAACTCTCAATGTTCCTC |
| | 12,714-12,749 | CGCCCCCGGACCCCGGTAGGGCCAGCACTTCCGGGAC |
| | MV L ORF: nt | ACCCATTAAAAAGGGCACAGACGCGAGATTAGCCTCA |
| | 12,804-19,355 | TTTGGAACGGAGATCGCGTCTTTATTGACAGGTGGTGC |
| | MV trailer: nt | AACCCAATGTGCTCGAAAGTCACCCTCGGAACCATCAG |
| | 19,428-19,464 | GGCCAGGTGCACCTGCGGGGAATGTCCCCGAGTGTGTG |
| | | AGCAATGCCGCACTGATACAGGAGTGGACACCCGAAT |
| | | CTGGTACCACAATCTCCCCGAGATCCCAGAATAATGAA |
| | | GAAGGGGGAGACTATTATGATGATGAGCTGTTCTCTGA |
| | | TGTCCAAGATATTAAAACAGCCTTGGCCAAAATACACG |
| | | AGGATAATCAGAAGATAATCTCCAAGCTAGAATCACT |
| | | GCTGTTATTGAAGGGAGAAGTTGAGTCAATTAAGAAG |
| | | CAGATCAACAGGCAAAATATCAGCATATCCACCCTGG |
| | | AAGGACACCTCTCAAGCATCATGATCGCCATTCCTGGA |
| | | CTTGGGAAGGATCCCAACGACCCCACTGCAGATGTCGA |
| | | AATCAATCCCGACTTGAAACCCATCATAGGCAGAGATT |
| | | CAGGCCGAGCACTGGCCGAAGTTCTCAAGAAACCCGTT |
| | | GCCAGCCGACAACTCCAAGGAATGACAAATGGACGGA |
| | | CCAGTTCCAGAGGACAGCTGCTGAAGGAATTTCAGCTA |
| | | AAGCCGATCGGGAAAAAGATGAGCTCAGCCGTCGGGT |
| | | TTGTTCCTGACACCGGCCCTGCATCACGCAGTGTAATC |
| | | CGCTCCATTATAAAATCCAGCCGGCTAGAGGAGGATCG |
| | | GAAGCGTTACCTGATGACTCTCCTTGATGATATCAAAG |
| | | GAGCCAATGATCTTGCCAAGTTCCACCAGATGCTGATG |
| | | AAGATAATAATGAAGTAGCTACAGCTCAACTTACCTGC |
| | | CAACCCCATGCCAGTCGACCCAACTAGTACAACCTAAA |
| | | TCCATTATAAAAAACTTAGGAGCAAAGTGATTGCCTCC |
| | | CAAGGTCCACAATGACAGAGACCTACGACTTCGACAA |
| | | GTCGGCATGGGACATCAAAGGGTCGATCGCTCCGATAC |
| | | AACCCACCACCTACAGTGATGGCAGGCTGGTGCCCCAG |
| | | GTCAGAGTCATAGATCCTGGTCTAGGCGACAGGAAGG |
| | | ATGAATGCTTTATGTACATGTTTCTGCTGGGGGTTGTTG |
| | | AGGACAGCGATTCCCTAGGGCCTCCAATCGGGCGAGC |
| | | ATTTGGGTTCCTGCCCTTAGGTGTTGGCAGATCCACAG |
| | | CAAAGCCCGAAAAACTCCTCAAAGAGGCCACTGAGCT |
| | | TGACATAGTTGTTAGACGTACAGCAGGGCTCAATGAAA |
| | | AACTGGTGTTCTACAACAACACCCCACTAACTCTCCTC |
| | | ACACCTTGGAGAAAGGTCCTAACAACAGGGAGTGTCTT |
| | | CAACGCAAACCAAGTGTGCAATGCGGTTAATCTGATAC |
| | | CGCTCGATACCCCGCAGAGGTTCCGTGTTGTTTATATG |
| | | AGCATCACCCGTCTTTCGGATAACGGGTATTACACCGT |
| | | TCCTAGAAGAATGCTGGAATTCAGATCGGTCAATGCAG |
| | | TGGCCTTCAACCTGCTGGTGACCCTTAGGATTGACAAG |
| | | GCGATAGGCCCTGGGAAGATCATCGACAATACAGAGC |
| | | AACTTCCTGAGGCAACATTTATGGTCCACATCGGGAAC |
| | | TTCAGGAGAAAGAAGAGTGAAGTCTACTCTGCCGATTA |
| | | TTGCAAAATGAAAATCGAAAAGATGGGCCTGGTTTTTG |
| | | CACTTGGTGGGATAGGGGGCACCAGTCTTCACATTAGA |
| | | AGCACAGGCAAAATGAGCAAGACTCTCCATGCACAAC |
| | | TCGGGTTCAAGAAGACCTTATGTTACCCGCTGATGGAT |
| | | ATCAATGAAGACCTTAATCGATTACTCTGGAGGAGCAG |
| | | ATGCAAGATAGTAAGAATCCAGGCAGTTTTGCAGCCAT |
| | | CAGTTCCTCAAGAATTCCGCATTTACGACGACGTGATC |
| | | ATAAATGATGACCAAGGACTATTCAAAGTTCTGTAGAC |
| | | CGTAGTGCCCAGCAATGCCCGAAAACGACCCCCCTCAC |
| | | AATGACAGCCAGAAGGCCCGGACAAAAAAGCCCCCTC |
| | | CGAAAGACTCCACGGACCAAGCGAGAGGCCAGCCAGC |
| | | AGCCGACGGCAAGCGCGAACACCAGGCGGCCCCAGCA |
| | | CAGAACAGCCCTGACACAAGGCCACCACCAGCCACCC |
| | | CAATCTGCATCCTCCTCGTGGGACCCCCGAGGACCAAC |
| | | CCCCAAGGCTGCCCCCGATCCAAACCACCAACCGCATC |
| | | CCCACCACCCCCGGGAAAGAAACCCCCAGCAATTGGA |
| | | AGGCCCCTCCCCCTCTTCCTCAACACAAGAACTCCACA |
| | | ACCGAACCGCACAAGCGACCGAGGTGACCCAACCGCA |
| | | GGCATCCGACTCCCTAGACAGATCCTCTCTCCCCGGCA |
| | | AACTAAACAAAACTTAGGGCCAAGGAACATACACACC |
| | | CAACAGAACCCAGACCCCGGCCCACGGCGCCGCGCCC |
| | | CCAACCCCCGACAACCAGAGGGAGCCCCCAACCAATC |
| | | CCGCCGGCTCCCCCGGTGCCCACAGGCAGGGACACCA |
| | | ACCCCCGAACAGACCCAGCACCCAACCATCGACAATC |
| | | CAAGACGGGGGGGCCCCCCCAAAAAAAGGCCCCCAGG |
| | | GGCCGACAGCCAGCACCGCGAGGAAGCCCACCCACCC |
| | | CACACACGACCACGGCAACCAAACCAGAACCCAGACC |
| | | ACCCTGGGCCACCAGCTCCCAGACTCGGCCATCACCCC |
| | | GCAGAAAGGAAAGGCCACAACCCGCGCACCCCAGCCC |
| | | CGATCCGGCGGGGAGCCACCCAACCCGAACCAGCACC |
| | | CAAGAGCGATCCCCGAAGGACCCCCGAACCGCAAAGG |
| | | ACATCAGTATCCCACAGCCTCTCCAAGTCCCCCGGTCT |
| | | CCTCCTCTTCTCGAAGGGACCAAAAGATCAATCCACCA |
| | | CACCCGACGACACTCAACTCCCCACCCCTAAAGGAGAC |
| | | ACCGGGAATCCCAGAATCAAGACTCATCCAATGTCCAT |
| | | CATGGGTCTCAAGGTGAACGTCTCTGCCATATTCATGG |
| | | CAGTACTGTTAACTCTCCAAACACCCACCGGTCAAATC |
| | | CATTGGGGCAATCTCTCTAAGATAGGGGTGGTAGGAAT |
| | | AGGAAGTGCAAGCTACAAAGTTATGACTCGTTCCAGCC |
| | | ATCAATCATTAGTCATAAAATTAATGCCCAATATAACT |
| | | CTCCTCAATAACTGCACGAGGGTAGAGATTGCAGAATA |
| | | CAGGAGACTACTGAGAACAGTTTTGGAACCAATTAGA |
| | | GATGCACTTAATGCAATGACCCAGAATATAAGACCGGT |
| | | TCAGAGTGTAGCTTCAAGTAGGAGACACAAGAGATTT |
| | | GCGGGAGTAGTCCTGGCAGGTGCGGCCCTAGGCGTTGC |
| | | CACAGCTGCTCAGATAACAGCCGGCATTGCACTTCACC |
| | | AGTCCATGCTGAACTCTCAAGCCATCGACAATCTGAGA |
| | | GCGAGCCTGGAAACTACTAATCAGGCAATTGAGACAA |
| | | TCAGACAAGCAGGGCAGGAGATGATATTGGCTGTTCA |
| | | GGGTGTCCAAGACTACATCAATAATGAGCTGATACCGT |
| | | CTATGAACCAACTATCTTGTGATTTAATCGGCCAGAAG |
| | | CTCGGGCTCAAATTGCTCAGATACTATACAGAAATCCT |
| | | GTCATTATTTGGCCCCAGTTTACGGGACCCCATATCTG |
| | | CGGAGATATCTATCCAGGCTTTGAGCTATGCGCTTGGA |
| | | GGAGACATCAATAAGGTGTTAGAAAAGCTCGGATACA |
| | | GTGGAGGTGATTTACTGGGCATCTTAGAGAGCGGAGG |
| | | CATCAAGGCCCGGATAACTCACGTCGACACAGAGTCCT |
| | | ACTTCATTGTCCTCAGTATAGCCTATCCGACGCTGTCCG |
| | | AGATTAAGGGGGTGATTGTCCACCGGCTAGAGGGGGT |
| | | CTCGTACAACATAGGCTCTCAAGAGTGGTATACCACTG |
| | | TGCCCAAGTATGTTGCAACCCAAGGGTACCTTATCTCG |
| | | AATTTTGATGAGTCATCGTGTACTTTCATGCCAGAGGG |
| | | GACTGTGTGCAGCCAAAATGCCTTGTACCCGATGAGTC |
| | | CTCTGCTCCAAGAATGCCTCCGGGGGTACACCAAGTCC |
| | | TGTGCTCGTACACTCGTATCCGGGTCTTTTGGGAACCG |
| | | GTTCATTTTATCACAAGGGAACCTAATAGCCAATTGTG |
| | | CATCAATCCTTTGCAAGTGTTACACAACAGGAACGATC |
| | | ATTAATCAAGACCCTGACAAGATCCTAACATACATTGC |
| | | TGCCGATCACTGCCCGGTAGTCGAGGTGAACGGCGTGA |
| | | CCATCCAAGTCGGGAGCAGGAGGTATCCAGACGCTGT |
| | | GTACTTGCACAGAATTGACCTCGGTCCTCCCATATCAT |
| | | TGGAGAGGTTGGACGTAGGGACAAATCTGGGGAATGC |
| | | AATTGCTAAGTTGGAGGATGCCAAGGAATTGTTGGAGT |
| | | CATCGGACCAGATATTGAGGAGTATGAAAGGTTTATCG |
| | | AGCACTAGCATAGTCTACATCCTGATTGCAGTGTGTCT |
| | | TGGAGGGTTGATAGGGATCCCCGCTTTAATATGTTGCT |
| | | GCAGGGGGCGTTGTAACAAAAAGGGAGAACAAGTTGG |
| | | TATGTCAAGACCAGGCCTAAAGCCTGATCTTACGGGAA |
| | | CATCAAAATCCTATGTAAGGTCGCTCTGATCCTCTACA |
| | | ACTCTTGAAACACAAATGTCCCACAAGTCTCCTCTTCG |
| | | TCATCAAGCAACCACCGCACCCAGCATCAAGCCCACCT |
| | | GAAATTATCTCCGGCTTCCCTCTGGCCGAACAATATCG |
| | | GTAGTTAATCAAAACTTAGGGTGCAAGATCATCCACAA |
| | | TGTCACCACAACGAGACCGGATAAATGCCTTCTACAAA |
| | | GATAACCCCCATCCCAAGGGAAGTAGGATAGTCATTA |
| | | ACAGAGAACATCTTATGATTGATAGACCTTATGTTTTG |
| | | CTGGCTGTTCTGTTTGTCATGTTTCTGAGCTTGATCGGG |
| | | TTGCTAGCCATTGCAGGCATTAGACTTCATCGGGCAGC |
| | | CATCTACACCGCAGAGATCCATAAAAGCCTCAGCACCA |
| | | ATCTAGATGTAACTAACTCAATCGAGCATCAGGTCAAG |
| | | GACGTGCTGACACCACTCTTCAAAATCATCGGTGATGA |
| | | AGTGGGCCTGAGGACACCTCAGAGATTCACTGACCTAG |
| | | TGAAATTAATCTCTGACAAGATCAAGTTCCTTAATCCG |
| | | GATAGGGAGTACGACTTCAGAGATCTCACTTGGTGTAT |
| | | CAACCCGCCAGAGAGAATCAAATTGGATTATGATCAAT |
| | | ACTGTGCAGATGTGGCTGCTGAAGAGCTCATGAATGCA |
| | | TTGGTGAACTCAACTCTACTGGAGACCAGAACAACCAA |
| | | TCAGTTCCTAGCTGTCTCAAAGGGAAACTGCTCAGGGC |
| | | CCACTACAATCAGAGGTCAATTCTCAAACATGTCGCTG |
| | | TCCCTGTTAGACTTGTATTTAGGTCGAGGTTACAATGT |
| | | GTCATCTATAGTCACTATGACATCCCAGGGAATGTATG |
| | | GGGGAACTTACCTAGTGGAAAAGCCTAATCTGAGCAG |
| | | CAAAAGGTCAGAGTTGTCACAACTGAGCATGTACCGA |
| | | GTGTTTGAAGTAGGTGTTATCAGAAATCCGGGTTTGGG |
| | | GGCTCCGGTGTTCCATATGACAAACTATCTTGAGCAAC |
| | | CAGTCAGTAATGATCTCAGCAACTGTATGGTGGCTTTG |
| | | GGGGAGCTCAAACTCGCAGCCCTTTGTCACGGGGAAG |
| | | ATTCTATCACAATTCCCTATCAGGGATCAGGGAAAGGT |
| | | GTCAGCTTCCAGCTCGTCAAGCTAGGTGTCTGGAAATC |
| | | CCCAACCGACATGCAATCCTGGGTCCCCTTATCAACGG |
| | | ATGATCCAGTGATAGACAGGCTTTACCTCTCATCTCAC |
| | | AGAGGTGTTATCGCTGACAATCAAGCAAAATGGGCTGT |
| | | CCCGACAACACGAACAGATGACAAGTTGCGAATGGAG |
| | | ACATGCTTCCAACAGGCGTGTAAGGGTAAAATCCAAG |
| | | CACTCTGCGAGAATCCCGAGTGGGCACCATTGAAGGAT |
| | | AACAGGATTCCTTCATACGGGGTCTTGTCTGTTGATCT |
| | | GAGTCTGACAGTTGAGCTTAAAATCAAAATTGCTTCGG |
| | | GATTCGGGCCATTGATCACACACGGTTCAGGGATGGAC |
| | | CTATACAAATCCAACCACAACAATGTGTATTGGCTGAC |
| | | TATCCCGCCAATGAAGAACCTAGCCTTAGGTGTAATCA |
| | | ACACATTGGAGTGGATACCGAGATTCAAGGTTAGTCCC |
| | | TACCTCTTCACTGTCCCAATTAAGGAAGCAGGCGAAGA |
| | | CTGCCATGCCCCAACATACCTACCTGCGGAGGTGGATG |
| | | GTGATGTCAAACTCAGTTCCAATCTGGTGATTCTACCT |
| | | GGTCAAGATCTCCAATATGTTTTGGCAACCTACGATAC |
| | | TTCCAGGGTTGAACATGCTGTGGTTTATTACGTTTACA |
| | | GCCCAAGCCGCTCATTTTCTTACTTTTATCCTTTTAGGT |
| | | TGCCTATAAAGGGGGTCCCCATCGAATTACAAGTGGAA |
| | | TGCTTCACATGGGACCAAAAACTCTGGTGCCGTCACTT |
| | | CTGTGTGCTTGCGGACTCAGAATCTGGTGGACATATCA |
| | | CTCACTCTGGGATGGTGGGCATGGGAGTCAGCTGCACA |
| | | GTCACCCGGGAAGATGGAACCAATCGCAGATAGGGCT |
| | | GCTAGTGAACCAATCACATGATGTCACCCAGACATCAG |
| | | GCATACCCACTAGTCTACCCTCCATCATTGTTATAAAA |
| | | AACTTAGGAACCAGGTCCACACAGCCGCCAGCCCATC |
| | | AACGCGTACGGCCACCATGCAACTCCTCTGCGTCTTTT |
| | | GCTTGGTGCTTTTGTGGGAGGTAGGAGCGGCCTCATTG |
| | | TCAGAAGTTAAGCTGCATCTCGATATTGAAGGCCACGC |
| | | TTCACATTACACTATCCCTTGGACCGAACTCATGGCTA |
| | | AGGTGCCGGGTTTGTCTCCGGAAGCACTTTGGAGAGAG |
| | | GCAAACGTTACCGAAGACCTGGCCTCAATGCTGAATCG |
| | | CTACAAGCTGATATACAAGACTTCCGGAACCCTGGGTA |
| | | TAGCACTCGCAGAGCCCGTCGATATCCCCGCTGTCAGC |
| | | GAAGGTAGCATGCAGGTGGACGCTAGTAAAGTTCACC |
| | | CAGGAGTTATCTCAGGTCTGAATAGCCCAGCTTGTATG |
| | | CTGAGCGCTCCACTGGAGAAACAGCTCTTCTACTATAT |
| | | AGGCACAATGCTCCCTAACACTCGACCTCACAGCTACG |
| | | TCTTCTATCAGCTGCGCTGTCACCTGTCATACGTAGCCT |
| | | TGTCCATTAATGGTGATAAGTTCCAGTACACAGGTGCC |
| | | ATGACCTCCAAGTTCCTTATGGGGACCTATAAACGGGT |
| | | CACTGAGAAAGGAGACGAGCATGTCCTTAGTTTGGTTT |
| | | TTGGGAAGACAAAAGACCTGCCGGATCTGAGGGGCCC |
| | | GTTCTCTTACCCCTCACTCACATCTGCACAGAGTGGGG |
| | | ACTATTCATTGGTGATTGTGACAACCTTCGTTCACTACG |
| | | CCAATTTTCACAACTATTTTGTCCCGAACCTTAAAGAC |
| | | ATGTTTTCACGCGCCGTGACCATGACAGCTGCTTCCTA |
| | | CGCTAGGTATGTGCTCCAAAAGCTGGTGCTGCTGGAGA |
| | | TGAAGGGCGGATGCAGGGAACCTGAACTGGACACAGA |
| | | GACTCTCACCACTATGTTCGAGGTTTCCGTGGCGTTCTT |
| | | CAAGGTGGGTCATGCTGTGGGGGAGACTGGGAACGGC |
| | | TGCGTAGATCTTAGGTGGCTGGCTAAATCTTTCTTCGA |
| | | GCTGACCGTGTTGAAAGATATAATTGGAATTTGCTACG |
| | | GGGCTACGGTTAAAGGAATGCAGTCTTACGGGCTGGA |
| | | AAGGCTGGCCGCTATGCTCATGGCTACTGTGAAGATGG |
| | | AGGAACTCGGCCACCTCACCACAGAAAAACAGGAGTA |
| | | CGCTTTGCGCCTGGCCACTGTTGGCTACCCTAAGGCAG |
| | | GCGTTTACTCTGGATTGATTGGGGGGGCCACATCTGTG |
| | | CTCTTGTCAGCCTACAATCGCCATCCCCTGTTCCAGCCT |
| | | CTGCACACGGTGATGCGAGAGACTTTGTTTATTGGCAG |
| | | CCACGTTGTGCTGAGAGAACTGCGCCTTAACGTTACCA |
| | | CTCAGGGACCTAACCTTGCGCTGTATCAGCTGCTTAGC |
| | | ACCGCTTTGTGCTCCGCTCTTGAAATTGGAGAAGTACT |
| | | CAGAGGGCTCGCACTGGGTACTGAGTCAGGGCTGTTCT |
| | | CCCCCTGCTATCTGTCTCTCCGGTTCGACCTCACTCGGG |
| | | ATAAGCTGTTGTCAATGGCTCCGCAGGAAGCCACATTG |
| | | GACCAGGCCGCAGTGAGTAACGCAGTGGACGGGTTCC |
| | | TGGGTAGACTGTCTCTTGAGAGGGAAGACAGAGACGC |
| | | TTGGCATCTGCCTGCGTATAAGTGTGTTGATAGACTGG |
| | | ACAAAGTACTGATGATCATTCCCCTGATTAATGTGACA |
| | | TTTATTATATCTAGTGACCGAGAGGTGAGAGGCTCCGC |
| | | TCTCTATGAAGCATCCACAACGTACTTGAGCAGTTCCC |
| | | TGTTTCTGTCACCGGTTATTATGAACAAATGTTCTCAGG |
| | | GAGCTGTTGCTGGTGAACCGCGACAGATTCCAAAGATT |
| | | CAGAACTTCACTAGGACACAAAAGAGCTGCATTTTCTG |
| | | CGGGTTCGCACTGCTGAGTTACGACGAAAAGGAGGGA |
| | | CTGGAGACTACTACATATATTACTTCACAGGAGGTGCA |
| | | GAATAGCATCCTGAGCTCCAATTATTTTGACTTTGACA |
| | | ACCTCCATGTCCATTATTTGCTGTTGACCACAAACGGC |
| | | ACTGTGATGGAGATCGCTGGCCTGTACGAGGAGCGAG |
| | | CCCACGTAGTACTGGCCATTATTCTGTACTTCATCGCTT |
| | | TCGCACTTGGGATTTTCCTCGTCCACAAAATTGTTATGT |
| | | TTTTCCTCTAATGATTCGAACTACAGCTCAACTTACCTG |
| | | CCAACCCCATGCCAGTCGACCCAACTAGTACAACCTAA |
| | | ATCCATTATAAAAAACTTAGGAACCAGGTCCACACAGC |
| | | CGCCAGCCCATCAACCATCCACTCCCACGATTGGAGGC |
| | | CGGCCATGGTGAGTTTTAAGCAGGTCCGGGTACCTCTG |
| | | TTCACCGCCATTGCGCTGGTGATTGTTCTGCTGTTGGCC |
| | | TACTTTCTTCCACCTAGAGTCAGAGGAGGTGGCAGGGT |
| | | TGCTGCAGCTGCTATTACCTGGGTACCGAAACCCAACG |
| | | TGGAGGTTTGGCCAGTGGATCCGCCCCCACCAGTGAAC |
| | | TTTAATAAAACAGCCGAACAAGAATACGGGGATAAAG |
| | | AAGTAAAGCTGCCACACTGGACCCCTACCCTCCACACT |
| | | TTCCAGGTGCCTCAAAACTATACAAAGGCCAATTGCAC |
| | | ATACTGTAACACAAGAGAATACACTTTTTCTTATAAAG |
| | | GGTGCTGCTTCTACTTCACTAAGAAGAAGCATACTTGG |
| | | AACGGATGTTTCCAGGCATGCGCTGAGTTGTATCCCTG |
| | | TACCTATTTCTACGGTCCTACACCAGACATTCTGCCGGT |
| | | CGTGACTCGCAATCTGAACGCCATAGAATCTCTGTGGG |
| | | TCGGGGTGTATCGGGTTGGGGAAGGAAATTGGACTAG |
| | | CCTGGACGGCGGTACTTTCAAGGTGTACCAAATATTCG |
| | | GCAGTCACTGTACTTACGTGTCAAAATTCTCAACAGTT |
| | | CCCGTATCCCACCACGAGTGTTCTTTTTTGAAGCCTTGC |
| | | CTGTGCGTGTCACAGAGGTCAAATTCCTAACTATTCGA |
| | | ACTACTCAGCTAGTCTACGCGCCGTAGCCTACCCTCCA |
| | | TCATTGTTATAAAAAACTTAGGATCCAAGAGCATACAG |
| | | CCGCCGATAAGGTCCACAATGAGAGCCGTAGGTGTCTT |
| | | TCTGGCTATTTGTCTGGTAACAATCTTCGTGCTGCCCAC |
| | | TTGGGGCAACTGGGCCTATCCTTGCTGCCATGTTACAC |
| | | AGTTGAGAGCGCAGCACCTCCTGGCGTTGGAGAACATT |
| | | TCAGACATCTACCTGGTGAGTAACCAAACCTGCGACGG |
| | | GTTCTCTTTGGCCAGTCTCAATAGTCCAAAAAACGGAA |
| | | GCAATCAGCTGGTAATTTCACGGTGTGCAAACGGACTT |
| | | AACGTGGTCTCTTTTTTTATATCAATCCTGAAACGCAGT |
| | | TCATCTGCATTGACCGGCCACTTGAGGGAGCTCTTGAC |
| | | CACTCTCGAGACACTTTACGGGTCATTCTCCGTGGAGG |
| | | ATCTGTTCGGGGCAAATCTGAACCGGTACGCCTGGCAT |
| | | AGAGGCGGGTAAGCGCGCAGCGCTTAGACGTCTCGCG |
| | | ATCGATGCTAGTGTGAAATAGACATCAGAATTAAGAA |
| | | AAACGTAGGGTCCAAGTGGTTCCCCGTTATGGACTCGC |
| | | TATCTGTCAACCAGATCTTATACCCTGAAGTTCACCTA |
| | | GATAGCCCGATAGTTACCAATAAGATAGTAGCCATCCT |
| | | GGAGTATGCTCGAGTCCCTCACGCTTACAGCCTGGAGG |
| | | ACCCTACACTGTGTCAGAACATCAAGCACCGCCTAAAA |
| | | AACGGATTTTCCAACCAAATGATTATAAACAATGTGGA |
| | | AGTTGGGAATGTCATCAAGTCCAAGCTTAGGAGTTATC |
| | | CGGCCCACTCTCATATTCCATATCCAAATTGTAATCAG |
| | | GATTTATTTAACATAGAAGACAAAGAGTCAACGAGGA |
| | | AGATCCGTGAACTCCTCAAAAAGGGGAATTCGCTGTAC |
| | | TCCAAAGTCAGTGATAAGGTTTTCCAATGCTTAAGGGA |
| | | CACTAACTCACGGCTTGGCCTAGGCTCCGAATTGAGGG |
| | | AGGACATCAAGGAGAAAGTTATTAACTTGGGAGTTTAC |
| | | ATGCACAGCTCCCAGTGGTTTGAGCCCTTTCTGTTTTGG |
| | | TTTACAGTCAAGACTGAGATGAGGTCAGTGATTAAATC |
| | | ACAAACCCATACTTGCCATAGGAGGAGACACACACCT |
| | | GTATTCTTCACTGGTAGTTCAGTTGAGTTGCTAATCTCT |
| | | CGTGACCTTGTTGCTATAATCAGTAAAGAGTCTCAACA |
| | | TGTATATTACCTGACATTTGAACTGGTTTTGATGTATTG |
| | | TGATGTCATAGAGGGGAGGTTAATGACAGAGACCGCT |
| | | ATGACTATTGATGCTAGGTATACAGAGCTTCTAGGAAG |
| | | AGTCAGATACATGTGGAAACTGATAGATGGTTTCTTCC |
| | | CTGCACTCGGGAATCCAACTTATCAAATTGTAGCCATG |
| | | CTGGAGCCTCTTTCACTTGCTTACCTGCAGCTGAGGGA |
| | | TATAACAGTAGAACTCAGAGGTGCTTTCCTTAACCACT |
| | | GCTTTACTGAAATACATGATGTTCTTGACCAAAACGGG |
| | | TTTTCTGATGAAGGTACTTATCATGAGTTAACTGAAGC |
| | | TCTAGATTACATTTTCATAACTGATGACATACATCTGA |
| | | CAGGGGAGATTTTCTCATTTTTCAGAAGTTTCGGCCAC |
| | | CCCAGACTTGAAGCAGTAACGGCTGCTGAAAATGTTAG |
| | | GAAATACATGAATCAGCCTAAAGTCATTGTGTATGAGA |
| | | CTCTGATGAAAGGTCATGCCATATTTTGTGGAATCATA |
| | | ATCAACGGCTATCGTGACAGGCACGGAGGCAGTTGGC |
| | | CACCGCTGACCCTCCCCCTGCATGCTGCAGACACAATC |
| | | CGGAATGCTCAAGCTTCAGGTGAAGGGTTAACACATG |
| | | AGCAGTGCGTTGATAACTGGAAATCTTTTGCTGGAGTG |
| | | AAATTTGGCTGCTTTATGCCTCTTAGCCTGGATAGTGAT |
| | | CTGACAATGTACCTAAAGGACAAGGCACTTGCTGCTCT |
| | | CCAAAGGGAATGGGATTCAGTTTACCCGAAAGAGTTCC |
| | | TGCGTTACGACCCTCCCAAGGGAACCGGGTCACGGAG |
| | | GCTTGTAGATGTTTTCCTTAATGATTCGAGCTTTGACCC |
| | | ATATGATGTGATAATGTATGTTGTAAGTGGAGCTTACC |
| | | TCCATGACCCTGAGTTCAACCTGTCTTACAGCCTGAAA |
| | | GAAAAGGAGATCAAGGAAACAGGTAGACTTTTTGCTA |
| | | AAATGACTTACAAAATGAGGGCATGCCAAGTGATTGCT |
| | | GAAAATCTAATCTCAAACGGGATTGGCAAATATTTTAA |
| | | GGACAATGGGATGGCCAAGGATGAGCACGATTTGACT |
| | | AAGGCACTCCACACTCTAGCTGTCTCAGGAGTCCCCAA |
| | | AGATCTCAAAGAAAGTCACAGGGGGGGGCCAGTCTTA |
| | | AAAACCTACTCCCGAAGCCCAGTCCACACAAGTACCA |
| | | GGAACGTGAGAGCAGCAAAAGGGTTTATAGGGTTCCC |
| | | TCAAGTAATTCGGCAGGACCAAGACACTGATCATCCGG |
| | | AGAATATGGAAGCTTACGAGACAGTCAGTGCATTTATC |
| | | ACGACTGATCTCAAGAAGTACTGCCTTAATTGGAGATA |
| | | TGAGACCATCAGCTTGTTTGCACAGAGGCTAAATGAGA |
| | | TTTACGGATTGCCCTCATTTTTCCAGTGGCTGCATAAGA |
| | | GGCTTGAGACCTCTGTCCTGTATGTAAGTGACCCTCAT |
| | | TGCCCCCCCGACCTTGACGCCCATATCCCGTTATATAA |
| | | AGTCCCCAATGATCAAATCTTCATTAAGTACCCTATGG |
| | | GAGGTATAGAAGGGTATTGTCAGAAGCTGTGGACCAT |
| | | CAGCACCATTCCCTATCTATACCTGGCTGCTTATGAGA |
| | | GCGGAGTAAGGATTGCTTCGTTAGTGCAAGGGGACAA |
| | | TCAGACCATAGCCGTAACAAAAAGGGTACCCAGCACA |
| | | TGGCCCTACAACCTTAAGAAACGGGAAGCTGCTAGAG |
| | | TAACTAGAGATTACTTTGTAATTCTTAGGCAAAGGCTA |
| | | CATGATATTGGCCATCACCTCAAGGCAAATGAGACAAT |
| | | TGTTTCATCACATTTTTTTGTCTATTCAAAAGGAATATA |
| | | TTATGATGGGCTACTTGTGTCCCAATCACTCAAGAGCA |
| | | TCGCAAGATGTGTATTCTGGTCAGAGACTATAGTTGAT |
| | | GAAACAAGGGCAGCATGCAGTAATATTGCTACAACAA |
| | | TGGCTAAAAGCATCGAGAGAGGTTATGACCGTTACCTT |
| | | GCATATTCCCTGAACGTCCTAAAAGTGATACAGCAAAT |
| | | TCTGATCTCTCTTGGCTTCACAATCAATTCAACCATGAC |
| | | CCGGGATGTAGTCATACCCCTCCTCACAAACAACGACC |
| | | TCTTAATAAGGATGGCACTGTTGCCCGCTCCTATTGGG |
| | | GGGATGAATTATCTGAATATGAGCAGGCTGTTTGTCAG |
| | | AAACATCGGTGATCCAGTAACATCATCAATTGCTGATC |
| | | TCAAGAGAATGATTCTCGCCTCACTAATGCCTGAAGAG |
| | | ACCCTCCATCAAGTAATGACACAACAACCGGGGGACT |
| | | CTTCATTCCTAGACTGGGCTAGCGACCCTTACTCAGCA |
| | | AATCTTGTATGTGTCCAGAGCATCACTAGACTCCTCAA |
| | | GAACATAACTGCAAGGTTTGTCCTGATCCATAGTCCAA |
| | | ACCCAATGTTAAAAGGATTATTCCATGATGACAGTAAA |
| | | GAAGAGGACGAGGGACTGGCGGCATTCCTCATGGACA |
| | | GGCATATTATAGTACCTAGGGCAGCTCATGAAATCCTG |
| | | GATCATAGTGTCACAGGGGCAAGAGAGTCTATTGCAG |
| | | GCATGCTGGATACCACAAAAGGCTTGATTCGAGCCAGC |
| | | ATGAGGAAGGGGGGGTTAACCTCTCGAGTGATAACCA |
| | | GATTGTCCAATTATGACTATGAACAATTCAGAGCAGGG |
| | | ATGGTGCTATTGACAGGAAGAAAGAGAAATGTCCTCA |
| | | TTGACAAAGAGTCATGTTCAGTGCAGCTGGCGAGAGCT |
| | | CTAAGAAGCCATATGTGGGCGAGGCTAGCTCGAGGAC |
| | | GGCCTATTTACGGCCTTGAGGTCCCTGATGTACTAGAA |
| | | TCTATGCGAGGCCACCTTATTCGGCGTCATGAGACATG |
| | | TGTCATCTGCGAGTGTGGATCAGTCAACTACGGATGGT |
| | | TTTTTGTCCCCTCGGGTTGCCAACTGGATGATATTGACA |
| | | AGGAAACATCATCCTTGAGAGTCCCATATATTGGTTCT |
| | | ACCACTGATGAGAGAACAGACATGAAGCTTGCCTTCGT |
| | | AAGAGCCCCAAGTCGATCCTTGCGATCTGCTGTTAGAA |
| | | TAGCAACAGTGTACTCATGGGCTTACGGTGATGATGAT |
| | | AGCTCTTGGAACGAAGCCTGGTTGTTGGCTAGGCAAAG |
| | | GGCCAATGTGAGCCTGGAGGAGCTAAGGGTGATCACT |
| | | CCCATCTCAACTTCGACTAATTTAGCGCATAGGTTGAG |
| | | GGATCGTAGCACTCAAGTGAAATACTCAGGTACATCCC |
| | | TTGTCCGAGTGGCGAGGTATACCACAATCTCCAACGAC |
| | | AATCTCTCATTTGTCATATCAGATAAGAAGGTTGATAC |
| | | TAACTTTATATACCAACAAGGAATGCTTCTAGGGTTGG |
| | | GTGTTTTAGAAACATTGTTTCGACTCGAGAAAGATACC |
| | | GGATCATCTAACACGGTATTACATCTTCACGTCGAAAC |
| | | AGATTGTTGCGTGATCCCGATGATAGATCATCCCAGGA |
| | | TACCCAGCTCCCGCAAGCTAGAGCTGAGGGCAGAGCT |
| | | ATGTACCAACCCATTGATATATGATAATGCACCTTTAA |
| | | TTGACAGAGATGCAACAAGGCTATACACCCAGAGCCA |
| | | TAGGAGGCACCTTGTGGAATTTGTTACATGGTCCACAC |
| | | CCCAACTATATCACATTTTAGCTAAGTCCACAGCACTA |
| | | TCTATGATTGACCTGGTAACAAAATTTGAGAAGGACCA |
| | | TATGAATGAAATTTCAGCTCTCATAGGGGATGACGATA |
| | | TCAATAGTTTCATAACTGAGTTTCTGCTCATAGAGCCA |
| | | AGATTATTCACTATCTACTTGGGCCAGTGTGCGGCCAT |
| | | CAATTGGGCATTTGATGTACATTATCATAGACCATCAG |
| | | GGAAATATCAGATGGGTGAGCTGTTGTCATCGTTCCTT |
| | | TCTAGAATGAGCAAAGGAGTGTTTAAGGTGCTTGTCAA |
| | | TGCTCTAAGCCACCCAAAGATCTACAAGAAATTCTGGC |
| | | ATTGTGGTATTATAGAGCCTATCCATGGTCCTTCACTTG |
| | | ATGCTCAAAACTTGCACACAACTGTGTGCAACATGGTT |
| | | TACACATGCTATATGACCTACCTCGACCTGTTGTTGAA |
| | | TGAAGAGTTAGAAGAGTTCACATTTCTCTTGTGTGAAA |
| | | GCGACGAGGATGTAGTACCGGACAGATTCGACAACAT |
| | | CCAGGCAAAACACTTATGTGTTCTGGCAGATTTGTACT |
| | | GTCAACCAGGGACCTGCCCACCAATTCGAGGTCTAAGA |
| | | CCGGTAGAGAAATGTGCAGTTCTAACCGACCATATCAA |
| | | GGCAGAGGCTATGTTATCTCCAGCAGGATCTTCGTGGA |
| | | ACATAAATCCAATTATTGTAGACCATTACTCATGCTCT |
| | | CTGACTTATCTCCGGCGAGGATCGATCAAACAGATAAG |
| | | ATTGAGAGTTGATCCAGGATTCATTTTCGACGCCCTCG |
| | | CTGAGGTAAATGTCAGTCAGCCAAAGATCGGCAGCAA |
| | | CAACATCTCAAATATGAGCATCAAGGCTTTCAGACCCC |
| | | CACACGATGATGTTGCAAAATTGCTCAAAGATATCAAC |
| | | ACAAGCAAGCACAATCTTCCCATTTCAGGGGGCAATCT |
| | | CGCCAATTATGAAATCCATGCTTTCCGCAGAATCGGGT |
| | | TGAACTCATCTGCTTGCTACAAAGCTGTTGAGATATCA |
| | | ACATTAATTAGGAGATGCCTTGAGCCAGGGGAGGACG |
| | | GCTTGTTCTTGGGTGAGGGATCGGGTTCTATGTTGATC |
| | | ACTTATAAAGAGATACTTAAACTAAACAAGTGCTTCTA |
| | | TAATAGTGGGGTTTCCGCCAATTCTAGATCTGGTCAAA |
| | | GGGAATTAGCACCCTATCCCTCCGAAGTTGGCCTTGTC |
| | | GAACACAGAATGGGAGTAGGTAATATTGTCAAAGTGC |
| | | TCTTTAACGGGAGGCCCGAAGTCACGTGGGTAGGCAGT |
| | | GTAGATTGCTTCAATTTCATAGTTAGTAATATCCCTACC |
| | | TCTAGTGTGGGGTTTATCCATTCAGATATAGAGACCTT |
| | | GCCTGACAAAGATACTATAGAGAAGCTAGAGGAATTG |
| | | GCAGCCATCTTATCGATGGCTCTGCTCCTGGGCAAAAT |
| | | AGGATCAATACTGGTGATTAAGCTTATGCCTTTCAGCG |
| | | GGGATTTTGTTCAGGGATTTATAAGTTATGTAGGGTCT |
| | | CATTATAGAGAAGTGAACCTTGTATACCCTAGATACAG |
| | | CAACTTCATCTCTACTGAATCTTATTTGGTTATGACAGA |
| | | TCTCAAGGCTAACCGGCTAATGAATCCTGAAAAGATTA |
| | | AGCAGCAGATAATTGAATCATCTGTGAGGACTTCACCT |
| | | GGACTTATAGGTCACATCCTATCCATTAAGCAACTAAG |
| | | CTGCATACAAGCAATTGTGGGAGACGCAGTTAGTAGA |
| | | GGTGATATCAATCCTACTCTGAAAAAACTTACACCTAT |
| | | AGAGCAGGTGCTGATCAATTGCGGGTTGGCAATTAACG |
| | | GACCTAAGCTGTGCAAAGAATTGATCCACCATGATGTT |
| | | GCCTCAGGGCAAGATGGATTGCTTAATTCTATACTCAT |
| | | CCTCTACAGGGAGTTGGCAAGATTCAAAGACAACCAA |
| | | AGAAGTCAACAAGGGATGTTCCACGCTTACCCCGTATT |
| | | GGTAAGTAGCAGGCAACGAGAACTTATATCTAGGATC |
| | | ACCCGCAAATTCTGGGGGCACATTCTTCTTTACTCCGG |
| | | GAACAAAAAGTTGATAAATAAGTTTATCCAGAATCTCA |
| | | AGTCCGGCTATCTGATACTAGACTTACACCAGAATATC |
| | | TTCGTTAAGAATCTATCCAAGTCAGAGAAACAGATTAT |
| | | TATGACGGGGGGTTTGAAACGTGAGTGGGTTTTTAAGG |
| | | TAACAGTCAAGGAGACCAAAGAATGGTATAAGTTAGT |
| | | CGGATACAGTGCCCTGATTAAGGACTAATTGGTTGAAC |
| | | TCCGGAACCCTAATCCTGCCCTAGGTGGTTAGGCATTA |
| | | TTTGCAATATATTAAAGAAAACTTTGAAAATACGAAGT |
| | | TTCTATTCCCAGCTTTGTCTGGT |
| 87 | T-09 | ACCAAACAAAGTTGGGTAAGGATAGTTCAATCAATGA |
| | EBV_gH_Fur- | TCATCTTCTAGTGCACTTAGGATTCAAGATCCTATTATC |
| | P2A_gL_Fur- | AGGGACAAGAGCAGGATTAGGGATATCCGAGATGGCC |
| | T2A_gp42 at | ACACTTTTAAGGAGCTTAGCATTGTTCAAAAGAAACAA |
| | ATU3 | GGACAAACCACCCATTACATCAGGATCCGGTGGAGCC |
| | MV leader: nt | ATCAGAGGAATCAAACACATTATTATAGTACCAATCCC |
| | 1-55 55 | TGGAGATTCCTCAATTACCACTCGATCCAGACTTCTGG |
| | MV N ORF: nt | ACCGGTTGGTGAGGTTAATTGGAAACCCGGATGTGAGC |
| | 108-1,685 | GGGCCCAAACTAACAGGGGCACTAATAGGTATATTATC |
| | MV P ORF: nt | CTTATTTGTGGAGTCTCCAGGTCAATTGATTCAGAGGA |
| | 1,807-3,330 | TCACCGATGACCCTGACGTTAGCATAAGGCTGTTAGAG |
| | MV M ORF: nt | GTTGTCCAGAGTGACCAGTCACAATCTGGCCTTACCTT |
| | 3,438-4,445 | CGCATCAAGAGGTACCAACATGGAGGATGAGGCGGAC |
| | MV F ORF: nt | CAATACTTTTCACATGATGATCCAATTAGTAGTGATCA |
| | 5,449-7,110 | ATCCAGGTTCGGATGGTTCGGGAACAAGGAAATCTCA |
| | MV H ORF: nt | GATATTGAAGTGCAAGACCCTGAGGGATTCAACATGAT |
| | 7,271-9,124 | TCTGGGTACCATCCTAGCCCAAATTTGGGTCTTGCTCG |
| | ATU upstream | CAAAGGCGGTTACGGCCCCAGACACGGCAGCTGATTC |
| | motif: nt 9,180- | GGAGCTAAGAAGGTGGATAAAGTACACCCAACAAAGA |
| | 9,250 | AGGGTAGTTGGTGAATTTAGATTGGAGAGAAAATGGTT |
| | GE of N gene: | GGATGTGGTGAGGAACAGGATTGCCGAGGACCTCTCCT |
| | nt 9,196-9,206 | TACGCCGATTCATGGTCGCTCTAATCCTGGATATCAAG |
| | Conserved | AGAACACCCGGAAACAAACCCAGGATTGCTGAAATGA |
| | trinucleotide | TATGTGACATTGATACATATATCGTAGAGGCAGGATTA |
| | motif: nt 9,207- | GCCAGTTTTATCCTGACTATTAAGTTTGGGATAGAAAC |
| | 9,209 | TATGTATCCTGCTCTTGGACTGCATGAATTTGCTGGTGA |
| | GS of P gene: | GTTATCCACACTTGAGTCCTTGATGAACCTTTACCAGC |
| | nt 9,210-9,226 | AAATGGGGGAAACTGCACCCTACATGGTAATCCTGGA |
| | BsiWI | GAACTCAATTCAGAACAAGTTCAGTGCAGGATCATACC |
| | restriction site: | CTCTGCTCTGGAGCTATGCCATGGGAGTAGGAGTGGAA |
| | nt 9,245-9,250 | CTTGAAAACTCCATGGGAGGTTTGAACTTTGGCCGATC |
| | EBV gH ORF: | TTACTTTGATCCAGCATATTTTAGATTAGGGCAAGAGA |
| | nt 9,251-11,368 | TGGTAAGGAGGTCAGCTGGAAAGGTCAGTTCCACATTG |
| | Furin cleavage | GCATCTGAACTCGGTATCACTGCCGAGGATGCAAGGCT |
| | site: nt 11,369- | TGTTTCAGAGATTGCAATGCATACTACTGAGGACAAGA |
| | 11,380 | TCAGTAGAGCGGTTGGACCCAGACAAGCCCAAGTATC |
| | P2A: nt 11,381- | ATTTCTACACGGTGATCAAAGTGAGAATGAGCTACCGA |
| | 11,437 | GATTGGGGGGCAAGGAAGATAGGAGGGTCAAACAGAG |
| | EBV gL ORF: | TCGAGGAGAAGCCAGGGAGAGCTACAGAGAAACCGGG |
| | nt 11,438- | CCCAGCAGAGCAAGTGATGCGAGAGCTGCCCATCTTCC |
| | 11848 | AACCGGCACACCCCTAGACATTGACACTGCAACGGAG |
| | Furin cleavage | TCCAGCCAAGATCCGCAGGACAGTCGAAGGTCAGCTG |
| | site: nt 11,849- | ACGCCCTGCTTAGGCTGCAAGCCATGGCAGGAATCTCG |
| | 11,860 | GAAGAACAAGGCTCAGACACGGACACCCCTATAGTGT |
| | T2A: nt 11,861- | ACAATGACAGAAATCTTCTAGACTAGGTGCGAGAGGC |
| | 11,914 | CGAGGGCCAGAACAACATCCGCCTACCATCCATCATTG |
| | EBV gp42 | TTATAAAAAACTTAGGAACCAGGTCCACACAGCCGCC |
| | ORF: nt | AGCCCATCAACCATCCACTCCCACGATTGGAGCCAATG |
| | 11,915-12,586 | GCAGAAGAGCAGGCACGCCATGTCAAAAACGGACTGG |
| | BssHII | AATGCATCCGGGCTCTCAAGGCCGAGCCCATCGGCTCA |
| | restriction site: | CTGGCCATCGAGGAAGCTATGGCAGCATGGTCAGAAA |
| | nt 12,587- | TATCAGACAACCCAGGACAGGAGCGAGCCACCTGCAG |
| | 12,592 | GGAAGAGAAGGCAGGCAGTTCGGGTCTCAGCAAACCA |
| | ATU | TGCCTCTCAGCAATTGGATCAACTGAAGGCGGTGCACC |
| | downstream | TCGCATCCGCGGTCAGGGACCTGGAGAGAGCGATGAC |
| | motif: nt | GACGCTGAAACTTTGGGAATCCCCCCAAGAAATCTCCA |
| | 12,587-12,622 | GGCATCAAGCACTGGGTTACAGTGTTATTACGTTTATG |
| | MV L ORF: nt | ATCACAGCGGTGAAGCGGTTAAGGGAATCCAAGATGC |
| | 12,677-19,228 | TGACTCTATCATGGTTCAATCAGGCCTTGATGGTGATA |
| | MV trailer: nt | GCACCCTCTCAGGAGGAGACAATGAATCTGAAAACAG |
| | 19,301-19,337 | CGATGTGGATATTGGCGAACCTGATACCGAGGGATATG |
| | | CTATCACTGACCGGGGATCTGCTCCCATCTCTATGGGG |
| | | TTCAGGGCTTCTGATGTTGAAACTGCAGAAGGAGGGG |
| | | AGATCCACGAGCTCCTGAGACTCCAATCCAGAGGCAA |
| | | CAACTTTCCGAAGCTTGGGAAAACTCTCAATGTTCCTC |
| | | CGCCCCCGGACCCCGGTAGGGCCAGCACTTCCGGGAC |
| | | ACCCATTAAAAAGGGCACAGACGCGAGATTAGCCTCA |
| | | TTTGGAACGGAGATCGCGTCTTTATTGACAGGTGGTGC |
| | | AACCCAATGTGCTCGAAAGTCACCCTCGGAACCATCAG |
| | | GGCCAGGTGCACCTGCGGGGAATGTCCCCGAGTGTGTG |
| | | AGCAATGCCGCACTGATACAGGAGTGGACACCCGAAT |
| | | CTGGTACCACAATCTCCCCGAGATCCCAGAATAATGAA |
| | | GAAGGGGGAGACTATTATGATGATGAGCTGTTCTCTGA |
| | | TGTCCAAGATATTAAAACAGCCTTGGCCAAAATACACG |
| | | AGGATAATCAGAAGATAATCTCCAAGCTAGAATCACT |
| | | GCTGTTATTGAAGGGAGAAGTTGAGTCAATTAAGAAG |
| | | CAGATCAACAGGCAAAATATCAGCATATCCACCCTGG |
| | | AAGGACACCTCTCAAGCATCATGATCGCCATTCCTGGA |
| | | CTTGGGAAGGATCCCAACGACCCCACTGCAGATGTCGA |
| | | AATCAATCCCGACTTGAAACCCATCATAGGCAGAGATT |
| | | CAGGCCGAGCACTGGCCGAAGTTCTCAAGAAACCCGTT |
| | | GCCAGCCGACAACTCCAAGGAATGACAAATGGACGGA |
| | | CCAGTTCCAGAGGACAGCTGCTGAAGGAATTTCAGCTA |
| | | AAGCCGATCGGGAAAAAGATGAGCTCAGCCGTCGGGT |
| | | TTGTTCCTGACACCGGCCCTGCATCACGCAGTGTAATC |
| | | CGCTCCATTATAAAATCCAGCCGGCTAGAGGAGGATCG |
| | | GAAGCGTTACCTGATGACTCTCCTTGATGATATCAAAG |
| | | GAGCCAATGATCTTGCCAAGTTCCACCAGATGCTGATG |
| | | AAGATAATAATGAAGTAGCTACAGCTCAACTTACCTGC |
| | | CAACCCCATGCCAGTCGACCCAACTAGTACAACCTAAA |
| | | TCCATTATAAAAAACTTAGGAGCAAAGTGATTGCCTCC |
| | | CAAGGTCCACAATGACAGAGACCTACGACTTCGACAA |
| | | GTCGGCATGGGACATCAAAGGGTCGATCGCTCCGATAC |
| | | AACCCACCACCTACAGTGATGGCAGGCTGGTGCCCCAG |
| | | GTCAGAGTCATAGATCCTGGTCTAGGCGACAGGAAGG |
| | | ATGAATGCTTTATGTACATGTTTCTGCTGGGGGTTGTTG |
| | | AGGACAGCGATTCCCTAGGGCCTCCAATCGGGCGAGC |
| | | ATTTGGGTTCCTGCCCTTAGGTGTTGGCAGATCCACAG |
| | | CAAAGCCCGAAAAACTCCTCAAAGAGGCCACTGAGCT |
| | | TGACATAGTTGTTAGACGTACAGCAGGGCTCAATGAAA |
| | | AACTGGTGTTCTACAACAACACCCCACTAACTCTCCTC |
| | | ACACCTTGGAGAAAGGTCCTAACAACAGGGAGTGTCTT |
| | | CAACGCAAACCAAGTGTGCAATGCGGTTAATCTGATAC |
| | | CGCTCGATACCCCGCAGAGGTTCCGTGTTGTTTATATG |
| | | AGCATCACCCGTCTTTCGGATAACGGGTATTACACCGT |
| | | TCCTAGAAGAATGCTGGAATTCAGATCGGTCAATGCAG |
| | | TGGCCTTCAACCTGCTGGTGACCCTTAGGATTGACAAG |
| | | GCGATAGGCCCTGGGAAGATCATCGACAATACAGAGC |
| | | AACTTCCTGAGGCAACATTTATGGTCCACATCGGGAAC |
| | | TTCAGGAGAAAGAAGAGTGAAGTCTACTCTGCCGATTA |
| | | TTGCAAAATGAAAATCGAAAAGATGGGCCTGGTTTTTG |
| | | CACTTGGTGGGATAGGGGGCACCAGTCTTCACATTAGA |
| | | AGCACAGGCAAAATGAGCAAGACTCTCCATGCACAAC |
| | | TCGGGTTCAAGAAGACCTTATGTTACCCGCTGATGGAT |
| | | ATCAATGAAGACCTTAATCGATTACTCTGGAGGAGCAG |
| | | ATGCAAGATAGTAAGAATCCAGGCAGTTTTGCAGCCAT |
| | | CAGTTCCTCAAGAATTCCGCATTTACGACGACGTGATC |
| | | ATAAATGATGACCAAGGACTATTCAAAGTTCTGTAGAC |
| | | CGTAGTGCCCAGCAATGCCCGAAAACGACCCCCCTCAC |
| | | AATGACAGCCAGAAGGCCCGGACAAAAAAGCCCCCTC |
| | | CGAAAGACTCCACGGACCAAGCGAGAGGCCAGCCAGC |
| | | AGCCGACGGCAAGCGCGAACACCAGGCGGCCCCAGCA |
| | | CAGAACAGCCCTGACACAAGGCCACCACCAGCCACCC |
| | | CAATCTGCATCCTCCTCGTGGGACCCCCGAGGACCAAC |
| | | CCCCAAGGCTGCCCCCGATCCAAACCACCAACCGCATC |
| | | CCCACCACCCCCGGGAAAGAAACCCCCAGCAATTGGA |
| | | AGGCCCCTCCCCCTCTTCCTCAACACAAGAACTCCACA |
| | | ACCGAACCGCACAAGCGACCGAGGTGACCCAACCGCA |
| | | GGCATCCGACTCCCTAGACAGATCCTCTCTCCCCGGCA |
| | | AACTAAACAAAACTTAGGGCCAAGGAACATACACACC |
| | | CAACAGAACCCAGACCCCGGCCCACGGCGCCGCGCCC |
| | | CCAACCCCCGACAACCAGAGGGAGCCCCCAACCAATC |
| | | CCGCCGGCTCCCCCGGTGCCCACAGGCAGGGACACCA |
| | | ACCCCCGAACAGACCCAGCACCCAACCATCGACAATC |
| | | CAAGACGGGGGGGCCCCCCCAAAAAAAGGCCCCCAGG |
| | | GGCCGACAGCCAGCACCGCGAGGAAGCCCACCCACCC |
| | | CACACACGACCACGGCAACCAAACCAGAACCCAGACC |
| | | ACCCTGGGCCACCAGCTCCCAGACTCGGCCATCACCCC |
| | | GCAGAAAGGAAAGGCCACAACCCGCGCACCCCAGCCC |
| | | CGATCCGGCGGGGAGCCACCCAACCCGAACCAGCACC |
| | | CAAGAGCGATCCCCGAAGGACCCCCGAACCGCAAAGG |
| | | ACATCAGTATCCCACAGCCTCTCCAAGTCCCCCGGTCT |
| | | CCTCCTCTTCTCGAAGGGACCAAAAGATCAATCCACCA |
| | | CACCCGACGACACTCAACTCCCCACCCCTAAAGGAGAC |
| | | ACCGGGAATCCCAGAATCAAGACTCATCCAATGTCCAT |
| | | CATGGGTCTCAAGGTGAACGTCTCTGCCATATTCATGG |
| | | CAGTACTGTTAACTCTCCAAACACCCACCGGTCAAATC |
| | | CATTGGGGCAATCTCTCTAAGATAGGGGTGGTAGGAAT |
| | | AGGAAGTGCAAGCTACAAAGTTATGACTCGTTCCAGCC |
| | | ATCAATCATTAGTCATAAAATTAATGCCCAATATAACT |
| | | CTCCTCAATAACTGCACGAGGGTAGAGATTGCAGAATA |
| | | CAGGAGACTACTGAGAACAGTTTTGGAACCAATTAGA |
| | | GATGCACTTAATGCAATGACCCAGAATATAAGACCGGT |
| | | TCAGAGTGTAGCTTCAAGTAGGAGACACAAGAGATTT |
| | | GCGGGAGTAGTCCTGGCAGGTGCGGCCCTAGGCGTTGC |
| | | CACAGCTGCTCAGATAACAGCCGGCATTGCACTTCACC |
| | | AGTCCATGCTGAACTCTCAAGCCATCGACAATCTGAGA |
| | | GCGAGCCTGGAAACTACTAATCAGGCAATTGAGACAA |
| | | TCAGACAAGCAGGGCAGGAGATGATATTGGCTGTTCA |
| | | GGGTGTCCAAGACTACATCAATAATGAGCTGATACCGT |
| | | CTATGAACCAACTATCTTGTGATTTAATCGGCCAGAAG |
| | | CTCGGGCTCAAATTGCTCAGATACTATACAGAAATCCT |
| | | GTCATTATTTGGCCCCAGTTTACGGGACCCCATATCTG |
| | | CGGAGATATCTATCCAGGCTTTGAGCTATGCGCTTGGA |
| | | GGAGACATCAATAAGGTGTTAGAAAAGCTCGGATACA |
| | | GTGGAGGTGATTTACTGGGCATCTTAGAGAGCGGAGG |
| | | CATCAAGGCCCGGATAACTCACGTCGACACAGAGTCCT |
| | | ACTTCATTGTCCTCAGTATAGCCTATCCGACGCTGTCCG |
| | | AGATTAAGGGGGTGATTGTCCACCGGCTAGAGGGGGT |
| | | CTCGTACAACATAGGCTCTCAAGAGTGGTATACCACTG |
| | | TGCCCAAGTATGTTGCAACCCAAGGGTACCTTATCTCG |
| | | AATTTTGATGAGTCATCGTGTACTTTCATGCCAGAGGG |
| | | GACTGTGTGCAGCCAAAATGCCTTGTACCCGATGAGTC |
| | | CTCTGCTCCAAGAATGCCTCCGGGGGTACACCAAGTCC |
| | | TGTGCTCGTACACTCGTATCCGGGTCTTTTGGGAACCG |
| | | GTTCATTTTATCACAAGGGAACCTAATAGCCAATTGTG |
| | | CATCAATCCTTTGCAAGTGTTACACAACAGGAACGATC |
| | | ATTAATCAAGACCCTGACAAGATCCTAACATACATTGC |
| | | TGCCGATCACTGCCCGGTAGTCGAGGTGAACGGCGTGA |
| | | CCATCCAAGTCGGGAGCAGGAGGTATCCAGACGCTGT |
| | | GTACTTGCACAGAATTGACCTCGGTCCTCCCATATCAT |
| | | TGGAGAGGTTGGACGTAGGGACAAATCTGGGGAATGC |
| | | AATTGCTAAGTTGGAGGATGCCAAGGAATTGTTGGAGT |
| | | CATCGGACCAGATATTGAGGAGTATGAAAGGTTTATCG |
| | | AGCACTAGCATAGTCTACATCCTGATTGCAGTGTGTCT |
| | | TGGAGGGTTGATAGGGATCCCCGCTTTAATATGTTGCT |
| | | GCAGGGGGCGTTGTAACAAAAAGGGAGAACAAGTTGG |
| | | TATGTCAAGACCAGGCCTAAAGCCTGATCTTACGGGAA |
| | | CATCAAAATCCTATGTAAGGTCGCTCTGATCCTCTACA |
| | | ACTCTTGAAACACAAATGTCCCACAAGTCTCCTCTTCG |
| | | TCATCAAGCAACCACCGCACCCAGCATCAAGCCCACCT |
| | | GAAATTATCTCCGGCTTCCCTCTGGCCGAACAATATCG |
| | | GTAGTTAATCAAAACTTAGGGTGCAAGATCATCCACAA |
| | | TGTCACCACAACGAGACCGGATAAATGCCTTCTACAAA |
| | | GATAACCCCCATCCCAAGGGAAGTAGGATAGTCATTA |
| | | ACAGAGAACATCTTATGATTGATAGACCTTATGTTTTG |
| | | CTGGCTGTTCTGTTTGTCATGTTTCTGAGCTTGATCGGG |
| | | TTGCTAGCCATTGCAGGCATTAGACTTCATCGGGCAGC |
| | | CATCTACACCGCAGAGATCCATAAAAGCCTCAGCACCA |
| | | ATCTAGATGTAACTAACTCAATCGAGCATCAGGTCAAG |
| | | GACGTGCTGACACCACTCTTCAAAATCATCGGTGATGA |
| | | AGTGGGCCTGAGGACACCTCAGAGATTCACTGACCTAG |
| | | TGAAATTAATCTCTGACAAGATCAAGTTCCTTAATCCG |
| | | GATAGGGAGTACGACTTCAGAGATCTCACTTGGTGTAT |
| | | CAACCCGCCAGAGAGAATCAAATTGGATTATGATCAAT |
| | | ACTGTGCAGATGTGGCTGCTGAAGAGCTCATGAATGCA |
| | | TTGGTGAACTCAACTCTACTGGAGACCAGAACAACCAA |
| | | TCAGTTCCTAGCTGTCTCAAAGGGAAACTGCTCAGGGC |
| | | CCACTACAATCAGAGGTCAATTCTCAAACATGTCGCTG |
| | | TCCCTGTTAGACTTGTATTTAGGTCGAGGTTACAATGT |
| | | GTCATCTATAGTCACTATGACATCCCAGGGAATGTATG |
| | | GGGGAACTTACCTAGTGGAAAAGCCTAATCTGAGCAG |
| | | CAAAAGGTCAGAGTTGTCACAACTGAGCATGTACCGA |
| | | GTGTTTGAAGTAGGTGTTATCAGAAATCCGGGTTTGGG |
| | | GGCTCCGGTGTTCCATATGACAAACTATCTTGAGCAAC |
| | | CAGTCAGTAATGATCTCAGCAACTGTATGGTGGCTTTG |
| | | GGGGAGCTCAAACTCGCAGCCCTTTGTCACGGGGAAG |
| | | ATTCTATCACAATTCCCTATCAGGGATCAGGGAAAGGT |
| | | GTCAGCTTCCAGCTCGTCAAGCTAGGTGTCTGGAAATC |
| | | CCCAACCGACATGCAATCCTGGGTCCCCTTATCAACGG |
| | | ATGATCCAGTGATAGACAGGCTTTACCTCTCATCTCAC |
| | | AGAGGTGTTATCGCTGACAATCAAGCAAAATGGGCTGT |
| | | CCCGACAACACGAACAGATGACAAGTTGCGAATGGAG |
| | | ACATGCTTCCAACAGGCGTGTAAGGGTAAAATCCAAG |
| | | CACTCTGCGAGAATCCCGAGTGGGCACCATTGAAGGAT |
| | | AACAGGATTCCTTCATACGGGGTCTTGTCTGTTGATCT |
| | | GAGTCTGACAGTTGAGCTTAAAATCAAAATTGCTTCGG |
| | | GATTCGGGCCATTGATCACACACGGTTCAGGGATGGAC |
| | | CTATACAAATCCAACCACAACAATGTGTATTGGCTGAC |
| | | TATCCCGCCAATGAAGAACCTAGCCTTAGGTGTAATCA |
| | | ACACATTGGAGTGGATACCGAGATTCAAGGTTAGTCCC |
| | | TACCTCTTCACTGTCCCAATTAAGGAAGCAGGCGAAGA |
| | | CTGCCATGCCCCAACATACCTACCTGCGGAGGTGGATG |
| | | GTGATGTCAAACTCAGTTCCAATCTGGTGATTCTACCT |
| | | GGTCAAGATCTCCAATATGTTTTGGCAACCTACGATAC |
| | | TTCCAGGGTTGAACATGCTGTGGTTTATTACGTTTACA |
| | | GCCCAAGCCGCTCATTTTCTTACTTTTATCCTTTTAGGT |
| | | TGCCTATAAAGGGGGTCCCCATCGAATTACAAGTGGAA |
| | | TGCTTCACATGGGACCAAAAACTCTGGTGCCGTCACTT |
| | | CTGTGTGCTTGCGGACTCAGAATCTGGTGGACATATCA |
| | | CTCACTCTGGGATGGTGGGCATGGGAGTCAGCTGCACA |
| | | GTCACCCGGGAAGATGGAACCAATCGCAGATAGGGCT |
| | | GCTAGTGAACCAATCACATGATGTCACCCAGACATCAG |
| | | GCATACCCACTAGTCTACCCTCCATCATTGTTATAAAA |
| | | AACTTAGGAACCAGGTCCACACAGCCGCCAGCCCATC |
| | | AACCGTACGATGCAACTCCTCTGCGTCTTTTGCTTGGTG |
| | | CTTTTGTGGGAGGTAGGAGCGGCCTCATTGTCAGAAGT |
| | | TAAGCTGCATCTCGATATTGAAGGCCACGCTTCACATT |
| | | ACACTATCCCTTGGACCGAACTCATGGCTAAGGTGCCG |
| | | GGTTTGTCTCCGGAAGCACTTTGGAGAGAGGCAAACGT |
| | | TACCGAAGACCTGGCCTCAATGCTGAATCGCTACAAGC |
| | | TGATATACAAGACTTCCGGAACCCTGGGTATAGCACTC |
| | | GCAGAGCCCGTCGATATCCCCGCTGTCAGCGAAGGTAG |
| | | CATGCAGGTGGACGCTAGTAAAGTTCACCCAGGAGTTA |
| | | TCTCAGGTCTGAATAGCCCAGCTTGTATGCTGAGCGCT |
| | | CCACTGGAGAAACAGCTCTTCTACTATATAGGCACAAT |
| | | GCTCCCTAACACTCGACCTCACAGCTACGTCTTCTATC |
| | | AGCTGCGCTGTCACCTGTCATACGTAGCCTTGTCCATT |
| | | AATGGTGATAAGTTCCAGTACACAGGTGCCATGACCTC |
| | | CAAGTTCCTTATGGGGACCTATAAACGGGTCACTGAGA |
| | | AAGGAGACGAGCATGTCCTTAGTTTGGTTTTTGGGAAG |
| | | ACAAAAGACCTGCCGGATCTGAGGGGCCCGTTCTCTTA |
| | | CCCCTCACTCACATCTGCACAGAGTGGGGACTATTCAT |
| | | TGGTGATTGTGACAACCTTCGTTCACTACGCCAATTTTC |
| | | ACAACTATTTTGTCCCGAACCTTAAAGACATGTTTTCA |
| | | CGCGCCGTGACCATGACAGCTGCTTCCTACGCTAGGTA |
| | | TGTGCTCCAAAAGCTGGTGCTGCTGGAGATGAAGGGC |
| | | GGATGCAGGGAACCTGAACTGGACACAGAGACTCTCA |
| | | CCACTATGTTCGAGGTTTCCGTGGCGTTCTTCAAGGTG |
| | | GGTCATGCTGTGGGGGAGACTGGGAACGGCTGCGTAG |
| | | ATCTTAGGTGGCTGGCTAAATCTTTCTTCGAGCTGACC |
| | | GTGTTGAAAGATATAATTGGAATTTGCTACGGGGCTAC |
| | | GGTTAAAGGAATGCAGTCTTACGGGCTGGAAAGGCTG |
| | | GCCGCTATGCTCATGGCTACTGTGAAGATGGAGGAACT |
| | | CGGCCACCTCACCACAGAAAAACAGGAGTACGCTTTG |
| | | CGCCTGGCCACTGTTGGCTACCCTAAGGCAGGCGTTTA |
| | | CTCTGGATTGATTGGGGGGGCCACATCTGTGCTCTTGT |
| | | CAGCCTACAATCGCCATCCCCTGTTCCAGCCTCTGCAC |
| | | ACGGTGATGCGAGAGACTTTGTTTATTGGCAGCCACGT |
| | | TGTGCTGAGAGAACTGCGCCTTAACGTTACCACTCAGG |
| | | GACCTAACCTTGCGCTGTATCAGCTGCTTAGCACCGCT |
| | | TTGTGCTCCGCTCTTGAAATTGGAGAAGTACTCAGAGG |
| | | GCTCGCACTGGGTACTGAGTCAGGGCTGTTCTCCCCCT |
| | | GCTATCTGTCTCTCCGGTTCGACCTCACTCGGGATAAG |
| | | CTGTTGTCAATGGCTCCGCAGGAAGCCACATTGGACCA |
| | | GGCCGCAGTGAGTAACGCAGTGGACGGGTTCCTGGGT |
| | | AGACTGTCTCTTGAGAGGGAAGACAGAGACGCTTGGC |
| | | ATCTGCCTGCGTATAAGTGTGTTGATAGACTGGACAAA |
| | | GTACTGATGATCATTCCCCTGATTAATGTGACATTTATT |
| | | ATATCTAGTGACCGAGAGGTGAGAGGCTCCGCTCTCTA |
| | | TGAAGCATCCACAACGTACTTGAGCAGTTCCCTGTTTC |
| | | TGTCACCGGTTATTATGAACAAATGTTCTCAGGGAGCT |
| | | GTTGCTGGTGAACCGCGACAGATTCCAAAGATTCAGAA |
| | | CTTCACTAGGACACAAAAGAGCTGCATTTTCTGCGGGT |
| | | TCGCACTGCTGAGTTACGACGAAAAGGAGGGACTGGA |
| | | GACTACTACATATATTACTTCACAGGAGGTGCAGAATA |
| | | GCATCCTGAGCTCCAATTATTTTGACTTTGACAACCTCC |
| | | ATGTCCATTATTTGCTGTTGACCACAAACGGCACTGTG |
| | | ATGGAGATCGCTGGCCTGTACGAGGAGCGAGCCCACG |
| | | TAGTACTGGCCATTATTCTGTACTTCATCGCTTTCGCAC |
| | | TTGGGATTTTCCTCGTCCACAAAATTGTTATGTTTTTCC |
| | | TCAGAGCAAAGAGAGCGACGAACTTCTCACTGCTTAA |
| | | GCAAGCAGGAGACGTGGAGGAGAATCCGGGCCCAATG |
| | | AGAGCCGTAGGTGTCTTTCTGGCTATTTGTCTGGTAAC |
| | | AATCTTCGTGCTGCCCACTTGGGGCAACTGGGCCTATC |
| | | CTTGCTGCCATGTTACACAGTTGAGAGCGCAGCACCTC |
| | | CTGGCGTTGGAGAACATTTCAGACATCTACCTGGTGAG |
| | | TAACCAAACCTGCGACGGGTTCTCTTTGGCCAGTCTCA |
| | | ATAGTCCAAAAAACGGAAGCAATCAGCTGGTAATTTC |
| | | ACGGTGTGCAAACGGACTTAACGTGGTCTCTTTTTTTAT |
| | | ATCAATCCTGAAACGCAGTTCATCTGCATTGACCGGCC |
| | | ACTTGAGGGAGCTCTTGACCACTCTCGAGACACTTTAC |
| | | GGGTCATTCTCCGTGGAGGATCTGTTCGGGGCAAATCT |
| | | GAACCGGTACGCCTGGCATAGAGGCGGGAGAGCAAAG |
| | | AGAGAGGGAAGAGGCTCCTTGCTGACCTGCGGTGACG |
| | | TGGAAGAGAATCCCGGCCCTATGGTGAGTTTTAAGCAG |
| | | GTCCGGGTACCTCTGTTCACCGCCATTGCGCTGGTGAT |
| | | TGTTCTGCTGTTGGCCTACTTTCTTCCACCTAGAGTCAG |
| | | AGGAGGTGGCAGGGTTGCTGCAGCTGCTATTACCTGGG |
| | | TACCGAAACCCAACGTGGAGGTTTGGCCAGTGGATCCG |
| | | CCCCCACCAGTGAACTTTAATAAAACAGCCGAACAAG |
| | | AATACGGGGATAAAGAAGTAAAGCTGCCACACTGGAC |
| | | CCCTACCCTCCACACTTTCCAGGTGCCTCAAAACTATA |
| | | CAAAGGCCAATTGCACATACTGTAACACAAGAGAATA |
| | | CACTTTTTCTTATAAAGGGTGCTGCTTCTACTTCACTAA |
| | | GAAGAAGCATACTTGGAACGGATGTTTCCAGGCATGC |
| | | GCTGAGTTGTATCCCTGTACCTATTTCTACGGTCCTACA |
| | | CCAGACATTCTGCCGGTCGTGACTCGCAATCTGAACGC |
| | | CATAGAATCTCTGTGGGTCGGGGTGTATCGGGTTGGGG |
| | | AAGGAAATTGGACTAGCCTGGACGGCGGTACTTTCAA |
| | | GGTGTACCAAATATTCGGCAGTCACTGTACTTACGTGT |
| | | CAAAATTCTCAACAGTTCCCGTATCCCACCACGAGTGT |
| | | TCTTTTTTGAAGCCTTGCCTGTGCGTGTCACAGAGGTCA |
| | | AATTCCTAAGCGCGCAGCGCTTAGACGTCTCGCGATCG |
| | | ATGCTAGTGTGAAATAGACATCAGAATTAAGAAAAAC |
| | | GTAGGGTCCAAGTGGTTCCCCGTTATGGACTCGCTATC |
| | | TGTCAACCAGATCTTATACCCTGAAGTTCACCTAGATA |
| | | GCCCGATAGTTACCAATAAGATAGTAGCCATCCTGGAG |
| | | TATGCTCGAGTCCCTCACGCTTACAGCCTGGAGGACCC |
| | | TACACTGTGTCAGAACATCAAGCACCGCCTAAAAAAC |
| | | GGATTTTCCAACCAAATGATTATAAACAATGTGGAAGT |
| | | TGGGAATGTCATCAAGTCCAAGCTTAGGAGTTATCCGG |
| | | CCCACTCTCATATTCCATATCCAAATTGTAATCAGGATT |
| | | TATTTAACATAGAAGACAAAGAGTCAACGAGGAAGAT |
| | | CCGTGAACTCCTCAAAAAGGGGAATTCGCTGTACTCCA |
| | | AAGTCAGTGATAAGGTTTTCCAATGCTTAAGGGACACT |
| | | AACTCACGGCTTGGCCTAGGCTCCGAATTGAGGGAGG |
| | | ACATCAAGGAGAAAGTTATTAACTTGGGAGTTTACATG |
| | | CACAGCTCCCAGTGGTTTGAGCCCTTTCTGTTTTGGTTT |
| | | ACAGTCAAGACTGAGATGAGGTCAGTGATTAAATCAC |
| | | AAACCCATACTTGCCATAGGAGGAGACACACACCTGT |
| | | ATTCTTCACTGGTAGTTCAGTTGAGTTGCTAATCTCTCG |
| | | TGACCTTGTTGCTATAATCAGTAAAGAGTCTCAACATG |
| | | TATATTACCTGACATTTGAACTGGTTTTGATGTATTGTG |
| | | ATGTCATAGAGGGGAGGTTAATGACAGAGACCGCTAT |
| | | GACTATTGATGCTAGGTATACAGAGCTTCTAGGAAGAG |
| | | TCAGATACATGTGGAAACTGATAGATGGTTTCTTCCCT |
| | | GCACTCGGGAATCCAACTTATCAAATTGTAGCCATGCT |
| | | GGAGCCTCTTTCACTTGCTTACCTGCAGCTGAGGGATA |
| | | TAACAGTAGAACTCAGAGGTGCTTTCCTTAACCACTGC |
| | | TTTACTGAAATACATGATGTTCTTGACCAAAACGGGTT |
| | | TTCTGATGAAGGTACTTATCATGAGTTAACTGAAGCTC |
| | | TAGATTACATTTTCATAACTGATGACATACATCTGACA |
| | | GGGGAGATTTTCTCATTTTTCAGAAGTTTCGGCCACCC |
| | | CAGACTTGAAGCAGTAACGGCTGCTGAAAATGTTAGG |
| | | AAATACATGAATCAGCCTAAAGTCATTGTGTATGAGAC |
| | | TCTGATGAAAGGTCATGCCATATTTTGTGGAATCATAA |
| | | TCAACGGCTATCGTGACAGGCACGGAGGCAGTTGGCC |
| | | ACCGCTGACCCTCCCCCTGCATGCTGCAGACACAATCC |
| | | GGAATGCTCAAGCTTCAGGTGAAGGGTTAACACATGA |
| | | GCAGTGCGTTGATAACTGGAAATCTTTTGCTGGAGTGA |
| | | AATTTGGCTGCTTTATGCCTCTTAGCCTGGATAGTGATC |
| | | TGACAATGTACCTAAAGGACAAGGCACTTGCTGCTCTC |
| | | CAAAGGGAATGGGATTCAGTTTACCCGAAAGAGTTCCT |
| | | GCGTTACGACCCTCCCAAGGGAACCGGGTCACGGAGG |
| | | CTTGTAGATGTTTTCCTTAATGATTCGAGCTTTGACCCA |
| | | TATGATGTGATAATGTATGTTGTAAGTGGAGCTTACCT |
| | | CCATGACCCTGAGTTCAACCTGTCTTACAGCCTGAAAG |
| | | AAAAGGAGATCAAGGAAACAGGTAGACTTTTTGCTAA |
| | | AATGACTTACAAAATGAGGGCATGCCAAGTGATTGCTG |
| | | AAAATCTAATCTCAAACGGGATTGGCAAATATTTTAAG |
| | | GACAATGGGATGGCCAAGGATGAGCACGATTTGACTA |
| | | AGGCACTCCACACTCTAGCTGTCTCAGGAGTCCCCAAA |
| | | GATCTCAAAGAAAGTCACAGGGGGGGGCCAGTCTTAA |
| | | AAACCTACTCCCGAAGCCCAGTCCACACAAGTACCAG |
| | | GAACGTGAGAGCAGCAAAAGGGTTTATAGGGTTCCCT |
| | | CAAGTAATTCGGCAGGACCAAGACACTGATCATCCGG |
| | | AGAATATGGAAGCTTACGAGACAGTCAGTGCATTTATC |
| | | ACGACTGATCTCAAGAAGTACTGCCTTAATTGGAGATA |
| | | TGAGACCATCAGCTTGTTTGCACAGAGGCTAAATGAGA |
| | | TTTACGGATTGCCCTCATTTTTCCAGTGGCTGCATAAGA |
| | | GGCTTGAGACCTCTGTCCTGTATGTAAGTGACCCTCAT |
| | | TGCCCCCCCGACCTTGACGCCCATATCCCGTTATATAA |
| | | AGTCCCCAATGATCAAATCTTCATTAAGTACCCTATGG |
| | | GAGGTATAGAAGGGTATTGTCAGAAGCTGTGGACCAT |
| | | CAGCACCATTCCCTATCTATACCTGGCTGCTTATGAGA |
| | | GCGGAGTAAGGATTGCTTCGTTAGTGCAAGGGGACAA |
| | | TCAGACCATAGCCGTAACAAAAAGGGTACCCAGCACA |
| | | TGGCCCTACAACCTTAAGAAACGGGAAGCTGCTAGAG |
| | | TAACTAGAGATTACTTTGTAATTCTTAGGCAAAGGCTA |
| | | CATGATATTGGCCATCACCTCAAGGCAAATGAGACAAT |
| | | TGTTTCATCACATTTTTTTGTCTATTCAAAAGGAATATA |
| | | TTATGATGGGCTACTTGTGTCCCAATCACTCAAGAGCA |
| | | TCGCAAGATGTGTATTCTGGTCAGAGACTATAGTTGAT |
| | | GAAACAAGGGCAGCATGCAGTAATATTGCTACAACAA |
| | | TGGCTAAAAGCATCGAGAGAGGTTATGACCGTTACCTT |
| | | GCATATTCCCTGAACGTCCTAAAAGTGATACAGCAAAT |
| | | TCTGATCTCTCTTGGCTTCACAATCAATTCAACCATGAC |
| | | CCGGGATGTAGTCATACCCCTCCTCACAAACAACGACC |
| | | TCTTAATAAGGATGGCACTGTTGCCCGCTCCTATTGGG |
| | | GGGATGAATTATCTGAATATGAGCAGGCTGTTTGTCAG |
| | | AAACATCGGTGATCCAGTAACATCATCAATTGCTGATC |
| | | TCAAGAGAATGATTCTCGCCTCACTAATGCCTGAAGAG |
| | | ACCCTCCATCAAGTAATGACACAACAACCGGGGGACT |
| | | CTTCATTCCTAGACTGGGCTAGCGACCCTTACTCAGCA |
| | | AATCTTGTATGTGTCCAGAGCATCACTAGACTCCTCAA |
| | | GAACATAACTGCAAGGTTTGTCCTGATCCATAGTCCAA |
| | | ACCCAATGTTAAAAGGATTATTCCATGATGACAGTAAA |
| | | GAAGAGGACGAGGGACTGGCGGCATTCCTCATGGACA |
| | | GGCATATTATAGTACCTAGGGCAGCTCATGAAATCCTG |
| | | GATCATAGTGTCACAGGGGCAAGAGAGTCTATTGCAG |
| | | GCATGCTGGATACCACAAAAGGCTTGATTCGAGCCAGC |
| | | ATGAGGAAGGGGGGGTTAACCTCTCGAGTGATAACCA |
| | | GATTGTCCAATTATGACTATGAACAATTCAGAGCAGGG |
| | | ATGGTGCTATTGACAGGAAGAAAGAGAAATGTCCTCA |
| | | TTGACAAAGAGTCATGTTCAGTGCAGCTGGCGAGAGCT |
| | | CTAAGAAGCCATATGTGGGCGAGGCTAGCTCGAGGAC |
| | | GGCCTATTTACGGCCTTGAGGTCCCTGATGTACTAGAA |
| | | TCTATGCGAGGCCACCTTATTCGGCGTCATGAGACATG |
| | | TGTCATCTGCGAGTGTGGATCAGTCAACTACGGATGGT |
| | | TTTTTGTCCCCTCGGGTTGCCAACTGGATGATATTGACA |
| | | AGGAAACATCATCCTTGAGAGTCCCATATATTGGTTCT |
| | | ACCACTGATGAGAGAACAGACATGAAGCTTGCCTTCGT |
| | | AAGAGCCCCAAGTCGATCCTTGCGATCTGCTGTTAGAA |
| | | TAGCAACAGTGTACTCATGGGCTTACGGTGATGATGAT |
| | | AGCTCTTGGAACGAAGCCTGGTTGTTGGCTAGGCAAAG |
| | | GGCCAATGTGAGCCTGGAGGAGCTAAGGGTGATCACT |
| | | CCCATCTCAACTTCGACTAATTTAGCGCATAGGTTGAG |
| | | GGATCGTAGCACTCAAGTGAAATACTCAGGTACATCCC |
| | | TTGTCCGAGTGGCGAGGTATACCACAATCTCCAACGAC |
| | | AATCTCTCATTTGTCATATCAGATAAGAAGGTTGATAC |
| | | TAACTTTATATACCAACAAGGAATGCTTCTAGGGTTGG |
| | | GTGTTTTAGAAACATTGTTTCGACTCGAGAAAGATACC |
| | | GGATCATCTAACACGGTATTACATCTTCACGTCGAAAC |
| | | AGATTGTTGCGTGATCCCGATGATAGATCATCCCAGGA |
| | | TACCCAGCTCCCGCAAGCTAGAGCTGAGGGCAGAGCT |
| | | ATGTACCAACCCATTGATATATGATAATGCACCTTTAA |
| | | TTGACAGAGATGCAACAAGGCTATACACCCAGAGCCA |
| | | TAGGAGGCACCTTGTGGAATTTGTTACATGGTCCACAC |
| | | CCCAACTATATCACATTTTAGCTAAGTCCACAGCACTA |
| | | TCTATGATTGACCTGGTAACAAAATTTGAGAAGGACCA |
| | | TATGAATGAAATTTCAGCTCTCATAGGGGATGACGATA |
| | | TCAATAGTTTCATAACTGAGTTTCTGCTCATAGAGCCA |
| | | AGATTATTCACTATCTACTTGGGCCAGTGTGCGGCCAT |
| | | CAATTGGGCATTTGATGTACATTATCATAGACCATCAG |
| | | GGAAATATCAGATGGGTGAGCTGTTGTCATCGTTCCTT |
| | | TCTAGAATGAGCAAAGGAGTGTTTAAGGTGCTTGTCAA |
| | | TGCTCTAAGCCACCCAAAGATCTACAAGAAATTCTGGC |
| | | ATTGTGGTATTATAGAGCCTATCCATGGTCCTTCACTTG |
| | | ATGCTCAAAACTTGCACACAACTGTGTGCAACATGGTT |
| | | TACACATGCTATATGACCTACCTCGACCTGTTGTTGAA |
| | | TGAAGAGTTAGAAGAGTTCACATTTCTCTTGTGTGAAA |
| | | GCGACGAGGATGTAGTACCGGACAGATTCGACAACAT |
| | | CCAGGCAAAACACTTATGTGTTCTGGCAGATTTGTACT |
| | | GTCAACCAGGGACCTGCCCACCAATTCGAGGTCTAAGA |
| | | CCGGTAGAGAAATGTGCAGTTCTAACCGACCATATCAA |
| | | GGCAGAGGCTATGTTATCTCCAGCAGGATCTTCGTGGA |
| | | ACATAAATCCAATTATTGTAGACCATTACTCATGCTCT |
| | | CTGACTTATCTCCGGCGAGGATCGATCAAACAGATAAG |
| | | ATTGAGAGTTGATCCAGGATTCATTTTCGACGCCCTCG |
| | | CTGAGGTAAATGTCAGTCAGCCAAAGATCGGCAGCAA |
| | | CAACATCTCAAATATGAGCATCAAGGCTTTCAGACCCC |
| | | CACACGATGATGTTGCAAAATTGCTCAAAGATATCAAC |
| | | ACAAGCAAGCACAATCTTCCCATTTCAGGGGGCAATCT |
| | | CGCCAATTATGAAATCCATGCTTTCCGCAGAATCGGGT |
| | | TGAACTCATCTGCTTGCTACAAAGCTGTTGAGATATCA |
| | | ACATTAATTAGGAGATGCCTTGAGCCAGGGGAGGACG |
| | | GCTTGTTCTTGGGTGAGGGATCGGGTTCTATGTTGATC |
| | | ACTTATAAAGAGATACTTAAACTAAACAAGTGCTTCTA |
| | | TAATAGTGGGGTTTCCGCCAATTCTAGATCTGGTCAAA |
| | | GGGAATTAGCACCCTATCCCTCCGAAGTTGGCCTTGTC |
| | | GAACACAGAATGGGAGTAGGTAATATTGTCAAAGTGC |
| | | TCTTTAACGGGAGGCCCGAAGTCACGTGGGTAGGCAGT |
| | | GTAGATTGCTTCAATTTCATAGTTAGTAATATCCCTACC |
| | | TCTAGTGTGGGGTTTATCCATTCAGATATAGAGACCTT |
| | | GCCTGACAAAGATACTATAGAGAAGCTAGAGGAATTG |
| | | GCAGCCATCTTATCGATGGCTCTGCTCCTGGGCAAAAT |
| | | AGGATCAATACTGGTGATTAAGCTTATGCCTTTCAGCG |
| | | GGGATTTTGTTCAGGGATTTATAAGTTATGTAGGGTCT |
| | | CATTATAGAGAAGTGAACCTTGTATACCCTAGATACAG |
| | | CAACTTCATCTCTACTGAATCTTATTTGGTTATGACAGA |
| | | TCTCAAGGCTAACCGGCTAATGAATCCTGAAAAGATTA |
| | | AGCAGCAGATAATTGAATCATCTGTGAGGACTTCACCT |
| | | GGACTTATAGGTCACATCCTATCCATTAAGCAACTAAG |
| | | CTGCATACAAGCAATTGTGGGAGACGCAGTTAGTAGA |
| | | GGTGATATCAATCCTACTCTGAAAAAACTTACACCTAT |
| | | AGAGCAGGTGCTGATCAATTGCGGGTTGGCAATTAACG |
| | | GACCTAAGCTGTGCAAAGAATTGATCCACCATGATGTT |
| | | GCCTCAGGGCAAGATGGATTGCTTAATTCTATACTCAT |
| | | CCTCTACAGGGAGTTGGCAAGATTCAAAGACAACCAA |
| | | AGAAGTCAACAAGGGATGTTCCACGCTTACCCCGTATT |
| | | GGTAAGTAGCAGGCAACGAGAACTTATATCTAGGATC |
| | | ACCCGCAAATTCTGGGGGCACATTCTTCTTTACTCCGG |
| | | GAACAAAAAGTTGATAAATAAGTTTATCCAGAATCTCA |
| | | AGTCCGGCTATCTGATACTAGACTTACACCAGAATATC |
| | | TTCGTTAAGAATCTATCCAAGTCAGAGAAACAGATTAT |
| | | TATGACGGGGGGTTTGAAACGTGAGTGGGTTTTTAAGG |
| | | TAACAGTCAAGGAGACCAAAGAATGGTATAAGTTAGT |
| | | CGGATACAGTGCCCTGATTAAGGACTAATTGGTTGAAC |
| | | TCCGGAACCCTAATCCTGCCCTAGGTGGTTAGGCATTA |
| | | TTTGCAATATATTAAAGAAAACTTTGAAAATACGAAGT |
| | | TTCTATTCCCAGCTTTGTCTGGT |
| 88 | T-10 | ACCAAACAAAGTTGGGTAAGGATAGTTCAATCAATGA |
| | EBV_gH-Fur- | TCATCTTCTAGTGCACTTAGGATTCAAGATCCTATTATC |
| | P2A_gL_Fur- | AGGGACAAGAGCAGGATTAGGGATATCCGAGATGGCC |
| | T2A_gp42 at | ACACTTTTAAGGAGCTTAGCATTGTTCAAAAGAAACAA |
| | ATU2 | GGACAAACCACCCATTACATCAGGATCCGGTGGAGCC |
| | MV leader: nt | ATCAGAGGAATCAAACACATTATTATAGTACCAATCCC |
| | 1-55 | TGGAGATTCCTCAATTACCACTCGATCCAGACTTCTGG |
| | MV N ORF: nt | ACCGGTTGGTGAGGTTAATTGGAAACCCGGATGTGAGC |
| | 108-1,685 | GGGCCCAAACTAACAGGGGCACTAATAGGTATATTATC |
| | GE of N gene: | CTTATTTGTGGAGTCTCCAGGTCAATTGATTCAGAGGA |
| | nt 1,734-1,744 | TCACCGATGACCCTGACGTTAGCATAAGGCTGTTAGAG |
| | GS of P gene: | GTTGTCCAGAGTGACCAGTCACAATCTGGCCTTACCTT |
| | nt 1,748-1,764 | CGCATCAAGAGGTACCAACATGGAGGATGAGGCGGAC |
| | MV P ORF: nt | CAATACTTTTCACATGATGATCCAATTAGTAGTGATCA |
| | 1,807-3,330 | ATCCAGGTTCGGATGGTTCGGGAACAAGGAAATCTCA |
| | ATU upstream | GATATTGAAGTGCAAGACCCTGAGGGATTCAACATGAT |
| | motif: nt 3,378- | TCTGGGTACCATCCTAGCCCAAATTTGGGTCTTGCTCG |
| | 3,455 | CAAAGGCGGTTACGGCCCCAGACACGGCAGCTGATTC |
| | GE of N gene: | GGAGCTAAGAAGGTGGATAAAGTACACCCAACAAAGA |
| | nt 3,394-3,404 | AGGGTAGTTGGTGAATTTAGATTGGAGAGAAAATGGTT |
| | Conserved | GGATGTGGTGAGGAACAGGATTGCCGAGGACCTCTCCT |
| | trinucleotide | TACGCCGATTCATGGTCGCTCTAATCCTGGATATCAAG |
| | motif: nt 3,405- | AGAACACCCGGAAACAAACCCAGGATTGCTGAAATGA |
| | 3,407 | TATGTGACATTGATACATATATCGTAGAGGCAGGATTA |
| | GS of P gene: | GCCAGTTTTATCCTGACTATTAAGTTTGGGATAGAAAC |
| | nt 3,408-3,424 | TATGTATCCTGCTCTTGGACTGCATGAATTTGCTGGTGA |
| | BsiWI | GTTATCCACACTTGAGTCCTTGATGAACCTTTACCAGC |
| | restriction site: | AAATGGGGGAAACTGCACCCTACATGGTAATCCTGGA |
| | nt 3,444-3,4496 | GAACTCAATTCAGAACAAGTTCAGTGCAGGATCATACC |
| | BsiWI | CTCTGCTCTGGAGCTATGCCATGGGAGTAGGAGTGGAA |
| | restriction site: | CTTGAAAACTCCATGGGAGGTTTGAACTTTGGCCGATC |
| | nt 3,456-3,4616 | TTACTTTGATCCAGCATATTTTAGATTAGGGCAAGAGA |
| | EBV gH ORF: | TGGTAAGGAGGTCAGCTGGAAAGGTCAGTTCCACATTG |
| | nt 3,462 | GCATCTGAACTCGGTATCACTGCCGAGGATGCAAGGCT |
| | Furin cleavage | TGTTTCAGAGATTGCAATGCATACTACTGAGGACAAGA |
| | site: nt 5,580- | TCAGTAGAGCGGTTGGACCCAGACAAGCCCAAGTATC |
| | 5,591 | ATTTCTACACGGTGATCAAAGTGAGAATGAGCTACCGA |
| | P2A: nt 5,592- | GATTGGGGGGCAAGGAAGATAGGAGGGTCAAACAGAG |
| | 5,648 | TCGAGGAGAAGCCAGGGAGAGCTACAGAGAAACCGGG |
| | EBV gL ORF: | CCCAGCAGAGCAAGTGATGCGAGAGCTGCCCATCTTCC |
| | nt 5,649-6059 | AACCGGCACACCCCTAGACATTGACACTGCAACGGAG |
| | Furin cleavage | TCCAGCCAAGATCCGCAGGACAGTCGAAGGTCAGCTG |
| | site: nt 6,060- | ACGCCCTGCTTAGGCTGCAAGCCATGGCAGGAATCTCG |
| | 6,071 | GAAGAACAAGGCTCAGACACGGACACCCCTATAGTGT |
| | T2A: nt 6,072- | ACAATGACAGAAATCTTCTAGACTAGGTGCGAGAGGC |
| | 6,125 | CGAGGGCCAGAACAACATCCGCCTACCATCCATCATTG |
| | EBV gp42 | TTATAAAAAACTTAGGAACCAGGTCCACACAGCCGCC |
| | ORF: nt 6,126- | AGCCCATCAACCATCCACTCCCACGATTGGAGCCAATG |
| | 6,797 | GCAGAAGAGCAGGCACGCCATGTCAAAAACGGACTGG |
| | ATU | AATGCATCCGGGCTCTCAAGGCCGAGCCCATCGGCTCA |
| | downstream | CTGGCCATCGAGGAAGCTATGGCAGCATGGTCAGAAA |
| | motif: nt 6,798- | TATCAGACAACCCAGGACAGGAGCGAGCCACCTGCAG |
| | 6,833 | GGAAGAGAAGGCAGGCAGTTCGGGTCTCAGCAAACCA |
| | BssHII | TGCCTCTCAGCAATTGGATCAACTGAAGGCGGTGCACC |
| | restriction site: | TCGCATCCGCGGTCAGGGACCTGGAGAGAGCGATGAC |
| | nt 6,798-6,803 | GACGCTGAAACTTTGGGAATCCCCCCAAGAAATCTCCA |
| | GE of P gene: | GGCATCAAGCACTGGGTTACAGTGTTATTACGTTTATG |
| | nt 6,848-6,858 | ATCACAGCGGTGAAGCGGTTAAGGGAATCCAAGATGC |
| | GS of M gene: | TGACTCTATCATGGTTCAATCAGGCCTTGATGGTGATA |
| | nt 6,862-6,878 | GCACCCTCTCAGGAGGAGACAATGAATCTGAAAACAG |
| | MV M ORF: nt | CGATGTGGATATTGGCGAACCTGATACCGAGGGATATG |
| | 6,894-7,901 | CTATCACTGACCGGGGATCTGCTCCCATCTCTATGGGG |
| | MV F ORF: nt | TTCAGGGCTTCTGATGTTGAAACTGCAGAAGGAGGGG |
| | 8,905-10,566 | AGATCCACGAGCTCCTGAGACTCCAATCCAGAGGCAA |
| | MV H ORF: nt | CAACTTTCCGAAGCTTGGGAAAACTCTCAATGTTCCTC |
| | 10,727-12,580 | CGCCCCCGGACCCCGGTAGGGCCAGCACTTCCGGGAC |
| | MV L ORF: nt | ACCCATTAAAAAGGGCACAGACGCGAGATTAGCCTCA |
| | 12,690-19,241 | TTTGGAACGGAGATCGCGTCTTTATTGACAGGTGGTGC |
| | MV trailer: nt | AACCCAATGTGCTCGAAAGTCACCCTCGGAACCATCAG |
| | 19,314-19,350 | GGCCAGGTGCACCTGCGGGGAATGTCCCCGAGTGTGTG |
| | | AGCAATGCCGCACTGATACAGGAGTGGACACCCGAAT |
| | | CTGGTACCACAATCTCCCCGAGATCCCAGAATAATGAA |
| | | GAAGGGGGAGACTATTATGATGATGAGCTGTTCTCTGA |
| | | TGTCCAAGATATTAAAACAGCCTTGGCCAAAATACACG |
| | | AGGATAATCAGAAGATAATCTCCAAGCTAGAATCACT |
| | | GCTGTTATTGAAGGGAGAAGTTGAGTCAATTAAGAAG |
| | | CAGATCAACAGGCAAAATATCAGCATATCCACCCTGG |
| | | AAGGACACCTCTCAAGCATCATGATCGCCATTCCTGGA |
| | | CTTGGGAAGGATCCCAACGACCCCACTGCAGATGTCGA |
| | | AATCAATCCCGACTTGAAACCCATCATAGGCAGAGATT |
| | | CAGGCCGAGCACTGGCCGAAGTTCTCAAGAAACCCGTT |
| | | GCCAGCCGACAACTCCAAGGAATGACAAATGGACGGA |
| | | CCAGTTCCAGAGGACAGCTGCTGAAGGAATTTCAGCTA |
| | | AAGCCGATCGGGAAAAAGATGAGCTCAGCCGTCGGGT |
| | | TTGTTCCTGACACCGGCCCTGCATCACGCAGTGTAATC |
| | | CGCTCCATTATAAAATCCAGCCGGCTAGAGGAGGATCG |
| | | GAAGCGTTACCTGATGACTCTCCTTGATGATATCAAAG |
| | | GAGCCAATGATCTTGCCAAGTTCCACCAGATGCTGATG |
| | | AAGATAATAATGAAGTAGCTACAGCTCAACTTACCTGC |
| | | CAACCCCATGCCAGTCGACCCAACTAGCCTACCCTCCA |
| | | TCATTGTTATAAAAAACTTAGGAACCAGGTCCACACAG |
| | | CCGCCAGCCCATCAACGCGTACGGCCACCCGTACGATG |
| | | CAACTCCTCTGCGTCTTTTGCTTGGTGCTTTTGTGGGAG |
| | | GTAGGAGCGGCCTCATTGTCAGAAGTTAAGCTGCATCT |
| | | CGATATTGAAGGCCACGCTTCACATTACACTATCCCTT |
| | | GGACCGAACTCATGGCTAAGGTGCCGGGTTTGTCTCCG |
| | | GAAGCACTTTGGAGAGAGGCAAACGTTACCGAAGACC |
| | | TGGCCTCAATGCTGAATCGCTACAAGCTGATATACAAG |
| | | ACTTCCGGAACCCTGGGTATAGCACTCGCAGAGCCCGT |
| | | CGATATCCCCGCTGTCAGCGAAGGTAGCATGCAGGTGG |
| | | ACGCTAGTAAAGTTCACCCAGGAGTTATCTCAGGTCTG |
| | | AATAGCCCAGCTTGTATGCTGAGCGCTCCACTGGAGAA |
| | | ACAGCTCTTCTACTATATAGGCACAATGCTCCCTAACA |
| | | CTCGACCTCACAGCTACGTCTTCTATCAGCTGCGCTGTC |
| | | ACCTGTCATACGTAGCCTTGTCCATTAATGGTGATAAG |
| | | TTCCAGTACACAGGTGCCATGACCTCCAAGTTCCTTAT |
| | | GGGGACCTATAAACGGGTCACTGAGAAAGGAGACGAG |
| | | CATGTCCTTAGTTTGGTTTTTGGGAAGACAAAAGACCT |
| | | GCCGGATCTGAGGGGCCCGTTCTCTTACCCCTCACTCA |
| | | CATCTGCACAGAGTGGGGACTATTCATTGGTGATTGTG |
| | | ACAACCTTCGTTCACTACGCCAATTTTCACAACTATTTT |
| | | GTCCCGAACCTTAAAGACATGTTTTCACGCGCCGTGAC |
| | | CATGACAGCTGCTTCCTACGCTAGGTATGTGCTCCAAA |
| | | AGCTGGTGCTGCTGGAGATGAAGGGCGGATGCAGGGA |
| | | ACCTGAACTGGACACAGAGACTCTCACCACTATGTTCG |
| | | AGGTTTCCGTGGCGTTCTTCAAGGTGGGTCATGCTGTG |
| | | GGGGAGACTGGGAACGGCTGCGTAGATCTTAGGTGGC |
| | | TGGCTAAATCTTTCTTCGAGCTGACCGTGTTGAAAGAT |
| | | ATAATTGGAATTTGCTACGGGGCTACGGTTAAAGGAAT |
| | | GCAGTCTTACGGGCTGGAAAGGCTGGCCGCTATGCTCA |
| | | TGGCTACTGTGAAGATGGAGGAACTCGGCCACCTCACC |
| | | ACAGAAAAACAGGAGTACGCTTTGCGCCTGGCCACTGT |
| | | TGGCTACCCTAAGGCAGGCGTTTACTCTGGATTGATTG |
| | | GGGGGGCCACATCTGTGCTCTTGTCAGCCTACAATCGC |
| | | CATCCCCTGTTCCAGCCTCTGCACACGGTGATGCGAGA |
| | | GACTTTGTTTATTGGCAGCCACGTTGTGCTGAGAGAAC |
| | | TGCGCCTTAACGTTACCACTCAGGGACCTAACCTTGCG |
| | | CTGTATCAGCTGCTTAGCACCGCTTTGTGCTCCGCTCTT |
| | | GAAATTGGAGAAGTACTCAGAGGGCTCGCACTGGGTA |
| | | CTGAGTCAGGGCTGTTCTCCCCCTGCTATCTGTCTCTCC |
| | | GGTTCGACCTCACTCGGGATAAGCTGTTGTCAATGGCT |
| | | CCGCAGGAAGCCACATTGGACCAGGCCGCAGTGAGTA |
| | | ACGCAGTGGACGGGTTCCTGGGTAGACTGTCTCTTGAG |
| | | AGGGAAGACAGAGACGCTTGGCATCTGCCTGCGTATA |
| | | AGTGTGTTGATAGACTGGACAAAGTACTGATGATCATT |
| | | CCCCTGATTAATGTGACATTTATTATATCTAGTGACCG |
| | | AGAGGTGAGAGGCTCCGCTCTCTATGAAGCATCCACAA |
| | | CGTACTTGAGCAGTTCCCTGTTTCTGTCACCGGTTATTA |
| | | TGAACAAATGTTCTCAGGGAGCTGTTGCTGGTGAACCG |
| | | CGACAGATTCCAAAGATTCAGAACTTCACTAGGACACA |
| | | AAAGAGCTGCATTTTCTGCGGGTTCGCACTGCTGAGTT |
| | | ACGACGAAAAGGAGGGACTGGAGACTACTACATATAT |
| | | TACTTCACAGGAGGTGCAGAATAGCATCCTGAGCTCCA |
| | | ATTATTTTGACTTTGACAACCTCCATGTCCATTATTTGC |
| | | TGTTGACCACAAACGGCACTGTGATGGAGATCGCTGGC |
| | | CTGTACGAGGAGCGAGCCCACGTAGTACTGGCCATTAT |
| | | TCTGTACTTCATCGCTTTCGCACTTGGGATTTTCCTCGT |
| | | CCACAAAATTGTTATGTTTTTCCTCAGAGCAAAGAGAG |
| | | CGACGAACTTCTCACTGCTTAAGCAAGCAGGAGACGTG |
| | | GAGGAGAATCCGGGCCCAATGAGAGCCGTAGGTGTCT |
| | | TTCTGGCTATTTGTCTGGTAACAATCTTCGTGCTGCCCA |
| | | CTTGGGGCAACTGGGCCTATCCTTGCTGCCATGTTACA |
| | | CAGTTGAGAGCGCAGCACCTCCTGGCGTTGGAGAACAT |
| | | TTCAGACATCTACCTGGTGAGTAACCAAACCTGCGACG |
| | | GGTTCTCTTTGGCCAGTCTCAATAGTCCAAAAAACGGA |
| | | AGCAATCAGCTGGTAATTTCACGGTGTGCAAACGGACT |
| | | TAACGTGGTCTCTTTTTTTATATCAATCCTGAAACGCAG |
| | | TTCATCTGCATTGACCGGCCACTTGAGGGAGCTCTTGA |
| | | CCACTCTCGAGACACTTTACGGGTCATTCTCCGTGGAG |
| | | GATCTGTTCGGGGCAAATCTGAACCGGTACGCCTGGCA |
| | | TAGAGGCGGGAGAGCAAAGAGAGAGGGAAGAGGCTC |
| | | CTTGCTGACCTGCGGTGACGTGGAAGAGAATCCCGGCC |
| | | CTATGGTGAGTTTTAAGCAGGTCCGGGTACCTCTGTTC |
| | | ACCGCCATTGCGCTGGTGATTGTTCTGCTGTTGGCCTAC |
| | | TTTCTTCCACCTAGAGTCAGAGGAGGTGGCAGGGTTGC |
| | | TGCAGCTGCTATTACCTGGGTACCGAAACCCAACGTGG |
| | | AGGTTTGGCCAGTGGATCCGCCCCCACCAGTGAACTTT |
| | | AATAAAACAGCCGAACAAGAATACGGGGATAAAGAAG |
| | | TAAAGCTGCCACACTGGACCCCTACCCTCCACACTTTC |
| | | CAGGTGCCTCAAAACTATACAAAGGCCAATTGCACATA |
| | | CTGTAACACAAGAGAATACACTTTTTCTTATAAAGGGT |
| | | GCTGCTTCTACTTCACTAAGAAGAAGCATACTTGGAAC |
| | | GGATGTTTCCAGGCATGCGCTGAGTTGTATCCCTGTAC |
| | | CTATTTCTACGGTCCTACACCAGACATTCTGCCGGTCGT |
| | | GACTCGCAATCTGAACGCCATAGAATCTCTGTGGGTCG |
| | | GGGTGTATCGGGTTGGGGAAGGAAATTGGACTAGCCT |
| | | GGACGGCGGTACTTTCAAGGTGTACCAAATATTCGGCA |
| | | GTCACTGTACTTACGTGTCAAAATTCTCAACAGTTCCC |
| | | GTATCCCACCACGAGTGTTCTTTTTTGAAGCCTTGCCTG |
| | | TGCGTGTCACAGAGGTCAAATTCCTAAGCGCGCAGCGC |
| | | TTAGACGTCTCGCGATCGATACTAGTACAACCTAAATC |
| | | CATTATAAAAAACTTAGGAGCAAAGTGATTGCCTCCCA |
| | | AGGTCCACAATGACAGAGACCTACGACTTCGACAAGT |
| | | CGGCATGGGACATCAAAGGGTCGATCGCTCCGATACA |
| | | ACCCACCACCTACAGTGATGGCAGGCTGGTGCCCCAGG |
| | | TCAGAGTCATAGATCCTGGTCTAGGCGACAGGAAGGA |
| | | TGAATGCTTTATGTACATGTTTCTGCTGGGGGTTGTTGA |
| | | GGACAGCGATTCCCTAGGGCCTCCAATCGGGCGAGCAT |
| | | TTGGGTTCCTGCCCTTAGGTGTTGGCAGATCCACAGCA |
| | | AAGCCCGAAAAACTCCTCAAAGAGGCCACTGAGCTTG |
| | | ACATAGTTGTTAGACGTACAGCAGGGCTCAATGAAAA |
| | | ACTGGTGTTCTACAACAACACCCCACTAACTCTCCTCA |
| | | CACCTTGGAGAAAGGTCCTAACAACAGGGAGTGTCTTC |
| | | AACGCAAACCAAGTGTGCAATGCGGTTAATCTGATACC |
| | | GCTCGATACCCCGCAGAGGTTCCGTGTTGTTTATATGA |
| | | GCATCACCCGTCTTTCGGATAACGGGTATTACACCGTT |
| | | CCTAGAAGAATGCTGGAATTCAGATCGGTCAATGCAGT |
| | | GGCCTTCAACCTGCTGGTGACCCTTAGGATTGACAAGG |
| | | CGATAGGCCCTGGGAAGATCATCGACAATACAGAGCA |
| | | ACTTCCTGAGGCAACATTTATGGTCCACATCGGGAACT |
| | | TCAGGAGAAAGAAGAGTGAAGTCTACTCTGCCGATTAT |
| | | TGCAAAATGAAAATCGAAAAGATGGGCCTGGTTTTTGC |
| | | ACTTGGTGGGATAGGGGGCACCAGTCTTCACATTAGAA |
| | | GCACAGGCAAAATGAGCAAGACTCTCCATGCACAACT |
| | | CGGGTTCAAGAAGACCTTATGTTACCCGCTGATGGATA |
| | | TCAATGAAGACCTTAATCGATTACTCTGGAGGAGCAGA |
| | | TGCAAGATAGTAAGAATCCAGGCAGTTTTGCAGCCATC |
| | | AGTTCCTCAAGAATTCCGCATTTACGACGACGTGATCA |
| | | TAAATGATGACCAAGGACTATTCAAAGTTCTGTAGACC |
| | | GTAGTGCCCAGCAATGCCCGAAAACGACCCCCCTCACA |
| | | ATGACAGCCAGAAGGCCCGGACAAAAAAGCCCCCTCC |
| | | GAAAGACTCCACGGACCAAGCGAGAGGCCAGCCAGCA |
| | | GCCGACGGCAAGCGCGAACACCAGGCGGCCCCAGCAC |
| | | AGAACAGCCCTGACACAAGGCCACCACCAGCCACCCC |
| | | AATCTGCATCCTCCTCGTGGGACCCCCGAGGACCAACC |
| | | CCCAAGGCTGCCCCCGATCCAAACCACCAACCGCATCC |
| | | CCACCACCCCCGGGAAAGAAACCCCCAGCAATTGGAA |
| | | GGCCCCTCCCCCTCTTCCTCAACACAAGAACTCCACAA |
| | | CCGAACCGCACAAGCGACCGAGGTGACCCAACCGCAG |
| | | GCATCCGACTCCCTAGACAGATCCTCTCTCCCCGGCAA |
| | | ACTAAACAAAACTTAGGGCCAAGGAACATACACACCC |
| | | AACAGAACCCAGACCCCGGCCCACGGCGCCGCGCCCC |
| | | CAACCCCCGACAACCAGAGGGAGCCCCCAACCAATCC |
| | | CGCCGGCTCCCCCGGTGCCCACAGGCAGGGACACCAA |
| | | CCCCCGAACAGACCCAGCACCCAACCATCGACAATCC |
| | | AAGACGGGGGGGCCCCCCCAAAAAAAGGCCCCCAGGG |
| | | GCCGACAGCCAGCACCGCGAGGAAGCCCACCCACCCC |
| | | ACACACGACCACGGCAACCAAACCAGAACCCAGACCA |
| | | CCCTGGGCCACCAGCTCCCAGACTCGGCCATCACCCCG |
| | | CAGAAAGGAAAGGCCACAACCCGCGCACCCCAGCCCC |
| | | GATCCGGCGGGGAGCCACCCAACCCGAACCAGCACCC |
| | | AAGAGCGATCCCCGAAGGACCCCCGAACCGCAAAGGA |
| | | CATCAGTATCCCACAGCCTCTCCAAGTCCCCCGGTCTC |
| | | CTCCTCTTCTCGAAGGGACCAAAAGATCAATCCACCAC |
| | | ACCCGACGACACTCAACTCCCCACCCCTAAAGGAGAC |
| | | ACCGGGAATCCCAGAATCAAGACTCATCCAATGTCCAT |
| | | CATGGGTCTCAAGGTGAACGTCTCTGCCATATTCATGG |
| | | CAGTACTGTTAACTCTCCAAACACCCACCGGTCAAATC |
| | | CATTGGGGCAATCTCTCTAAGATAGGGGGGTAGGAAT |
| | | AGGAAGTGCAAGCTACAAAGTTATGACTCGTTCCAGCC |
| | | ATCAATCATTAGTCATAAAATTAATGCCCAATATAACT |
| | | CTCCTCAATAACTGCACGAGGGTAGAGATTGCAGAATA |
| | | CAGGAGACTACTGAGAACAGTTTTGGAACCAATTAGA |
| | | GATGCACTTAATGCAATGACCCAGAATATAAGACCGGT |
| | | TCAGAGTGTAGCTTCAAGTAGGAGACACAAGAGATTT |
| | | GCGGGAGTAGTCCTGGCAGGTGCGGCCCTAGGCGTTGC |
| | | CACAGCTGCTCAGATAACAGCCGGCATTGCACTTCACC |
| | | AGTCCATGCTGAACTCTCAAGCCATCGACAATCTGAGA |
| | | GCGAGCCTGGAAACTACTAATCAGGCAATTGAGACAA |
| | | TCAGACAAGCAGGGCAGGAGATGATATTGGCTGTTCA |
| | | GGGTGTCCAAGACTACATCAATAATGAGCTGATACCGT |
| | | CTATGAACCAACTATCTTGTGATTTAATCGGCCAGAAG |
| | | CTCGGGCTCAAATTGCTCAGATACTATACAGAAATCCT |
| | | GTCATTATTTGGCCCCAGTTTACGGGACCCCATATCTG |
| | | CGGAGATATCTATCCAGGCTTTGAGCTATGCGCTTGGA |
| | | GGAGACATCAATAAGGTGTTAGAAAAGCTCGGATACA |
| | | GTGGAGGTGATTTACTGGGCATCTTAGAGAGCGGAGG |
| | | AATAAAGGCCCGGATAACTCACGTCGACACAGAGTCC |
| | | TACTTCATTGTCCTCAGTATAGCCTATCCGACGCTGTCC |
| | | GAGATTAAGGGGGTGATTGTCCACCGGCTAGAGGGGG |
| | | TCTCGTACAACATAGGCTCTCAAGAGTGGTATACCACT |
| | | GTGCCCAAGTATGTTGCAACCCAAGGGTACCTTATCTC |
| | | GAATTTTGATGAGTCATCGTGTACTTTCATGCCAGAGG |
| | | GGACTGTGTGCAGCCAAAATGCCTTGTACCCGATGAGT |
| | | CCTCTGCTCCAAGAATGCCTCCGGGGGTACACCAAGTC |
| | | CTGTGCTCGTACACTCGTATCCGGGTCTTTTGGGAACC |
| | | GGTTCATTTTATCACAAGGGAACCTAATAGCCAATTGT |
| | | GCATCAATCCTTTGCAAGTGTTACACAACAGGAACGAT |
| | | CATTAATCAAGACCCTGACAAGATCCTAACATACATTG |
| | | CTGCCGATCACTGCCCGGTAGTCGAGGTGAACGGCGTG |
| | | ACCATCCAAGTCGGGAGCAGGAGGTATCCAGACGCTG |
| | | TGTACTTGCACAGAATTGACCTCGGTCCTCCCATATCA |
| | | TTGGAGAGGTTGGACGTAGGGACAAATCTGGGGAATG |
| | | CAATTGCTAAGTTGGAGGATGCCAAGGAATTGTTGGAG |
| | | TCATCGGACCAGATATTGAGGAGTATGAAAGGTTTATC |
| | | GAGCACTAGCATAGTCTACATCCTGATTGCAGTGTGTC |
| | | TTGGAGGGTTGATAGGGATCCCCGCTTTAATATGTTGC |
| | | TGCAGGGGGCGTTGTAACAAAAAGGGAGAACAAGTTG |
| | | GTATGTCAAGACCAGGCCTAAAGCCTGATCTTACGGGA |
| | | ACATCAAAATCCTATGTAAGGTCGCTCTGATCCTCTAC |
| | | AACTCTTGAAACACAAATGTCCCACAAGTCTCCTCTTC |
| | | GTCATCAAGCAACCACCGCACCCAGCATCAAGCCCACC |
| | | TGAAATTATCTCCGGCTTCCCTCTGGCCGAACAATATC |
| | | GGTAGTTAATCAAAACTTAGGGTGCAAGATCATCCACA |
| | | ATGTCACCACAACGAGACCGGATAAATGCCTTCTACAA |
| | | AGATAACCCCCATCCCAAGGGAAGTAGGATAGTCATT |
| | | AACAGAGAACATCTTATGATTGATAGACCTTATGTTTT |
| | | GCTGGCTGTTCTGTTTGTCATGTTTCTGAGCTTGATCGG |
| | | GTTGCTAGCCATTGCAGGCATTAGACTTCATCGGGCAG |
| | | CCATCTACACCGCAGAGATCCATAAAAGCCTCAGCACC |
| | | AATCTAGATGTAACTAACTCAATCGAGCATCAGGTCAA |
| | | GGACGTGCTGACACCACTCTTCAAAATCATCGGTGATG |
| | | AAGTGGGCCTGAGGACACCTCAGAGATTCACTGACCTA |
| | | GTGAAATTAATCTCTGACAAGATTAAATTCCTTAATCC |
| | | GGATAGGGAGTACGACTTCAGAGATCTCACTTGGTGTA |
| | | TCAACCCGCCAGAGAGAATCAAATTGGATTATGATCAA |
| | | TACTGTGCAGATGTGGCTGCTGAAGAGCTCATGAATGC |
| | | ATTGGTGAACTCAACTCTACTGGAGACCAGAACAACCA |
| | | ATCAGTTCCTAGCTGTCTCAAAGGGAAACTGCTCAGGG |
| | | CCCACTACAATCAGAGGTCAATTCTCAAACATGTCGCT |
| | | GTCCCTGTTAGACTTGTATTTAGGTCGAGGTTACAATG |
| | | TGTCATCTATAGTCACTATGACATCCCAGGGAATGTAT |
| | | GGGGGAACTTACCTAGTGGAAAAGCCTAATCTGAGCA |
| | | GCAAAAGGTCAGAGTTGTCACAACTGAGCATGTACCG |
| | | AGTGTTTGAAGTAGGTGTTATCAGAAATCCGGGTTTGG |
| | | GGGCTCCGGTGTTCCATATGACAAACTATCTTGAGCAA |
| | | CCAGTCAGTAATGATCTCAGCAACTGTATGGTGGCTTT |
| | | GGGGGAGCTCAAACTCGCAGCCCTTTGTCACGGGGAA |
| | | GATTCTATCACAATTCCCTATCAGGGATCAGGGAAAGG |
| | | TGTCAGCTTCCAGCTCGTCAAGCTAGGTGTCTGGAAAT |
| | | CCCCAACCGACATGCAATCCTGGGTCCCCTTATCAACG |
| | | GATGATCCAGTGATAGACAGGCTTTACCTCTCATCTCA |
| | | CAGAGGTGTTATCGCTGACAATCAAGCAAAATGGGCT |
| | | GTCCCGACAACACGAACAGATGACAAGTTGCGAATGG |
| | | AGACATGCTTCCAACAGGCGTGTAAGGGTAAAATCCA |
| | | AGCACTCTGCGAGAATCCCGAGTGGGCACCATTGAAG |
| | | GATAACAGGATTCCTTCATACGGGGTCTTGTCTGTTGA |
| | | TCTGAGTCTGACAGTTGAGCTTAAAATCAAAATTGCTT |
| | | CGGGATTCGGGCCATTGATCACACACGGTTCAGGGATG |
| | | GACCTATACAAATCCAACCACAACAATGTGTATTGGCT |
| | | GACTATCCCGCCAATGAAGAACCTAGCCTTAGGTGTAA |
| | | TCAACACATTGGAGTGGATACCGAGATTCAAGGTTAGT |
| | | CCCTACCTCTTCACTGTCCCAATTAAGGAAGCAGGCGA |
| | | AGACTGCCATGCCCCAACATACCTACCTGCGGAGGTGG |
| | | ATGGTGATGTCAAACTCAGTTCCAATCTGGTGATTCTA |
| | | CCTGGTCAAGATCTCCAATATGTTTTGGCAACCTACGA |
| | | TACTTCCAGGGTTGAACATGCTGTGGTTTATTACGTTTA |
| | | CAGCCCAAGCCGCTCATTTTCTTACTTTTATCCTTTTAG |
| | | GTTGCCTATAAAGGGGGTCCCCATCGAATTACAAGTGG |
| | | AATGCTTCACATGGGACCAAAAACTCTGGTGCCGTCAC |
| | | TTCTGTGTGCTTGCGGACTCAGAATCTGGTGGACATAT |
| | | CACTCACTCTGGGATGGTGGGCATGGGAGTCAGCTGCA |
| | | CAGTCACCCGGGAAGATGGAACCAATCGCAGATAGGG |
| | | CTGCTAGTGAACCAATCACATGATGTCACCCAGACATC |
| | | AGGCATACCCACTAGTGTGAAATAGACATCAGAATTA |
| | | AGAAAAACGTAGGGTCCAAGTGGTTCCCCGTTATGGAC |
| | | TCGCTATCTGTCAACCAGATCTTATACCCTGAAGTTCA |
| | | CCTAGATAGCCCGATAGTTACCAATAAGATAGTAGCCA |
| | | TCCTGGAGTATGCTCGAGTCCCTCACGCTTACAGCCTG |
| | | GAGGACCCTACACTGTGTCAGAACATCAAGCACCGCCT |
| | | AAAAAACGGATTTTCCAACCAAATGATTATAAACAATG |
| | | TGGAAGTTGGGAATGTCATCAAGTCCAAGCTTAGGAGT |
| | | TATCCGGCCCACTCTCATATTCCATATCCAAATTGTAAT |
| | | CAGGATTTATTTAACATAGAAGACAAAGAGTCAACGA |
| | | GGAAGATCCGTGAACTCCTCAAAAAGGGGAATTCGCT |
| | | GTACTCCAAAGTCAGTGATAAGGTTTTCCAATGCTTAA |
| | | GGGACACTAACTCACGGCTTGGCCTAGGCTCCGAATTG |
| | | AGGGAGGACATCAAGGAGAAAGTTATTAACTTGGGAG |
| | | TTTACATGCACAGCTCCCAGTGGTTTGAGCCCTTTCTGT |
| | | TTTGGTTTACAGTCAAGACTGAGATGAGGTCAGTGATT |
| | | AAATCACAAACCCATACTTGCCATAGGAGGAGACACA |
| | | CACCTGTATTCTTCACTGGTAGTTCAGTTGAGTTGCTAA |
| | | TCTCTCGTGACCTTGTTGCTATAATCAGTAAAGAGTCTC |
| | | AACATGTATATTACCTGACATTTGAACTGGTTTTGATGT |
| | | ATTGTGATGTCATAGAGGGGAGGTTAATGACAGAGAC |
| | | CGCTATGACTATTGATGCTAGGTATACAGAGCTTCTAG |
| | | GAAGAGTCAGATACATGTGGAAACTGATAGATGGTTTC |
| | | TTCCCTGCACTCGGGAATCCAACTTATCAAATTGTAGC |
| | | CATGCTGGAGCCTCTTTCACTTGCTTACCTGCAGCTGA |
| | | GGGATATAACAGTAGAACTCAGAGGTGCTTTCCTTAAC |
| | | CACTGCTTTACTGAAATACATGATGTTCTTGACCAAAA |
| | | CGGGTTTTCTGATGAAGGTACTTATCATGAGTTAACTG |
| | | AAGCTCTAGATTACATTTTCATAACTGATGACATACAT |
| | | CTGACAGGGGAGATTTTCTCATTTTTCAGAAGTTTCGG |
| | | CCACCCCAGACTTGAAGCAGTAACGGCTGCTGAAAAT |
| | | GTTAGGAAATACATGAATCAGCCTAAAGTCATTGTGTA |
| | | TGAGACTCTGATGAAAGGTCATGCCATATTTTGTGGAA |
| | | TCATAATCAACGGCTATCGTGACAGGCACGGAGGCAG |
| | | TTGGCCACCGCTGACCCTCCCCCTGCATGCTGCAGACA |
| | | CAATCCGGAATGCTCAAGCTTCAGGTGAAGGGTTAACA |
| | | CATGAGCAGTGCGTTGATAACTGGAAATCTTTTGCTGG |
| | | AGTGAAATTTGGCTGCTTTATGCCTCTTAGCCTGGATA |
| | | GTGATCTGACAATGTACCTAAAGGACAAGGCACTTGCT |
| | | GCTCTCCAAAGGGAATGGGATTCAGTTTACCCGAAAGA |
| | | GTTCCTGCGTTACGACCCTCCCAAGGGAACCGGGTCAC |
| | | GGAGGCTTGTAGATGTTTTCCTTAATGATTCGAGCTTTG |
| | | ACCCATATGATGTGATAATGTATGTTGTAAGTGGAGCT |
| | | TACCTCCATGACCCTGAGTTCAACCTGTCTTACAGCCT |
| | | GAAAGAAAAGGAGATCAAGGAAACAGGTAGACTTTTT |
| | | GCTAAAATGACTTACAAAATGAGGGCATGCCAAGTGA |
| | | TTGCTGAAAATCTAATCTCAAACGGGATTGGCAAATAT |
| | | TTTAAGGACAATGGGATGGCCAAGGATGAGCACGATT |
| | | TGACTAAGGCACTCCACACTCTAGCTGTCTCAGGAGTC |
| | | CCCAAAGATCTCAAAGAAAGTCACAGGGGGGGGCCAG |
| | | TCTTAAAAACCTACTCCCGAAGCCCAGTCCACACAAGT |
| | | ACCAGGAACGTGAGAGCAGCAAAAGGGTTTATAGGGT |
| | | TCCCTCAAGTAATTCGGCAGGACCAAGACACTGATCAT |
| | | CCGGAGAATATGGAAGCTTACGAGACAGTCAGTGCAT |
| | | TTATCACGACTGATCTCAAGAAGTACTGCCTTAATTGG |
| | | AGATATGAGACCATCAGCTTGTTTGCACAGAGGCTAAA |
| | | TGAGATTTACGGATTGCCCTCATTTTTCCAGTGGCTGCA |
| | | TAAGAGGCTTGAGACCTCTGTCCTGTATGTAAGTGACC |
| | | CTCATTGCCCCCCCGACCTTGACGCCCATATCCCGTTAT |
| | | ATAAAGTCCCCAATGATCAAATCTTCATTAAGTACCCT |
| | | ATGGGAGGTATAGAAGGGTATTGTCAGAAGCTGTGGA |
| | | CCATCAGCACCATTCCCTATCTATACCTGGCTGCTTATG |
| | | AGAGCGGAGTAAGGATTGCTTCGTTAGTGCAAGGGGA |
| | | CAATCAGACCATAGCCGTAACAAAAAGGGTACCCAGC |
| | | ACATGGCCCTACAACCTTAAGAAACGGGAAGCTGCTA |
| | | GAGTAACTAGAGATTACTTTGTAATTCTTAGGCAAAGG |
| | | CTACATGATATTGGCCATCACCTCAAGGCAAATGAGAC |
| | | AATTGTTTCATCACATTTTTTTGTCTATTCAAAAGGAAT |
| | | ATATTATGATGGGCTACTTGTGTCCCAATCACTCAAGA |
| | | GCATCGCAAGATGTGTATTCTGGTCAGAGACTATAGTT |
| | | GATGAAACAAGGGCAGCATGCAGTAATATTGCTACAA |
| | | CAATGGCTAAAAGCATCGAGAGAGGTTATGACCGTTA |
| | | CCTTGCATATTCCCTGAACGTCCTAAAAGTGATACAGC |
| | | AAATTCTGATCTCTCTTGGCTTCACAATCAATTCAACCA |
| | | TGACCCGGGATGTAGTCATACCCCTCCTCACAAACAAC |
| | | GACCTCTTAATAAGGATGGCACTGTTGCCCGCTCCTAT |
| | | TGGGGGGATGAATTATCTGAATATGAGCAGGCTGTTTG |
| | | TCAGAAACATCGGTGATCCAGTAACATCATCAATTGCT |
| | | GATCTCAAGAGAATGATTCTCGCCTCACTAATGCCTGA |
| | | AGAGACCCTCCATCAAGTAATGACACAACAACCGGGG |
| | | GACTCTTCATTCCTAGACTGGGCTAGCGACCCTTACTC |
| | | AGCAAATCTTGTATGTGTCCAGAGCATCACTAGACTCC |
| | | TCAAGAACATAACTGCAAGGTTTGTCCTGATCCATAGT |
| | | CCAAACCCAATGTTAAAAGGATTATTCCATGATGACAG |
| | | TAAAGAAGAGGACGAGGGACTGGCGGCATTCCTCATG |
| | | GACAGGCATATTATAGTACCTAGGGCAGCTCATGAAAT |
| | | CCTGGATCATAGTGTCACAGGGGCAAGAGAGTCTATTG |
| | | CAGGCATGCTGGATACCACAAAAGGCTTGATTCGAGCC |
| | | AGCATGAGGAAGGGGGGGTTAACCTCTCGAGTGATAA |
| | | CCAGATTGTCCAATTATGACTATGAACAATTCAGAGCA |
| | | GGGATGGTGCTATTGACAGGAAGAAAGAGAAATGTCC |
| | | TCATTGACAAAGAGTCATGTTCAGTGCAGCTGGCGAGA |
| | | GCTCTAAGAAGCCATATGTGGGCGAGGCTAGCTCGAG |
| | | GACGGCCTATTTACGGCCTTGAGGTCCCTGATGTACTA |
| | | GAATCTATGCGAGGCCACCTTATTCGGCGTCATGAGAC |
| | | ATGTGTCATCTGCGAGTGTGGATCAGTCAACTACGGAT |
| | | GGTTTTTTGTCCCCTCGGGTTGCCAACTGGATGATATTG |
| | | ACAAGGAAACATCATCCTTGAGAGTCCCATATATTGGT |
| | | TCTACCACTGATGAGAGAACAGACATGAAGCTTGCCTT |
| | | CGTAAGAGCCCCAAGTCGATCCTTGCGATCTGCTGTTA |
| | | GAATAGCAACAGTGTACTCATGGGCTTACGGTGATGAT |
| | | GATAGCTCTTGGAACGAAGCCTGGTTGTTGGCTAGGCA |
| | | AAGGGCCAATGTGAGCCTGGAGGAGCTAAGGGTGATC |
| | | ACTCCCATCTCAACTTCGACTAATTTAGCGCATAGGTT |
| | | GAGGGATCGTAGCACTCAAGTGAAATACTCAGGTACA |
| | | TCCCTTGTCCGAGTGGCGAGGTATACCACAATCTCCAA |
| | | CGACAATCTCTCATTTGTCATATCAGATAAGAAGGTTG |
| | | ATACTAACTTTATATACCAACAAGGAATGCTTCTAGGG |
| | | TTGGGTGTTTTAGAAACATTGTTTCGACTCGAGAAAGA |
| | | TACCGGATCATCTAACACGGTATTACATCTTCACGTCG |
| | | AAACAGATTGTTGCGTGATCCCGATGATAGATCATCCC |
| | | AGGATACCCAGCTCCCGCAAGCTAGAGCTGAGGGCAG |
| | | AGCTATGTACCAACCCATTGATATATGATAATGCACCT |
| | | TTAATTGACAGAGATGCAACAAGGCTATACACCCAGA |
| | | GCCATAGGAGGCACCTTGTGGAATTTGTTACATGGTCC |
| | | ACACCCCAACTATATCACATTTTAGCTAAGTCCACAGC |
| | | ACTATCTATGATTGACCTGGTAACAAAATTTGAGAAGG |
| | | ACCATATGAATGAAATTTCAGCTCTCATAGGGGATGAC |
| | | GATATCAATAGTTTCATAACTGAGTTTCTGCTCATAGA |
| | | GCCAAGATTATTCACTATCTACTTGGGCCAGTGTGCGG |
| | | CCATCAATTGGGCATTTGATGTACATTATCATAGACCA |
| | | TCAGGGAAATATCAGATGGGTGAGCTGTTGTCATCGTT |
| | | CCTTTCTAGAATGAGCAAAGGAGTGTTTAAGGTGCTTG |
| | | TCAATGCTCTAAGCCACCCAAAGATCTACAAGAAATTC |
| | | TGGCATTGTGGTATTATAGAGCCTATCCATGGTCCTTC |
| | | ACTTGATGCTCAAAACTTGCACACAACTGTGTGCAACA |
| | | TGGTTTACACATGCTATATGACCTACCTCGACCTGTTGT |
| | | TGAATGAAGAGTTAGAAGAGTTCACATTTCTCTTGTGT |
| | | GAAAGCGACGAGGATGTAGTACCGGACAGATTCGACA |
| | | ACATCCAGGCAAAACACTTATGTGTTCTGGCAGATTTG |
| | | TACTGTCAACCAGGGACCTGCCCACCAATTCGAGGTCT |
| | | AAGACCGGTAGAGAAATGTGCAGTTCTAACCGACCAT |
| | | ATCAAGGCAGAGGCTATGTTATCTCCAGCAGGATCTTC |
| | | GTGGAACATAAATCCAATTATTGTAGACCATTACTCAT |
| | | GCTCTCTGACTTATCTCCGGCGAGGATCGATCAAACAG |
| | | ATAAGATTGAGAGTTGATCCAGGATTCATTTTCGACGC |
| | | CCTCGCTGAGGTAAATGTCAGTCAGCCAAAGATCGGCA |
| | | GCAACAACATCTCAAATATGAGCATCAAGGCTTTCAGA |
| | | CCCCCACACGATGATGTTGCAAAATTGCTCAAAGATAT |
| | | CAACACAAGCAAGCACAATCTTCCCATTTCAGGGGGCA |
| | | ATCTCGCCAATTATGAAATCCATGCTTTCCGCAGAATC |
| | | GGGTTGAACTCATCTGCTTGCTACAAAGCTGTTGAGAT |
| | | ATCAACATTAATTAGGAGATGCCTTGAGCCAGGGGAG |
| | | GACGGCTTGTTCTTGGGTGAGGGATCGGGTTCTATGTT |
| | | GATCACTTATAAAGAGATACTTAAACTAAACAAGTGCT |
| | | TCTATAATAGTGGGGTTTCCGCCAATTCTAGATCTGGT |
| | | CAAAGGGAATTAGCACCCTATCCCTCCGAAGTTGGCCT |
| | | TGTCGAACACAGAATGGGAGTAGGTAATATTGTCAAA |
| | | GTGCTCTTTAACGGGAGGCCCGAAGTCACGTGGGTAGG |
| | | CAGTGTAGATTGCTTCAATTTCATAGTTAGTAATATCCC |
| | | TACCTCTAGTGTGGGGTTTATCCATTCAGATATAGAGA |
| | | CCTTGCCTGACAAAGATACTATAGAGAAGCTAGAGGA |
| | | ATTGGCAGCCATCTTATCGATGGCTCTGCTCCTGGGCA |
| | | AAATAGGATCAATACTGGTGATTAAGCTTATGCCTTTC |
| | | AGCGGGGATTTTGTTCAGGGATTTATAAGTTATGTAGG |
| | | GTCTCATTATAGAGAAGTGAACCTTGTATACCCTAGAT |
| | | ACAGCAACTTCATCTCTACTGAATCTTATTTGGTTATGA |
| | | CAGATCTCAAGGCTAACCGGCTAATGAATCCTGAAAA |
| | | GATTAAGCAGCAGATAATTGAATCATCTGTGAGGACTT |
| | | CACCTGGACTTATAGGTCACATCCTATCCATTAAGCAA |
| | | CTAAGCTGCATACAAGCAATTGTGGGAGACGCAGTTA |
| | | GTAGAGGTGATATCAATCCTACTCTGAAAAAACTTACA |
| | | CCTATAGAGCAGGTGCTGATCAATTGCGGGTTGGCAAT |
| | | TAACGGACCTAAGCTGTGCAAAGAATTGATCCACCATG |
| | | ATGTTGCCTCAGGGCAAGATGGATTGCTTAATTCTATA |
| | | CTCATCCTCTACAGGGAGTTGGCAAGATTCAAAGACAA |
| | | CCAAAGAAGTCAACAAGGGATGTTCCACGCTTACCCCG |
| | | TATTGGTAAGTAGCAGGCAACGAGAACTTATATCTAGG |
| | | ATCACCCGCAAATTCTGGGGGCACATTCTTCTTTACTCC |
| | | GGGAACAAAAAGTTGATAAATAAGTTTATCCAGAATCT |
| | | CAAGTCCGGCTATCTGATACTAGACTTACACCAGAATA |
| | | TCTTCGTTAAGAATCTATCCAAGTCAGAGAAACAGATT |
| | | ATTATGACGGGGGGTTTGAAACGTGAGTGGGTTTTTAA |
| | | GGTAACAGTCAAGGAGACCAAAGAATGGTATAAGTTA |
| | | GTCGGATACAGTGCCCTGATTAAGGACTAATTGGTTGA |
| | | ACTCCGGAACCCTAATCCTGCCCTAGGTGGTTAGGCAT |
| | | TATTTGCAATATATTAAAGAAAACTTTGAAAATACGAA |
| | | GTTTCTATTCCCAGCTTTGTCTGGT |
| 89 | T-09 cDNA in | ACCAAACAAAGTTGGGTAAGGATAGTTCAATCAATGA |
| | CMV vector | TCATCTTCTAGTGCACTTAGGATTCAAGATCCTATTATC |
| | MV leader: nt1- | AGGGACAAGAGCAGGATTAGGGATATCCGAGATGGCC |
| | 55 | ACACTTTTAAGGAGCTTAGCATTGTTCAAAAGAAACAA |
| | MV N ORF: nt | GGACAAACCACCCATTACATCAGGATCCGGTGGAGCC |
| | 108-1,685 | ATCAGAGGAATCAAACACATTATTATAGTACCAATCCC |
| | MV P ORF: nt | TGGAGATTCCTCAATTACCACTCGATCCAGACTTCTGG |
| | 1,807-3,330 | ACCGGTTGGTGAGGTTAATTGGAAACCCGGATGTGAGC |
| | MV M ORF: nt | GGGCCCAAACTAACAGGGGCACTAATAGGTATATTATC |
| | 3,438-4,445 | CTTATTTGTGGAGTCTCCAGGTCAATTGATTCAGAGGA |
| | MV F ORF: nt | TCACCGATGACCCTGACGTTAGCATAAGGCTGTTAGAG |
| | 5,449-7,110 | GTTGTCCAGAGTGACCAGTCACAATCTGGCCTTACCTT |
| | MV H ORF: nt | CGCATCAAGAGGTACCAACATGGAGGATGAGGCGGAC |
| | 7,271-9,124 | CAATACTTTTCACATGATGATCCAATTAGTAGTGATCA |
| | ATU upstream | ATCCAGGTTCGGATGGTTCGGGAACAAGGAAATCTCA |
| | motif: nt 9,180- | GATATTGAAGTGCAAGACCCTGAGGGATTCAACATGAT |
| | 9,251 | TCTGGGTACCATCCTAGCCCAAATTTGGGTCTTGCTCG |
| | GE of N gene: | CAAAGGCGGTTACGGCCCCAGACACGGCAGCTGATTC |
| | nt 9,196-9,206 | GGAGCTAAGAAGGTGGATAAAGTACACCCAACAAAGA |
| | Conserved | AGGGTAGTTGGTGAATTTAGATTGGAGAGAAAATGGTT |
| | trinucleotide | GGATGTGGTGAGGAACAGGATTGCCGAGGACCTCTCCT |
| | motif: nt 9,207- | TACGCCGATTCATGGTCGCTCTAATCCTGGATATCAAG |
| | 9,209 | AGAACACCCGGAAACAAACCCAGGATTGCTGAAATGA |
| | GS of P gene: | TATGTGACATTGATACATATATCGTAGAGGCAGGATTA |
| | nt 9,210-9,226 | GCCAGTTTTATCCTGACTATTAAGTTTGGGATAGAAAC |
| | BsiWI | TATGTATCCTGCTCTTGGACTGCATGAATTTGCTGGTGA |
| | Restriction site: | GTTATCCACACTTGAGTCCTTGATGAACCTTTACCAGC |
| | nt 9,246-9,251 | AAATGGGGGAAACTGCACCCTACATGGTAATCCTGGA |
| | EBV gH ORF: | GAACTCAATTCAGAACAAGTTCAGTGCAGGATCATACC |
| | nt 9,252-11,369 | CTCTGCTCTGGAGCTATGCCATGGGAGTAGGAGTGGAA |
| | Furin cleavage | CTTGAAAACTCCATGGGAGGTTTGAACTTTGGCCGATC |
| | site: nt 11,370- | TTACTTTGATCCAGCATATTTTAGATTAGGGCAAGAGA |
| | 11,381 | TGGTAAGGAGGTCAGCTGGAAAGGTCAGTTCCACATTG |
| | P2A: nt 11,382- | GCATCTGAACTCGGTATCACTGCCGAGGATGCAAGGCT |
| | 11,438 | TGTTTCAGAGATTGCAATGCATACTACTGAGGACAAGA |
| | EBV gL ORF: | TCAGTAGAGCGGTTGGACCCAGACAAGCCCAAGTATC |
| | nt 11,439- | ATTTCTACACGGTGATCAAAGTGAGAATGAGCTACCGA |
| | 11,849 | GATTGGGGGGCAAGGAAGATAGGAGGGTCAAACAGAG |
| | Furin cleavage | TCGAGGAGAAGCCAGGGAGAGCTACAGAGAAACCGGG |
| | site: nt 11,850- | CCCAGCAGAGCAAGTGATGCGAGAGCTGCCCATCTTCC |
| | 11,861 | AACCGGCACACCCCTAGACATTGACACTGCAACGGAG |
| | T2A: nt 11,862- | TCCAGCCAAGATCCGCAGGACAGTCGAAGGTCAGCTG |
| | 11,915 | ACGCCCTGCTTAGGCTGCAAGCCATGGCAGGAATCTCG |
| | EBV gp42 | GAAGAACAAGGCTCAGACACGGACACCCCTATAGTGT |
| | ORF: nt | ACAATGACAGAAATCTTCTAGACTAGGTGCGAGAGGC |
| | 11,916-12,587 | CGAGGGCCAGAACAACATCCGCCTACCATCCATCATTG |
| | BssHII | TTATAAAAAACTTAGGAACCAGGTCCACACAGCCGCC |
| | Restriction site: | AGCCCATCAACCATCCACTCCCACGATTGGAGCCAATG |
| | nt 12,588- | GCAGAAGAGCAGGCACGCCATGTCAAAAACGGACTGG |
| | 12,593 | AATGCATCCGGGCTCTCAAGGCCGAGCCCATCGGCTCA |
| | ATU | CTGGCCATCGAGGAAGCTATGGCAGCATGGTCAGAAA |
| | downstream | TATCAGACAACCCAGGACAGGAGCGAGCCACCTGCAG |
| | motif: nt | GGAAGAGAAGGCAGGCAGTTCGGGTCTCAGCAAACCA |
| | 12,588-12,623 | TGCCTCTCAGCAATTGGATCAACTGAAGGCGGTGCACC |
| | MV L ORF: nt | TCGCATCCGCGGTCAGGGACCTGGAGAGAGCGATGAC |
| | 12,678-19,229 | GACGCTGAAACTTTGGGAATCCCCCCAAGAAATCTCCA |
| | CMV promoter: | GGCATCAAGCACTGGGTTACAGTGTTATTACGTTTATG |
| | nt 22,764- | ATCACAGCGGTGAAGCGGTTAAGGGAATCCAAGATGC |
| | 23,067 | TGACTCTATCATGGTTCAATCAGGCCTTGATGGTGATA |
| | | GCACCCTCTCAGGAGGAGACAATGAATCTGAAAACAG |
| | | CGATGTGGATATTGGCGAACCTGATACCGAGGGATATG |
| | | CTATCACTGACCGGGGATCTGCTCCCATCTCTATGGGG |
| | | TTCAGGGCTTCTGATGTTGAAACTGCAGAAGGAGGGG |
| | | AGATCCACGAGCTCCTGAGACTCCAATCCAGAGGCAA |
| | | CAACTTTCCGAAGCTTGGGAAAACTCTCAATGTTCCTC |
| | | CGCCCCCGGACCCCGGTAGGGCCAGCACTTCCGGGAC |
| | | ACCCATTAAAAAGGGCACAGACGCGAGATTAGCCTCA |
| | | TTTGGAACGGAGATCGCGTCTTTATTGACAGGTGGTGC |
| | | AACCCAATGTGCTCGAAAGTCACCCTCGGAACCATCAG |
| | | GGCCAGGTGCACCTGCGGGGAATGTCCCCGAGTGTGTG |
| | | AGCAATGCCGCACTGATACAGGAGTGGACACCCGAAT |
| | | CTGGTACCACAATCTCCCCGAGATCCCAGAATAATGAA |
| | | GAAGGGGGAGACTATTATGATGATGAGCTGTTCTCTGA |
| | | TGTCCAAGATATTAAAACAGCCTTGGCCAAAATACACG |
| | | AGGATAATCAGAAGATAATCTCCAAGCTAGAATCACT |
| | | GCTGTTATTGAAGGGAGAAGTTGAGTCAATTAAGAAG |
| | | CAGATCAACAGGCAAAATATCAGCATATCCACCCTGG |
| | | AAGGACACCTCTCAAGCATCATGATCGCCATTCCTGGA |
| | | CTTGGGAAGGATCCCAACGACCCCACTGCAGATGTCGA |
| | | AATCAATCCCGACTTGAAACCCATCATAGGCAGAGATT |
| | | CAGGCCGAGCACTGGCCGAAGTTCTCAAGAAACCCGTT |
| | | GCCAGCCGACAACTCCAAGGAATGACAAATGGACGGA |
| | | CCAGTTCCAGAGGACAGCTGCTGAAGGAATTTCAGCTA |
| | | AAGCCGATCGGGAAAAAGATGAGCTCAGCCGTCGGGT |
| | | TTGTTCCTGACACCGGCCCTGCATCACGCAGTGTAATC |
| | | CGCTCCATTATAAAATCCAGCCGGCTAGAGGAGGATCG |
| | | GAAGCGTTACCTGATGACTCTCCTTGATGATATCAAAG |
| | | GAGCCAATGATCTTGCCAAGTTCCACCAGATGCTGATG |
| | | AAGATAATAATGAAGTAGCTACAGCTCAACTTACCTGC |
| | | CAACCCCATGCCAGTCGACCCAACTAGTACAACCTAAA |
| | | TCCATTATAAAAAACTTAGGAGCAAAGTGATTGCCTCC |
| | | CAAGGTCCACAATGACAGAGACCTACGACTTCGACAA |
| | | GTCGGCATGGGACATCAAAGGGTCGATCGCTCCGATAC |
| | | AACCCACCACCTACAGTGATGGCAGGCTGGTGCCCCAG |
| | | GTCAGAGTCATAGATCCTGGTCTAGGCGACAGGAAGG |
| | | ATGAATGCTTTATGTACATGTTTCTGCTGGGGGTTGTTG |
| | | AGGACAGCGATTCCCTAGGGCCTCCAATCGGGCGAGC |
| | | ATTTGGGTTCCTGCCCTTAGGTGTTGGCAGATCCACAG |
| | | CAAAGCCCGAAAAACTCCTCAAAGAGGCCACTGAGCT |
| | | TGACATAGTTGTTAGACGTACAGCAGGGCTCAATGAAA |
| | | AACTGGTGTTCTACAACAACACCCCACTAACTCTCCTC |
| | | ACACCTTGGAGAAAGGTCCTAACAACAGGGAGTGTCTT |
| | | CAACGCAAACCAAGTGTGCAATGCGGTTAATCTGATAC |
| | | CGCTCGATACCCCGCAGAGGTTCCGTGTTGTTTATATG |
| | | AGCATCACCCGTCTTTCGGATAACGGGTATTACACCGT |
| | | TCCTAGAAGAATGCTGGAATTCAGATCGGTCAATGCAG |
| | | TGGCCTTCAACCTGCTGGTGACCCTTAGGATTGACAAG |
| | | GCGATAGGCCCTGGGAAGATCATCGACAATACAGAGC |
| | | AACTTCCTGAGGCAACATTTATGGTCCACATCGGGAAC |
| | | TTCAGGAGAAAGAAGAGTGAAGTCTACTCTGCCGATTA |
| | | TTGCAAAATGAAAATCGAAAAGATGGGCCTGGTTTTTG |
| | | CACTTGGTGGGATAGGGGGCACCAGTCTTCACATTAGA |
| | | AGCACAGGCAAAATGAGCAAGACTCTCCATGCACAAC |
| | | TCGGGTTCAAGAAGACCTTATGTTACCCGCTGATGGAT |
| | | ATCAATGAAGACCTTAATCGATTACTCTGGAGGAGCAG |
| | | ATGCAAGATAGTAAGAATCCAGGCAGTTTTGCAGCCAT |
| | | CAGTTCCTCAAGAATTCCGCATTTACGACGACGTGATC |
| | | ATAAATGATGACCAAGGACTATTCAAAGTTCTGTAGAC |
| | | CGTAGTGCCCAGCAATGCCCGAAAACGACCCCCCTCAC |
| | | AATGACAGCCAGAAGGCCCGGACAAAAAAGCCCCCTC |
| | | CGAAAGACTCCACGGACCAAGCGAGAGGCCAGCCAGC |
| | | AGCCGACGGCAAGCGCGAACACCAGGCGGCCCCAGCA |
| | | CAGAACAGCCCTGACACAAGGCCACCACCAGCCACCC |
| | | CAATCTGCATCCTCCTCGTGGGACCCCCGAGGACCAAC |
| | | CCCCAAGGCTGCCCCCGATCCAAACCACCAACCGCATC |
| | | CCCACCACCCCCGGGAAAGAAACCCCCAGCAATTGGA |
| | | AGGCCCCTCCCCCTCTTCCTCAACACAAGAACTCCACA |
| | | ACCGAACCGCACAAGCGACCGAGGTGACCCAACCGCA |
| | | GGCATCCGACTCCCTAGACAGATCCTCTCTCCCCGGCA |
| | | AACTAAACAAAACTTAGGGCCAAGGAACATACACACC |
| | | CAACAGAACCCAGACCCCGGCCCACGGCGCCGCGCCC |
| | | CCAACCCCCGACAACCAGAGGGAGCCCCCAACCAATC |
| | | CCGCCGGCTCCCCCGGTGCCCACAGGCAGGGACACCA |
| | | ACCCCCGAACAGACCCAGCACCCAACCATCGACAATC |
| | | CAAGACGGGGGGGCCCCCCCAAAAAAAGGCCCCCAGG |
| | | GGCCGACAGCCAGCACCGCGAGGAAGCCCACCCACCC |
| | | CACACACGACCACGGCAACCAAACCAGAACCCAGACC |
| | | ACCCTGGGCCACCAGCTCCCAGACTCGGCCATCACCCC |
| | | GCAGAAAGGAAAGGCCACAACCCGCGCACCCCAGCCC |
| | | CGATCCGGCGGGGAGCCACCCAACCCGAACCAGCACC |
| | | CAAGAGCGATCCCCGAAGGACCCCCGAACCGCAAAGG |
| | | ACATCAGTATCCCACAGCCTCTCCAAGTCCCCCGGTCT |
| | | CCTCCTCTTCTCGAAGGGACCAAAAGATCAATCCACCA |
| | | CACCCGACGACACTCAACTCCCCACCCCTAAAGGAGAC |
| | | ACCGGGAATCCCAGAATCAAGACTCATCCAATGTCCAT |
| | | CATGGGTCTCAAGGTGAACGTCTCTGCCATATTCATGG |
| | | CAGTACTGTTAACTCTCCAAACACCCACCGGTCAAATC |
| | | CATTGGGGCAATCTCTCTAAGATAGGGGGGTAGGAAT |
| | | AGGAAGTGCAAGCTACAAAGTTATGACTCGTTCCAGCC |
| | | ATCAATCATTAGTCATAAAATTAATGCCCAATATAACT |
| | | CTCCTCAATAACTGCACGAGGGTAGAGATTGCAGAATA |
| | | CAGGAGACTACTGAGAACAGTTTTGGAACCAATTAGA |
| | | GATGCACTTAATGCAATGACCCAGAATATAAGACCGGT |
| | | TCAGAGTGTAGCTTCAAGTAGGAGACACAAGAGATTT |
| | | GCGGGAGTAGTCCTGGCAGGTGCGGCCCTAGGCGTTGC |
| | | CACAGCTGCTCAGATAACAGCCGGCATTGCACTTCACC |
| | | AGTCCATGCTGAACTCTCAAGCCATCGACAATCTGAGA |
| | | GCGAGCCTGGAAACTACTAATCAGGCAATTGAGACAA |
| | | TCAGACAAGCAGGGCAGGAGATGATATTGGCTGTTCA |
| | | GGGTGTCCAAGACTACATCAATAATGAGCTGATACCGT |
| | | CTATGAACCAACTATCTTGTGATTTAATCGGCCAGAAG |
| | | CTCGGGCTCAAATTGCTCAGATACTATACAGAAATCCT |
| | | GTCATTATTTGGCCCCAGTTTACGGGACCCCATATCTG |
| | | CGGAGATATCTATCCAGGCTTTGAGCTATGCGCTTGGA |
| | | GGAGACATCAATAAGGTGTTAGAAAAGCTCGGATACA |
| | | GTGGAGGTGATTTACTGGGCATCTTAGAGAGCGGAGG |
| | | CATCAAGGCCCGGATAACTCACGTCGACACAGAGTCCT |
| | | ACTTCATTGTCCTCAGTATAGCCTATCCGACGCTGTCCG |
| | | AGATTAAGGGGGTGATTGTCCACCGGCTAGAGGGGGT |
| | | CTCGTACAACATAGGCTCTCAAGAGTGGTATACCACTG |
| | | TGCCCAAGTATGTTGCAACCCAAGGGTACCTTATCTCG |
| | | AATTTTGATGAGTCATCGTGTACTTTCATGCCAGAGGG |
| | | GACTGTGTGCAGCCAAAATGCCTTGTACCCGATGAGTC |
| | | CTCTGCTCCAAGAATGCCTCCGGGGGTACACCAAGTCC |
| | | TGTGCTCGTACACTCGTATCCGGGTCTTTTGGGAACCG |
| | | GTTCATTTTATCACAAGGGAACCTAATAGCCAATTGTG |
| | | CATCAATCCTTTGCAAGTGTTACACAACAGGAACGATC |
| | | ATTAATCAAGACCCTGACAAGATCCTAACATACATTGC |
| | | TGCCGATCACTGCCCGGTAGTCGAGGTGAACGGCGTGA |
| | | CCATCCAAGTCGGGAGCAGGAGGTATCCAGACGCTGT |
| | | GTACTTGCACAGAATTGACCTCGGTCCTCCCATATCAT |
| | | TGGAGAGGTTGGACGTAGGGACAAATCTGGGGAATGC |
| | | AATTGCTAAGTTGGAGGATGCCAAGGAATTGTTGGAGT |
| | | CATCGGACCAGATATTGAGGAGTATGAAAGGTTTATCG |
| | | AGCACTAGCATAGTCTACATCCTGATTGCAGTGTGTCT |
| | | TGGAGGGTTGATAGGGATCCCCGCTTTAATATGTTGCT |
| | | GCAGGGGGCGTTGTAACAAAAAGGGAGAACAAGTTGG |
| | | TATGTCAAGACCAGGCCTAAAGCCTGATCTTACGGGAA |
| | | CATCAAAATCCTATGTAAGGTCGCTCTGATCCTCTACA |
| | | ACTCTTGAAACACAAATGTCCCACAAGTCTCCTCTTCG |
| | | TCATCAAGCAACCACCGCACCCAGCATCAAGCCCACCT |
| | | GAAATTATCTCCGGCTTCCCTCTGGCCGAACAATATCG |
| | | GTAGTTAATCAAAACTTAGGGTGCAAGATCATCCACAA |
| | | TGTCACCACAACGAGACCGGATAAATGCCTTCTACAAA |
| | | GATAACCCCCATCCCAAGGGAAGTAGGATAGTCATTA |
| | | ACAGAGAACATCTTATGATTGATAGACCTTATGTTTTG |
| | | CTGGCTGTTCTGTTTGTCATGTTTCTGAGCTTGATCGGG |
| | | TTGCTAGCCATTGCAGGCATTAGACTTCATCGGGCAGC |
| | | CATCTACACCGCAGAGATCCATAAAAGCCTCAGCACCA |
| | | ATCTAGATGTAACTAACTCAATCGAGCATCAGGTCAAG |
| | | GACGTGCTGACACCACTCTTCAAAATCATCGGTGATGA |
| | | AGTGGGCCTGAGGACACCTCAGAGATTCACTGACCTAG |
| | | TGAAATTAATCTCTGACAAGATCAAGTTCCTTAATCCG |
| | | GATAGGGAGTACGACTTCAGAGATCTCACTTGGTGTAT |
| | | CAACCCGCCAGAGAGAATCAAATTGGATTATGATCAAT |
| | | ACTGTGCAGATGTGGCTGCTGAAGAGCTCATGAATGCA |
| | | TTGGTGAACTCAACTCTACTGGAGACCAGAACAACCAA |
| | | TCAGTTCCTAGCTGTCTCAAAGGGAAACTGCTCAGGGC |
| | | CCACTACAATCAGAGGTCAATTCTCAAACATGTCGCTG |
| | | TCCCTGTTAGACTTGTATTTAGGTCGAGGTTACAATGT |
| | | GTCATCTATAGTCACTATGACATCCCAGGGAATGTATG |
| | | GGGGAACTTACCTAGTGGAAAAGCCTAATCTGAGCAG |
| | | CAAAAGGTCAGAGTTGTCACAACTGAGCATGTACCGA |
| | | GTGTTTGAAGTAGGTGTTATCAGAAATCCGGGTTTGGG |
| | | GGCTCCGGTGTTCCATATGACAAACTATCTTGAGCAAC |
| | | CAGTCAGTAATGATCTCAGCAACTGTATGGTGGCTTTG |
| | | GGGGAGCTCAAACTCGCAGCCCTTTGTCACGGGGAAG |
| | | ATTCTATCACAATTCCCTATCAGGGATCAGGGAAAGGT |
| | | GTCAGCTTCCAGCTCGTCAAGCTAGGTGTCTGGAAATC |
| | | CCCAACCGACATGCAATCCTGGGTCCCCTTATCAACGG |
| | | ATGATCCAGTGATAGACAGGCTTTACCTCTCATCTCAC |
| | | AGAGGTGTTATCGCTGACAATCAAGCAAAATGGGCTGT |
| | | CCCGACAACACGAACAGATGACAAGTTGCGAATGGAG |
| | | ACATGCTTCCAACAGGCGTGTAAGGGTAAAATCCAAG |
| | | CACTCTGCGAGAATCCCGAGTGGGCACCATTGAAGGAT |
| | | AACAGGATTCCTTCATACGGGGTCTTGTCTGTTGATCT |
| | | GAGTCTGACAGTTGAGCTTAAAATCAAAATTGCTTCGG |
| | | GATTCGGGCCATTGATCACACACGGTTCAGGGATGGAC |
| | | CTATACAAATCCAACCACAACAATGTGTATTGGCTGAC |
| | | TATCCCGCCAATGAAGAACCTAGCCTTAGGTGTAATCA |
| | | ACACATTGGAGTGGATACCGAGATTCAAGGTTAGTCCC |
| | | TACCTCTTCACTGTCCCAATTAAGGAAGCAGGCGAAGA |
| | | CTGCCATGCCCCAACATACCTACCTGCGGAGGTGGATG |
| | | GTGATGTCAAACTCAGTTCCAATCTGGTGATTCTACCT |
| | | GGTCAAGATCTCCAATATGTTTTGGCAACCTACGATAC |
| | | TTCCAGGGTTGAACATGCTGTGGTTTATTACGTTTACA |
| | | GCCCAAGCCGCTCATTTTCTTACTTTTATCCTTTTAGGT |
| | | TGCCTATAAAGGGGGTCCCCATCGAATTACAAGTGGAA |
| | | TGCTTCACATGGGACCAAAAACTCTGGTGCCGTCACTT |
| | | CTGTGTGCTTGCGGACTCAGAATCTGGTGGACATATCA |
| | | CTCACTCTGGGATGGTGGGCATGGGAGTCAGCTGCACA |
| | | GTCACCCGGGAAGATGGAACCAATCGCAGATAGGGCT |
| | | GCTAGTGAACCAATCACATGATGTCACCCAGACATCAG |
| | | GCATACCCACTAGTCTACCCTCCATCATTGTTATAAAA |
| | | AACTTAGGAACCAGGTCCACACAGCCGCCAGCCCATC |
| | | AACGCGTACGATGCAACTCCTCTGCGTCTTTTGCTTGGT |
| | | GCTTTTGTGGGAGGTAGGAGCGGCCTCATTGTCAGAAG |
| | | TTAAGCTGCATCTCGATATTGAAGGCCACGCTTCACAT |
| | | TACACTATCCCTTGGACCGAACTCATGGCTAAGGTGCC |
| | | GGGTTTGTCTCCGGAAGCACTTTGGAGAGAGGCAAAC |
| | | GTTACCGAAGACCTGGCCTCAATGCTGAATCGCTACAA |
| | | GCTGATATACAAGACTTCCGGAACCCTGGGTATAGCAC |
| | | TCGCAGAGCCCGTCGATATCCCCGCTGTCAGCGAAGGT |
| | | AGCATGCAGGTGGACGCTAGTAAAGTTCACCCAGGAG |
| | | TTATCTCAGGTCTGAATAGCCCAGCTTGTATGCTGAGC |
| | | GCTCCACTGGAGAAACAGCTCTTCTACTATATAGGCAC |
| | | AATGCTCCCTAACACTCGACCTCACAGCTACGTCTTCT |
| | | ATCAGCTGCGCTGTCACCTGTCATACGTAGCCTTGTCC |
| | | ATTAATGGTGATAAGTTCCAGTACACAGGTGCCATGAC |
| | | CTCCAAGTTCCTTATGGGGACCTATAAACGGGTCACTG |
| | | AGAAAGGAGACGAGCATGTCCTTAGTTTGGTTTTTGGG |
| | | AAGACAAAAGACCTGCCGGATCTGAGGGGCCCGTTCT |
| | | CTTACCCCTCACTCACATCTGCACAGAGTGGGGACTAT |
| | | TCATTGGTGATTGTGACAACCTTCGTTCACTACGCCAA |
| | | TTTTCACAACTATTTTGTCCCGAACCTTAAAGACATGTT |
| | | TTCACGCGCCGTGACCATGACAGCTGCTTCCTACGCTA |
| | | GGTATGTGCTCCAAAAGCTGGTGCTGCTGGAGATGAAG |
| | | GGCGGATGCAGGGAACCTGAACTGGACACAGAGACTC |
| | | TCACCACTATGTTCGAGGTTTCCGTGGCGTTCTTCAAG |
| | | GTGGGTCATGCTGTGGGGGAGACTGGGAACGGCTGCG |
| | | TAGATCTTAGGTGGCTGGCTAAATCTTTCTTCGAGCTG |
| | | ACCGTGTTGAAAGATATAATTGGAATTTGCTACGGGGC |
| | | TACGGTTAAAGGAATGCAGTCTTACGGGCTGGAAAGG |
| | | CTGGCCGCTATGCTCATGGCTACTGTGAAGATGGAGGA |
| | | ACTCGGCCACCTCACCACAGAAAAACAGGAGTACGCT |
| | | TTGCGCCTGGCCACTGTTGGCTACCCTAAGGCAGGCGT |
| | | TTACTCTGGATTGATTGGGGGGGCCACATCTGTGCTCT |
| | | TGTCAGCCTACAATCGCCATCCCCTGTTCCAGCCTCTGC |
| | | ACACGGTGATGCGAGAGACTTTGTTTATTGGCAGCCAC |
| | | GTTGTGCTGAGAGAACTGCGCCTTAACGTTACCACTCA |
| | | GGGACCTAACCTTGCGCTGTATCAGCTGCTTAGCACCG |
| | | CTTTGTGCTCCGCTCTTGAAATTGGAGAAGTACTCAGA |
| | | GGGCTCGCACTGGGTACTGAGTCAGGGCTGTTCTCCCC |
| | | CTGCTATCTGTCTCTCCGGTTCGACCTCACTCGGGATAA |
| | | GCTGTTGTCAATGGCTCCGCAGGAAGCCACATTGGACC |
| | | AGGCCGCAGTGAGTAACGCAGTGGACGGGTTCCTGGG |
| | | TAGACTGTCTCTTGAGAGGGAAGACAGAGACGCTTGG |
| | | CATCTGCCTGCGTATAAGTGTGTTGATAGACTGGACAA |
| | | AGTACTGATGATCATTCCCCTGATTAATGTGACATTTAT |
| | | TATATCTAGTGACCGAGAGGTGAGAGGCTCCGCTCTCT |
| | | ATGAAGCATCCACAACGTACTTGAGCAGTTCCCTGTTT |
| | | CTGTCACCGGTTATTATGAACAAATGTTCTCAGGGAGC |
| | | TGTTGCTGGTGAACCGCGACAGATTCCAAAGATTCAGA |
| | | ACTTCACTAGGACACAAAAGAGCTGCATTTTCTGCGGG |
| | | TTCGCACTGCTGAGTTACGACGAAAAGGAGGGACTGG |
| | | AGACTACTACATATATTACTTCACAGGAGGTGCAGAAT |
| | | AGCATCCTGAGCTCCAATTATTTTGACTTTGACAACCTC |
| | | CATGTCCATTATTTGCTGTTGACCACAAACGGCACTGT |
| | | GATGGAGATCGCTGGCCTGTACGAGGAGCGAGCCCAC |
| | | GTAGTACTGGCCATTATTCTGTACTTCATCGCTTTCGCA |
| | | CTTGGGATTTTCCTCGTCCACAAAATTGTTATGTTTTTC |
| | | CTCAGAGCAAAGAGAGCGACGAACTTCTCACTGCTTAA |
| | | GCAAGCAGGAGACGTGGAGGAGAATCCGGGCCCAATG |
| | | AGAGCCGTAGGTGTCTTTCTGGCTATTTGTCTGGTAAC |
| | | AATCTTCGTGCTGCCCACTTGGGGCAACTGGGCCTATC |
| | | CTTGCTGCCATGTTACACAGTTGAGAGCGCAGCACCTC |
| | | CTGGCGTTGGAGAACATTTCAGACATCTACCTGGTGAG |
| | | TAACCAAACCTGCGACGGGTTCTCTTTGGCCAGTCTCA |
| | | ATAGTCCAAAAAACGGAAGCAATCAGCTGGTAATTTC |
| | | ACGGTGTGCAAACGGACTTAACGTGGTCTCTTTTTTTAT |
| | | ATCAATCCTGAAACGCAGTTCATCTGCATTGACCGGCC |
| | | ACTTGAGGGAGCTCTTGACCACTCTCGAGACACTTTAC |
| | | GGGTCATTCTCCGTGGAGGATCTGTTCGGGGCAAATCT |
| | | GAACCGGTACGCCTGGCATAGAGGCGGGAGAGCAAAG |
| | | AGAGAGGGAAGAGGCTCCTTGCTGACCTGCGGTGACG |
| | | TGGAAGAGAATCCCGGCCCTATGGTGAGTTTTAAGCAG |
| | | GTCCGGGTACCTCTGTTCACCGCCATTGCGCTGGTGAT |
| | | TGTTCTGCTGTTGGCCTACTTTCTTCCACCTAGAGTCAG |
| | | AGGAGGTGGCAGGGTTGCTGCAGCTGCTATTACCTGGG |
| | | TACCGAAACCCAACGTGGAGGTTTGGCCAGTGGATCCG |
| | | CCCCCACCAGTGAACTTTAATAAAACAGCCGAACAAG |
| | | AATACGGGGATAAAGAAGTAAAGCTGCCACACTGGAC |
| | | CCCTACCCTCCACACTTTCCAGGTGCCTCAAAACTATA |
| | | CAAAGGCCAATTGCACATACTGTAACACAAGAGAATA |
| | | CACTTTTTCTTATAAAGGGTGCTGCTTCTACTTCACTAA |
| | | GAAGAAGCATACTTGGAACGGATGTTTCCAGGCATGC |
| | | GCTGAGTTGTATCCCTGTACCTATTTCTACGGTCCTACA |
| | | CCAGACATTCTGCCGGTCGTGACTCGCAATCTGAACGC |
| | | CATAGAATCTCTGTGGGTCGGGGTGTATCGGGTTGGGG |
| | | AAGGAAATTGGACTAGCCTGGACGGCGGTACTTTCAA |
| | | GGTGTACCAAATATTCGGCAGTCACTGTACTTACGTGT |
| | | CAAAATTCTCAACAGTTCCCGTATCCCACCACGAGTGT |
| | | TCTTTTTTGAAGCCTTGCCTGTGCGTGTCACAGAGGTCA |
| | | AATTCCTAAGCGCGCAGCGCTTAGACGTCTCGCGATCG |
| | | ATGCTAGTGTGAAATAGACATCAGAATTAAGAAAAAC |
| | | GTAGGGTCCAAGTGGTTCCCCGTTATGGACTCGCTATC |
| | | TGTCAACCAGATCTTATACCCTGAAGTTCACCTAGATA |
| | | GCCCGATAGTTACCAATAAGATAGTAGCCATCCTGGAG |
| | | TATGCTCGAGTCCCTCACGCTTACAGCCTGGAGGACCC |
| | | TACACTGTGTCAGAACATCAAGCACCGCCTAAAAAAC |
| | | GGATTTTCCAACCAAATGATTATAAACAATGTGGAAGT |
| | | TGGGAATGTCATCAAGTCCAAGCTTAGGAGTTATCCGG |
| | | CCCACTCTCATATTCCATATCCAAATTGTAATCAGGATT |
| | | TATTTAACATAGAAGACAAAGAGTCAACGAGGAAGAT |
| | | CCGTGAACTCCTCAAAAAGGGGAATTCGCTGTACTCCA |
| | | AAGTCAGTGATAAGGTTTTCCAATGCTTAAGGGACACT |
| | | AACTCACGGCTTGGCCTAGGCTCCGAATTGAGGGAGG |
| | | ACATCAAGGAGAAAGTTATTAACTTGGGAGTTTACATG |
| | | CACAGCTCCCAGTGGTTTGAGCCCTTTCTGTTTTGGTTT |
| | | ACAGTCAAGACTGAGATGAGGTCAGTGATTAAATCAC |
| | | AAACCCATACTTGCCATAGGAGGAGACACACACCTGT |
| | | ATTCTTCACTGGTAGTTCAGTTGAGTTGCTAATCTCTCG |
| | | TGACCTTGTTGCTATAATCAGTAAAGAGTCTCAACATG |
| | | TATATTACCTGACATTTGAACTGGTTTTGATGTATTGTG |
| | | ATGTCATAGAGGGGAGGTTAATGACAGAGACCGCTAT |
| | | GACTATTGATGCTAGGTATACAGAGCTTCTAGGAAGAG |
| | | TCAGATACATGTGGAAACTGATAGATGGTTTCTTCCCT |
| | | GCACTCGGGAATCCAACTTATCAAATTGTAGCCATGCT |
| | | GGAGCCTCTTTCACTTGCTTACCTGCAGCTGAGGGATA |
| | | TAACAGTAGAACTCAGAGGTGCTTTCCTTAACCACTGC |
| | | TTTACTGAAATACATGATGTTCTTGACCAAAACGGGTT |
| | | TTCTGATGAAGGTACTTATCATGAGTTAACTGAAGCTC |
| | | TAGATTACATTTTCATAACTGATGACATACATCTGACA |
| | | GGGGAGATTTTCTCATTTTTCAGAAGTTTCGGCCACCC |
| | | CAGACTTGAAGCAGTAACGGCTGCTGAAAATGTTAGG |
| | | AAATACATGAATCAGCCTAAAGTCATTGTGTATGAGAC |
| | | TCTGATGAAAGGTCATGCCATATTTTGTGGAATCATAA |
| | | TCAACGGCTATCGTGACAGGCACGGAGGCAGTTGGCC |
| | | ACCGCTGACCCTCCCCCTGCATGCTGCAGACACAATCC |
| | | GGAATGCTCAAGCTTCAGGTGAAGGGTTAACACATGA |
| | | GCAGTGCGTTGATAACTGGAAATCTTTTGCTGGAGTGA |
| | | AATTTGGCTGCTTTATGCCTCTTAGCCTGGATAGTGATC |
| | | TGACAATGTACCTAAAGGACAAGGCACTTGCTGCTCTC |
| | | CAAAGGGAATGGGATTCAGTTTACCCGAAAGAGTTCCT |
| | | GCGTTACGACCCTCCCAAGGGAACCGGGTCACGGAGG |
| | | CTTGTAGATGTTTTCCTTAATGATTCGAGCTTTGACCCA |
| | | TATGATGTGATAATGTATGTTGTAAGTGGAGCTTACCT |
| | | CCATGACCCTGAGTTCAACCTGTCTTACAGCCTGAAAG |
| | | AAAAGGAGATCAAGGAAACAGGTAGACTTTTTGCTAA |
| | | AATGACTTACAAAATGAGGGCATGCCAAGTGATTGCTG |
| | | AAAATCTAATCTCAAACGGGATTGGCAAATATTTTAAG |
| | | GACAATGGGATGGCCAAGGATGAGCACGATTTGACTA |
| | | AGGCACTCCACACTCTAGCTGTCTCAGGAGTCCCCAAA |
| | | GATCTCAAAGAAAGTCACAGGGGGGGGCCAGTCTTAA |
| | | AAACCTACTCCCGAAGCCCAGTCCACACAAGTACCAG |
| | | GAACGTGAGAGCAGCAAAAGGGTTTATAGGGTTCCCT |
| | | CAAGTAATTCGGCAGGACCAAGACACTGATCATCCGG |
| | | AGAATATGGAAGCTTACGAGACAGTCAGTGCATTTATC |
| | | ACGACTGATCTCAAGAAGTACTGCCTTAATTGGAGATA |
| | | TGAGACCATCAGCTTGTTTGCACAGAGGCTAAATGAGA |
| | | TTTACGGATTGCCCTCATTTTTCCAGTGGCTGCATAAGA |
| | | GGCTTGAGACCTCTGTCCTGTATGTAAGTGACCCTCAT |
| | | TGCCCCCCCGACCTTGACGCCCATATCCCGTTATATAA |
| | | AGTCCCCAATGATCAAATCTTCATTAAGTACCCTATGG |
| | | GAGGTATAGAAGGGTATTGTCAGAAGCTGTGGACCAT |
| | | CAGCACCATTCCCTATCTATACCTGGCTGCTTATGAGA |
| | | GCGGAGTAAGGATTGCTTCGTTAGTGCAAGGGGACAA |
| | | TCAGACCATAGCCGTAACAAAAAGGGTACCCAGCACA |
| | | TGGCCCTACAACCTTAAGAAACGGGAAGCTGCTAGAG |
| | | TAACTAGAGATTACTTTGTAATTCTTAGGCAAAGGCTA |
| | | CATGATATTGGCCATCACCTCAAGGCAAATGAGACAAT |
| | | TGTTTCATCACATTTTTTTGTCTATTCAAAAGGAATATA |
| | | TTATGATGGGCTACTTGTGTCCCAATCACTCAAGAGCA |
| | | TCGCAAGATGTGTATTCTGGTCAGAGACTATAGTTGAT |
| | | GAAACAAGGGCAGCATGCAGTAATATTGCTACAACAA |
| | | TGGCTAAAAGCATCGAGAGAGGTTATGACCGTTACCTT |
| | | GCATATTCCCTGAACGTCCTAAAAGTGATACAGCAAAT |
| | | TCTGATCTCTCTTGGCTTCACAATCAATTCAACCATGAC |
| | | CCGGGATGTAGTCATACCCCTCCTCACAAACAACGACC |
| | | TCTTAATAAGGATGGCACTGTTGCCCGCTCCTATTGGG |
| | | GGGATGAATTATCTGAATATGAGCAGGCTGTTTGTCAG |
| | | AAACATCGGTGATCCAGTAACATCATCAATTGCTGATC |
| | | TCAAGAGAATGATTCTCGCCTCACTAATGCCTGAAGAG |
| | | ACCCTCCATCAAGTAATGACACAACAACCGGGGGACT |
| | | CTTCATTCCTAGACTGGGCTAGCGACCCTTACTCAGCA |
| | | AATCTTGTATGTGTCCAGAGCATCACTAGACTCCTCAA |
| | | GAACATAACTGCAAGGTTTGTCCTGATCCATAGTCCAA |
| | | ACCCAATGTTAAAAGGATTATTCCATGATGACAGTAAA |
| | | GAAGAGGACGAGGGACTGGCGGCATTCCTCATGGACA |
| | | GGCATATTATAGTACCTAGGGCAGCTCATGAAATCCTG |
| | | GATCATAGTGTCACAGGGGCAAGAGAGTCTATTGCAG |
| | | GCATGCTGGATACCACAAAAGGCTTGATTCGAGCCAGC |
| | | ATGAGGAAGGGGGGGTTAACCTCTCGAGTGATAACCA |
| | | GATTGTCCAATTATGACTATGAACAATTCAGAGCAGGG |
| | | ATGGTGCTATTGACAGGAAGAAAGAGAAATGTCCTCA |
| | | TTGACAAAGAGTCATGTTCAGTGCAGCTGGCGAGAGCT |
| | | CTAAGAAGCCATATGTGGGCGAGGCTAGCTCGAGGAC |
| | | GGCCTATTTACGGCCTTGAGGTCCCTGATGTACTAGAA |
| | | TCTATGCGAGGCCACCTTATTCGGCGTCATGAGACATG |
| | | TGTCATCTGCGAGTGTGGATCAGTCAACTACGGATGGT |
| | | TTTTTGTCCCCTCGGGTTGCCAACTGGATGATATTGACA |
| | | AGGAAACATCATCCTTGAGAGTCCCATATATTGGTTCT |
| | | ACCACTGATGAGAGAACAGACATGAAGCTTGCCTTCGT |
| | | AAGAGCCCCAAGTCGATCCTTGCGATCTGCTGTTAGAA |
| | | TAGCAACAGTGTACTCATGGGCTTACGGTGATGATGAT |
| | | AGCTCTTGGAACGAAGCCTGGTTGTTGGCTAGGCAAAG |
| | | GGCCAATGTGAGCCTGGAGGAGCTAAGGGTGATCACT |
| | | CCCATCTCAACTTCGACTAATTTAGCGCATAGGTTGAG |
| | | GGATCGTAGCACTCAAGTGAAATACTCAGGTACATCCC |
| | | TTGTCCGAGTGGCGAGGTATACCACAATCTCCAACGAC |
| | | AATCTCTCATTTGTCATATCAGATAAGAAGGTTGATAC |
| | | TAACTTTATATACCAACAAGGAATGCTTCTAGGGTTGG |
| | | GTGTTTTAGAAACATTGTTTCGACTCGAGAAAGATACC |
| | | GGATCATCTAACACGGTATTACATCTTCACGTCGAAAC |
| | | AGATTGTTGCGTGATCCCGATGATAGATCATCCCAGGA |
| | | TACCCAGCTCCCGCAAGCTAGAGCTGAGGGCAGAGCT |
| | | ATGTACCAACCCATTGATATATGATAATGCACCTTTAA |
| | | TTGACAGAGATGCAACAAGGCTATACACCCAGAGCCA |
| | | TAGGAGGCACCTTGTGGAATTTGTTACATGGTCCACAC |
| | | CCCAACTATATCACATTTTAGCTAAGTCCACAGCACTA |
| | | TCTATGATTGACCTGGTAACAAAATTTGAGAAGGACCA |
| | | TATGAATGAAATTTCAGCTCTCATAGGGGATGACGATA |
| | | TCAATAGTTTCATAACTGAGTTTCTGCTCATAGAGCCA |
| | | AGATTATTCACTATCTACTTGGGCCAGTGTGCGGCCAT |
| | | CAATTGGGCATTTGATGTACATTATCATAGACCATCAG |
| | | GGAAATATCAGATGGGTGAGCTGTTGTCATCGTTCCTT |
| | | TCTAGAATGAGCAAAGGAGTGTTTAAGGTGCTTGTCAA |
| | | TGCTCTAAGCCACCCAAAGATCTACAAGAAATTCTGGC |
| | | ATTGTGGTATTATAGAGCCTATCCATGGTCCTTCACTTG |
| | | ATGCTCAAAACTTGCACACAACTGTGTGCAACATGGTT |
| | | TACACATGCTATATGACCTACCTCGACCTGTTGTTGAA |
| | | TGAAGAGTTAGAAGAGTTCACATTTCTCTTGTGTGAAA |
| | | GCGACGAGGATGTAGTACCGGACAGATTCGACAACAT |
| | | CCAGGCAAAACACTTATGTGTTCTGGCAGATTTGTACT |
| | | GTCAACCAGGGACCTGCCCACCAATTCGAGGTCTAAGA |
| | | CCGGTAGAGAAATGTGCAGTTCTAACCGACCATATCAA |
| | | GGCAGAGGCTATGTTATCTCCAGCAGGATCTTCGTGGA |
| | | ACATAAATCCAATTATTGTAGACCATTACTCATGCTCT |
| | | CTGACTTATCTCCGGCGAGGATCGATCAAACAGATAAG |
| | | ATTGAGAGTTGATCCAGGATTCATTTTCGACGCCCTCG |
| | | CTGAGGTAAATGTCAGTCAGCCAAAGATCGGCAGCAA |
| | | CAACATCTCAAATATGAGCATCAAGGCTTTCAGACCCC |
| | | CACACGATGATGTTGCAAAATTGCTCAAAGATATCAAC |
| | | ACAAGCAAGCACAATCTTCCCATTTCAGGGGGCAATCT |
| | | CGCCAATTATGAAATCCATGCTTTCCGCAGAATCGGGT |
| | | TGAACTCATCTGCTTGCTACAAAGCTGTTGAGATATCA |
| | | ACATTAATTAGGAGATGCCTTGAGCCAGGGGAGGACG |
| | | GCTTGTTCTTGGGTGAGGGATCGGGTTCTATGTTGATC |
| | | ACTTATAAAGAGATACTTAAACTAAACAAGTGCTTCTA |
| | | TAATAGTGGGGTTTCCGCCAATTCTAGATCTGGTCAAA |
| | | GGGAATTAGCACCCTATCCCTCCGAAGTTGGCCTTGTC |
| | | GAACACAGAATGGGAGTAGGTAATATTGTCAAAGTGC |
| | | TCTTTAACGGGAGGCCCGAAGTCACGTGGGTAGGCAGT |
| | | GTAGATTGCTTCAATTTCATAGTTAGTAATATCCCTACC |
| | | TCTAGTGTGGGGTTTATCCATTCAGATATAGAGACCTT |
| | | GCCTGACAAAGATACTATAGAGAAGCTAGAGGAATTG |
| | | GCAGCCATCTTATCGATGGCTCTGCTCCTGGGCAAAAT |
| | | AGGATCAATACTGGTGATTAAGCTTATGCCTTTCAGCG |
| | | GGGATTTTGTTCAGGGATTTATAAGTTATGTAGGGTCT |
| | | CATTATAGAGAAGTGAACCTTGTATACCCTAGATACAG |
| | | CAACTTCATCTCTACTGAATCTTATTTGGTTATGACAGA |
| | | TCTCAAGGCTAACCGGCTAATGAATCCTGAAAAGATTA |
| | | AGCAGCAGATAATTGAATCATCTGTGAGGACTTCACCT |
| | | GGACTTATAGGTCACATCCTATCCATTAAGCAACTAAG |
| | | CTGCATACAAGCAATTGTGGGAGACGCAGTTAGTAGA |
| | | GGTGATATCAATCCTACTCTGAAAAAACTTACACCTAT |
| | | AGAGCAGGTGCTGATCAATTGCGGGTTGGCAATTAACG |
| | | GACCTAAGCTGTGCAAAGAATTGATCCACCATGATGTT |
| | | GCCTCAGGGCAAGATGGATTGCTTAATTCTATACTCAT |
| | | CCTCTACAGGGAGTTGGCAAGATTCAAAGACAACCAA |
| | | AGAAGTCAACAAGGGATGTTCCACGCTTACCCCGTATT |
| | | GGTAAGTAGCAGGCAACGAGAACTTATATCTAGGATC |
| | | ACCCGCAAATTCTGGGGGCACATTCTTCTTTACTCCGG |
| | | GAACAAAAAGTTGATAAATAAGTTTATCCAGAATCTCA |
| | | AGTCCGGCTATCTGATACTAGACTTACACCAGAATATC |
| | | TTCGTTAAGAATCTATCCAAGTCAGAGAAACAGATTAT |
| | | TATGACGGGGGGTTTGAAACGTGAGTGGGTTTTTAAGG |
| | | TAACAGTCAAGGAGACCAAAGAATGGTATAAGTTAGT |
| | | CGGATACAGTGCCCTGATTAAGGACTAATTGGTTGAAC |
| | | TCCGGAACCCTAATCCTGCCCTAGGTGGTTAGGCATTA |
| | | TTTGCAATATATTAAAGAAAACTTTGAAAATACGAAGT |
| | | TTCTATTCCCAGCTTTGTCTGGTGGCCGGCATGGTCCCA |
| | | GCCTCCTCGCTGGCGCCGGCTGGGCAACATTCCGAGGG |
| | | GACCGTCCCCTCGGTAATGGCGAATGGGACGCGGCCG |
| | | GTCGATCGACGATCCGGCTGCTAACAAAGCCCGAAAG |
| | | GAAGCTGAGTTGGCTGCTGCCACCGCTGAGCAATAACT |
| | | AGCATAACCCCTTGGGGCCTCTAAACGGGTCTTGAGGG |
| | | GTTTTTTGCTGAAAGGAGGAACTATATCCGGATCGAGA |
| | | TCAATTCTGTGAGCGTATGGCAAACGAAGGAAAAATA |
| | | GTTATAGTAGCCGCACTCGATGGGACATTTCAACGTAA |
| | | ACCGTTTAATAATATTTTGAATCTTATTCCATTATCTGA |
| | | AATGGTGGTAAAACTAACTGCTGTGTGTATGAAATGCT |
| | | TTAAGGAGGCTTCCTTTTCTAAACGATTGGGTGAGGAA |
| | | ACCGAGATAGAAATAATAGGAGGTAATGATATGTATC |
| | | AATCGGTGTGTAGAAAGTGTTACATCGACTCATAATAT |
| | | TATATTTTTTATCTAAAAAACTAAAAATAAACATTGAT |
| | | TAAATTTTAATATAATACTTAAAAATGGATGTTGTGTC |
| | | GTTAGATAAACCGTTTATGTATTTTGAGGAAATTGATA |
| | | ATGAGTTAGATTACGAACCAGAAAGTGCAAATGAGGT |
| | | CGCAAAAAAACTGCCGTATCAAGGACAGTTAAAACTA |
| | | TTACTAGGAGAATTATTTTTTCTTAGTAAGTTACAGCG |
| | | ACACGGTATATTAGATGGTGCCACCGTAGTGTATATAG |
| | | GATCTGCTCCCGGTACACATATACGTTATTTGAGAGAT |
| | | CATTTCTATAATTTAGGAGTGATCCCGAAAGGAAGCTG |
| | | AGTTGGCTGCTGCCACCGCTGAGCAATAACTAGCATAA |
| | | CCCCTTGGGGCCTCTAAACGGGTCTTGAGGGGTTTTTT |
| | | GCTGAAAGGAGGAACGCGCCTGATGCGGTATTTTCTCC |
| | | TTACGCATCTGTGCGGTATTTCACACCGCATATATGGT |
| | | GCACTCTCAGTACAATCTGCTCTGATGCCGCATAGTTA |
| | | AGCCAGCCCCGACACCCGCCAACACCCGCTGACGCGC |
| | | CCTGACGGGCTTGTCTGCTCCCGGCATCCGCTTACAGA |
| | | CAAGCTGTGACCGTCTCCGGGAGCTGCATGTGTCAGAG |
| | | GTTTTCACCGTCATCACCGAAACGCGCGAGACGAAAG |
| | | GGCCTCGTGATACGCCTATTTTTATAGGTTAATGTCATG |
| | | ATAATAATGGTTTCTTAGACGTCAGGTGGCACTTTTCG |
| | | GGGAAATGTGCGCGGAACCCCTATTTGTTTATTTTTCTA |
| | | AATACATTCAAATATGTATCCGCTCATGAGACAATAAC |
| | | CCTGATAAATGCTTCAATAATATTGAAAAAGGAAGAGT |
| | | ATGAGTATTCAACATTTCCGTGTCGCCCTTATTCCCTTT |
| | | TTTGCGGCATTTTGCCTTCCTGTTTTTGCTCACCCAGAA |
| | | ACGCTGGTGAAAGTAAAAGATGCTGAAGATCAGTTGG |
| | | GTGCACGAGTGGGTTACATCGAACTGGATCTCAACAGC |
| | | GGTAAGATCCTTGAGAGTTTTCGCCCCGAAGAACGTTT |
| | | TCCAATGATGAGCACTTTTAAAGTTCTGCTATGTGGCG |
| | | CGGTATTATCCCGTATTGACGCCGGGCAAGAGCAACTC |
| | | GGTCGCCGCATACACTATTCTCAGAATGACTTGGTTGA |
| | | GTACTCACCAGTCACAGAAAAGCATCTTACGGATGGCA |
| | | TGACAGTAAGAGAATTATGCAGTGCTGCCATAACCATG |
| | | AGTGATAACACTGCGGCCAACTTACTTCTGACAACGAT |
| | | CGGAGGACCGAAGGAGCTAACCGCTTTTTTGCACAACA |
| | | TGGGGGATCATGTAACTCGCCTTGATCGTTGGGAACCG |
| | | GAGCTGAATGAAGCCATACCAAACGACGAGCGTGACA |
| | | CCACGATGCCTGTAGCAATGGCAACAACGTTGCGCAA |
| | | ACTATTAACTGGCGAACTACTTACTCTAGCTTCCCGGC |
| | | AACAATTAATAGACTGGATGGAGGCGGATAAAGTTGC |
| | | AGGACCACTTCTGCGCTCGGCCCTTCCGGCTGGCTGGT |
| | | TTATTGCTGATAAATCTGGAGCCGGTGAGCGTGGGTCT |
| | | CGCGGTATCATTGCAGCACTGGGGCCAGATGGTAAGCC |
| | | CTCCCGTATCGTAGTTATCTACACGACGGGGAGTCAGG |
| | | CAACTATGGATGAACGAAATAGACAGATCGCTGAGAT |
| | | AGGTGCCTCACTGATTAAGCATTGGTAACTGTCAGACC |
| | | AAGTTTACTCATATATACTTTAGATTGATTTAAAACTTC |
| | | ATTTTTAATTTAAAAGGATCTAGGTGAAGATCCTTTTTG |
| | | ATAATCTCATGACCAAAATCCCTTAACGTGAGTTTTCG |
| | | TTCCACTGAGCGTCAGACCCCGTAGAAAAGATCAAAG |
| | | GATCTTCTTGAGATCCTTTTTTTCTGCGCGTAATCTGCT |
| | | GCTTGCAAACAAAAAAACCACCGCTACCAGCGGTGGT |
| | | TTGTTTGCCGGATCAAGAGCTACCAACTCTTTTTCCGA |
| | | AGGTAACTGGCTTCAGCAGAGCGCAGATACCAAATAC |
| | | TGTCCTTCTAGTGTAGCCGTAGTTAGGCCACCACTTCA |
| | | AGAACTCTGTAGCACCGCCTACATACCTCGCTCTGCTA |
| | | ATCCTGTTACCAGTGGCTGCTGCCAGTGGCGATAAGTC |
| | | GTGTCTTACCGGGTTGGACTCAAGACGATAGTTACCGG |
| | | ATAAGGCGCAGCGGTCGGGCTGAACGGGGGGTTCGTG |
| | | CACACAGCCCAGCTTGGAGCGAACGACCTACACCGAA |
| | | CTGAGATACCTACAGCGTGAGCATTGAGAAAGCGCCA |
| | | CGCTTCCCGAAGGGAGAAAGGCGGACAGGTATCCGGT |
| | | AAGCGGCAGGGTCGGAACAGGAGAGCGCACGAGGGA |
| | | GCTTCCAGGGGGAAACGCCTGGTATCTTTATAGTCCTG |
| | | TCGGGTTTCGCCACCTCTGACTTGAGCGTCGATTTTTGT |
| | | GATGCTCGTCAGGGGGGCGGAGCCTATGGAAAAACGC |
| | | CAGCAACGCGGCCTTTTTACGGTTCCTGGCCTTTTGCTG |
| | | GCCTTTTGCTCACATGTTCTTTCCTGCGTTATCCCCTGA |
| | | TTCTGTGGATAACCGTATTACCGCCTTTGAGTGAGCTG |
| | | ATACCGCTCGCCGCAGCCGAACGACCGAGCGCAGCGA |
| | | GTCAGTGAGCGAGGAAGCGGAAGAGCGCCCAATACGC |
| | | AAACCGCCTCTCCCCGCGCGTTGGCCGATTCATTAATG |
| | | CAGCTGGCACGACAGGTTTCCCGACTGGAAAGCGGGC |
| | | AGTGAGCGCAACGCAATTAATGTGAGTTAGCTCACTCA |
| | | TTAGGCACCCCAGGCTTTACACTTTATGCTTCCGGCTCG |
| | | TATGTTGTGTGGAATTGTGAGCGGATAACAATTTCACA |
| | | CAGGAAACAGCTATGACCATGATTACGCCAAGCTTACG |
| | | CGTCCTGGCATTATGCCCAGTACATGACCTTATGGGAC |
| | | TTTCCTACTTGGCAGTACATCTACGTATTAGTCATCGCT |
| | | ATTACCATGGTGATGCGGTTTTGGCAGTACATCAATGG |
| | | GCGTGGATAGCGGTTTGACTCACGGGGATTTCCAAGTC |
| | | TCCACCCCATTGACGTCAATGGGAGTTTGTTTTGGCAC |
| | | CAAAATCAACGGGACTTTCCAAAATGTCGTAACAACTC |
| | | CGCCCCATTGACGCAAATGGGCGGTAGGCGTGTACGGT |
| | | GGGAGGTCTATATAAGCAGAGCTCGTTTAGTGAACCGT |
| | | GG |
| 90 | MV_F2 | TCGAGTCCCTCACGCTTACAG |
| | forward primer | |
| 91 | MV_R2 reverse | GGCGGTGCTTGATGTTCTGA |
| | primer | |
| 92 | MV_P2_Probe | CTGGAGGACCCTACACTG |

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. An isolated nucleic acid molecule comprising:
a) a cDNA encoding a full length, antigenomic (+) RNA strand of an attenuated strain of measles virus (MV-cDNA);
b) one or more cDNAs encoding an Epstein-Barr virus (EBV) protein (EBV cDNA) independently selected from the group consisting of gp350, LMP2, gH, gL, gp42, and variants thereof;
c) an upstream additional transcriptional unit (ATU) cDNA operably linked to the EBV cDNA that is 5′ of the EBV cDNA (upstream ATU cDNA); and
d) a downstream ATU cDNA operably linked to the EBV cDNA that is 3′ of the EBV cDNA (downstream ATU cDNA);
wherein the upstream ATU cDNA, the EBV cDNA, and the downstream ATU cDNA are between the P and M genes of the MV-cDNA (ATU2) or between the H and L genes of the MV-cDNA (ATU3).

2. The isolated nucleic acid molecule of claim 1, wherein each of the one or more EBV cDNAs encodes an EBV protein sequence independently selected from the group consisting of: SEQ ID NO: 54 (EBV gp350); SEQ ID NO: 55 (EBV LMP2); SEQ ID NO: 56 (EBV gH); SEQ ID NO: 57 (EBV gL); and SEQ ID NO: 58 (EBV gp42).

3. The isolated nucleic acid of claim 1, wherein the upstream ATU cDNA, the EBV cDNA, and the downstream ATU cDNA are at ATU2 or ATU3 in the MV-cDNA.

4. (canceled)

5. The isolated nucleic acid molecule of claim 1, wherein the upstream ATU cDNA sequence is set forth in SEQ ID NO: 69 or 72.

6. (canceled)

7. The isolated nucleic acid molecule of claim 1, comprising a sequence selected from the group consisting of SEQ ID NOs: 59, 60, 61, 83, 84, 85, 86, 87, 88.

8. An isolated nucleic acid molecule comprising:
a) a cDNA encoding a full length, antigenomic (+) RNA strand of an attenuated strain of measles virus (MV-cDNA);
b) a first cDNA encoding a Epstein-Barr virus (EBV) protein (EBV cDNA) selected from the group consisting of gp350, LMP2, gH, gL, gp42, and variants thereof,
c) a second EBV cDNA selected from the group consisting of gp350, LMP2, gH, gL, gp42, and variants thereof, wherein the first and second EBV cDNAs do not have the same sequence;
d) an upstream additional transcriptional unit (ATU) cDNA operably linked to the EBV cDNA that is 5′ of the first EBV cDNA (upstream ATU cDNA);
e) a downstream ATU cDNA that is 3′ of the second EBV cDNA encoding the EBV protein; and
f) an interstitial ATU cDNA between the first and second EBV cDNAs (interstitial ATU cDNA);
wherein the upstream ATU cDNA, the first and second EBV cDNAs, the interstitial ATU cDNA and the downstream ATU cDNA are operably linked; and
wherein the upstream ATU cDNA, the first and second EBV cDNAs, the interstitial ATU, and the downstream ATU cDNA are between the P and M genes of the MV-cDNA (ATU2) or between the H and L genes of the MV-cDNA (ATU3).

9. The isolated nucleic acid molecule of claim 8, wherein the first and second EBV cDNA each encode an EBV protein sequence independently selected from the group consisting of: SEQ ID NO: 54 (EBV gp350); SEQ ID NO: 55 (EBV LMP2); SEQ ID NO: 56 (EBV gH); SEQ ID NO: 57 (EBV gL); and SEQ ID NO: 58 (EBV gp42).

10. The isolated nucleic acid of claim 8, wherein the upstream ATU cDNA, the first and second EBV cDNA, the interstitial ATU cDNA and the downstream ATU cDNA are at ATU2 or ATU3 in the MV-cDNA.

11. (canceled)

12. The isolated nucleic acid molecule of claim 8, wherein the upstream ATU cDNA sequence is set forth in SEQ ID NO: 69 or 72.

13. (canceled)

14. The isolated nucleic acid molecule of claim 8, wherein the interstitial ATU cDNA sequence is selected from the group consisting of SEQ ID NOs: 65, 69, 72, 75, 78, and 79.

15. The isolated nucleic acid molecule of claim 8, comprising the sequence set forth in SEQ ID NO: 60.

16. An isolated nucleic acid molecule comprising:
a) a cDNA encoding a full length, antigenomic (+) RNA strand of an attenuated strain of measles virus (MV-cDNA);
b) a first cDNA encoding an Epstein-Barr virus (EBV) protein (EBV cDNA) selected from the group consisting of gp350, LMP2, gH, gL, gp42, and variants thereof,
c) a second EBV cDNA selected from the group consisting of gp350, LMP2, gH, gL, gp42, and variants thereof, wherein the first and second EBV cDNAs do not have the same sequence;
d) an upstream additional transcriptional unit (ATU) cDNA that is 5′ of the first EBV cDNA (upstream ATU cDNA);
e) a downstream ATU cDNA that is 3′ of the second EBV cDNA (downstream ATU cDNA); and
f) a furin cleavage site cDNA and 2A peptide cDNA (Fur-2A cDNA) between the first and second EBV cDNAs;
wherein the upstream ATU cDNA, the first and second EBV cDNAs, the Fur-2A cDNA, and the downstream ATU cDNA are operably linked; and
wherein the upstream ATU cDNA, the first and second EBV cDNAs, the Fur-2A cDNA, and the downstream ATU cDNA are between the P and M genes of the MV-cDNA (ATU2) or between the H and L genes of the MV-cDNA (ATU3).

17. The isolated nucleic acid molecule of claim 16, wherein the first and second EBV cDNA each encode an EBV protein sequence independently selected from the group consisting of: SEQ ID NO: 54 (EBV gp350); SEQ ID NO: 55 (EBV LMP2); SEQ ID NO: 56 (EBV gH); SEQ ID NO: 57 (EBV gL); and SEQ ID NO: 58 (EBV gp42).

18. The isolated nucleic acid of claim 16, wherein the upstream ATU cDNA, the first and second EBV cDNAs, the Fur-2A cDNA, and the downstream ATU cDNA are at ATU2 or ATU3 in the MV-cDNA.

19. (canceled)

20. The isolated nucleic acid molecule of claim 16, wherein the upstream ATU cDNA sequence is set forth in SEQ ID NO: 69 or 72.

21. (canceled)

22. The isolated nucleic acid molecule of claim 16, wherein the furin cDNA of the Fur-2A cDNA encodes a protein sequence selected from the group consisting of SEQ ID NOs: 14-53, and wherein the 2A peptide cDNA of the Fur-2A cDNA encodes a protein sequence independently selected from the group consisting of SEQ ID NOs: 4-11.

23-48. (canceled)

49. A vector for the rescue of a recombinant measles virus, comprising the isolated nucleic acid molecule of claim 1.

50-53. (canceled)

54. A recombinant measles virus comprising in its genome a cDNA sequence comprising the nucleic acid molecule of claim 1.

55. An immunogenic composition comprising (i) an effective amount of the recombinant measles virus of claim 54, and (ii) a pharmaceutically acceptable carrier.

56. A method for treating or preventing an Epstein-Barr virus (EBV) infection in a subject, comprising administering an effective amount of the immunogenic composition according to claim 55 to the subject.

57. A method for inducing a protective immune response against Epstein-Barr (EBV) in a subject, comprising administering an effective amount of the immunogenic composition of claim 55 to the subject.

58-64. (canceled)
